(19) United States
(12) Patent Application Publication
(10) Pub. No.: US 2025/0042926 A1
(43) Pub. Date: Feb. 6, 2025

(54) THERAPEUTIC COMPOUNDS FOR HIV

(71) Applicant: Gilead Sciences, Inc., Foster City, CA (US)

(72) Inventors: Julie Farand, San Mateo, CA (US); Darryl Kato, San Francisco, CA (US); James B.C. Mack, San Mateo, CA (US)

(21) Appl. No.: 18/678,444

(22) Filed: May 30, 2024

Related U.S. Application Data
(60) Provisional application No. 63/598,398, filed on Nov. 13, 2023.
(60) Provisional application No. 63/505,255, filed on May 31, 2023.

(51) Int. Cl. C07F 9/6558; A61K 31/675; C07F 9/6561

(52) U.S. Cl. CPC C07F 9/65583; A61K 31/675; C07F 9/6561

(57) ABSTRACT

The present disclosure relates generally to certain compounds, pharmaceutical compositions comprising said compounds, and methods of making and using said compounds and pharmaceutical compositions. The compounds and compositions provided herein may be used for the treatment or prevention of a Retroviridae infection, including an HIV infection.

### CROSS REFERENCE TO RELATED APPLICATIONS

This application claims the benefit of U.S. Provisional Application No. 63/505,255 filed on May 31, 2023, and U.S. Provisional Application No. 63/598,398, filed on Nov. 13, 2023, the entire contents of each of which is hereby incorporated by reference in their entireties.

### TECHNICAL FIELD

This disclosure relates generally to novel compounds and pharmaceutical compositions comprising said compounds for use in the prevention or treatment of a Retroviridae viral infection, including an infection caused by the human immunodeficiency virus (HIV). This disclosure also relates to methods of making said compounds and intermediates in the preparation of said compounds.

### BACKGROUND

Positive-single stranded RNA viruses comprising the Retroviridae family include those of the subfamily Orthoretrovirinae and genera Alpharetrovirus, Betaretrovirus, Gammaretrovirus, Deltaretrovirus, Epsilonretrovirus, Lentivirus, and Spumavirus which cause many human and animal diseases. Among the Lentivirus, HIV-1 infection in humans leads to depletion of T helper cells and immune dysfunction, producing immunodeficiency and vulnerability to opportunistic infections. Treating HIV-1 infections with highly active antiretroviral therapies (HAART) has proven to be effective at reducing viral load and significantly delaying disease progression (Hammer, S. M., et al.; JAMA 2008, 300: 555-570). However, these treatments could lead to the emergence of HIV strains that are resistant to current therapies (Taiwo, B., International Journal of Infectious Diseases 2009, 13:552-559; Smith, R. J., et al., Science 2010, 327:697-701). Therefore, there is a pressing need to discover new antiretroviral agents that are active against emerging drug-resistant HIV variants.

Also of interest in the area of HIV therapies and treatments is providing regimens to patients with improved pharmacokinetic properties, including, for example, increased potency, long-acting pharmacokinetics, low solubility, low clearance, and/or other properties. While current regimens for treating HIV have progressed enough that patients no longer have to take multiple pills multiple times a day, patients today still are required to take a pill every day for the foreseeable span of their life. Thus, it would be beneficial to have HIV therapies that require patients take medication less than once a day (e.g. once every couple of days, once a week, once every other week, once a month, and so forth) or take a smaller effective dose of the medication(s) on a daily, weekly, monthly, or longer basis.

### SUMMARY

In some embodiments, provided herein is a compound of Formula I.

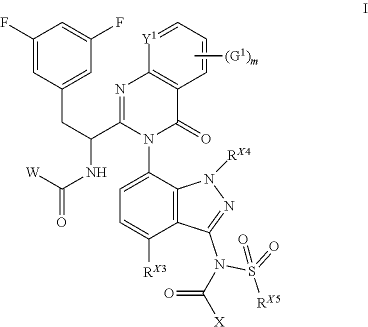

or a pharmaceutically acceptable salt thereof, wherein constituent members are defined herein.

In some embodiments, provided herein is a pharmaceutical composition comprising a therapeutically effective amount of a compound provided herein, or a pharmaceutically acceptable salt thereof, and a pharmaceutically acceptable excipient.

In some embodiments, provided herein is a method of treating or preventing a human immunodeficiency virus (HIV) infection in a patient in need thereof comprising administering to the patient a therapeutically effective amount of a compound provided herein, or a pharmaceutically acceptable salt thereof, or a pharmaceutical composition provided herein.

In some embodiments, provided herein is a method of treating a human immunodeficiency virus (HIV) infection in a heavily treatment-experienced patient, the method comprising administering to the patient a therapeutically effective amount of a compound provided herein, or a pharmaceutically acceptable salt thereof, or a pharmaceutical composition provided herein.

In some embodiments, provided herein is a therapeutically effective amount of a compound provided herein, or a pharmaceutically acceptable salt thereof, or a pharmaceutical composition provided herein for use in therapy.

In some embodiments, provided herein is a compound provided herein, or a pharmaceutically acceptable salt thereof, or a pharmaceutical composition provided herein for use in a method of treating or preventing a human immunodeficiency virus (HIV) infection in a patient in need thereof, the method comprising administering to the patient a therapeutically effective amount of the compound, or a pharmaceutically acceptable salt thereof, or the pharmaceutical composition.

In some embodiments, provided herein is a compound provided herein, or a pharmaceutically acceptable salt thereof, or a pharmaceutical composition provided herein for use in a method of treating a human immunodeficiency virus (HIV) infection in a heavily treatment-experienced patient, the method comprising administering to the patient a therapeutically effective amount of the compound, or a pharmaceutically acceptable salt thereof, or the pharmaceutical composition.

### DETAILED DESCRIPTION

### I. Definitions

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

As used in the present disclosure, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. A solid line coming out of the center of a ring (including a fused, bridged or spirocyclic ring system) indicates that the point of attachment for a substituent on the ring can be at any ring atom. For example, R^{aa} in the below structure can be attached to any of the five carbon ring atoms or R^{aa} can replace the hydrogen attached to the nitrogen ring atom:

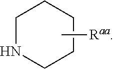

As another example, in the below below fused bicyclic heterocyclic structure:

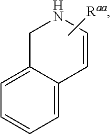

R^{aa} can be attached to any of the numbered positions shown below:

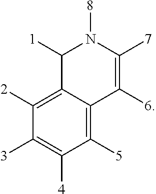

A solid line coming out of the center of a ring (including a fused, bridged, or spirocyclic ring system) indicates that the point of attachment for the ring system to the rest of the compound can be at any ring atom of the fused, bridged, or spirocyclic ring system. For example, in the below structure:

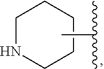

the monocyclic heterocyclyl can be attached to the rest of the compound at any of the numbered positions shown below:

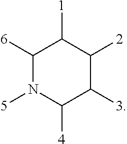

As another example, in the below fused bicyclic heterocyclic structure,

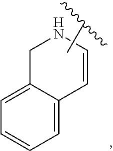

the fused bicyclic heterocyclyl can be attached to the rest of the compound at any of the eight numbered positions shown below:

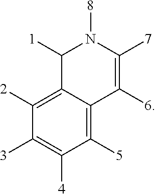

The prefix “Cᵤ₋ᵥ” indicates that the following group has from u to v carbon atoms. For example, “C₁₋₆ alkyl” indicates that the alkyl group has from 1 to 6 carbon atoms. Likewise, the term “x-y membered” rings, wherein x and y are numerical ranges, such as “3 to 12-membered heterocyclyl”, refers to a ring containing x-y atoms (i.e., 3-12), of which up to 80% may be heteroatoms, such as N, O, S, P, and the remaining atoms are carbon.

Also, certain commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group or an “arylenyl” group, or arylyl group, respectively.

“A compound disclosed herein” or “a compound of the present disclosure” or “a compound provided herein” or “a compound described herein” refers to the compounds of Formula I. Also included are the specific compounds of Examples 1 to 18.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl), 1 to 3 carbon atoms (i.e., C₁₋₃ alkyl), or 1 to 2 carbon atoms (i.e., C₁₋₂ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkylene” refers to a divalent and unbranched saturated hydrocarbon chain. As used herein, alkylene has 1 to 20 carbon atoms (i.e., C₁₋₂₀ alkylene), 1 to 12 carbon atoms (i.e., C₁₋₁₂ alkylene), 1 to 8 carbon atoms (i.e., C₁₋₈ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), or 1 to 2 carbon atoms (i.e., C₁₋₂ alkylene). Examples of alkylene groups include methylene, ethylene, propylene, butylene, pentylene, and hexylene. In some embodiments, an alkylene is optionally substituted with an alkyl group. Examples of substituted alkylene groups include —CH(CH₃)CH₂—, —CH₂CH(CH₃)—, —CH₂CH(CH₂CH₃)—, —CH₂C(CH₃)₂—, —C(CH₃)₂CH₂—, —CH(CH₃)CH(CH₃)—, —CH₂C(CH₂CH₃)(CH₃)—, and —CH₂C(CH₂CH₃)₂.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy. “Haloalkoxy” refers to an alkoxy group as defined above, wherein one or more hydrogen atoms are replaced by a halogen.

“Acyl” refers to a group —C(═O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(═O)NR^{y}R^{z} and an “N-amido” group which refers to the group —NR^{y}C(═O)R^{z}, wherein R^{y} and R^{z} are independently selected from the group consisting of hydrogen, alkyl, aryl, haloalkyl, heteroaryl, cycloalkyl, or heterocyclyl; each of which may be optionally substituted.

“Amino” refers to the group —NR^{y}R^{z} wherein R^{y} and R^{z} are independently selected from the group consisting of hydrogen, alkyl, haloalkyl, aryl, heteroaryl, cycloalkyl, or heterocyclyl; each of which may be optionally substituted.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl ring, the resulting ring system is heteroaryl.

“Cyano” or “carbonitrile” refers to the group —CN.

“Cycloalkyl” refers to a saturated or partially saturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Bridged” refers to a ring fusion wherein non-adjacent atoms on a ring are joined by a divalent substituent, such as an alkylenyl group, an alkylenyl group containing one or two heteroatoms, or a single heteroatom. Quinuclidinyl and admantanyl are examples of bridged ring systems.

The term “fused” refers to a ring which is bound to an adjacent ring.

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents.

“Halogen” or “halo” includes fluoro, chloro, bromo, and iodo. “Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkylene” refers to a divalent and unbranched saturated hydrocarbon chain having one, two, or three heteroatoms selected from NH, O, or S. As used herein, a heteroalkylene has 1 to 20 carbon atoms and one, two, or three heteroatoms selected from NH, O, and S (i.e., C₁₋₂₀ heteroalkylene); 1 to 8 carbon atoms and one, two, or three heteroatoms selected from NH, O, and S (i.e., C₁₋₈ heteroalkylene); 1 to 6 carbon atoms and one, two, or three heteroatoms selected from NH, O, and S S (i.e., C₁₋₆ heteroalkylene); 1 to 4 carbon atoms and one, two, or three heteroatoms selected from NH, O, and S (i.e., C₁₋₄ heteroalkylene); 1 to 3 carbon atoms and one, two, or three heteroatoms selected from NH, O, and S (i.e., C₁₋₃ heteroalkylene); or 1 to 2 carbon atoms and one, two, or three heteroatoms selected from NH, O, and S (i.e., C₁₋₃ heteroalkylene). For example, —CH₂O— is a C₁ heteroalkylene and —CH₂SCH₂— is a C₂ heteroalkylene. Examples of heteroalkylene groups include —CH₂CH₂OCH₂—, —CH₂SCH₂OCH₂—, —CH₂O—, and —CH₂NHCH₂—. In some embodiments, a heteroalkylene is optionally substituted with an alkyl group. Examples of substituted heteroalkylene groups include —CH(CH₃)N(CH₃)CH₂—, —CH₂OCH(CH₃)—, —CH₂CH(CH₂CH₃)S—, —CH₂NHC(CH₃)₂—, —C(CH₃)₂SCH₂—, —CH(CH₃)N(CH₃)CH(CH₃)O—, —CH₂SC(CH₂CH₃)(CH₃)—, and —CH₂C(CH₂CH₃)₂NH—.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 carbon ring atoms (i.e., C₁₋₂₀ heteroaryl), 3 to 12 carbon ring atoms (i.e., C₃₋₁₂ heteroaryl), or 3 to 8 carbon ring atoms (i.e., C₃₋₈ heteroaryl); and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Heteroaryl does not encompass or overlap with aryl as defined above.

“Heterocyclyl” or “heterocyclic ring” or “heterocycle” refers to a non-aromatic cyclic alkyl group, with one or more ring heteroatoms independently selected from nitrogen, oxygen and sulfur. As used herein, “heterocyclyl” or “heterocyclic ring” or “heterocycle” refer to rings that are saturated or partially saturated unless otherwise indicated, e.g., in some embodiments “heterocyclyl” or “heterocyclic ring” or “heterocycle” refers to rings that are partially saturated where specified. The term “heterocyclyl” or “heterocyclic ring” or “heterocycle” includes heterocycloalkenyl groups (i.e., the heterocyclyl group having at least one double bond). A heterocyclyl may be a single ring or multiple rings wherein the multiple rings may be fused, bridged, or spiro. As used herein, heterocyclyl has 2 to 20 carbon ring atoms (i.e., C₂₋₂₀ heterocyclyl), 2 to 12 carbon ring atoms (i.e., C₂₋₁₂ heterocyclyl), 2 to 10 carbon ring atoms (i.e., C₂₋₁₀ heterocyclyl), 2 to 8 carbon ring atoms (i.e., C₂₋₈ heterocyclyl), 3 to 12 carbon ring atoms (i.e., C₃₋₁₂ heterocyclyl), 3 to 8 carbon ring atoms (i.e., C₃₋₈ heterocyclyl), or 3 to 6 carbon ring atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, “bridged-heterocyclyl” includes bicyclic and tricyclic ring systems. Also as used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. As used herein, the terms “heterocycle”, “heterocyclyl”, and “heterocyclic ring” are used interchangeably. In some embodiments, a heterocyclyl is substituted with an oxo group.

“Hydroxy” or “hydroxyl” refers to the group —OH.

“Oxo” refers to the group (=O) or (O).

“Sulfonyl” refers to the group —S(O)₂R^{bb}, where R^{bb} is alkyl, haloalkyl, heterocyclyl, cycloalkyl, heteroaryl, or aryl. Examples of sulfonyl are methylsulfonyl, ethylsulfonyl, phenylsulfonyl, and toluenesulfonyl.

Whenever the graphical representation of a group terminates in a singly bonded nitrogen atom, that group represents an —NH group unless otherwise indicated. Similarly, unless otherwise expressed, hydrogen atom(s) are implied and deemed present where necessary in view of the knowledge of one of skill in the art to complete valency or provide stability.

The terms “optional” or “optionally” mean that the subsequently described event or circumstance may or may not occur, and that the description includes instances where said event or circumstance occurs and instances in which it does not. Also, the term “optionally substituted” means that any one or more hydrogen atoms on the designated atom or group may or may not be replaced by a moiety other than hydrogen.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “alkylaryl.” Unless specified otherwise, where a group is described as optionally substituted, any substituents of the group are themselves unsubstituted.

In some embodiments, a substituted cycloalkyl, a substituted heterocyclyl, a substituted aryl, and/or a substituted heteroaryl includes a cycloalkyl, a heterocyclyl, an aryl, and/or a heteroaryl that has a substituent on the ring atom to which the cycloalkyl, heterocyclyl, aryl, and/or heteroaryl is attached to the rest of the compound. For example, in the below moiety, the cyclopropyl is substituted with a methyl group:

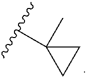 [SMILES: *C1(C)CC1]

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

A “tautomer” refers to a proton shift from one atom of a molecule to another atom of the same molecule. The present disclosure includes tautomers of any compounds provided herein.

Some of the compounds provided herein exist as tautomeric isomers. Tautomeric isomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

A “solvate” is formed by the interaction of a solvent and a compound. Solvates of salts of the compounds provided herein are also provided. Hydrates of the compounds provided herein are also provided.

Any formula or structure provided herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ²H, ³H, ¹³C and ¹⁴C are incorporated, are also provided herein. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The present disclosure also includes compounds of Formula I, in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the present disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example, increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure, any atom specifically designated as a deuterium (D) is meant to represent deuterium.

In many cases, the compounds of this disclosure are capable of forming acid and/or base salts by virtue of the presence of amino and/or carboxyl groups or groups similar thereto.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, mono, di or tri cycloalkyl amines, mono, di or tri arylamines or mixed amines, and the like. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (i.e., decreasing one or more symptoms resulting from the disease or condition, and/or diminishing the extent of the disease or condition); b) slowing or arresting the development of one or more clinical symptoms associated with the disease or condition (i.e., stabilizing the disease or condition, preventing or delaying the worsening or progression of the disease or condition, and/or preventing or delaying the spread (i.e., metastasis) of the disease or condition); and/or c) relieving the disease, that is, causing the regression of clinical symptoms (i.e., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or pharmaceutically acceptable salts, isomer, or a mixture thereof means an amount sufficient to effect treatment when administered to a subject, to provide a therapeutic benefit such as amelioration of symptoms or slowing of disease progression. For example, a therapeutically effective amount may be an amount sufficient to improve a symptom of a Retroviridae viral infection, including but not limited to HIV infection. The therapeutically effective amount may vary depending on the subject, and the disease or condition being treated, the weight and age of the subject, the severity of the disease or condition, and the manner of administering, which can readily be determined by one of ordinary skill in the art.

### II. Compounds

In some embodiments, provided herein is a compound of Formula I:

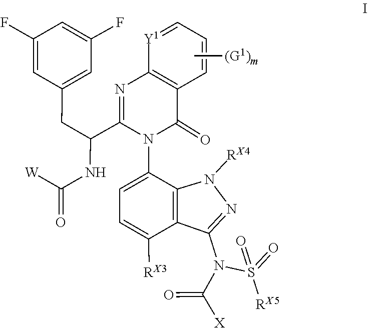

or a pharmaceutically acceptable salt thereof, wherein:
Y¹ is CH or N;
G¹ is C₁₋₆ alkyl, C₁₋₁₀ alkoxy, —O(phenyl substituted with 1-5 halogens), —N(R^{1a})₂, —SO₂R^{2a}, C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl,
   wherein the C₁₋₆ alkyl and C₁₋₁₀ alkoxy are each optionally substituted with 1-10 R^{3a} groups;
   wherein the C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl are each optionally substituted with 1-6 groups independently selected from —OH, —CN, halogen, —N(R^{1a})₂, —SO₂R^{2a}, R^{4a}, C₁₋₄ alkyl, C₁₋₄ alkoxy, and C₃₋₆ monocyclic cycloalkyl,
      wherein the C₁₋₄ alkyl, C₁₋₄ alkoxy, and C₃₋₆ monocyclic cycloalkyl are each optionally substituted with 1-6 halogens;
each R^{1a} independently is H or C₁₋₆ alkyl optionally substituted with 1-6 groups independently selected from —OH, —CN, halogen, —SO₂(C₁₋₆ alkyl), and C₁₋₆ alkoxy;
each R^{2a} independently is C₁₋₆ alkyl optionally substituted with 1-6 halogens;
each R^{3a} independently is —OH, —CN, halogen, —N(R^{1a})₂, —SO₂R^{2a}, C₁₋₅ alkoxy, C₃₋₆ monocyclic cycloalkyl, phenyl, 5-6 membered monocyclic heteroaryl, or —O(C₃₋₆ monocyclic cycloalkyl substituted with 1-5 halogens),
   wherein the C₁₋₅ alkoxy, C₃₋₆ monocyclic cycloalkyl, phenyl, and 5-6 membered monocyclic heteroaryl are each optionally substituted with 1-6 groups independently selected from halogen, C₁₋₃ alkyl, and C₁₋₃ alkoxy,
      wherein the C₁₋₃ alkyl and C₁₋₃ alkoxy are each optionally substituted with 1-4 halogens,
each R^{4a} independently is C₁₋₆ alkyl optionally substituted with 1-6 groups independently selected from —OH, —CN, halogen, —SO₂(C₁₋₆ alkyl), and C₁₋₆ alkoxy;
m is 1, 2, 3, or 4;
R^{X3} is H, F, Cl, —CH₃ or —OCH₃;
R^{X4} is H or C₁₋₃ alkyl, wherein the C₁₋₃ alkyl is optionally substituted with 1 to 3 fluorines;
R^{X5} is C₁₋₆ alkyl or C₃₋₆ cycloalkyl;
W is selected from:

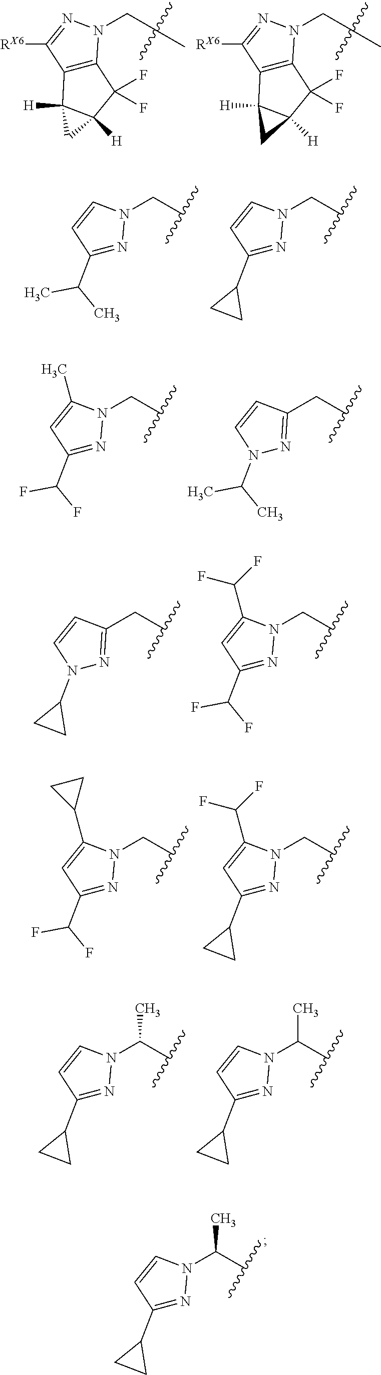

R^{X6} is methyl or C₃₋₅ monocyclic cycloalkyl, each of which is optionally substituted with 1 to 3 halogens;
X is —NR¹R², C₁₋₁₀ alkyl, or C₂₋₆ alkenyl,
   wherein the C₁₋₁₀ alkyl and C₂₋₆ alkenyl are each independently substituted with 1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl, wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
   wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
   wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{e} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R^{g} independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR⁸S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl independently have 1-4 ring heteroatoms independently selected from N, O, and S.

In some embodiments, the compound of Formula I is a compound of Formula II:

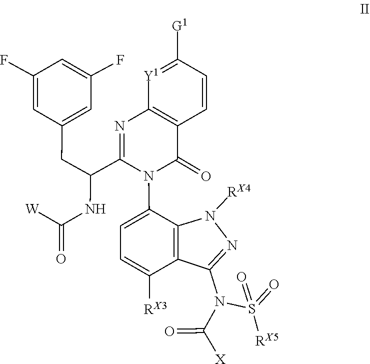

or a pharmaceutically acceptable salt thereof.

In some embodiments, provided herein is a compound of Formula II:

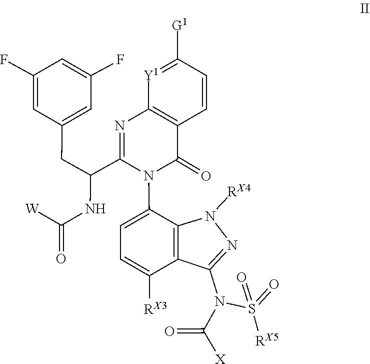

or a pharmaceutically acceptable salt thereof, wherein:
Y¹ is CH or N;
G¹ is C₁₋₆ alkoxy or phenyl, wherein the C₁₋₆ alkoxy is optionally substituted with 1-3 halogens and wherein the phenyl is substituted once with —N(CH₃)S(O₂)CH₃, —S(O₂)C(CH₃)₃, —CHF₂, —CF₃, —OCHF₂, —OCF₃, or —C(CH₃)₂OH;
or G¹ is one of the following:

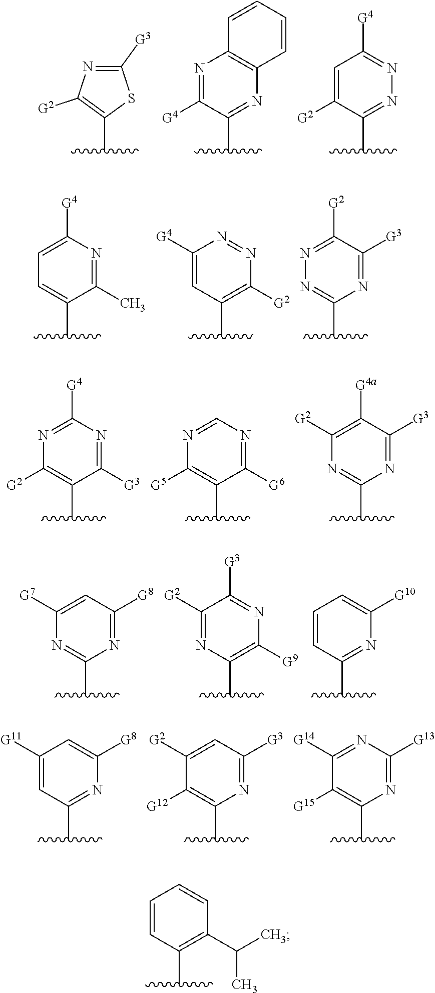

G² and G³ are independently selected from is H and —CH₃;
G⁴ is H, —CH₃, or —OCH₃;
G^{4a} is —CH₃ or —OCH₃;
G⁵ is —CH₃ or —CH₂CH₃;
G⁶ is H, —CH₃, or —CH₂CH₃;
G⁷ is ethyl, isopropyl, tert-butyl, —CHF₂, or —CF₃;
G⁸ is H, methyl, ethyl, —CHF₂, —CF₃, —OCH₃, or —OCH₂CH₃;
G⁹ is ethyl, isopropyl, cyclopropyl, —CH₂OH, or —OCH₃;
G¹⁰ is ethyl, isopropyl, cyclopropyl, tert-butyl, —CHF₂, or —CF₃;
G¹¹ is methyl, —OCH₃, —CHF₂, —CF₃, or —S(O₂)CH₃;
G¹² is F, —CH₃, —CHF₂, —CF₃, —OCH₃, or —S(O₂)CH₃;
G¹³ is C₁₋₄ alkyl, C₁₋₆ cycloalkyl, or —CH₂O(C₁₋₃ alkyl);
G¹⁴ is H, C₁₋₄ alkyl, —CHF₂, —CF₃, —O(C₁₋₃ alkyl);
G¹⁵ is H, F, —CH₃ or —OCH₃;
R^{X3} is H, F, Cl, —CH₃ or —OCH₃;
R^{X4} is H or C₁₋₃ alkyl, wherein the C₁₋₃ alkyl is optionally substituted with 1 to 3 fluorines;
R^{X5} is C₁₋₆ alkyl or C₃₋₆ cycloalkyl;
W is selected from:

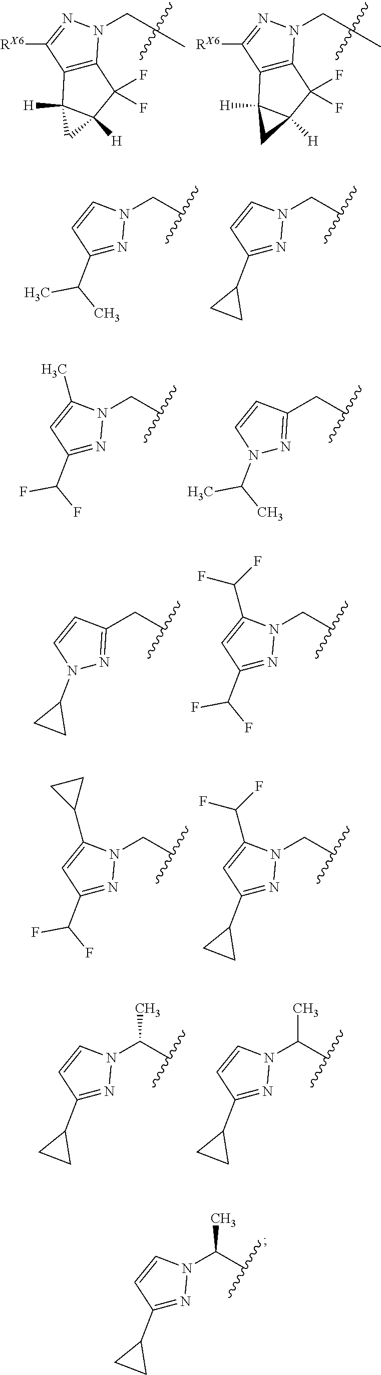

R^{X6} is methyl, which is optionally substituted with 1 to 3 fluorines;
X is —NR¹R², C₁₋₁₀ alkyl, or C₂₋₆ alkenyl,
   wherein the C₁₋₁₀ alkyl and C₂₋₆ alkenyl are each independently substituted with 1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl, wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
   wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
   wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{e} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R^{g} independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR⁸S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments, provided herein is a compound of Formula II:

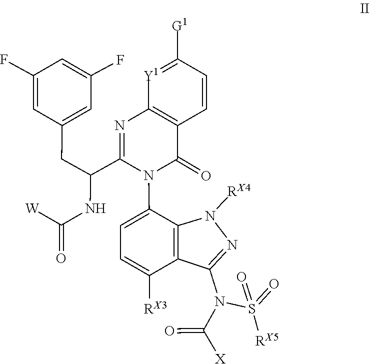

or a pharmaceutically acceptable salt thereof, wherein:
Y¹ is CH or N;
G¹ is phenyl substituted once with —N(CH₃)S(O₂)CH₃, —S(O₂)C(CH₃)₃, —CIF₂, —CF₃, —OCHF₂, —OCF₃, or —C(CH₃)₂OH;
or G¹ is one of the following:

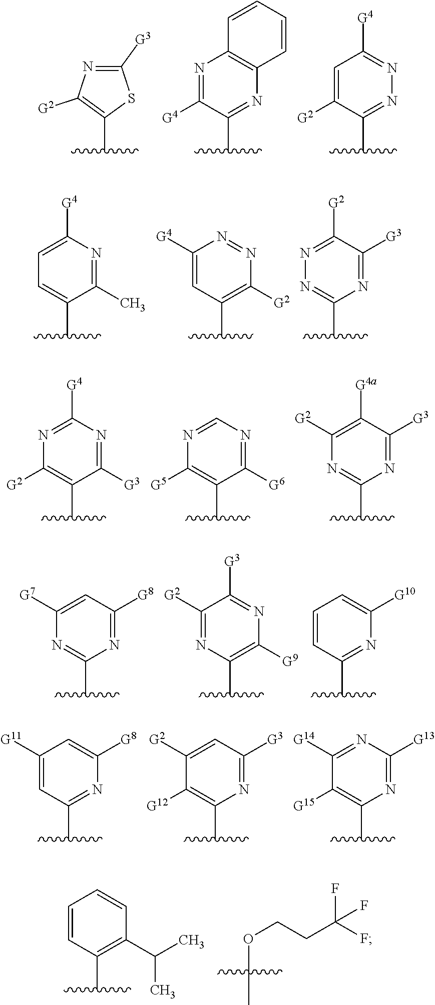

G² and G³ are independently selected from is H and —CH₃;
G⁴ is H, —CH₃, or —OCH₃;
G⁴a is —CH₃ or —OCH₃;
G⁵ is —CH₃ or —CH₂CH₃;
G⁶ is H, —CH₃, or —CH₂CH₃;
G⁷ is ethyl, isopropyl, tert-butyl, —CHF₂, or —CF₃;
G⁸ is H, methyl, ethyl, —CHF₂, —CF₃, —OCH₃, or —OCH₂CH₃;
G⁹ is ethyl, isopropyl, cyclopropyl, —CH₂OH, or —OCH₃;
G¹⁰ is ethyl, isopropyl, cyclopropyl, tert-butyl, —CHF₂, or —CF₃;
G¹¹ is methyl, —OCH₃, —CHF₂, —CF₃, or —S(O₂)CH₃;
G¹² is F, —CH₃, —CHF₂, —CF₃, —OCH₃, or —S(O₂)CH₃;
G¹³ is C₁₋₄ alkyl, C₁₋₆ cycloalkyl, or —CH₂O(C₁₋₃ alkyl);
G¹⁴ is H, C₁₋₄ alkyl, —CHF₂, —CF₃, —O(C₁₋₃ alkyl);
G¹⁵ is H, F, —CH₃ or —OCH₃;
R^{X3} is H, F, Cl, —CH₃ or —OCH₃;
R^{X4} is H or C₁₋₃ alkyl, wherein the C₁₋₃ alkyl is optionally substituted with 1 to 3 fluorines;
R^{X5} is C₁₋₆ alkyl or C₃₋₆ cycloalkyl;
W is selected from:

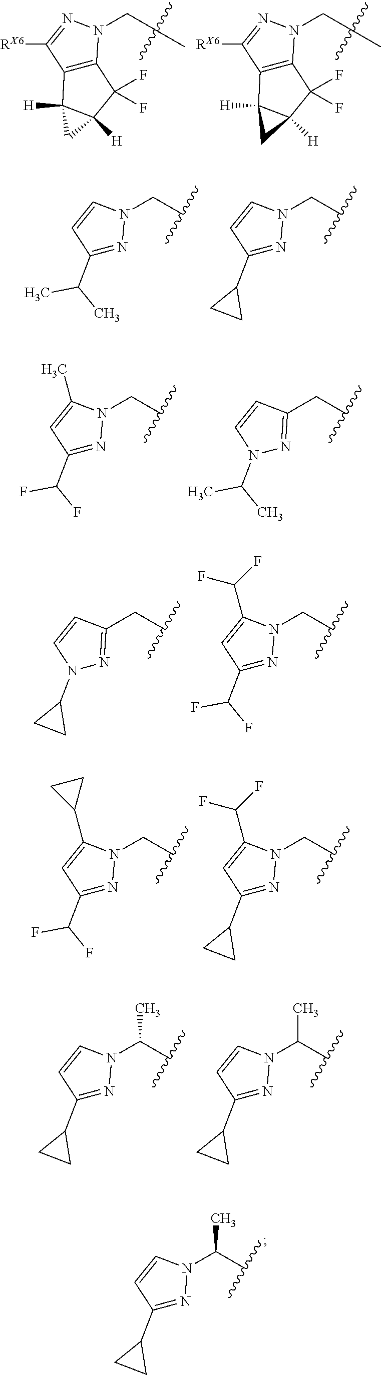

R^{X6} is methyl, which is optionally substituted with 1 to 3 fluorines;
X is —NR¹R², C₁₋₁₀ alkyl, or C₂₋₆ alkenyl, wherein the C₁₋₁₀ alkyl and C₂₋₆ alkenyl are each independently substituted with
   1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl,
   wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
   wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
   wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{c} independently is —OR⁶, —OC(O)R⁶, —OC(O)C(O)OR⁶, —NR⁷R⁷, —NRC(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R⁹ independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR'S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments, m is 1, 2, 3, or 4. In some embodiments, m is 1. In some embodiments, m is 2. In some embodiments, m is 3. In some embodiments, m is 4.

In some embodiments, the compound of Formula I is a compound of Formula IIa:

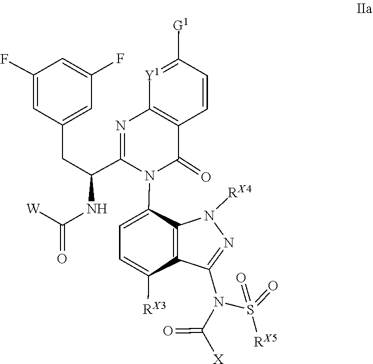

or a pharmaceutically acceptable salt thereof.

In some embodiments, the compound of Formula I is a compound of Formula III:

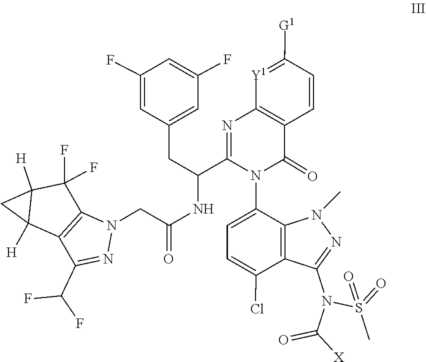

or a pharmaceutically acceptable salt thereof.

In some embodiments, the compound of Formula I is a compound of Formula III:

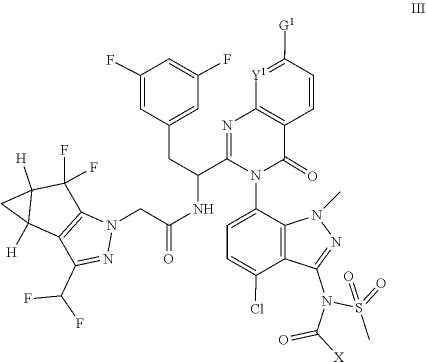

or a pharmaceutically acceptable salt thereof, wherein
Y¹ is CH or N;
G¹ is C₁₋₆ alkoxy optionally substituted with 1-3 halogens,

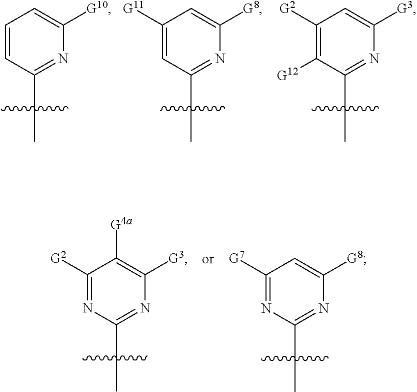

G² and G³ are independently H or CH₃;
G⁴a is —CH₃ or —OCH₃;
G⁷ is ethyl, isopropyl, tert-butyl, —CHF₂, or —CF₃;
G⁸ is H, methyl, ethyl, —CHF₂, —CF₃, —OCH₃, or —OCH₂CH₃;
G¹⁰ is ethyl, isopropyl, cyclopropyl, tert-butyl, —CHF₂, or —CF₃;
G¹¹ is methyl, —OCH₃, —CHF₂, —CF₃, or —S(O₂)CH₃;
G¹² is F, —CH₃, —CHF₂, —CF₃, —OCH₃, or —S(O₂)CH₃;
X is —NR¹R², C₁₋₁₀ alkyl, or C₂₋₆ alkenyl,
   wherein the C₁₋₁₀ alkyl and C₂₋₆ alkenyl are each independently substituted with 1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl,
   wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
   wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
   wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{c} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R^{g} independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR'S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments, the compound of Formula I is a compound of Formula IIIa:

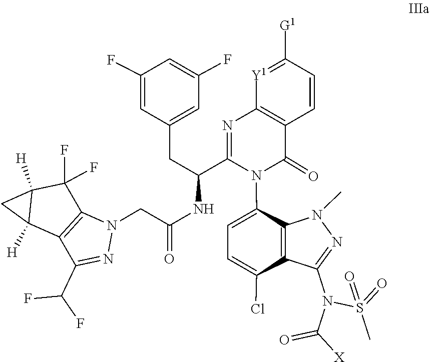

or a pharmaceutically acceptable salt thereof.

In some embodiments, the compound of Formula I is a compound of Formula IV:

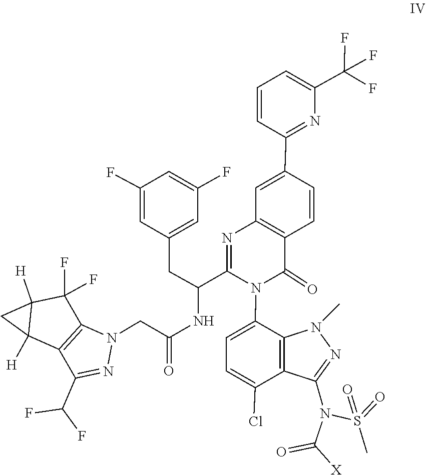

or a pharmaceutically acceptable salt thereof.

In some embodiments, the compound of Formula I is a compound of Formula IVa:

or a pharmaceutically acceptable salt thereof.

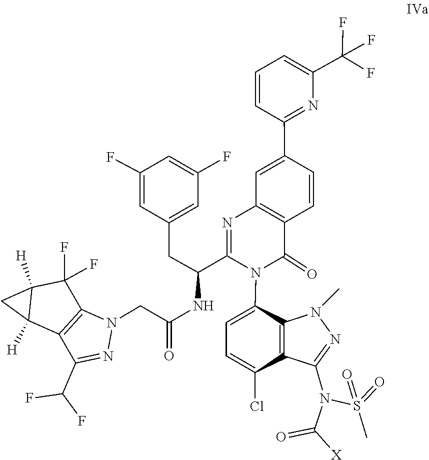

In some embodiments, the compound of Formula I is a compound of Formula V:

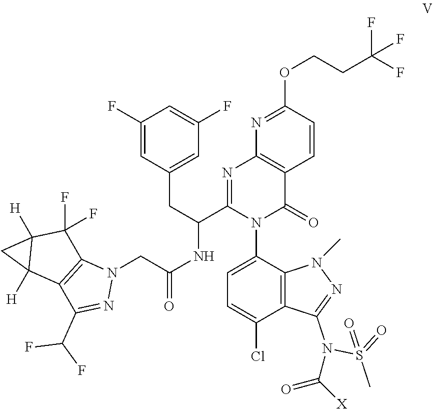

or a pharmaceutically acceptable salt thereof.

In some embodiments, the compound of Formula I is a compound of Formula Va:

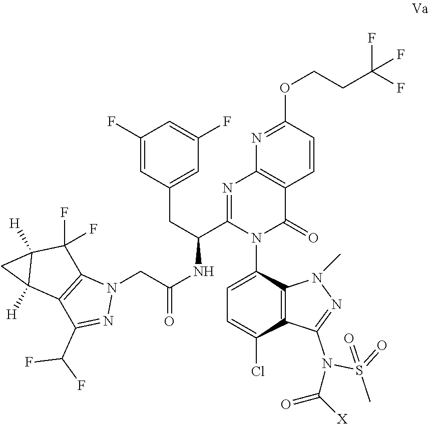

or a pharmaceutically acceptable salt thereof.

In some embodiments, the compound of Formula I is a compound of Formula VI:

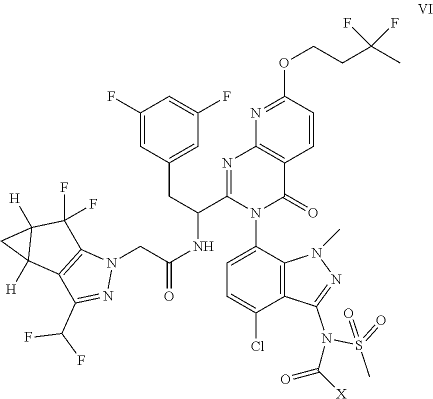

or a pharmaceutically acceptable salt thereof.

In some embodiments, the compound of Formula I is a compound of Formula VIa:

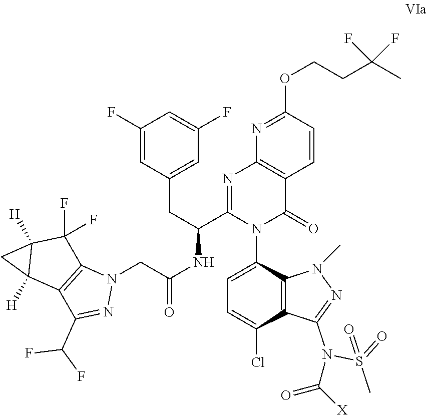

or a pharmaceutically acceptable salt thereof.

In some embodiments, the compound of Formula I is a compound of Formula VII:

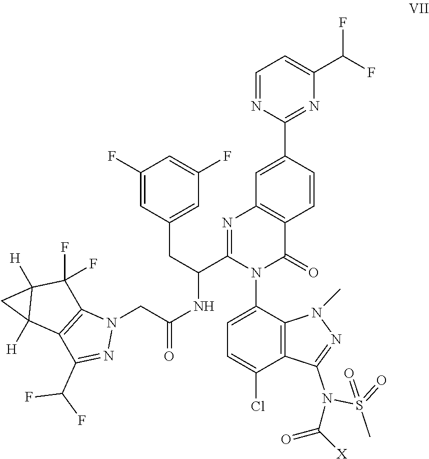

or a pharmaceutically acceptable salt thereof.

In some embodiments, the compound of Formula I is a compound of Formula VIIa:

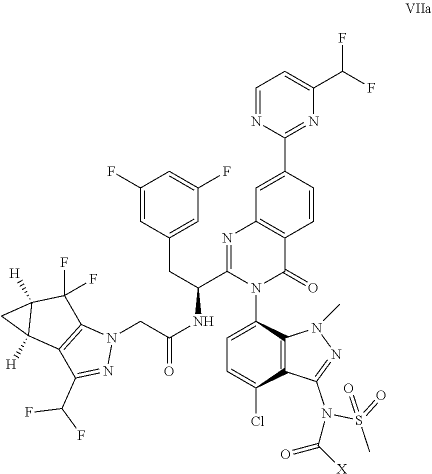

or a pharmaceutically acceptable salt thereof.

In some embodiments, W is:

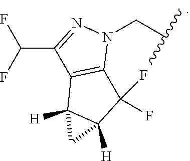

In some embodiments, W is:

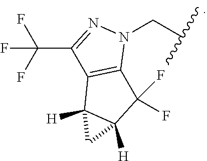

In some embodiments, W is selected from the group consisting of:

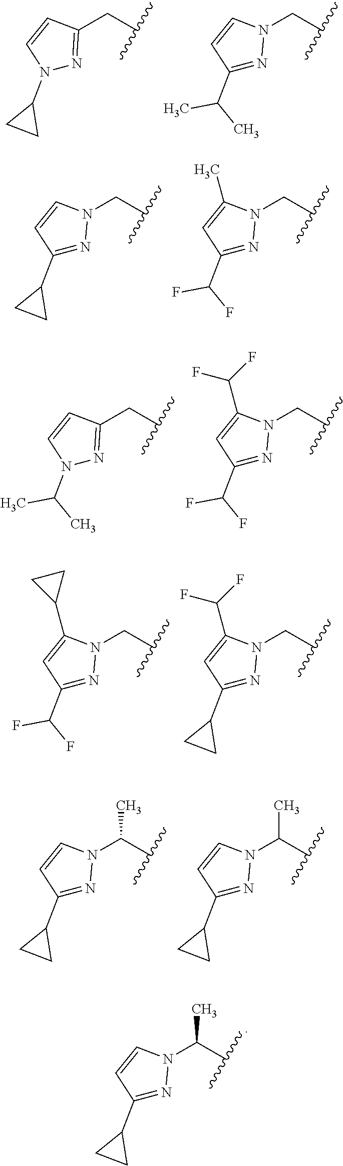

In some embodiments, R^{X3} is Cl.

In some embodiments, R^{X4} is methyl.

In some embodiments, R^{X5} is methyl.

In some embodiments, R^{X3} is Cl; R^{X4} is selected from the group consisting of —CH₃, —CH₂CHF₂, and —CH₂CF₃; and R^{X5} is selected from the group consisting of methyl and cyclopropyl.

In some embodiments, R^{X3} is —CH₃; R^{X4} is selected from the group consisting of —CH₃, —CH₂CHF₂, and —CH₂CF₃; and R^{X5} is selected from the group consisting of methyl and cyclopropyl.

In some embodiments, R^{X3} is Cl; R^{X4} is methyl; and R^{X5} is methyl.

In some embodiments, G¹ is C₁₋₆ alkyl, C₁₋₁₀ alkoxy, —O(phenyl substituted with 1-5 halogens), —N(R^{1a})₂, —SO₂R^{2a}, C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl,
wherein the C₁₋₆ alkyl and C₁₋₁₀ alkoxy are each optionally substituted with 1-10 R^{3a} groups;
wherein the C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl are each optionally substituted with 1-6 groups independently selected from —OH, —CN, halogen, —N(R^{1a})₂, —SO₂R^{2a}, R^{4a} C₁₋₄ alkyl, C₁₋₄ alkoxy, and C₃₋₆ monocyclic cycloalkyl,
   wherein the C₁₋₄ alkyl, C₁₋₄ alkoxy, and C₃₋₆ monocyclic cycloalkyl are each optionally substituted with 1-6 halogens, and
wherein each 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl independently have 1-4 ring heteroatoms independently selected from N, O, and S.

In some embodiments, each R^{1a} independently is H or C₁₋₆ alkyl optionally substituted with 1-6 groups independently selected from —OH, —CN, halogen, —SO₂(C₁₋₆ alkyl), and C₁₋₆ alkoxy.

In some embodiments, each R^{2a} independently is C₁₋₆ alkyl optionally substituted with 1-6 halogens.

In some embodiments, each R^{3a} independently is —OH, —CN, halogen, —N(R^{1a})₂, —SO₂R^{2a}, C₁₋₅ alkoxy, C₃₋₆ monocyclic cycloalkyl, phenyl, 5-6 membered monocyclic heteroaryl, or —O(C₃₋₆ monocyclic cycloalkyl substituted with 1-5 halogens),
wherein the C₁₋₅ alkoxy, C₃₋₆ monocyclic cycloalkyl, phenyl, and 5-6 membered monocyclic heteroaryl are each optionally substituted with 1-6 groups independently selected from halogen, C₁₋₃ alkyl, and C₁₋₃ alkoxy, and
wherein the C₁₋₃ alkyl and C₁₋₃ alkoxy are each optionally substituted with 1-4 halogens.

In some embodiments, each R^{4a} independently is C₁₋₆ alkyl optionally substituted with 1-6 groups independently selected from —OH, —CN, halogen, —SO₂(C₁₋₆ alkyl), and C₁₋₆ alkoxy.

In some embodiments, G¹ is C₁₋₆ alkoxy or phenyl,
wherein the C₁₋₆ alkoxy is optionally substituted with 1-3 halogens and
wherein the phenyl is substituted once with —N(CH₃)S(O₂)CH₃, —S(O₂)C(CH₃)₃, —CHF₂, —CF₃, —OCHF₂, —OCF₃, or —C(CH₃)₂OH;
or G¹ is one of the following:

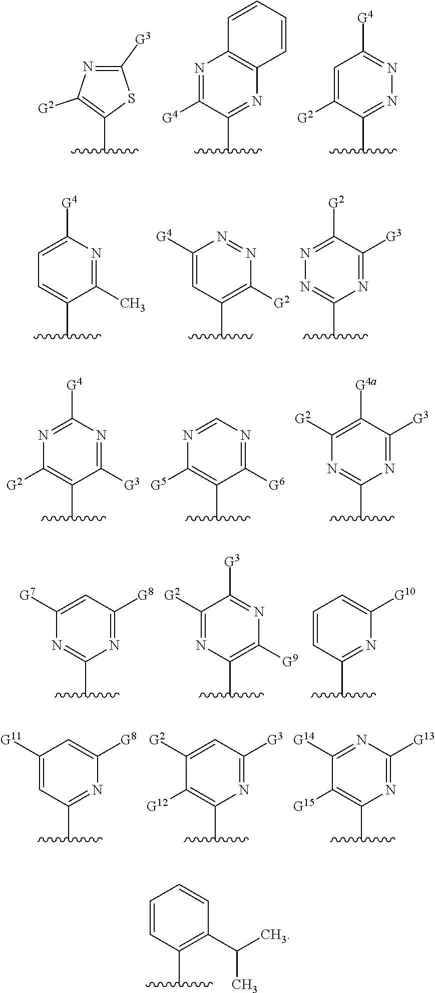

In some embodiments, G¹ is phenyl substituted once with —N(CH₃)S(O₂)CH₃, —S(O₂)C(CH₃)₃, —CHF₂, —CF₃, —OCHF₂, —OCF₃, or —C(CH₃)₂OH;
or G¹ is one of the following:

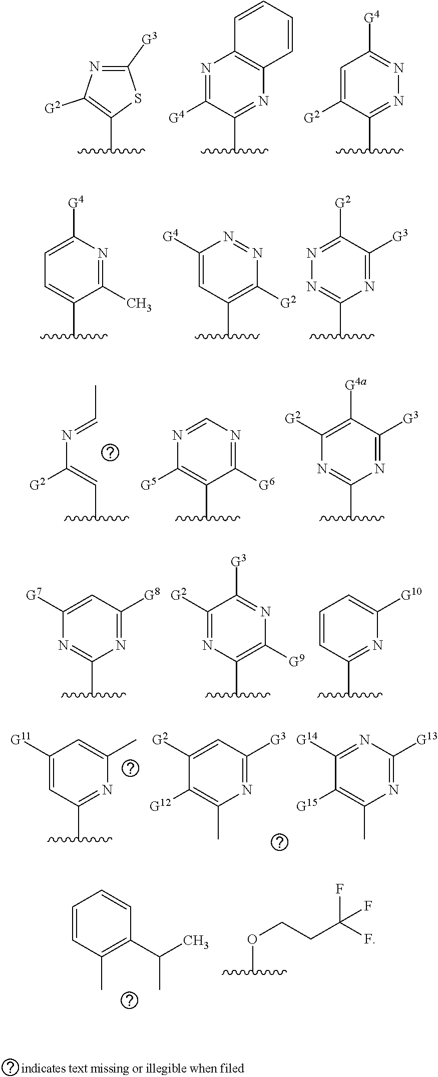

In some embodiments, G¹ is C₁₋₆ alkoxy optionally substituted with 1-3 halogens,

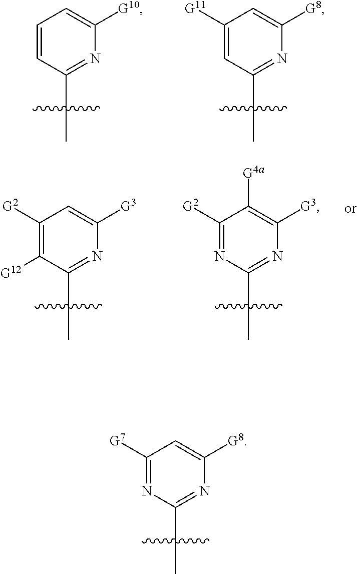

In some embodiments, G¹ is selected from the group consisting of:

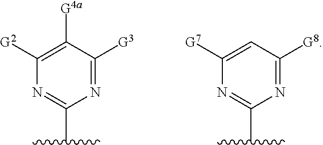

In some embodiments, G¹ is selected from the group consisting of:

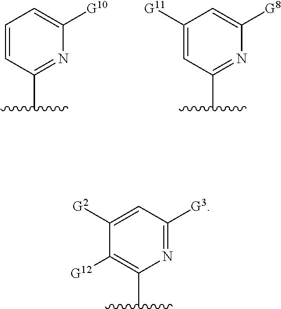

In some embodiments, G¹ is selected from the group consisting of:

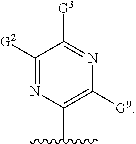

In some embodiments, G¹ is selected from the group consisting of:

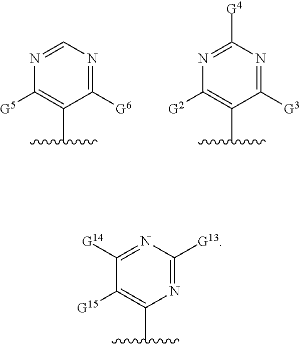

In some embodiments, G¹ is selected from the group consisting of:

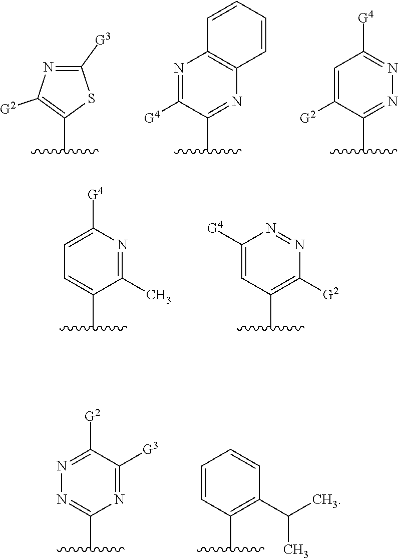

In some embodiments, G¹ is selected from the group consisting of:

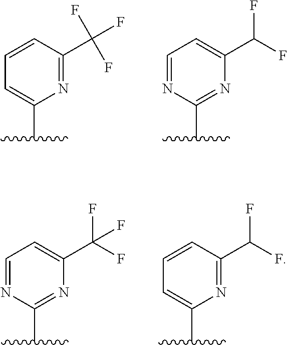

In some embodiments, G¹ is selected from the group consisting of:

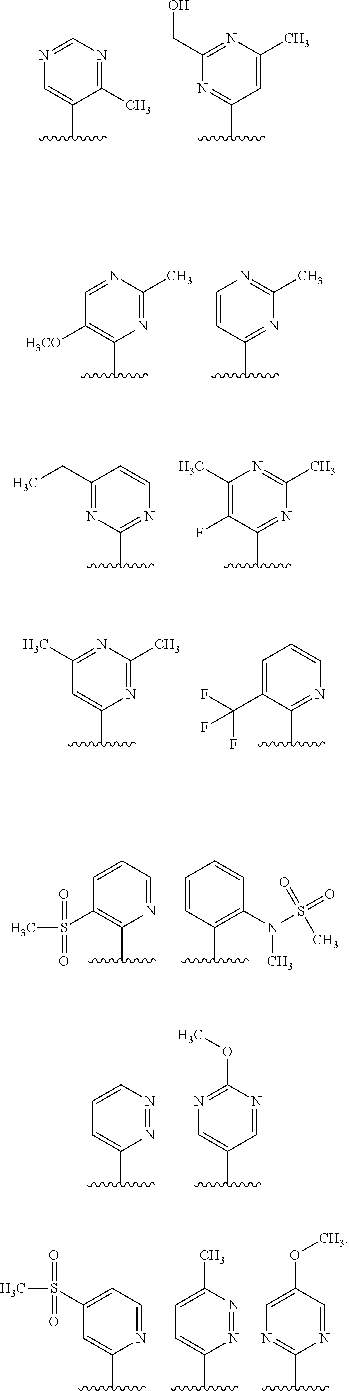

In some embodiments, G¹ is selected from the group consisting of:

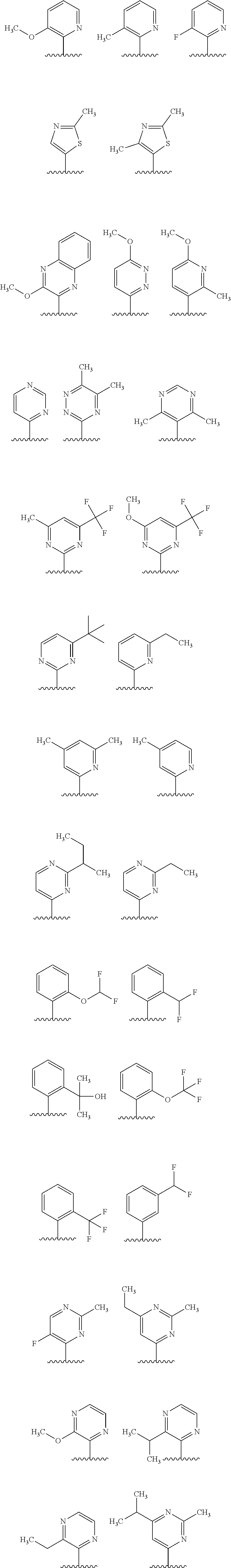

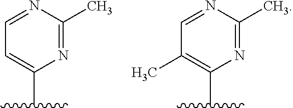

In some embodiments, G¹ is selected from the group consisting of:

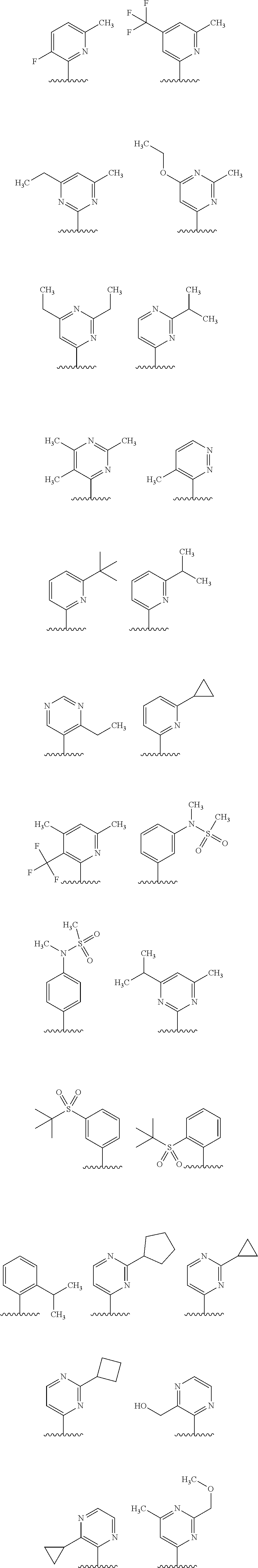

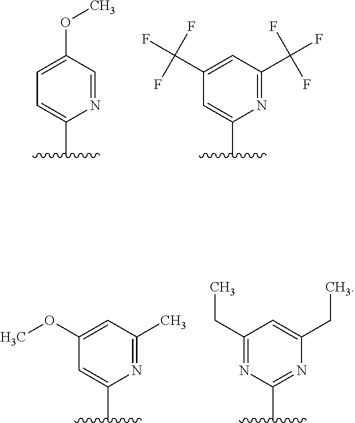

In some embodiments, G¹ is selected from the group consisting of:

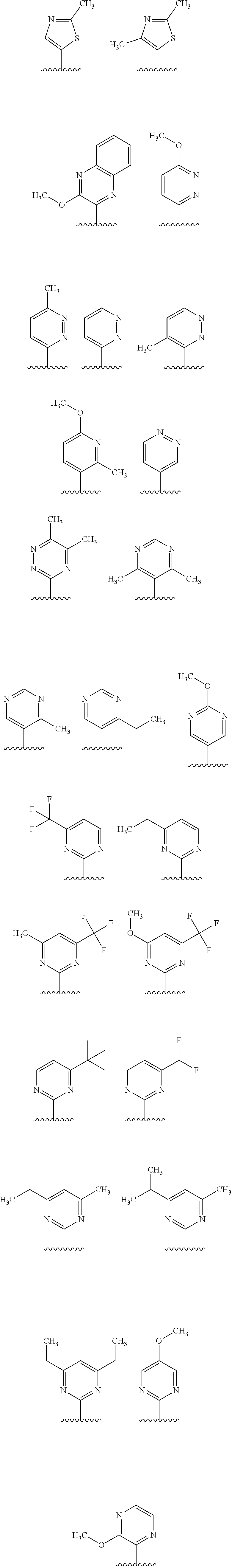

In some embodiments, G¹ is selected from the group consisting of:

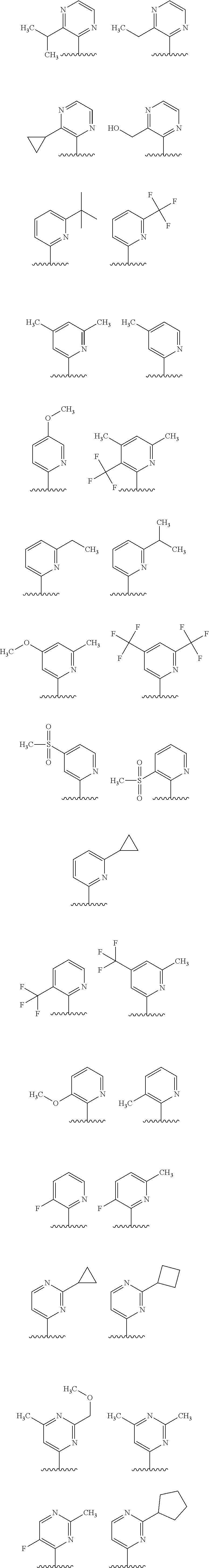

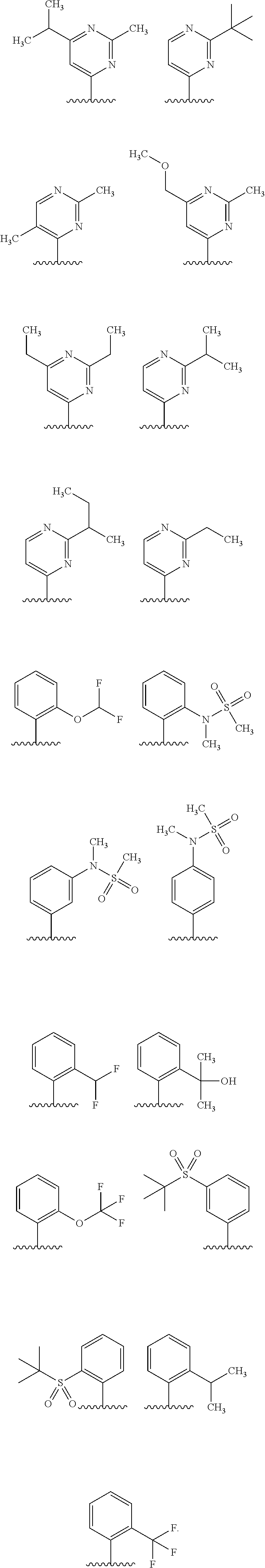

In some embodiments, G¹ comprises at least one fluorine atom.

In some embodiments, G¹ is:

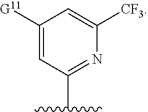

In some embodiments, G¹ is:

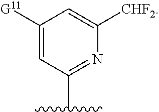

In some embodiments, G¹ is:

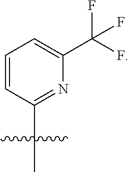 [SMILES: *c1cccc(C(F)(F)F)n1]

In some embodiments, G¹ is:

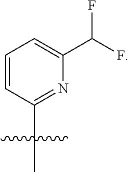 [SMILES: *c1cccc(C(F)F)n1]

In some embodiments, G¹ is:

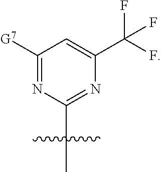

In some embodiments, G¹ is:

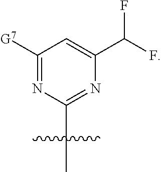

In some embodiments, G¹ is:

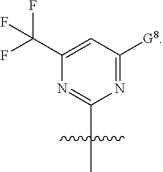

In some embodiments, G¹ is:

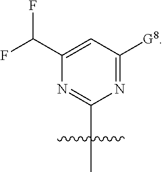

In some embodiments, G¹ is:

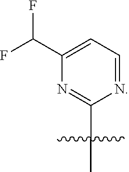 [SMILES: *c1nccc(C(F)F)n1]

In some embodiments, G¹ is C₁₋₆ alkoxy optionally substituted with 1-3 halogens. In some embodiments, G¹ is C₁₋₆ alkxoy optionally substituted with 1-3 fluorines. In some embodiments, G¹ is methoxy substituted with 1-3 fluorines. In some embodiments, G¹ is ethoxy substituted with 1-3 fluorines. In some embodiments, G¹ is propoxy substituted with 1-3 fluorines. In some embodiments, G¹ is butoxy substituted with 1-3 fluorines.

In some embodiments, G¹ is:

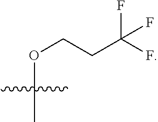 [SMILES: *OCCC(F)(F)F]

In some embodiments, G¹ is:

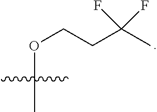 [SMILES: *OCCC(C)(F)F]

In some embodiments, G¹ is:

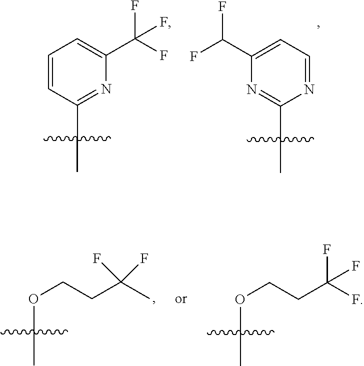

In some embodiments R^{X6} is methyl or C₃₋₅ monocyclic cycloalkyl, each of which is optionally substituted with 1 to 3 halogens.

In some embodiments:
Y¹ is CH or N;
G is

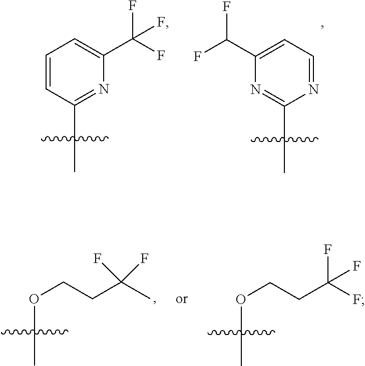

X is —NR¹R², C₁₋₁₀ alkyl, or C₂₋₆ alkenyl,
   wherein the C₁₋₁₀ alkyl and C₂₋₆ alkenyl are each independently substituted with 1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl,
   wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
   wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
   wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{c} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R^{g} independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR⁸S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments,
X is —NR¹R², C₁₋₁₀ alkyl, or C₂₋₆ alkenyl,
   wherein the C₁₋₁₀ alkyl and C₂₋₆ alkenyl are each independently substituted with 1-3 Y groups;
each Y independently is —CN, halogen, R^{a}, R^{b}, R^{c}, C₃₋₅ monocyclic cycloalkyl, phenyl, or naphthalenyl,
   wherein the phenyl and naphthalenyl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —OH, —CN, halogen, —C(O)OH, and R^{a};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl;
   wherein the C₁₋₆ alkyl is optionally substituted with 1-3 groups independently selected from —CN, halogen, R^{a}, R^{b}, and R^{c};
each R³ independently is R^{a}, R^{b}, R^{c}, or C₁₋₆ alkyl, wherein the C₁₋₆ alkyl is optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, or C₁₋₆ alkyl, wherein the C₁₋₆ alkyl is optionally substituted with 1-3 groups independently selected from —CN, halogen, =NR^{5a}R^{a}, R^{d}, R^{e}, phenyl, and naphthalenyl;
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{c} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from —OH, CN, halogen, —C(O)OH, and R^{a};
each R⁷ independently is H or C₁₋₆ alkyl, wherein the C₁₋₆ alkyl is optionally substituted with 1-3 groups independently selected from —OH, —CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments,
X is —NR¹R², C₁₋₁₀ alkyl, or C₂₋₄ alkenyl,
   wherein the C₁₋₁₀ alkyl and C₂₋₄ alkenyl are each independently substituted with 1-3 Y groups;
each Y independently is —OH, —CN, halogen, R^{a}, —NR⁵R⁵, —N⁺R⁵R⁵R^{5a}, —C(O)NR⁵R⁵, —C(O)OR⁴, —OC(O)R⁴, —(O(C₁₋₄ alkyl))ₙOR⁴, or phenyl,
   wherein the phenyl is substituted with 1-5 R³ groups, or
two Y groups on the same carbon, together with the carbon to which they are attached, form a C₃₋₅ monocyclic cycloalkyl;
R¹ is H or C₁₋₄ alkyl, wherein the C₁₋₄ alkyl is optionally substituted with 1-3 groups independently selected from —OH, —CN, halogen, —C(O)OH, and R^{a};
R² is phenyl or 5-6 membered monocyclic heteroaryl, wherein the phenyl and 5-6 membered monocyclic heteroaryl are each independently optionally substituted with 1-3 groups independently selected from —OH, —CN, halogen, —C(O)OH, R^{a}, and C₁₋₆ alkyl,
   wherein the C₁₋₆ alkyl is optionally substituted with 1-3 groups independently selected from —OH, —CN, halogen, —C(O)OH, —NR⁵R⁵, and R^{a};
each R³ independently is —OH, R^{a}, R^{b}, or C₁₋₆ alkyl, wherein the C₁₋₆ alkyl is optionally substituted with 1-3 groups independently selected from —OH, —CN, halogen, —C(O)OH, R^{a}, and R^{b};
each R^{a} independently is —P(O)(OH)₂ or —OP(O)(OH)₂;
each R^{b} independently is —C(O)OR⁴ or —C(O)NR⁵R⁵;
each R⁴ independently is H or C₁₋₆ alkyl, wherein the C₁₋₆ alkyl is optionally substituted with 1-3 groups independently selected from —OH, —CN, halogen, —C(O)OH, —NR⁷R⁷, and R^{a};
each R⁵ independently is H, —C(O)OR⁶, —C(O)C(O)OR⁶, or C₁₋₆ alkyl, wherein the C₁₋₆ alkyl is optionally substituted with 1-3 groups independently selected from —OH, —CN, halogen, —C(O)OR⁶, =NR^{5a}, —NR⁷R⁷, R^{a}, R^{b}, and phenyl;
each R^{5a} independently is H or C₁₋₃ alkyl;
each R⁶ independently is H or C₁₋₆ alkyl, wherein the C₁₋₆ alkyl is optionally substituted with 1-3 groups independently selected from —OH, CN, halogen, —C(O)OH, and R^{a};
each R⁷ independently is H or C₁₋₃ alkyl, wherein the C₁₋₃ alkyl is optionally substituted with 1-3 groups independently selected from —OH, —CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

In some embodiments,
X is —NR¹R², C₁₋₁₀ alkyl, or C₂₋₄ alkenyl, wherein the C₁₋₁₀ alkyl and C₂₋₄ alkenyl are each independently substituted with 1-3 Y groups;
each Y independently is —OH, —CN, halogen, R^{a}, —NR⁵R⁵, —N⁺R⁵R⁵R^{5a}, —C(O)NR⁵R⁵, —C(O)OR⁴, —OC(O)R⁴, —(O(C₁₋₄ alkyl))ₙOR⁴, C₃₋₅ monocyclic cycloalkyl, or phenyl,
   wherein the phenyl is substituted with 1-5 R³ groups, or
two Y groups on the same carbon, together with the carbon to which they are attached, form a C₃₋₅ monocyclic cycloalkyl;
R¹ is H or C₁₋₄ alkyl, wherein the C₁₋₄ alkyl is optionally substituted with 1-3 groups independently selected from —OH, —CN, halogen, —C(O)OH, and R^{a};
R² is phenyl or 5-6 membered monocyclic heteroaryl, wherein the phenyl and 5-6 membered monocyclic heteroaryl are each independently optionally substituted with 1-3 groups independently selected from —OH, —CN, halogen, —C(O)OH, R^{a}, and C₁₋₆ alkyl,
   wherein the C₁₋₆ alkyl is optionally substituted with 1-3 groups independently selected from —OH, —CN, halogen, —C(O)OH, —NR⁵R⁵, and R^{a};
each R³ independently is —OH, —C(O)OH, R^{a}, or C₁₋₆ alkyl, wherein the C₁₋₆ alkyl is optionally substituted with 1-3 groups independently selected from —OH, —CN, halogen, —C(O)OH, and R^{a};
each R^{a} independently is —P(O)(OH)₂ or —OP(O)(OH)₂;
each R⁴ independently is H or C₁₋₆ alkyl, wherein the C₁₋₆ alkyl is optionally substituted with 1-3 groups independently selected from —OH, —CN, halogen, —C(O)OH, —NR⁷R⁷, and R^{a};
each R⁵ independently is H, —C(O)OR⁶, —C(O)C(O)OR⁶, or C₁₋₆ alkyl, wherein the C₁₋₆ alkyl is optionally substituted with 1-3 groups independently selected from —OH, —CN, halogen, —C(O)OR⁶, =NR^{5a}, —NR⁷R⁷, R^{a}, and phenyl;
each R^{5a} independently is H or C₁₋₃ alkyl;
each R⁶ independently is H or C₁₋₆ alkyl, wherein the C₁₋₆ alkyl is optionally substituted with 1-3 groups independently selected from —OH, CN, halogen, —C(O)OH, and R^{a};
each R⁷ independently is H or C₁₋₃ alkyl, wherein the C₁₋₃ alkyl is optionally substituted with 1-3 groups independently selected from —OH, —CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

As used herein, a 5-8 membered monocyclic heterocyclyl has 1-4 ring heteroatoms independently selected from N, O, and S. As used herein, a 5-6 membered monocyclic heteroaryl has 1-4 ring heteroatoms independently selected from N, O, and S. As used herein, a 8-10 membered fused bicyclic heteroaryl has 1-4 ring heteroatoms independently selected from N, O, and S. As used herein, a 8-10 membered fused bicyclic heterocyclyl has 1-4 ring heteroatoms independently selected from N, O, and S. As used herein, a 8-10 membered bridged bicyclic heterocyclyl has 1-4 ring heteroatoms independently selected from N, O, and S. As used herein, a 7-10 membered spirocyclic heterocyclyl has 1-4 ring heteroatoms independently selected from N, O, and S.

In some embodiments, X is —NR¹R², C₁₋₁₀ alkyl, or C₂₋₆ alkenyl, wherein the C₁₋₁₀ alkyl and C₂₋₆ alkenyl are each independently substituted with 1-3 Y groups. In some embodiments, X is —NR¹R², C₁₋₁₀ alkyl, or C₂₋₄ alkenyl, wherein the C₁₋₁₀ alkyl and C₂₋₄ alkenyl are each independently substituted with 1-3 Y groups.

In some embodiments, X is —NR¹R².

In some embodiments, R¹ is H or C₁₋₆ alkyl, wherein the C₁₋₆ alkyl is optionally substituted with 1-3 groups independently selected from —CN, halogen, R^{a}, R^{b}, and R^{c}. In some embodiments, R¹ is H or C₁₋₆ alkyl, wherein the C₁₋₆ alkyl is optionally substituted with 1-3 groups independently selected from —OH, —CN, halogen, —C(O)OH, and R^{a}. In some embodiments, R¹ is H or C₁₋₄ alkyl, wherein the C₁₋₄ alkyl is optionally substituted with 1-3 groups independently selected from —OH, —CN, halogen, —C(O)OH, and R^{a}.

In some embodiments, R¹ is H.

In some embodiments, R¹ is C₁₋₆ alkyl, wherein the C₁₋₆ alkyl is optionally substituted with 1-3 groups independently selected from —CN, halogen, R^{a}, R^{b}, and R^{c}. In some embodiments, R¹ is C₁₋₆ alkyl, wherein the C₁₋₆ alkyl is optionally substituted with 1-3 groups independently selected from —OH, —CN, halogen, —C(O)OH, and R^{a}. In some embodiments, R¹ is C₁₋₄ alkyl, wherein the C₁₋₄ alkyl is optionally substituted with 1-3 groups independently selected from —OH, —CN, halogen, —C(O)OH, and R^{a}. In some embodiments, R¹ is C₁₋₃ alkyl, wherein the C₁₋₃ alkyl is optionally substituted with 1-3 groups independently selected from —C(O)OH and R^{a}. In some embodiments, R¹ is methyl, wherein the methyl is optionally substituted with 1-3 groups independently selected from —COOH and R^{a}.

In some embodiments, R¹ is C₁₋₆ alkyl, wherein the C₁₋₆ alkyl is substituted with 1-3 groups independently selected from —CN, halogen, R^{a}, R^{b}, and R^{c}. In some embodiments, R¹ is C₁₋₆ alkyl, wherein the C₁₋₆ alkyl is substituted with 1-3 groups independently selected from —OH, —CN, halogen, —C(O)OH, and R^{a}. In some embodiments, R¹ is C₁₋₄ alkyl, wherein the C₁₋₄ alkyl is substituted with 1-3 groups independently selected from —OH, —CN, halogen, —C(O)OH, and R^{a}. In some embodiments, R¹ is C₁₋₃ alkyl, wherein the C₁₋₃ alkyl is substituted with 1-3 groups independently selected from —C(O)OH and R^{a}. In some embodiments, R¹ is methyl, wherein the methyl is substituted with 1-3 groups independently selected from —COOH and R^{a}.

In some embodiments, R¹ is C₁₋₆ alkyl. In some embodiments, R¹ is C₁₋₄ alkyl. In some embodiments, R¹ is C₁₋₃ alkyl. In some embodiments, R¹ is methyl.

In some embodiments, R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
wherein the C₁₋₆ alkyl is optionally substituted with 1-3 groups independently selected from —CN, halogen, R^{a}, R^{b}, and R^{c}.

In some embodiments, R² is phenyl or 5-6 membered monocyclic heteroaryl, wherein the phenyl and 5-6 membered monocyclic heteroaryl are each independently optionally substituted with 1-3 groups independently selected from —OH, —CN, halogen, —C(O)OH, R^{a}, and C₁₋₆ alkyl,
wherein the C₁₋₆ alkyl is optionally substituted with 1-3 groups independently selected from —OH, —CN, halogen, —C(O)OH, —NR⁵R⁵, and R^{a}.

In some embodiments, R² is phenyl, wherein the phenyl is optionally substituted with 1-3 groups independently selected from —CN, halogen, R^{a}, R^{b}, R^{c}, and C₁₋₆ alkyl, wherein the C₁₋₆ alkyl is optionally substituted with 1-3 groups independently selected from —CN, halogen, R^{a}, R^{b}, and R^{c}. In some embodiments, R² is phenyl, wherein the phenyl is optionally substituted with 1-3 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, —NR⁵C(O)OR⁴, and R^{a}. In some embodiments, R² is phenyl, wherein the phenyl is optionally substituted with 1-3 groups independently selected from —OH, —CN, halogen, —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}.

In some embodiments, R² is phenyl, wherein the phenyl is
i) substituted with C₁₋₄ alkyl, wherein the C₁₋₄ alkyl is substituted with one group selected from R^{a} and —NR⁵C(O)OR⁴, and
ii) optionally substituted with 1-2 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR^{5R}, —NR⁵R⁵, —NR⁵C(O)OR⁴, and R^{a}.

In some embodiments, R² is phenyl, wherein the phenyl is optionally substituted with 1-3 groups independently selected from —CN, halogen, R^{a}, R^{b}, and R^{c}. In some embodiments, R² is phenyl, wherein the phenyl is optionally substituted with 1-2 groups independently selected from —C(O)OH and R^{a}.

In some embodiments, R² is phenyl, wherein the phenyl is substituted with 1-3 groups independently selected from —CN, halogen, R^{a}, R^{b}, R^{c}, and C₁₋₆ alkyl, wherein the C₁₋₆ alkyl is optionally substituted with 1-3 groups independently selected from —CN, halogen, R^{a}, R^{b}, and R^{c}. In some embodiments, R² is phenyl, wherein the phenyl is substituted with 1-3 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, —NR⁵C(O)OR⁴, and R^{a}. In some embodiments, R² is phenyl, wherein the phenyl is substituted with 1-3 groups independently selected from —OH, —CN, halogen, —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}.

In some embodiments, R² is phenyl, wherein the phenyl is
i) substituted with C₁₋₄ alkyl, wherein the C₁₋₄ alkyl is substituted with one group selected from R^{a} and —NR⁵C(O)OR⁴, and
ii) substituted with 1-2 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, —NR⁵C(O)OR⁴, and R^{a}.

In some embodiments, R² is phenyl, wherein the phenyl is substituted with 1-3 groups independently selected from —CN, halogen, R^{a}, R^{b}, and R^{c}. In some embodiments, R² is phenyl, wherein the phenyl is substituted with 1-2 groups independently selected from —C(O)OH and R^{a}.

In some embodiments, R² is phenyl.

In some embodiments, R² is 5-6 membered monocyclic heteroaryl, wherein the 5-6 membered monocyclic heteroaryl is optionally substituted with 1-3 groups independently selected from —CN, halogen, R^{a}, R^{b}, R^{c}, and C₁₋₆ alkyl, wherein the C₁₋₆ alkyl is optionally substituted with 1-3 groups independently selected from —CN, halogen, R^{a}, R^{b}, and R^{c}. In some embodiments, R² is 5-6 membered monocyclic heteroaryl, wherein the 5-6 membered monocyclic heteroaryl is optionally substituted with 1-3 groups independently selected from —OH, —CN, halogen, —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}.

In some embodiments, R² is 5-6 membered monocyclic heteroaryl, wherein the 5-6 membered monocyclic heteroaryl is substituted with 1-3 groups independently selected from —CN, halogen, R^{a}, R^{b}, R^{c}, and C₁₋₆ alkyl, wherein the C₁₋₆ alkyl is optionally substituted with 1-3 groups independently selected from —CN, halogen, R^{a}, R^{b}, and R^{c}. In some embodiments, R² is 5-6 membered monocyclic heteroaryl, wherein the 5-6 membered monocyclic heteroaryl is substituted with 1-3 groups independently selected from —OH, —CN, halogen, —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}.

In some embodiments, R² is 5-6 membered monocyclic heteroaryl.

In some embodiments, R² is 6-membered monocyclic heteroaryl, wherein the 6-membered monocyclic heteroaryl is optionally substituted with 1-3 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, —NR⁵C(O)OR⁴, and R^{a}.

In some embodiments, R² is 6-membered monocyclic heteroaryl, wherein the 6-membered monocyclic heteroaryl is
i) substituted with C₁₋₄ alkyl, wherein the C₁₋₄ alkyl is substituted with one group selected from R^{a} and —NR⁵C(O)OR⁴, and
ii) optionally substituted with 1-2 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, and R^{a}.

In some embodiments, R² is pyridinyl, wherein the pyridinyl is optionally substituted with 1-2 groups independently selected from —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}.

In some embodiments, R² is 6-membered monocyclic heteroaryl, wherein the 6-membered monocyclic heteroaryl is substituted with 1-3 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, —NR⁵C(O)OR⁴, and R^{a}.

In some embodiments, R² is 6-membered monocyclic heteroaryl, wherein the 6-membered monocyclic heteroaryl is
i) substituted with C₁₋₄ alkyl, wherein the C₁₋₄ alkyl is substituted with one group selected from R^{a} and —NR⁵C(O)OR⁴, and
ii) substituted with 1-2 groups independently selected from —OH, —CN, halogen, —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵C(O)OR⁴, —NR⁵S(O)₂R⁴, R^{a}, and C₁₋₆ alkyl, wherein the C₁₋₆ alkyl is optionally substituted with 1-3 groups independently selected from —OH, —CN, halogen, —C(O)OR⁴, —C(O)NR⁵R⁵, —NR⁵R⁵, and R^{a}.

In some embodiments, R² is pyridinyl, wherein the pyridinyl is substituted with 1-2 groups independently selected from —C(O)OH, R^{a}, and C₁₋₆ alkyl, wherein the C₁₋₆ alkyl is optionally substituted with 1-3 groups independently selected from —OH, —CN, halogen, —C(O)OH, —NR⁵R⁵, and R^{a}. In some embodiments, R² is pyridinyl, wherein the pyridinyl is substituted with C₁₋₃ alkyl, wherein the C₁₋₃ alkyl is substituted with 1-3 groups independently selected from —OH, —CN, halogen, —C(O)OH, —NR⁵R⁵, and R^{a}.

In some embodiments, X is C₁₋₁₀ alkyl, wherein the C₁₋₁₀ alkyl is substituted with 1-3 Y groups. In some embodiments, X is C₁₋₁₀ alkyl, wherein the C₁₋₁₀ alkyl is substituted with two Y groups. In some embodiments, X is C₁₋₁₀ alkyl, wherein the C₁₋₁₀ alkyl is substituted with one Y group. In some embodiments, X is C₁₋₈ alkyl, wherein the C₁₋₈ alkyl is substituted with 1-3 Y groups. In some embodiments, X is C₁₋₈ alkyl, wherein the C₁₋₈ alkyl is substituted with 1-2 Y groups. In some embodiments, X is C₁₋₈ alkyl, wherein the C₁₋₈ alkyl is substituted with three Y groups. In some embodiments, X is C₁₋₈ alkyl, wherein the C₁₋₈ alkyl is substituted with two Y groups. In some embodiments, X is C₁₋₈ alkyl, wherein the C₁₋₈ alkyl is substituted with one Y group. In some embodiments, X is C₁₋₆ alkyl, wherein the C₁₋₆ alkyl is substituted with 1-3 Y groups. In some embodiments, X is C₁₋₆ alkyl, wherein the C₁₋₆ alkyl is substituted with 1-2 Y groups. In some embodiments, X is C₁₋₆ alkyl, wherein the C₁₋₆ alkyl is substituted with three Y groups. In some embodiments, X is C₁₋₆ alkyl, wherein the C₁₋₆ alkyl is substituted with two Y groups. In some embodiments, X is C₁₋₆ alkyl, wherein the C₁₋₆ alkyl is substituted with one Y group.

In some embodiments, X substituted with Y is —CH₂Y, —CH₂CH₂Y, —CH₂CH₂CH₂Y, —CH₂CH₂CH₂CH₂Y,

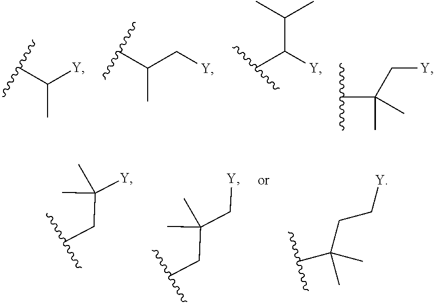

In some embodiments, X substituted with Y is

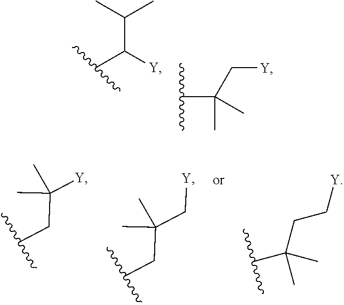

In some embodiments, X substituted with Y is

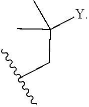

In some embodiments, X is C₂₋₆ alkenyl, wherein the C₂₋₆ alkenyl is substituted with 1-3 Y groups. In some embodiments, X is C₂₋₄ alkenyl, wherein the C₂₋₄ alkenyl is substituted with 1-3 Y groups.

In some embodiments,
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl.

In some embodiments,
each Y independently is —CN, halogen, R^{a}, R^{b}, R^{c}, phenyl, or naphthalenyl, wherein the phenyl and naphthalenyl are each independently substituted with 1-5 R³ groups, or
two Y groups on the same carbon, together with the carbon to which they are attached, form a C₃₋₅ monocyclic cycloalkyl.

In some embodiments,
each Y independently is —OH, —CN, halogen, R^{a}, —NR⁵R⁵, —N⁺R⁵R⁵R^{5a}, —C(O)NR⁵R⁵, —C(O)OR⁴, —OC(O)R⁴, —(O(C₁₋₄ alkyl))ₙOR⁴, or phenyl, wherein the phenyl is substituted with 1-5 R³ groups, or
two Y groups on the same carbon, together with the carbon to which they are attached, form a C₃₋₅ monocyclic cycloalkyl.

In some embodiments,
two Y groups on the same carbon, together with the carbon to which they are attached, form a C₃₋₅ monocyclic cycloalkyl, and
one Y is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
   wherein the phenyl, naphthalenyl, 5-6 membered monocyclic heteroaryl, and 8-10 membered fused bicyclic heteroaryl are each independently substituted with 1-5 R³ groups.

In some embodiments,
two Y groups on the same carbon, together with the carbon to which they are attached, form a C₃₋₅ monocyclic cycloalkyl, and
one Y is —CN, halogen, R^{a}, R^{b}, R^{c}, phenyl, or naphthalenyl, wherein the phenyl and naphthalenyl are each independently substituted with 1-5 R³ groups.

In some embodiments,
two Y groups on the same carbon, together with the carbon to which they are attached, form a C₃₋₅ monocyclic cycloalkyl, and
one Y is —OH, —CN, halogen, R^{a}, —NR⁵R⁵, —N⁺R⁵R⁵R^{5a}, —C(O)NR⁵R⁵, —C(O)OR⁴, —OC(O)R⁴, —(O(C₁₋₄ alkyl))ₙOR⁴, or phenyl, wherein the phenyl is substituted with 1-5 R³ groups.

In some embodiments, each Y independently is —B(OH)₂, —C(O)OR⁴, —C(O)NR⁵R⁵, —OC(O)R⁴, —(O(C₁₋₄ alkyl))ₙOR⁴, —NR⁵R⁵, —N⁺R⁵R⁵R^{5a}, —S(O)₂R⁴, —S(O)₂NR⁵R⁵, —S(O)₂OR⁴, —NR⁵C(O)R⁴, —NR⁵C(O)NR⁵R⁵, —NR⁵S(O)₂R⁴, R^{a}, 5-6 membered monocyclic heteroaryl, or 8-10 membered fused bicyclic heteroaryl,
wherein the 5-6 membered monocyclic heteroaryl and 8-10 membered fused bicyclic heteroaryl are each independently substituted with 1-3 groups independently selected from —OH, —CN, halogen, —C(O)OR⁴, —C(O)NR⁵R⁵, and R^{a}.

In some embodiments, each Y independently is R^{a}, —NR⁵R⁵, —N⁺R⁵R⁵R^{5a}, —C(O)OR⁴, —OC(O)R⁴, or —(O(C₁₋₄ alkyl))ₙOR⁴.

In some embodiments, one or more Y is —B(OH)₂. In some embodiments, one or more Y is —CN. In some embodiments, one or more Y is halogen. In some embodiments, one or more Y is R^{a}. In some embodiments, one or more Y is R^{b}. In some embodiments, one or more Y is R^{c}.

In some embodiments, one or more Y is —OH. In some embodiments, one or more Y is —NR⁵R⁵. In some embodiments, one or more Y is —N⁺R⁵R⁵R^{5a}. In some embodiments, one or more Y is —C(O)NR⁵R⁵. In some embodiments, one or more Y is —C(O)OR⁴. In some embodiments, one or more Y is —OC(O)R⁴. In some embodiments, one or more Y is —(O(C₁₋₄ alkyl))ₙOR⁴. In some embodiments, one or more Y is —(O(CH₂CH₂)ₙOR⁴. In some embodiments, one or more Y is —S(O)₂R⁴. In some embodiments, one or more Y is —S(O)₂NR⁵R⁵. In some embodiments, one or more Y is —S(O)₂OR⁴. In some embodiments, one or more Y is —NR⁵C(O)R⁴. In some embodiments, one or more Y is —NR⁵C(O)NR⁵R⁵. In some embodiments, one or more Y is —NR⁵S(O)₂R⁴.

In some embodiments, one or more Y is phenyl, wherein the phenyl is substituted with 1-5 R³ groups. In some embodiments, one or more Y is naphthalenyl, wherein the naphthalenyl is substituted with 1-5 R³ groups.

In some embodiments, one or more Y is 5-6 membered monocyclic heteroaryl, wherein the 5-6 membered monocyclic heteroaryl is substituted with 1-5 R³ groups. In some embodiments, one or more Y is 5-6 membered monocyclic heteroaryl, wherein the 5-6 membered monocyclic heteroaryl is substituted with 1-3 groups independently selected from —OH, —CN, halogen, —C(O)OR⁴, —C(O)NR⁵R⁵, and R^{a}.

In some embodiments, one or more Y is 8-10 membered fused bicyclic heteroaryl, wherein the 8-10 membered fused bicyclic heteroaryl is substituted with 1-5 R³ groups. In some embodiments, one or more Y is 8-10 membered fused bicyclic heteroaryl, wherein the 8-10 membered fused bicyclic heteroaryl is substituted with 1-3 groups independently selected from —OH, —CN, halogen, —C(O)OR⁴, —C(O)NR⁵R⁵, and R^{a}.

In some embodiments, n is 1, 2, 3, 4, or 5. In some embodiments, n is 1. In some embodiments, n is 2. In some embodiments, n is 3. In some embodiments, n is 4. In some embodiments, n is 5.

In some embodiments, one Y is —C(O)OH, —NH₂, or —N(CH₃)₂, and one Y is —NR⁵R⁵.

In some embodiments, two Y groups on the same carbon, together with the carbon to which they are attached, form a C₃₋₅ monocyclic cycloalkyl.

In some embodiments, X is substituted with three Y groups, wherein two of the three Y groups are on the same carbon and wherein the two Y groups on the same carbon, together with the carbon to which they are attached, form a cyclopropyl.

In some embodiments, X substituted with three Y groups is:

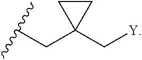

In some embodiments, X is substituted with three Y groups,
wherein two Y groups are on the same carbon and wherein the two Y groups on the same carbon, together with the carbon to which they are attached, form a cyclopropyl, and
the third Y group is —NR⁵R⁵.

In some embodiments, X substituted with three Y groups is:

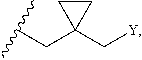

wherein Y is —NR⁵R⁵.

In some embodiments, one Y is phenyl, wherein the phenyl is substituted with 1-5 R³ groups. In some embodiments, one Y is phenyl, wherein the phenyl is substituted with 1-3 R³ groups. In some embodiments, one Y is phenyl, wherein the phenyl is substituted with three R³ groups.

In some embodiments, each R³ independently is R^{a}, R^{b}, R^{c}, C₁₋₆ alkyl, or 5-6 membered monocyclic heteroaryl, wherein the C₁₋₆ alkyl and 5-6 membered monocyclic heteroaryl are each independently optionally substituted with 1-3 groups independently selected from —CN, halogen, R^{a}, R^{b}, and R^{c}. In some embodiments, each R³ independently is R^{a}, R^{b}, R^{c}, or C₁₋₆ alkyl, wherein the C₁₋₆ alkyl is optionally substituted with 1-3 groups independently selected from —CN, halogen, R^{a}, R^{b}, and R^{c}. In some embodiments, each R³ independently is —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵S(O)₂R⁴, R^{a}, or C₁₋₆ alkyl, wherein the C₁₋₆ alkyl is optionally substituted with 1-3 groups independently selected from —OH, —CN, halogen, —C(O)OR⁴, —C(O)NR⁵R⁵, and R^{a}. In some embodiments, each R³ independently is —OH, —C(O)OH, R^{a}, or C₁₋₆ alkyl, wherein the C₁₋₆ alkyl is optionally substituted with 1-3 groups independently selected from —OH, —CN, halogen, —C(O)OH, and R^{a}. In some embodiments, each R³ independently is —OH, —C(O)OH, —C(O)NR⁵R⁵, R^{a}, or C₁₋₃ alkyl, wherein the C₁₋₃ alkyl is optionally substituted with 1-3 groups independently selected from —OH, —CN, halogen, —C(O)OH, —C(O)NR⁵R⁵, and R^{a}. In some embodiments, each R³ independently is —OH, —C(O)OH, R^{a}, or C₁₋₃ alkyl, wherein the C₁₋₃ alkyl is optionally substituted with 1-3 groups independently selected from —OH, —CN, halogen, —C(O)OH, and R^{a}. In some embodiments, each R³ independently is —OH, —C(O)OH, —C(O)NR⁵R⁵, R^{a}, methyl, —CH₂P(O)(OH)₂, —CH₂C(O)OH, or —CH₂C(O)NR⁵R⁵. In some embodiments, each R³ independently is —OH, —C(O)OH, R^{a}, methyl, —CH₂P(O)(OH)₂, or —CH₂C(O)OH.

In some embodiments, one or more R³ is R^{a}. In some embodiments, one or more R³ is R^{b}. In some embodiments, one or more R³ is R^{c}. In some embodiments, one or more R³ is —C(O)OR⁴. In some embodiments, one or more R³ is —C(O)OH. In some embodiments, one or more R³ is —C(O)NR⁵R⁵. In some embodiments, one or more R³ is —S(O)₂R⁴. In some embodiments, one or more R³ is —S(O)₂NR⁵R⁵. In some embodiments, one or more R³ is —S(O)₂OR⁴. In some embodiments, one or more R³ is —NR⁵C(O)R⁴. In some embodiments, one or more R³ is —NR⁵C(O)NR⁵R⁵. In some embodiments, one or more R³ is —NR⁵S(O)₂R⁴. In some embodiments, one or more R³ is —OH.

In some embodiments, one or more R³ is C₁₋₆ alkyl, wherein the C₁₋₆ alkyl is optionally substituted with 1-3 groups independently selected from —CN, halogen, R^{a}, R^{b}, and R^{c}. In some embodiments, one or more R³ is C₁₋₆ alkyl, wherein the C₁₋₆ alkyl is optionally substituted with 1-3 groups independently selected from —OH, —CN, halogen, —C(O)OR⁴, —C(O)NR⁵R⁵, and R. In some embodiments, one or more R³ is C₁₋₆ alkyl, wherein the C₁₋₆ alkyl is optionally substituted with 1-3 groups independently selected from —OH, —CN, halogen, —C(O)OH, and R^{a}. In some embodiments, one or more R³ is C₁₋₃ alkyl, wherein the C₁₋₃ alkyl is optionally substituted with 1-3 groups independently selected from —OH, —CN, halogen, —C(O)OH, and R^{a}.

In some embodiments, one or more R³ is C₁₋₆ alkyl, wherein the C₁₋₆ alkyl is substituted with 1-3 groups independently selected from —CN, halogen, R^{a}, R^{b}, and R^{c}. In some embodiments, one or more R³ is C₁₋₆ alkyl, wherein the C₁₋₆ alkyl is substituted with 1-3 groups independently selected from —OH, —CN, halogen, —C(O)OR⁴, —C(O)NR⁵R⁵, and R^{a}. In some embodiments, one or more R³ is C₁₋₆ alkyl, wherein the C₁₋₆ alkyl is substituted with 1-3 groups independently selected from —OH, —CN, halogen, —C(O)OH, and R^{a}. In some embodiments, one or more R³ is C₁₋₃ alkyl, wherein the C₁₋₃ alkyl is substituted with 1-3 groups independently selected from —OH, —CN, halogen, —C(O)OH, and R^{a}. In some embodiments, one or more R³ is —CH₂P(O)(OH)₂. In some embodiments, one or more R³ is —CH₂C(O)OH.

In some embodiments, one or more R³ is C₁₋₆ alkyl. In some embodiments, one or more R³ is C₁₋₃ alkyl. In some embodiments, one or more R³ is methyl.

In some embodiments, one R³ is —OP(O)(OH)₂ and 1-2 R³ is C₁₋₃ alkyl, wherein the C₁₋₃ alkyl is optionally substituted with 1-3 groups independently selected from —C(O)OH, —C(O)NR⁵R⁵, and R^{a}. In some embodiments, one R³ is —OP(O)(OH)₂ and 1-2 R³ is C₁₋₃ alkyl, wherein the C₁₋₃ alkyl is optionally substituted with 1-3 groups independently selected from —C(O)OH and R^{a}. In some embodiments, one R³ is —OP(O)(OH)₂, one R³ is unsubstituted C₁₋₃ alkyl, and one R³ is C₁₋₃ alkyl, wherein the C₁₋₃ alkyl is substituted with 1-3 groups independently selected from —C(O)OH and R^{a}.

In some embodiments, one or more R³ is 5-6 membered monocyclic heteroaryl, wherein the 5-6 membered monocyclic heteroaryl is optionally substituted with 1-3 groups independently selected from —CN, halogen, R^{a}, R^{b}, and R^{c}. In some embodiments, one or more R³ is 5-6 membered monocyclic heteroaryl, wherein the 5-6 membered monocyclic heteroaryl is substituted with 1-3 groups independently selected from —CN, halogen, R^{a}, R^{b}, and R^{c}. In some embodiments, one or more R³ is 5-6 membered monocyclic heteroaryl.

In some embodiments, one Y is phenyl, wherein the phenyl is substituted with methyl, —OP(O)(OH)₂, and —CH₂C(O)OH.

In some embodiments, X is C₂₋₆ alkenyl, wherein the C₂₋₆ alkenyl is substituted with 1-3 Y groups. In some embodiments, X is C₂₋₆ alkenyl, wherein the C₂₋₆ alkenyl is substituted with 1-2 Y groups. In some embodiments, X is C₂₋₆ alkenyl, wherein the C₂₋₆ alkenyl is substituted with two Y groups. In some embodiments, X is C₂₋₆ alkenyl, wherein the C₂₋₆ alkenyl is substituted with one Y group. In some embodiments, X is C₂₋₄ alkenyl, wherein the C₂₋₄ alkenyl is substituted with 1-3 Y groups. In some embodiments, X is C₂₋₄ alkenyl, wherein the C₂₋₄ alkenyl is substituted with 1-2 Y groups. In some embodiments, X is C₂₋₄ alkenyl, wherein the C₂₋₄ alkenyl is substituted with three Y groups. In some embodiments, X is C₂₋₄ alkenyl, wherein the C₂₋₄ alkenyl is substituted with two Y groups. In some embodiments, X is C₂₋₄ alkenyl, wherein the C₂₋₄ alkenyl is substituted with one Y group. In some embodiments, X is C₂ alkenyl, wherein the C₂ alkenyl is substituted with 1-2 Y groups. In some embodiments, X is C₂ alkenyl, wherein the C₂ alkenyl is substituted with two Y groups. In some embodiments, X is C₂ alkenyl, wherein the C₂ alkenyl is substituted with one Y group.

In some embodiments, X is C₂₋₆ alkenyl, wherein the C₂₋₆ alkenyl is substituted with 1-3 Y group and wherein one or more Y groups is —C(O)NR⁵R⁵. In some embodiments, X is C₂₋₆ alkenyl, wherein the C₂₋₆ alkenyl is substituted with two Y groups and wherein one or more Y groups is —C(O)NR⁵R⁵. In some embodiments, X is C₂₋₆ alkenyl, wherein the C₂₋₆ alkenyl is substituted with one Y group and wherein the Y group is —C(O)NR⁵R⁵. In some embodiments, X is C₂₋₄ alkenyl, wherein the C₂₋₄ alkenyl is substituted with 1-3 Y groups and wherein one or more Y groups is —C(O)NR⁵R⁵. In some embodiments, X is C₂₋₄ alkenyl, wherein the C₂₋₄ alkenyl is substituted with 1-2 Y groups and wherein one or more Y groups is —C(O)NR⁵R⁵. In some embodiments, X is C₂₋₄ alkenyl, wherein the C₂₋₄ alkenyl is substituted with three Y groups and wherein one or more Y groups is —C(O)NR⁵R⁵. In some embodiments, X is C₂₋₄ alkenyl, wherein the C₂₋₄ alkenyl is substituted with two Y groups and wherein one or more Y groups is —C(O)NR⁵R⁵. In some embodiments, X is C₂₋₄ alkenyl, wherein the C₂₋₄ alkenyl is substituted with one Y group and wherein the Y group is —C(O)NR⁵R⁵. In some embodiments, X is C₂ alkenyl, wherein the C₂ alkenyl is substituted with 1-2 Y groups and wherein one or more Y groups is —C(O)NR⁵R⁵. In some embodiments, X is C₂ alkenyl, wherein the C₂ alkenyl is substituted with two Y groups and wherein one or more Y groups is —C(O)NR⁵R⁵. In some embodiments, X is C₂ alkenyl, wherein the C₂ alkenyl is substituted with one Y group and wherein the Y group is —C(O)NR⁵R⁵.

In some embodiments, each R^{a} independently is —P(O)(OH)₂ or —OP(O)(OH)₂. In some embodiments, one or more R^{a} is —P(O)(OH)₂. In some embodiments, one or more R^{a} is —OP(O)(OH)₂.

In some embodiments, each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴. In some embodiments, one or more R^{b} is —C(O)R⁴. In some embodiments, one or more R^{b} is —C(O)OR⁴. In some embodiments, one or more R^{b} is —C(O)NR⁵R⁵. In some embodiments, one or more R^{b} is —C(O)C(O)OR⁴. In some embodiments, one or more R^{b} is —S(O)₂R⁴. In some embodiments, one or more R^{b} is —S(O)₂NR⁵R⁵. In some embodiments, one or more R^{b} is —S(O)₂OR⁴.

In some embodiments, each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴. In some embodiments, one or more R^{c} is —OR⁴. In some embodiments, one or more R^{c} is —OC(O)R⁴. In some embodiments, one or more R^{c} is —OC(O)C(O)OR⁴. In some embodiments, one or more R^{c} is —(O(C₁₋₄ alkyl))ₙOR⁴. In some embodiments, one or more R^{c} is —NR⁵R⁵. In some embodiments, one or more R^{c} is —N⁺R⁵R⁵R^{5a}In some embodiments, one or more R^{c} is —NR⁵C(O)R⁴. In some embodiments, one or more R^{c} is —NR⁵C(O)NR⁵R⁵. In some embodiments, one or more R^{c} is —NR⁵C(O)OR⁴. In some embodiments, one or more R^{c} is —NR⁵C(O)C(O)OR⁴. In some embodiments, one or more R^{c} is —NR⁵(O)₂R⁴.

In some embodiments, each R⁴ independently is H or C₁₋₆ alkyl, wherein the C₁₋₆ alkyl is optionally substituted with 1-3 groups independently selected from —CN, halogen, R^{a}, R^{d}, and R^{e}. In some embodiments, each R⁴ independently is H or C₁₋₆ alkyl, wherein the C₁₋₆ alkyl is optionally substituted with 1-3 groups independently selected from —OH, —CN, halogen, —C(O)OH, —NR⁷R⁷, and R^{a}. In some embodiments, each R⁴ independently is H or C₁₋₆ alkyl, wherein the C₁₋₆ alkyl is optionally substituted with 1-2 groups independently selected from —C(O)OH, —NR⁷R⁷, and R^{a}. In some embodiments, each R⁴ independently is C₁₋₄ alkyl, wherein the C₁₋₄ alkyl is optionally substituted with one group selected from —C(O)OH, —NR⁷R⁷, and R^{a}.

In some embodiments, one or more R⁴ is H. In some embodiments, one or more R⁴ is C₁₋₆ alkyl, wherein the C₁₋₆ alkyl is optionally substituted with 1-3 groups independently selected from —CN, halogen, R^{a}, R^{d}, and R^{e}. In some embodiments, one or more R⁴ is C₁₋₆ alkyl, wherein the C₁₋₆ alkyl is optionally substituted with 1-3 groups independently selected from —OH, —CN, halogen, —C(O)OH, —NR⁷R⁷, and R^{a}. In some embodiments, one or more R⁴ is C₁₋₆ alkyl, wherein the C₁₋₆ alkyl is optionally substituted with 1-2 groups independently selected from —C(O)OH, —NR⁷R⁷, and R^{a}. In some embodiments, one or more R⁴ is C₁₋₄ alkyl, wherein the C₁₋₄ alkyl is optionally substituted with one group selected from —C(O)OH, —NR⁷R⁷, and R^{a}. In some embodiments, one or more R⁴ is C₁₋₆ alkyl, wherein the C₁₋₆ alkyl is substituted with 1-3 groups independently selected from —CN, halogen, R^{a}, R^{d}, and R^{e}. In some embodiments, one or more R⁴ is C₁₋₆ alkyl, wherein the C₁₋₆ alkyl is substituted with 1-3 groups independently selected from —OH, —CN, halogen, —C(O)OH, —NR⁷R⁷, and R^{a}. In some embodiments, one or more R⁴ is C₁₋₆ alkyl, wherein the C₁₋₆ alkyl is substituted with 1-2 groups independently selected from —C(O)OH, —NR⁷R⁷, and R^{a}. In some embodiments, one or more R⁴ is C₁₋₄ alkyl, wherein the C₁₋₄ alkyl is substituted with one group selected from —C(O)OH, —NR⁷R⁷, and R^{a}. In some embodiments, one or more R⁴ is C₁₋₆ alkyl. In some embodiments, one or more R⁴ is C₁₋₄ alkyl. In some embodiments, one or more R⁴ is methyl.

In some embodiments, n is 1, 2, 3, or 4 and R⁴ is methyl. In some embodiments, n is 4 and R⁴ is methyl.

In some embodiments, each R⁵ independently is H, R^{d}, C₁₋₆ alkyl, or 5-6 membered monocyclic heteroaryl,
wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
wherein the 5-6 membered monocyclic heteroaryl is optionally substituted with 1-3 groups independently selected from —CN, halogen, R^{a}, R^{d}, and R^{e}.

In some embodiments, each R⁵ independently is H, R^{d}, or C₁₋₆ alkyl, wherein the C₁₋₆ alkyl is optionally substituted with 1-3 groups independently selected from —CN, halogen, =NR^{5a}, R^{a}, R^{d}, R^{e}, phenyl, and naphthalenyl.

In some embodiments, each R⁵ independently is H, —C(O)OR⁶, —C(O)C(O)OR⁶, or C₁₋₆ alkyl, wherein the C₁₋₆ alkyl is optionally substituted with 1-3 groups independently selected from —OH, —CN, halogen, —C(O)OR⁶, =NR^{5a}, —NR⁷R⁷, R^{a}, and phenyl.

In some embodiments, each R⁵ independently is H, —C(O)OR⁶, —C(O)C(O)OR⁶, or C₁₋₄ alkyl, wherein the C₁₋₄ alkyl is optionally substituted with 1-2 groups independently selected from —C(O)OH, —C(O)NH₂, =NR^{5a}, —NR⁷R⁷, R^{a}, and phenyl.

In some embodiments, each R⁵ independently is H, —C(O)OR⁶, —C(O)C(O)OR⁶, or C₁₋₄ alkyl, wherein the C₁₋₄ alkyl is optionally substituted with 1-2 groups independently selected from —C(O)OH, =NR^{5a}, —NR⁷R⁷, R^{a}, and phenyl.

In some embodiments, each R⁵ independently is H, methyl, —CH₂CO₂H, —CH₂P(O)(OH)₂, —CH₂CH₂CO₂H, —C(O)OCH₃, —C(═NH)NH₂, —C(O)C(O)OH,

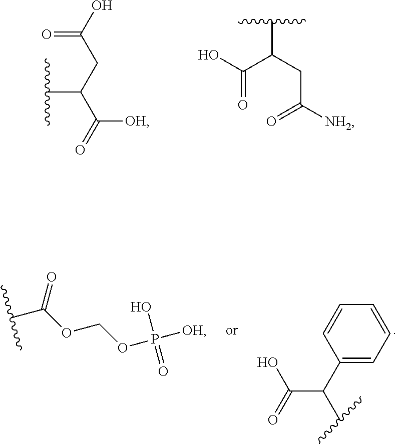 [SMILES: *C(CC(=O)O)C(=O)O ; *C(CC(N)=O)C(=O)O ; *C(=O)OCOP(=O)(O)O ; *C(C(=O)O)c1ccccc1]

In some embodiments, each R⁵ independently is H, methyl, —CH₂CO₂H, —CH₂CH₂CO₂H, —C(O)OCH₃, —C(═NH)NH₂, —C(O)C(O)OH,

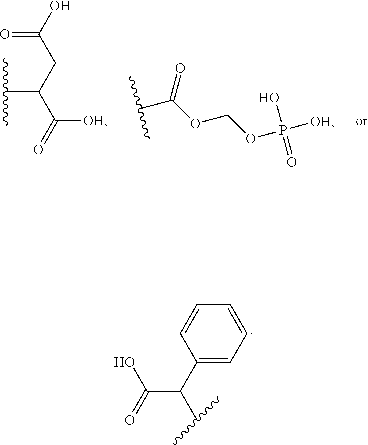 [SMILES: *C(CC(=O)O)C(=O)O ; *C(=O)OCOP(=O)(O)O ; *C(C(=O)O)c1ccccc1]

In some embodiments, one or more R⁵ is H. In some embodiments, one or more R⁵ is R^{d}. In some embodiments, one or more R⁵ is —C(O)OR⁶. In some embodiments, one or more R⁵ is —C(O)C(O)OR⁶.

In some embodiments, one or more R⁵ is C₁₋₆ alkyl, wherein the C₁₋₆ alkyl is optionally substituted with 1-3 groups independently selected from —CN, halogen, =NR^{5a}, R^{a}, R^{d}R^{e}, phenyl, naphthalenyl, and 8-10 membered fused bicyclic heteroaryl. In some embodiments, one or more R⁵ is C₁₋₆ alkyl, wherein the C₁₋₆ alkyl is optionally substituted with 1-3 groups independently selected from —CN, halogen, =NR^{5a}, R^{a}, R^{d}, R^{e}, phenyl, and naphthalenyl. In some embodiments, one or more R⁵ is C₁₋₆ alkyl, wherein the C₁₋₆ alkyl is optionally substituted with 1-3 groups independently selected from —OH, —CN, halogen, —C(O)OR⁶, NR^{5a}, —NR⁷R⁷, R^{a}, and phenyl. In some embodiments, one or more R⁵ is C₁₋₄ alkyl, wherein the C₁₋₄ alkyl is optionally substituted with 1-2 groups independently selected from —C(O)OH, =NR^{5a}, —NR⁷R⁷, R^{a}, and phenyl.

In some embodiments, one or more R⁵ is C₁₋₆ alkyl, wherein the C₁₋₆ alkyl is substituted with 1-3 groups independently selected from —CN, halogen, =NR^{5a}, R^{a}, R^{d}, R^{e}, phenyl, naphthalenyl, and 8-10 membered fused bicyclic heteroaryl. In some embodiments, one or more R⁵ is C₁₋₆ alkyl, wherein the C₁₋₆ alkyl is substituted with 1-3 groups independently selected from —CN, halogen, =NR^{5a}, R^{a}, R^{d}, R^{e}, phenyl, and naphthalenyl. In some embodiments, one or more R⁵ is C₁₋₆ alkyl, wherein the C₁₋₆ alkyl is substituted with 1-3 groups independently selected from —OH, —CN, halogen, —C(O)OR⁶, =NR^{5a}, —NR⁷R⁷, R^{a}, and phenyl. In some embodiments, one or more R⁵ is C₁₋₄ alkyl, wherein the C₁₋₄ alkyl is substituted with 1-2 groups independently selected from —C(O)OH, =NR^{5a}, —NR⁷R⁷, R^{a}, and phenyl.

In some embodiments, one or more R⁵ is C₁₋₆ alkyl. In some embodiments, one or more R⁵ is C₁₋₄ alkyl.

In some embodiments, one or more R⁵ is 5-6 membered monocyclic heteroaryl, wherein the 5-6 membered monocyclic heteroaryl is optionally substituted with 1-3 groups independently selected from —CN, halogen, R^{a}, R^{d}, and R^{e}. In some embodiments, one or more R⁵ is 5-6 membered monocyclic heteroaryl, wherein the 5-6 membered monocyclic heteroaryl is substituted with 1-3 groups independently selected from —CN, halogen, R^{a}, R^{d}, and R^{e}. In some embodiments, one or more R⁵ is 5-6 membered monocyclic heteroaryl.

In some embodiments, one R⁵ is

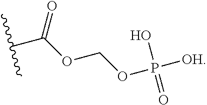 [SMILES: *C(=O)OCOP(=O)(O)O]

In some embodiments, one R⁵ is

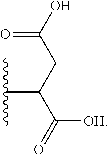 [SMILES: *C(CC(=O)O)C(=O)O]

In some embodiments, one R⁵ is

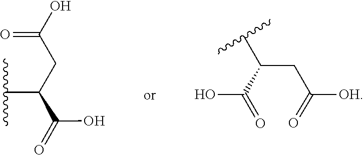 [SMILES: *[C@@H](CC(=O)O)C(=O)O ; *[C@H](CC(=O)O)C(=O)O]

In some embodiments, one R⁵ is

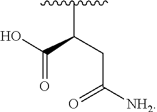 [SMILES: *[C@@H](CC(N)=O)C(=O)O]

In some embodiments, each R^{5a} independently is H or C₁₋₃ alkyl. In some embodiments, each R^{5a} independently is H or methyl. In some embodiments, one or more R^{5a} is H. In some embodiments, one or more R^{5a} is C₁₋₃ alkyl. In some embodiments, one or more R^{5a} is methyl.

In some embodiments, each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶. In some embodiments, one or more R^{d} is —C(O)R⁶. In some embodiments, one or more R^{d} is —C(O)OR⁶. In some embodiments, one or more R^{d} is —C(O)NR⁷R⁷. In some embodiments, one or more R^{d} is —C(O)C(O)OR⁶. In some embodiments, one or more R^{d} is —S(O)₂R⁶. In some embodiments, one or more R^{d} is —S(O)₂NR⁷R⁷. In some embodiments, one or more R^{d} is —S(O)₂OR⁶.

In some embodiments, each R^{c} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶. In some embodiments, one or more R^{c} is —OR⁶. In some embodiments, one or more R^{c} is —OC(O)R⁶. In some embodiments, one or more R^{c} is —OC(O)C(O)OR⁶. In some embodiments, one or more R^{c} is —NR⁷R⁷. In some embodiments, one or more R^{c} is —NR⁷C(O)R⁷. In some embodiments, one or more R^{c} is —NR⁷C(O)NR⁷R⁷. In some embodiments, one or more R^{e} is —NR⁷C(O)OR⁶. In some embodiments, one or more R^{c} is —NR⁷C(O)C(O)OR⁶. In some embodiments, one or more R^{c} is —NR'S(O)₂R⁶.

In some embodiments, each R⁶ independently is H or C₁₋₆ alkyl, wherein the C₁₋₆ alkyl is optionally substituted with 1-3 groups independently selected from CN, halogen, R^{a}, R^{f}, and R⁹. In some embodiments, each R⁶ independently is H or C₁₋₆ alkyl, wherein the C₁₋₆ alkyl is optionally substituted with 1-3 groups independently selected from —OH, CN, halogen, —C(O)OH, and R^{a}. In some embodiments, R⁶ is H or C₁₋₃ alkyl, wherein the C₁₋₃ alkyl is optionally substituted with 1-2 R^{a} groups.

In some embodiments, one or more R⁶ is H. In some embodiments, one or more R⁶ is C₁₋₆ alkyl, wherein the C₁₋₆ alkyl is optionally substituted with 1-3 groups independently selected from CN, halogen, R^{a}, R^{f}, and R⁹. In some embodiments, one or more R⁶ is C₁₋₆ alkyl, wherein the C₁₋₆ alkyl is optionally substituted with 1-3 groups independently selected from —OH, CN, halogen, —C(O)OH, and R. In some embodiments, one or more R⁶ is C₁₋₃ alkyl, wherein the C₁₋₃ alkyl is optionally substituted with 1-2 R^{a} groups.

In some embodiments, one or more R⁶ is C₁₋₆ alkyl, wherein the C₁₋₆ alkyl is substituted with 1-3 groups independently selected from CN, halogen, R^{a}, R^{f}, and R⁹. In some embodiments, one or more R⁶ is C₁₋₆ alkyl, wherein the C₁₋₆ alkyl is substituted with 1-3 groups independently selected from —OH, CN, halogen, —C(O)OH, and R^{a}. In some embodiments, one or more R⁶ is C₁₋₃ alkyl, wherein the C₁₋₃ alkyl is substituted with 1-2 R^{a} groups. In some embodiments, one or more R⁶ is C₁₋₆ alkyl. In some embodiments, one or more R⁶ is C₁₋₃ alkyl.

In some embodiments, each R⁷ independently is H, R^{f}, or C₁₋₆ alkyl, wherein the C₁₋₆ alkyl is optionally substituted with 1-3 groups independently selected from —CN, halogen, R^{a}, R^{f}, and R⁹. In some embodiments, each R⁷ independently is H or C₁₋₆ alkyl, wherein the C₁₋₆ alkyl is optionally substituted with 1-3 groups independently selected from —OH, —CN, halogen, —C(O)OH, and R^{a}. In some embodiments, each R⁷ independently is H or C₁₋₃ alkyl, wherein the C₁₋₃ alkyl is optionally substituted with 1-3 groups independently selected from —OH, —CN, halogen, —C(O)OH, and R^{a}.

In some embodiments, one or more R⁷ is H. In some embodiments, one R⁷ is H. In some embodiments, one or more R⁷ is R^{f}. In some embodiments, one or more R⁷ is C₁₋₆ alkyl, wherein the C₁₋₆ alkyl is optionally substituted with 1-3 groups independently selected from —CN, halogen, R^{a}, R^{f}, and R⁹. In some embodiments, one or more R⁷ is C₁₋₆ alkyl, wherein the C₁₋₆ alkyl is optionally substituted with 1-3 groups independently selected from —OH, —CN, halogen, —C(O)OH, and R^{a}. In some embodiments, one or more R⁷ is C₁₋₃ alkyl, wherein the C₁₋₃ alkyl is optionally substituted with 1-3 groups independently selected from —OH, —CN, halogen, —C(O)OH, and R^{a}. In some embodiments, one or more R⁷ is C₁₋₆ alkyl, wherein the C₁₋₆ alkyl is substituted with 1-3 groups independently selected from —CN, halogen, R^{a}, R^{f}, and R⁹. In some embodiments, one or more R⁷ is C₁₋₆ alkyl, wherein the C₁₋₆ alkyl is substituted with 1-3 groups independently selected from —OH, —CN, halogen, —C(O)OH, and R^{a}. In some embodiments, one or more R⁷ is C₁₋₃ alkyl, wherein the C₁₋₃ alkyl is substituted with 1-3 groups independently selected from —OH, —CN, halogen, —C(O)OH, and R. In some embodiments, one or more R⁷ is C₁₋₆ alkyl. In some embodiments, one or more R⁷ is C₁₋₃ alkyl.

In some embodiments, each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸. In some embodiments, one or more R^{f} is —C(O)R⁸. In some embodiments, one or more R^{f} is —C(O)OR⁸. In some embodiments, one or more R^{f} is —C(O)NR⁸R⁸. In some embodiments, one or more R^{f} is —C(O)C(O)OR⁸. In some embodiments, one or more R^{f} is —S(O)₂R⁸. In some embodiments, one or more R^{f} is —S(O)₂NR⁸R⁸. In some embodiments, one or more R^{f} is —S(O)₂OR⁸.

In some embodiments, each R⁹ independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR⁸S(O)₂R⁸. In some embodiments, one or more R⁹ is —OR⁸. In some embodiments, one or more R⁹ is —OC(O)R⁸. In some embodiments, one or more R⁹ is —OC(O)C(O)OR⁸. In some embodiments, one or more R⁹ is —NR⁸R⁸. In some embodiments, one or more R⁹ is —NR⁸C(O)R⁸. In some embodiments, one or more R⁹ is —NR⁸C(O)NR⁸R⁸. In some embodiments, one or more R⁹ is —NR⁸C(O)OR⁸. In some embodiments, one or more R⁹ is —NR⁸C(O)C(O)OR⁸. In some embodiments, one or more R⁹ is —NR'S(O)₂R⁸.

In some embodiments, each R⁸ independently is H or C₁₋₆ alkyl, wherein the C₁₋₆ alkyl is optionally substituted with 1-3 groups independently selected from —OH, CN, halogen, —C(O)OH, and R^{a}. In some embodiments, one or more R⁸ is H. In some embodiments, one or more R⁸ is C₁₋₆ alkyl, wherein the C₁₋₆ alkyl is optionally substituted with 1-3 groups independently selected from —OH, CN, halogen, —C(O)OH, and R^{a}. In some embodiments, one or more R⁸ is C₁₋₆ alkyl, wherein the C₁₋₆ alkyl is substituted with 1-3 groups independently selected from —OH, CN, halogen, —C(O)OH, and R^{a}. In some embodiments, one or more R⁸ is C₁₋₆ alkyl.

In some embodiments, the compound provided herein is a compound selected from the group consisting of:

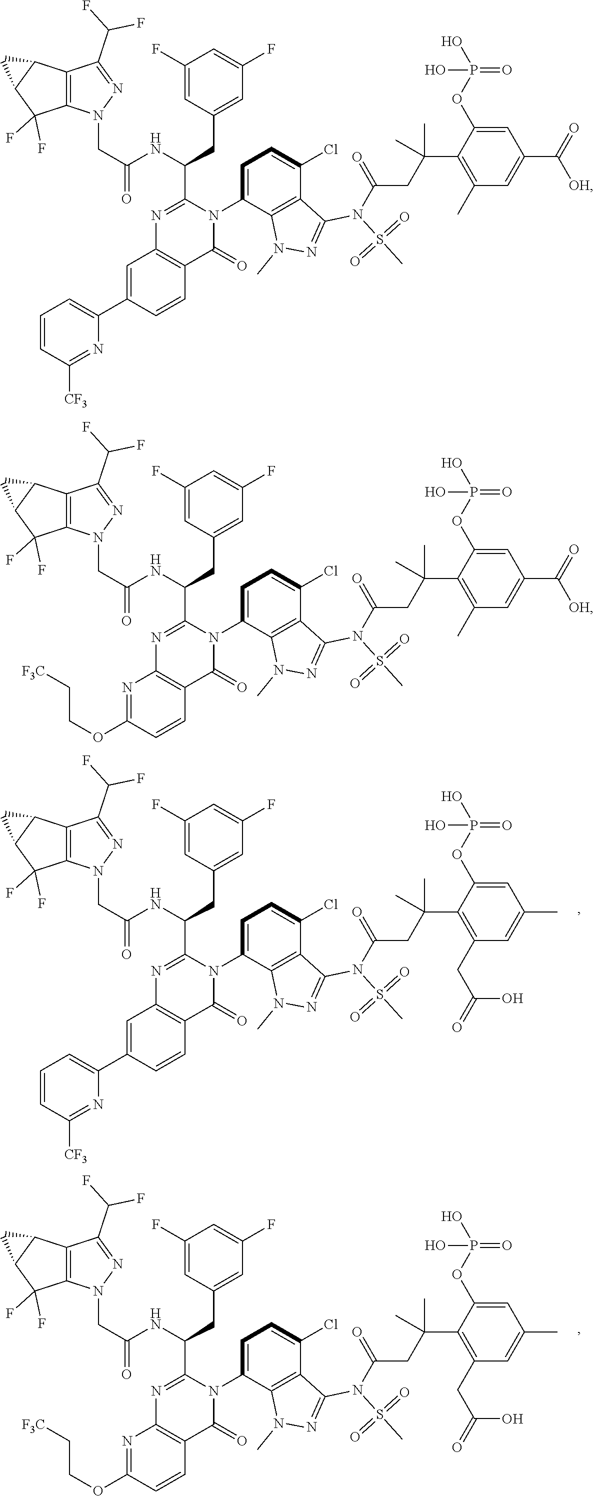

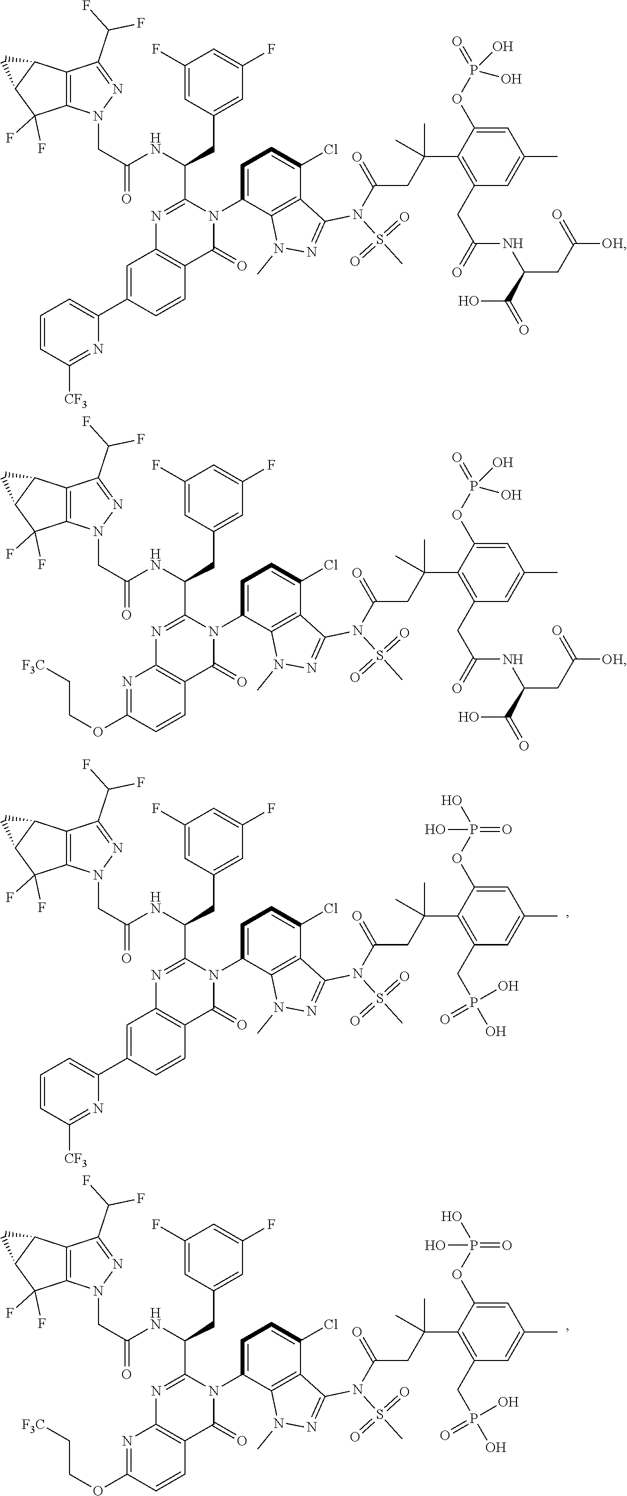

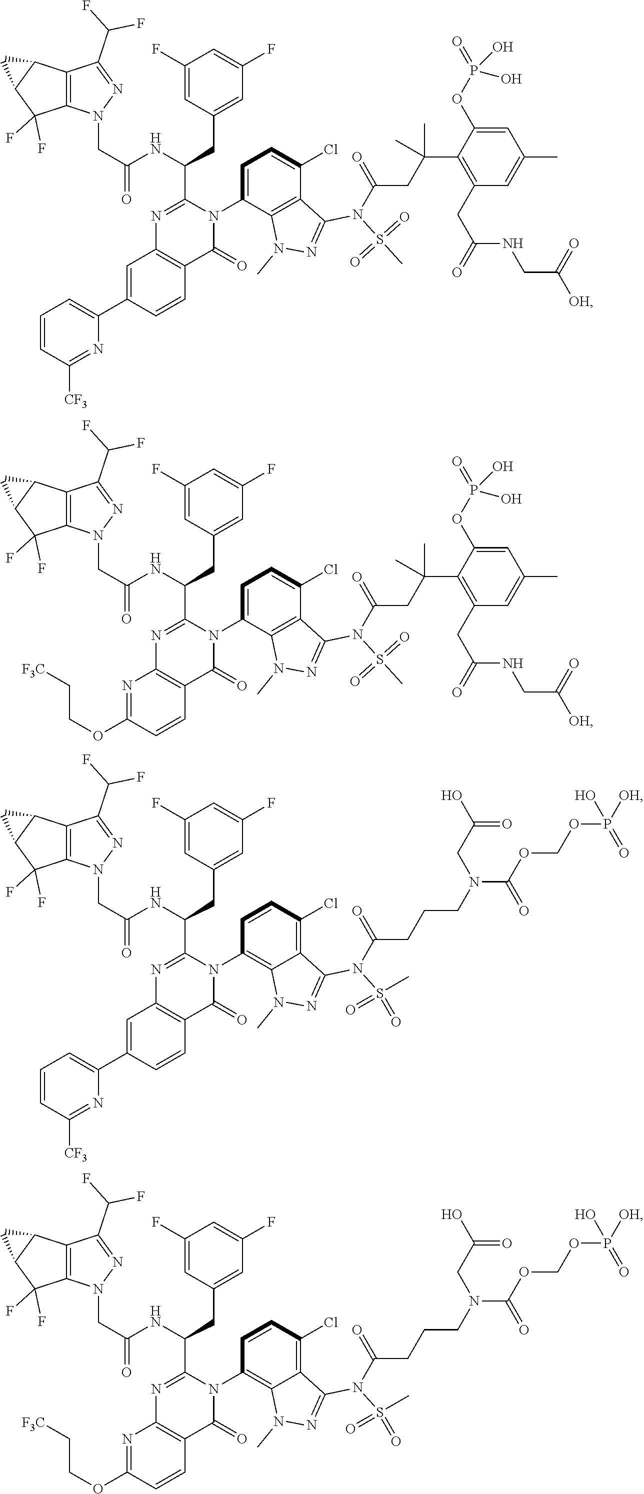

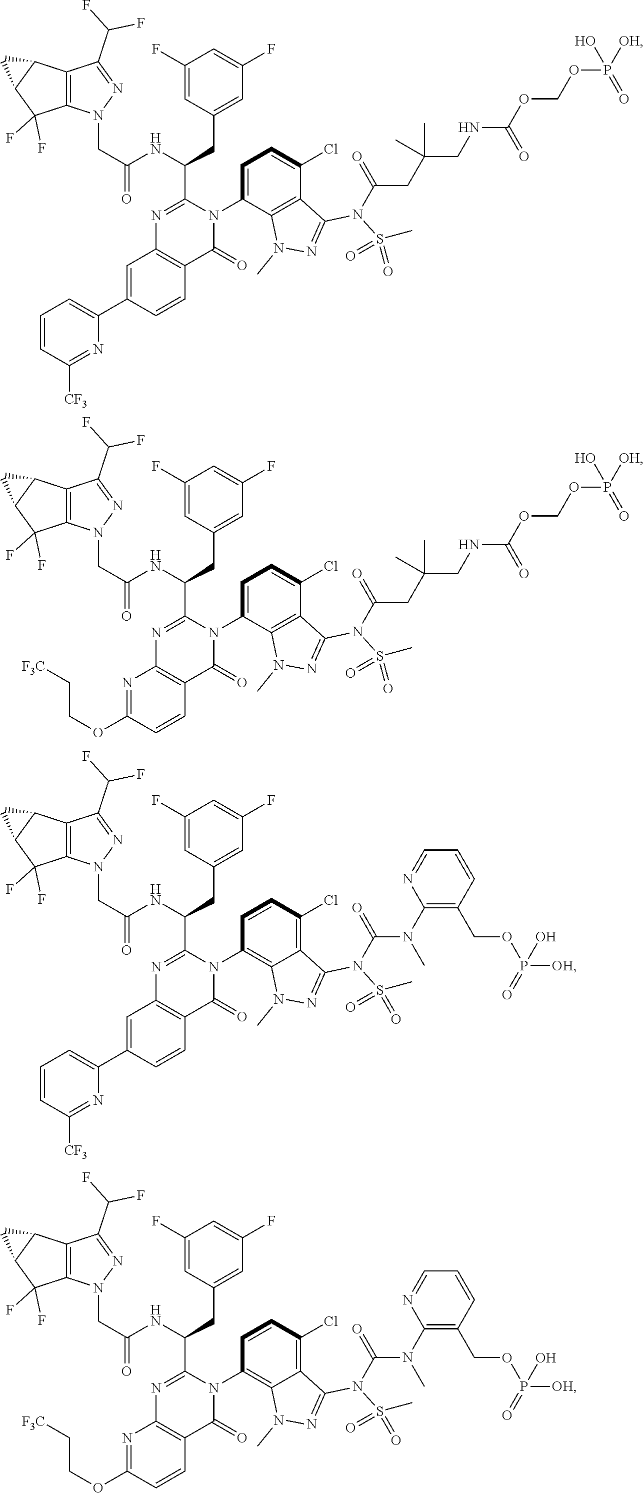

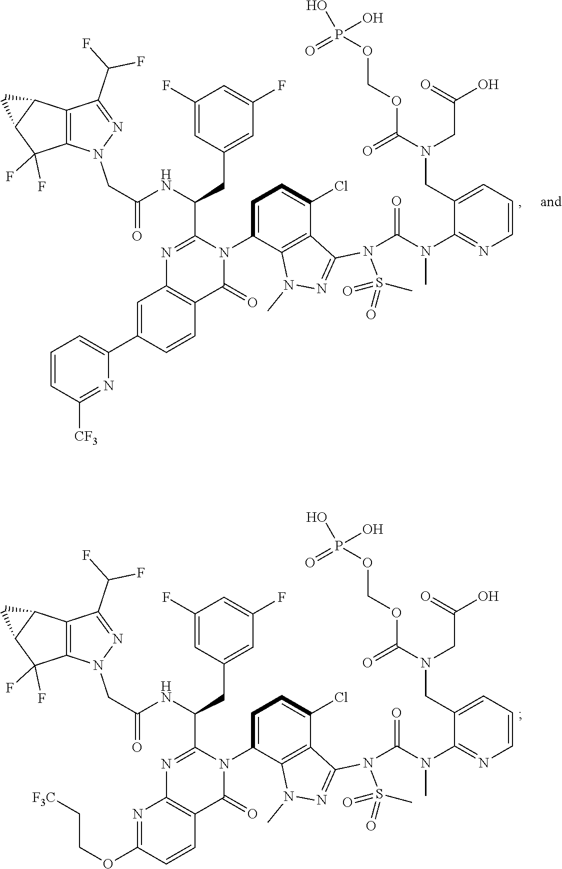

or a pharmaceutically acceptable salt thereof.

In some embodiments, the compound provided herein is a compound selected from the group consisting of:

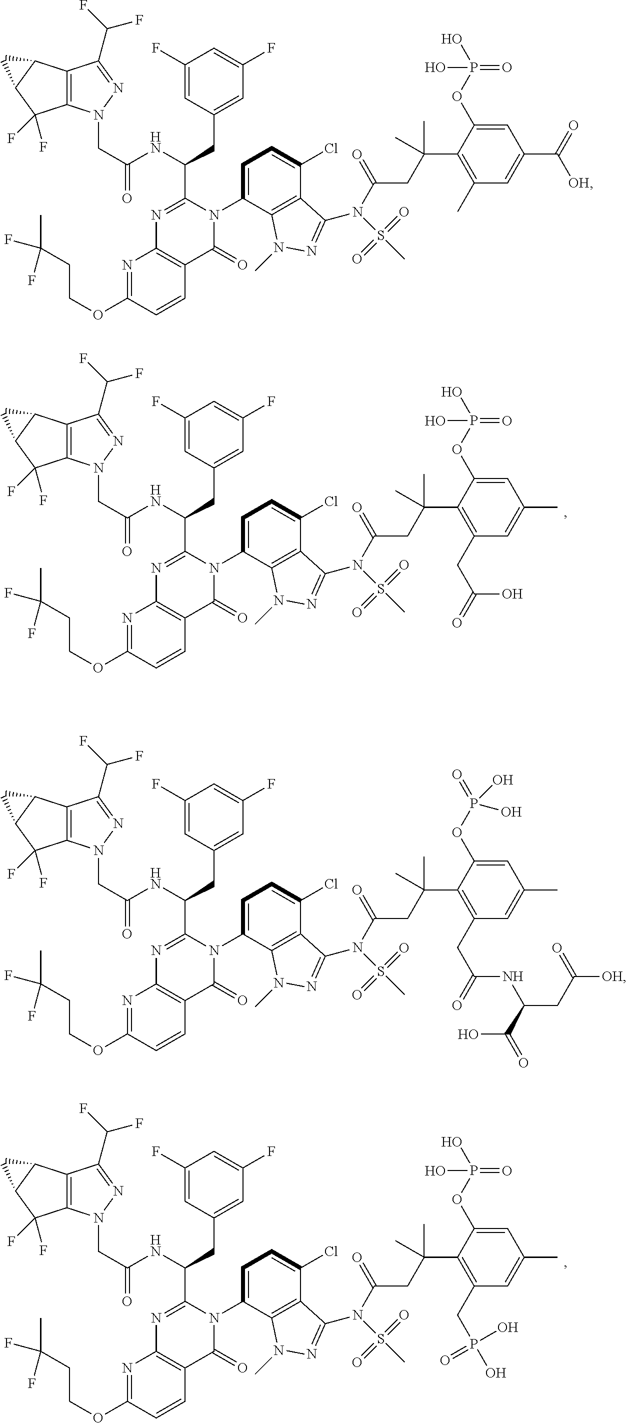

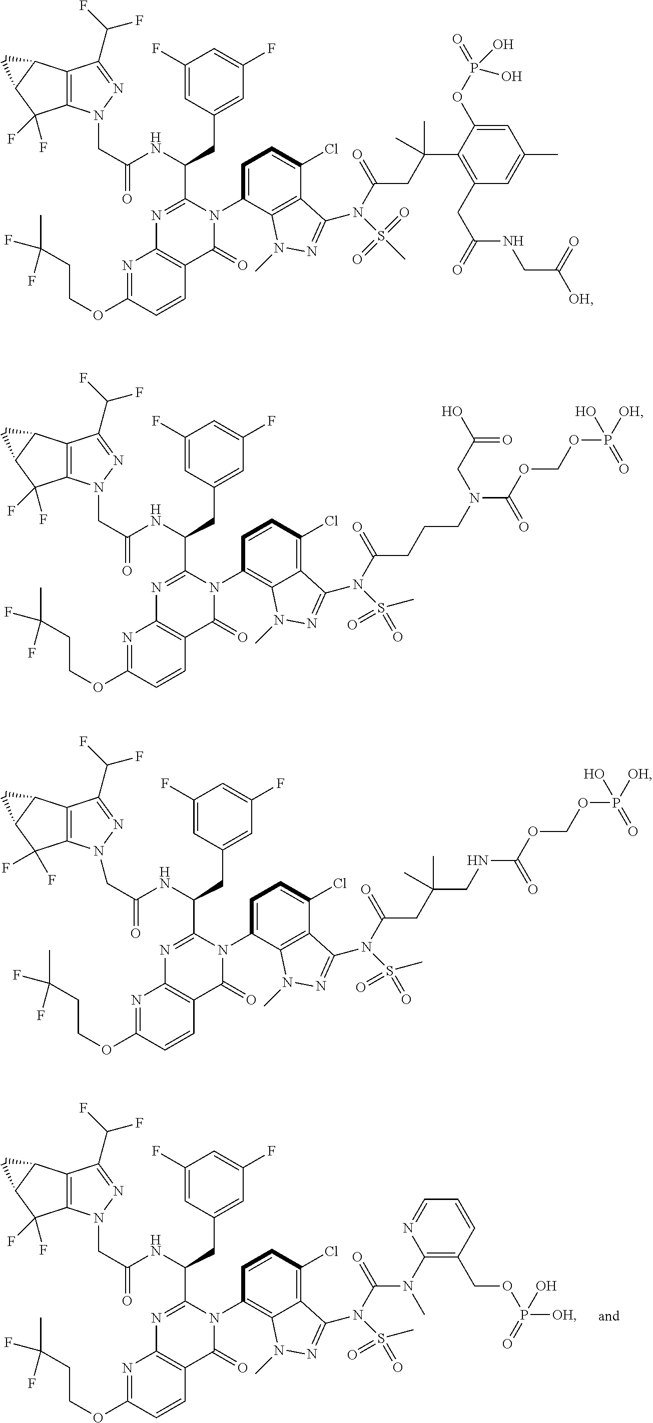

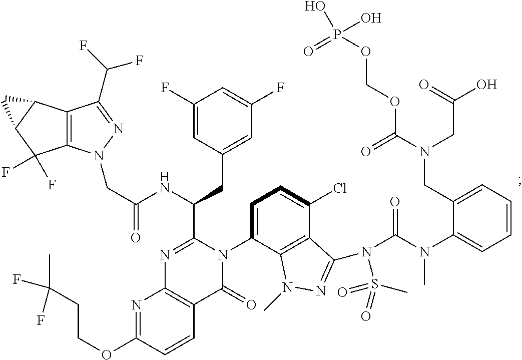

or a pharmaceutically acceptable salt thereof.

In some embodiments, the compound provided herein is a compound selected from the group consisting of:

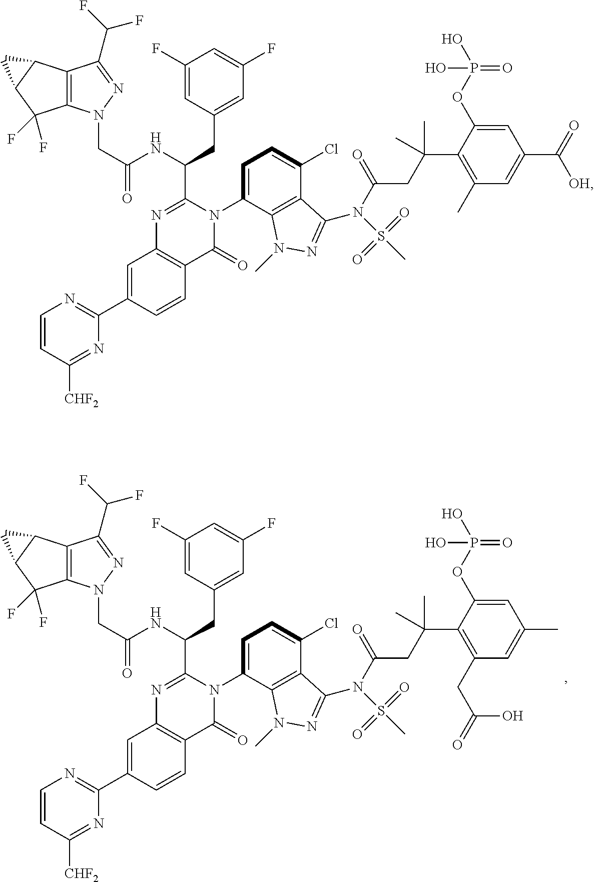

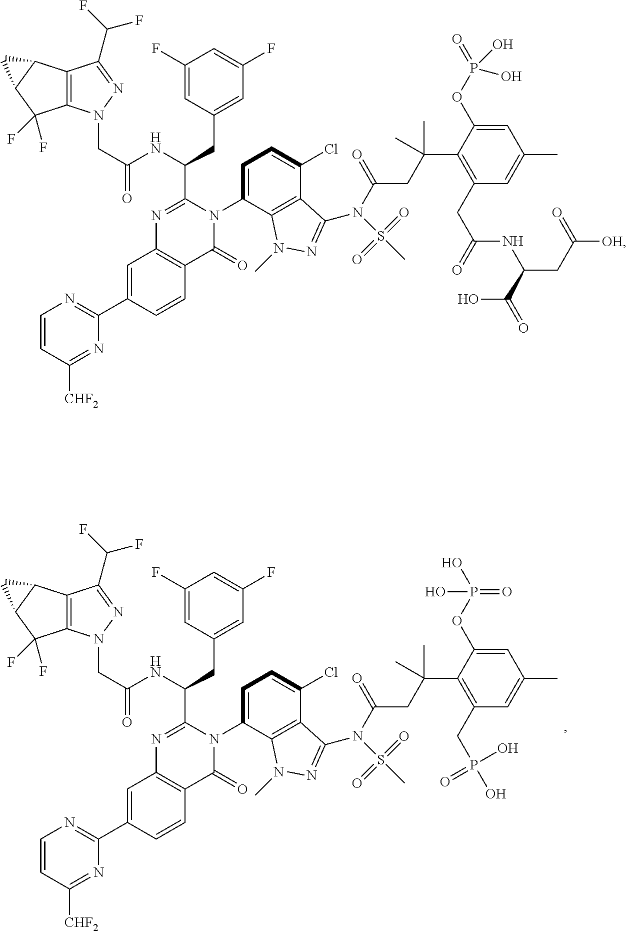

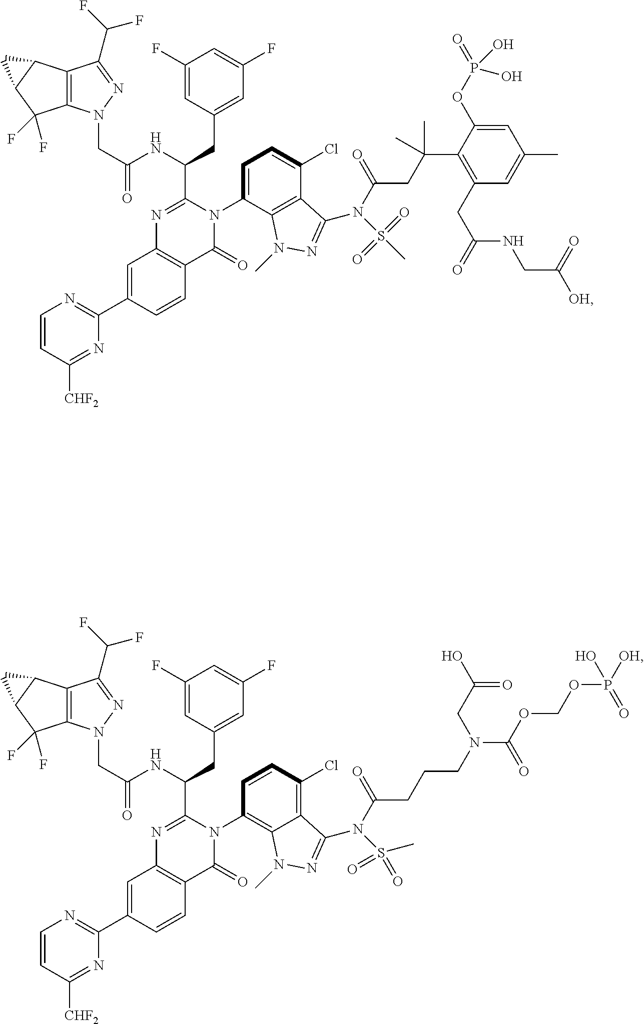

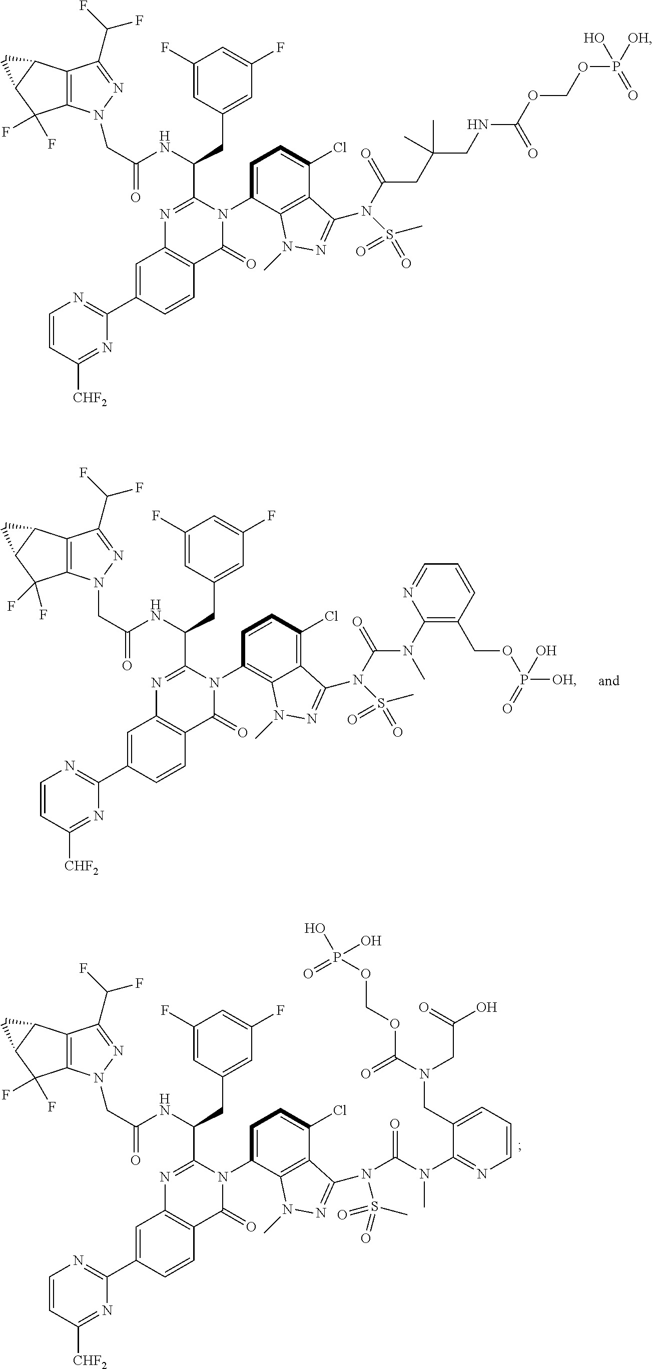

or a pharmaceutically acceptable salt thereof.

In some embodiments, the compounds of Formula I may metabolize to compounds of Intermediate A in the body (e.g., a compound of Formula I may metabolize to a compound of Intermediate A, which is a metabolite of the compound of Formula I, upon administration to a subject such as a human). A skilled artisan will readily recognize which specific compound of Formula I will metabolize to a specific compound of Intermediate A in the body (e.g., in a human body). In some embodiments, the compounds of Formula I are prodrugs of the compounds of Intermediate A. In some embodiments, the compounds of Intermediate A are metabolites of the compounds of Formula I. In some embodiments, the compounds of Formula I are more soluble than the compounds of Intermediate A in a given solvent (e.g., a compound of Formula I is more soluble than the corresponding compound of Intermediate A in a given solvent). In some embodiments, the compounds of Formula I can be orally administered at a lower dose than the compounds of Intermediate A while still achieving the requisite level of bioavailability in the body for biological activity. In some embodiments, the compounds of Intermediate A have activity against HIV. Non-limiting examples of compounds of Intermediate A are disclosed and described in U.S. Ser. No. 10/954,252, U.S. Ser. No. 11/505,543, US2022089598, US2021323961, U.S. Ser. No. 11/541,055, US2021395262, US2021393633, US2021403465, US2021379071, US2021395248, US2022105096, US2022211704, US2021323967, US2022409619, US2022389007, US2023013823, US2022370451, US2023045509, US2023106880, and US2023149408, the contents of each of which are hereby incorporated by reference in their entireties. Additional examples of Intermediate A, their methods of preparation, and their biological activities are disclosed and described in WO2021/176366 and in Gillis, E. et al. J. Med. Chem. 2023, 66 (3), 1941-1954 (https://pubs.acs.org/doi/pdf/10.1021/acs.jmedchem.2c01732).

### III. Compositions and Kits

Compounds provided herein, or pharmaceutically acceptable salts thereof, are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein, or pharmaceutically acceptable salts thereof, may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising a compound provided herein (i.e., a compound of Formula I), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (i.e., one, two, three, four; one or two; one to three; or one to four) additional therapeutic agents, or a pharmaceutically acceptable salt thereof. In some embodiments, the pharmaceutical compositions further comprise a therapeutically effective amount of the one or more (i.e., one, two, three, four; one or two; one to three; or one to four) additional therapeutic agents, or a pharmaceutically acceptable salt thereof.

In some embodiments, the one or more additional therapeutic agents include agents that are therapeutic for an HIV virus infection. In some embodiments, the one or more additional therapeutic agents is an anti-HIV agent. In some embodiments, the one or more additional therapeutic agents is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and any combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and any combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and any combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and any combinations thereof.

In some embodiments, the additional therapeutic agent or agents are chosen from dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, tenofovir alafenamide hemifumarate, and islatravir or a pharmaceutically acceptable salt thereof.

In some embodiments, the additional therapeutic agent or agents are chosen from dolutegravir, cabotegravir, darunavir, bictegravir, elsulfavirine, rilpivirine, and any combinations thereof, or a pharmaceutically acceptable salt thereof.

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir +abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

In one embodiment, provided herein are pharmaceutical compositions comprising a therapeutically effective amount of a compound provided herein, or a pharmaceutically acceptable salt thereof, and a pharmaceutically acceptable excipient.

In some embodiments, the pharmaceutical compositions provided herein further comprise one, two, three, or four additional therapeutic agents.

In some embodiments, the pharmaceutical compositions provided herein further comprise one, two, three, or four additional therapeutic agents, wherein the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MILK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments, the pharmaceutical compositions provided herein further comprise one, two, three, or four additional therapeutic agents, wherein the additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, “antibody-like” therapeutic proteins, or any combinations thereof.

In some embodiments, the pharmaceutical compositions provided herein further comprise one, two, three, or four additional therapeutic agents, wherein the additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, and tenofovir alafenamide hemifumarate, or a pharmaceutically acceptable salt thereof.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the compounds, or pharmaceutically acceptable salts thereof, and pharmaceutical compositions disclosed herein are administered by subcutaneous injection.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6^{th} edition 2009. Examples of solubilizing excipients in a parenteral formulation (e.g., an SC or IM formulation) include, but are not limited to, polysorbates (such as polysorbate 20 or 80) and poloxamers (such as poloxamer 338, 188, or 207).

In some embodiments, the compounds, or pharmaceutically acceptable salts thereof, and pharmaceutical compositions disclosed herein are administered with implants.

Oral administration may be another route for administration of the compounds provided herein or pharmaceutically acceptable salts thereof. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds provided herein or pharmaceutically acceptable salts thereof may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one embodiment, provided herein are kits that comprise a compound provided herein, (i.e., a compound of Formula I), or a pharmaceutically acceptable salt, stereoisomer, prodrug, or solvate thereof, and suitable packaging. In some embodiments, the kit further comprises instructions for use. In some embodiments, the kit comprises a compound provided herein (i.e., a compound of Formula I), or a pharmaceutically acceptable salt, stereoisomer, prodrug, or solvate thereof, and a label and/or instructions for use of the compounds in the treatment of the indications, including the diseases or conditions, described herein.

In some embodiments, the kits further comprise one or more (i.e., one, two, three, four; one or two; one to three; or one to four) additional therapeutic agents, or a pharmaceutically acceptable salt thereof.

In one embodiment, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

### IV. Methods

The methods provided herein may be applied to cell populations in vivo or ex vivo. “In vivo” means within a living individual, as within an animal or human. In this context, the methods provided herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. Exemplary tissue samples include tumors and biopsies thereof. In this context, the present disclosure may be used for a variety of purposes, including therapeutic and experimental purposes. For example, the present disclosure may be used ex vivo to determine the optimal schedule and/or dosing of administration of a compound as disclosed herein for a given cell type, individual, and other parameters. Information gleaned from such use may be used for experimental purposes or in the clinic to set protocols for in vivo treatment. Other ex vivo uses for which the present disclosure may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, the present disclosure provides a method of treating or preventing a human immunodeficiency virus (HIV) infection in a patient in need thereof comprising administering to the patient a therapeutically effective amount of a compound provided herein, or a pharmaceutically acceptable salt thereof, or a pharmaceutical composition provided herein.

In one embodiment, the present disclosure provides a method of treating a human immunodeficiency virus (HIV) infection in a heavily treatment-experienced patient, the method comprising administering to the patient a therapeutically effective amount of a compound provided herein, or a pharmaceutically acceptable salt thereof, or a pharmaceutical composition provided herein.

In some embodiments, the methods provided herein further comprise administering a therapeutically effective amount of one, two, three, or four additional therapeutic agents, or a pharmaceutically acceptable salt thereof.

In some embodiments, the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MILK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments, the one, two, three, or four additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, and “antibody-like” therapeutic proteins, or any combinations thereof.

In some embodiments, the one, two, three, or four additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, tenofovir alafenamide hemifumarate, and islatravir or a pharmaceutically acceptable salt thereof.

In some embodiments of the methods provided herein, the patient is a human.

In one embodiment, the present disclosure provides a therapeutically effective amount of a compound provided herein, or a pharmaceutically acceptable salt thereof, or a pharmaceutical composition provided herein for use in therapy.

In one embodiment, the present disclosure provides a compound provided herein, or a pharmaceutically acceptable salt, or a pharmaceutical composition provided herein for use in a method of treating or preventing a human immunodeficiency virus (HIV) infection in a patient in need thereof, the method comprising administering to the patient a therapeutically effective amount of the compound, or a pharmaceutically acceptable salt thereof, or the pharmaceutical composition.

In one embodiment, the present disclosure provides a compound provided herein, or a pharmaceutically acceptable salt thereof, or a pharmaceutical composition provided herein for use in a method of treating a human immunodeficiency virus (HIV) infection in a heavily treatment-experienced patient, the method comprising administering to the patient a therapeutically effective amount of the compound, or a pharmaceutically acceptable salt thereof, or the pharmaceutical composition.

In some embodiments, the uses provided herein further comprise administering a therapeutically effective amount of one, two, three, or four additional therapeutic agents, or a pharmaceutically acceptable salt thereof.

In some embodiments of the uses provided herein, the one, two, three, or four additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MILK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, and anti-HIV peptides, or any combinations thereof.

In some embodiments of the uses provided herein, the one, two, three, or four additional therapeutic agents are selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, bispecific antibodies, and “antibody-like” therapeutic proteins, or any combinations thereof.

In some embodiments of the uses provided herein, the one, two, three, or four additional therapeutic agents are selected from the group consisting of dolutegravir, cabotegravir, darunavir, bictegravir, elsulfavirine, rilpivirine, abacavir sulfate, tenofovir, tenofovir disoproxil, tenofovir disoproxil fumarate, tenofovir disoproxil hemifumarate, tenofovir alafenamide, tenofovir alafenamide hemifumarate, and islatravir, or a pharmaceutically acceptable salt thereof.

In some embodiments of the uses provided herein, the patient is a human.

### V. Administration

The compounds of the present disclosure or pharmaceutically acceptable salts thereof (also referred to herein as the active ingredients) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. An advantage of certain compounds disclosed herein, or pharmaceutically acceptable salts thereof, is that they are orally bioavailable and can be dosed orally.

A compound of the present disclosure, or a pharmaceutically acceptable salt thereof, may be administered to an individual in accordance with an effective dosing regimen for a desired period of time or duration, such as at least about one month, at least about 2 months, at least about 3 months, at least about 6 months, or at least about 12 months or longer. In some embodiments, the compound, or a pharmaceutically acceptable salt thereof, is administered on a daily or intermittent schedule for the duration of the individual's life.

The specific dose level of a compound of the present disclosure, or a pharmaceutically acceptable salt thereof, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound provided herein, or a pharmaceutically acceptable salt thereof, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein, or a pharmaceutically acceptable salt thereof, administered per dose. The dosage or dosing frequency of a compound of the present disclosure, or a pharmaceutically acceptable salt thereof, may be adjusted over the course of the treatment, based on the judgment of the administering physician.

The compounds of the present disclosure, or pharmaceutically acceptable salts thereof, may be administered to an individual (e.g., a human) in a therapeutically effective amount. In some embodiments, the compound of Formula I, or a pharmaceutically acceptable salt thereof, is administered once daily, once weekly, once monthly, once every two months, once every three months, or once every six months. In some embodiments, the compound of Formula I, or a pharmaceutically acceptable salt thereof, is administered once daily. In some embodiments, the compound of Formula I, or a pharmaceutically acceptable salt thereof, is administered once weekly. In some embodiments, the compound of Formula I, or a pharmaceutically acceptable salt thereof, is administered once monthly. In some embodiments, the compound of Formula I, or a pharmaceutically acceptable salt thereof, is administered once every two months. In some embodiments, the compound of Formula I, or a pharmaceutically acceptable salt thereof, is administered once every three months. In some embodiments, the compound of Formula I, or a pharmaceutically acceptable salt thereof, is administered once every six months.

The compounds provided herein, or pharmaceutically acceptable salts thereof, can be administered by any useful route and means, such as by oral or parenteral (e.g., intravenous) administration. Therapeutically effective amounts of the compound, or a pharmaceutically acceptable salt thereof, may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein, or pharmaceutically acceptable salts thereof, include from about 0.3 mg to about 30 mg per day, or from about 30 mg to about 300 mg per day, or from about 0.3 μg to about 30 mg per day, or from about 30 μg to about 300 μg per day.

A compound of the present disclosure, or a pharmaceutically acceptable salt thereof, may be combined with one or more additional therapeutic agents in any dosage amount of the compound of the present disclosure or a pharmaceutically acceptable salt thereof (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula I, or a pharmaceutically acceptable salt thereof, are about 50, 100, 125, 150, 175, 200, 225, 250, 275, or 300 mg per dose. Other therapeutically effective amounts of the compound of Formula I, or pharmaceutically acceptable salts thereof, are about 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, a therapeutically effective amount of the compound of Formula I, or a pharmaceutically acceptable salt thereof, is about 1 mg to about 1000 mg. In some embodiments, a therapeutically effective amount of the compound of Formula I, or a pharmaceutically acceptable salt thereof, is about 1 mg to about 900 mg. In some embodiments, a therapeutically effective amount of the compound of Formula I, or a pharmaceutically acceptable salt thereof, is about 1 mg to about 800 mg. In some embodiments, a therapeutically effective amount of the compound of Formula I, or a pharmaceutically acceptable salt thereof, is about 1 mg to about 700 mg. In some embodiments, a therapeutically effective amount of the compound of Formula I, or a pharmaceutically acceptable salt thereof, is about 1 mg to about 600 mg. In some embodiments, a therapeutically effective amount of the compound of Formula I, or a pharmaceutically acceptable salt thereof, is about 1 mg to about 500 mg. In some embodiments, a therapeutically effective amount of the compound of Formula I, or a pharmaceutically acceptable salt thereof, is about 1 mg to about 400 mg. In some embodiments, a therapeutically effective amount of the compound of Formula I, or a pharmaceutically acceptable salt thereof, is about 1 mg to about 300 mg. In some embodiments, a therapeutically effective amount of the compound of Formula I, or a pharmaceutically acceptable salt thereof, is about 1 mg to about 200 mg. In some embodiments, a therapeutically effective amount of the compound of Formula I, or a pharmaceutically acceptable salt thereof, is about 1 mg to about 100 mg. In some embodiments, a therapeutically effective amount of the compound of Formula I, or a pharmaceutically acceptable salt thereof, is about 1 mg to about 75 mg. In some embodiments, a therapeutically effective amount of the compound of Formula I, or a pharmaceutically acceptable salt thereof, is about 1 mg to about 50 mg. In some embodiments, a therapeutically effective amount of the compound of Formula I, or a pharmaceutically acceptable salt thereof, is about 1 mg to about 25 mg. In some embodiments, a therapeutically effective amount of the compound of Formula I, or a pharmaceutically acceptable salt thereof, is about 1 mg to about 20 mg. In some embodiments, a therapeutically effective amount of the compound of Formula I, or a pharmaceutically acceptable salt thereof, is about 1 mg to about 15 mg. In some embodiments, a therapeutically effective amount of the compound of Formula I, or a pharmaceutically acceptable salt thereof, is about 1 mg to about 10 mg. In some embodiments, a therapeutically effective amount of the compound of Formula I, or a pharmaceutically acceptable salt thereof, is about 1 mg to about 5 mg.

In some embodiments, a therapeutically effective amount of the compound of Formula I, or a pharmaceutically acceptable salt thereof, is about 50 mg, about 75 mg, about 100 mg, about 125 mg, about 150 mg, about 175 mg, about 200 mg, about 225 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or about 1050 mg. In some embodiments, a therapeutically effective amount of the compound of Formula I, or a pharmaceutically acceptable salt thereof, is about 5 mg. In some embodiments, a therapeutically effective amount of the compound of Formula I, or a pharmaceutically acceptable salt thereof, is about 100 mg. In some embodiments, a therapeutically effective amount of the compound of Formula I, or a pharmaceutically acceptable salt thereof, is about 150 mg. In some embodiments, a therapeutically effective amount of the compound of Formula I, or a pharmaceutically acceptable salt thereof, is about 200 mg. In some embodiments, a therapeutically effective amount of the compound of Formula I, or a pharmaceutically acceptable salt thereof, is about 250 mg. In some embodiments, a therapeutically effective amount of the compound of Formula I, or a pharmaceutically acceptable salt thereof, is about 300 mg. In some embodiments, a therapeutically effective amount of the compound of Formula I, or a pharmaceutically acceptable salt thereof, is about 350 mg. In some embodiments, a therapeutically effective amount of the compound of Formula I, or a pharmaceutically acceptable salt thereof, is about 400 mg. In some embodiments, a therapeutically effective amount of the compound of Formula I, or a pharmaceutically acceptable salt thereof, is about 450 mg. In some embodiments, a therapeutically effective amount of the compound of Formula I, or a pharmaceutically acceptable salt thereof, is about 500 mg. In some embodiments, a therapeutically effective amount of the compound of Formula I, or a pharmaceutically acceptable salt thereof, is about 550 mg. In some embodiments, a therapeutically effective amount of the compound of Formula I, or a pharmaceutically acceptable salt thereof, is about 600 mg. In some embodiments, a therapeutically effective amount of the compound of Formula I, or a pharmaceutically acceptable salt thereof, is about 650 mg. In some embodiments, a therapeutically effective amount of the compound of Formula I, or a pharmaceutically acceptable salt thereof, is about 700 mg. In some embodiments, a therapeutically effective amount of the compound of Formula I, or a pharmaceutically acceptable salt thereof, is about 750 mg. In some embodiments, a therapeutically effective amount of the compound of Formula I, or a pharmaceutically acceptable salt thereof, is about 800 mg. In some embodiments, a therapeutically effective amount of the compound of Formula I, or a pharmaceutically acceptable salt thereof, is about 850 mg. In some embodiments, a therapeutically effective amount of the compound of Formula I, or a pharmaceutically acceptable salt thereof, is about 900 mg. In some embodiments, a therapeutically effective amount of the compound of Formula I, or a pharmaceutically acceptable salt thereof, is about 950 mg. In some embodiments, a therapeutically effective amount of the compound of Formula I, or a pharmaceutically acceptable salt thereof, is about 1000 mg. In some embodiments, a therapeutically effective amount of the compound of Formula I, or a pharmaceutically acceptable salt thereof, is about 1050 mg.

When administered orally, the total weekly dosage for a human subject may be between about 1 mg and 1,000 mg/week, between about 10-500 mg/week, between about 50-300 mg/week, between about 75-200 mg/week, or between about 100-150 mg/week. In some embodiments, the total weekly dosage for a human subject may be about 100, 200, 300, 400, 500, 600, 700, 800, 900, or 1000 mg/week administered in a single dose. In some embodiments, the total weekly dosage for a human subject of a compound of Formula I, or a pharmaceutically acceptable salt thereof, may be about 100 mg administered in a single dose. In some embodiments, the total weekly dosage for a human subject of a compound of Formula I, or a pharmaceutically acceptable salt thereof, may be about 150 mg administered in a single dose. In some embodiments, the total weekly dosage for a human subject of a compound of Formula I, or a pharmaceutically acceptable salt thereof, may be about 200 mg administered in a single dose. In some embodiments, the total weekly dosage for a human subject of a compound of Formula I, or a pharmaceutically acceptable salt thereof, may be about 250 mg administered in a single dose. In some embodiments, the total weekly dosage for a human subject of a compound of Formula I, or a pharmaceutically acceptable salt thereof, may be about 300 mg administered in a single dose. In some embodiments, the total weekly dosage for a human subject of a compound of Formula I, or a pharmaceutically acceptable salt thereof, may be about 350 mg administered in a single dose. In some embodiments, the total weekly dosage for a human subject of a compound of Formula I, or a pharmaceutically acceptable salt thereof, may be about 400 mg administered in a single dose. In some embodiments, the total weekly dosage for a human subject of a compound of Formula I, or a pharmaceutically acceptable salt thereof, may be about 450 mg administered in a single dose. In some embodiments, the total weekly dosage for a human subject of a compound of Formula I, or a pharmaceutically acceptable salt thereof, may be about 500 mg administered in a single dose.

When administered orally, the total monthly dosage for a human subject of a compound of Formula I, or a pharmaceutically acceptable salt thereof, may be between about 500 mg and 1,000 mg/month, between about 600-900 mg/month, or between about 700-800 mg/month. In some embodiments, the total weekly dosage for a human subject of a compound of Formula I, or a pharmaceutically acceptable salt thereof, may be about 100, 200, 300, 400, 500, 600, 700, 800, 900, or 1000 mg/week administered in a single dose. In some embodiments, the total monthly dosage for a human subject of a compound of Formula I, or a pharmaceutically acceptable salt thereof, may be about 500 mg administered in a single dose. In some embodiments, the total monthly dosage for a human subject may be about 550 mg administered in a single dose. In some embodiments, the total monthly dosage for a human subject of a compound of Formula I, or a pharmaceutically acceptable salt thereof, may be about 600 mg administered in a single dose. In some embodiments, the total monthly dosage for a human subject of a compound of Formula I, or a pharmaceutically acceptable salt thereof, may be about 650 mg administered in a single dose. In some embodiments, the total monthly dosage for a human subject of a compound of Formula I, or a pharmaceutically acceptable salt thereof, may be about 700 mg administered in a single dose. In some embodiments, the total monthly dosage for a human subject of a compound of Formula I, or a pharmaceutically acceptable salt thereof, may be about 750 mg administered in a single dose. In some embodiments, the total monthly dosage for a human subject of a compound of Formula I, or a pharmaceutically acceptable salt thereof, may be about 800 mg administered in a single dose. In some embodiments, the total monthly dosage for a human subject of a compound of Formula I, or a pharmaceutically acceptable salt thereof, may be about 850 mg administered in a single dose. In some embodiments, the total monthly dosage for a human subject of a compound of Formula I, or a pharmaceutically acceptable salt thereof, may be about 900 mg administered in a single dose. In some embodiments, the total monthly dosage for a human subject of a compound of Formula I, or a pharmaceutically acceptable salt thereof, may be about 950 mg administered in a single dose. In some embodiments, the total monthly dosage for a human subject of a compound of Formula I, or a pharmaceutically acceptable salt thereof, may be about 1000 mg administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound provided herein, or a pharmaceutically acceptable salt thereof, is administered once daily in a method disclosed herein. In some embodiments, a compound provided herein, or a pharmaceutically acceptable salt thereof, is administered twice daily in a method disclosed herein.

In some embodiments, a compound provided herein, or a pharmaceutically acceptable salt thereof, is administered once daily in a method disclosed herein. In some embodiments, a compound provided herein, or a pharmaceutically acceptable salt thereof, is administered once weekly in a method disclosed herein. In some embodiments, a compound provided herein, or a pharmaceutically acceptable salt thereof, is administered once monthly in a method disclosed herein. In some embodiments, a compound provided herein, or a pharmaceutically acceptable salt thereof, is administered once every two months in a method disclosed herein. In some embodiments, a compound provided herein, or a pharmaceutically acceptable salt thereof, is administered once every three months in a method disclosed herein. In some embodiments, a compound provided herein, or a pharmaceutically acceptable salt thereof, is administered once every six months in a method disclosed herein.

In some embodiments, a compound provided herein, or a pharmaceutically acceptable salt thereof, is administered orally in a single dose of about 100 mg once weekly. In some embodiments, a compound provided herein, or a pharmaceutically acceptable salt thereof, is administered orally in a single dose of about 150 mg once weekly. In some embodiments, a compound provided herein, or a pharmaceutically acceptable salt thereof, is administered orally in a single dose of about 200 mg once weekly. In some embodiments, a compound provided herein, or a pharmaceutically acceptable salt thereof, is administered orally in a single dose of about 250 mg once weekly. In some embodiments, a compound provided herein, or a pharmaceutically acceptable salt thereof, is administered orally in a single dose of about 300 mg once weekly. In some embodiments, a compound provided herein, or a pharmaceutically acceptable salt thereof, is administered orally in a single dose of about 350 mg once weekly. In some embodiments, a compound provided herein, or a pharmaceutically acceptable salt thereof, is administered orally in a single dose of about 400 mg once weekly. In some embodiments, a compound provided herein, or a pharmaceutically acceptable salt thereof, is administered orally in a single dose of about 450 mg once weekly. In some embodiments, a compound provided herein, or a pharmaceutically acceptable salt thereof, is administered orally in a single dose of about 500 mg once weekly.

In some embodiments, a compound provided herein, or a pharmaceutically acceptable salt thereof, is administered orally in a single dose of about 500 mg once monthly. In some embodiments, a compound provided herein, or a pharmaceutically acceptable salt thereof, is administered orally in a single dose of about 550 mg once monthly. In some embodiments, a compound provided herein, or a pharmaceutically acceptable salt thereof, is administered orally in a single dose of about 600 mg once monthly. In some embodiments, a compound provided herein, or a pharmaceutically acceptable salt thereof, is administered orally in a single dose of about 650 mg once monthly. In some embodiments, a compound provided herein, or a pharmaceutically acceptable salt thereof, is administered orally in a single dose of about 700 mg once monthly. In some embodiments, a compound provided herein, or a pharmaceutically acceptable salt thereof, is administered orally in a single dose of about 750 mg once monthly. In some embodiments, a compound provided herein, or a pharmaceutically acceptable salt thereof, is administered orally in a single dose of about 800 mg once monthly. In some embodiments, a compound provided herein, or a pharmaceutically acceptable salt thereof, is administered orally in a single dose of about 850 mg once monthly. In some embodiments, a compound provided herein, or a pharmaceutically acceptable salt thereof, is administered orally in a single dose of about 900 mg once monthly. In some embodiments, a compound provided herein, or a pharmaceutically acceptable salt thereof, is administered orally in a single dose of about 950 mg once monthly. In some embodiments, a compound provided herein, or a pharmaceutically acceptable salt thereof, is administered orally in a single dose of about 1000 mg once monthly.

The frequency of dosage of the compound of the present disclosure, or a pharmaceutically acceptable salt thereof, will be determined by the needs of the individual patient and can be, for example, once per day, once per week, once per month, once per every two months, once per every three months, or once per every six months. Administration of the compound, or a pharmaceutically acceptable salt thereof, continues for as long as necessary to treat the Retroviridae infection, including an HIV infection, or any other indication described herein. For example, a compound, or a pharmaceutically acceptable salt thereof, can be administered to a human suffering from a Retroviridae infection, including an HIV infection, for the duration of the human's life.

Administration can be intermittent, with a period of several or more days during which a patient receives a daily dose of the compound of the present disclosure, or a pharmaceutically acceptable salt thereof, followed by a period of several or more days during which a patient does not receive a daily dose of the compound or a pharmaceutically acceptable salt thereof. For example, a patient can receive a dose of the compound, or a pharmaceutically acceptable salt thereof, every other day, or three times per week. Again by way of example, a patient can receive a dose of the compound, or a pharmaceutically acceptable salt thereof, each day for a period of from 1 to 14 days, followed by a period of 7 to 21 days during which the patient does not receive a dose of the compound, or a pharmaceutically acceptable salt thereof, followed by a subsequent period (e.g., from 1 to 14 days) during which the patient again receives a daily dose of the compound, or a pharmaceutically acceptable salt thereof. Alternating periods of administration of the compound, or a pharmaceutically acceptable salt thereof, followed by non-administration of the compound, or a pharmaceutically acceptable salt thereof, can be repeated as clinically required to treat the patient.

The compounds of the present disclosure, or pharmaceutically acceptable salts thereof, or the pharmaceutical compositions of the present disclosure may be administered once, twice, three, or four times daily, using any suitable mode described above. Also, administration or treatment with the compounds, or pharmaceutically acceptable salts thereof, may be continued for a number of days; for example, commonly treatment would continue for at least 7 days, 14 days, or 28 days, for one cycle of treatment. Treatment cycles are well known for Retroviridae infections, including an HIV infection. In some embodiments, treatment cycles are frequently alternated with resting periods of about 1 to 28 days, commonly about 7 days or about 14 days, between cycles. The treatment cycles, in other embodiments, may also be continuous.

### VI. Combination Therapy

Patients being treated by administration of the compounds provided herein, or pharmaceutically acceptable salts thereof, often exhibit diseases or conditions that benefit from treatment with other therapeutic agents, including agents that are therapeutic for Retroviridae infections, including an HIV infection. In some embodiments, the other therapeutic agent is an agent that is therapeutic for an HIV infection. Thus, one aspect of the disclosure is a method of treating an HIV infection comprising administering a compound of the present disclosure, or a pharmaceutically acceptable salt thereof, in combination with one or more compounds useful for the treatment of an HIV infection to a subject, particularly a human subject, in need thereof.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure, or a pharmaceutically acceptable salt thereof, is combined with one or more additional therapeutic agents as described herein, the components of the composition are administered as a simultaneous or sequential regimen. When administered sequentially, the combination may be administered in two or more administrations.

In some embodiments, a compound of the present disclosure, or a pharmaceutically acceptable salt thereof, is combined with one or more additional therapeutic agents in a unitary dosage form for simultaneous administration to a patient, for example as a solid dosage form for oral administration.

In some embodiments, a compound of the present disclosure, or a pharmaceutically acceptable salt thereof, is co-administered with one or more additional therapeutic agents.

Co-administration includes administration of unit dosages of the compounds provided herein, or pharmaceutically acceptable salts thereof, before or after administration of unit dosages of one or more additional therapeutic agents. The compounds provided herein, or pharmaceutically acceptable salts thereof, may be administered within seconds, minutes, or hours of the administration of one or more additional therapeutic agents. For example, in some embodiments, a unit dose of a compound provided herein, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of a compound provided herein, or a pharmaceutically acceptable salt thereof, within seconds or minutes. In some embodiments, a unit dose of a compound provided herein, or a pharmaceutically acceptable salt thereof, is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of a compound provided herein or a pharmaceutically acceptable salt thereof.

In some embodiments, a compound of Formula I, or a pharmaceutically acceptable salt thereof, is formulated as a tablet, which may optionally contain one or more other compounds useful for treating the disease being treated. In certain embodiments, the tablet can contain another active ingredient for treating a Retroviridae infection, including an HIV infection. In some embodiments, such tablets are suitable for once daily dosing. In some embodiments, such tablets are suitable for once weekly dosing. In some embodiments, such tablets are suitable for once monthly dosing. In some embodiments, such tablets are suitable for once every two months dosing. In some embodiments, such tablets are suitable for once every three months dosing. In some embodiments, such tablets are suitable for once every six months dosing.

Also provided herein are methods of treatment in which a compound of Formula I, or a tautomer or pharmaceutically acceptable salt thereof, is given to a patient in combination with one or more additional therapeutic agents or therapy. In some embodiments, the total daily dosage of a compound of Formula I, or a tautomer, or a pharmaceutically acceptable salt thereof, may be about 1 to about 500 mg administered in a single dose for a human subject.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, darunavir, bictegravir, elsulfavirine, rilpivirine, islatravir, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir +abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR—CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), elsulfavirine (long acting injectable nanosuspension, HIV infection), and elsulfavirine (VM-1500). Additional non-limiting examples of non-nucleoside or non-nucleotide inhibitors of reverse transcriptase include the compounds disclosed in U.S. Pat. No. 10,548,898.

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017. Additional non-limiting examples of HIV integrase inhibitors include the compounds disclosed in U.S. Pat. No. 11,084,832.

Examples of HIV non-catalytic site, or allosteric, integrase inhibitors (NCINI) include, but are not limited to, CX-05045, CX-05168, and CX-14442.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

### Examples of CD4 Attachment Inhibitors Include, but are not Limited to, Ibalizumab and CADA Analogs

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HIBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

Additional examples of HIV capsid inhibitors include, but are not limited to, those described in U.S. Patent Application Publication Nos. US2014221356 and US2016016973.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD 112); CD226 (DNAM-1); Poliovirus receptor (PVR) cell adhesion molecule (PVR, CD155); PVR related immunoglobulin domain containing (PVRIG, CD 112R); T cell immunoreceptor with Ig and ITIM domains (TIGIT); T cell immunoglobulin and mucin domain containing 4 (TEVID4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KTR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSFi4 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KTR2DL1); killer cell immunoglobulin like receptor, two Ig domains and long cytoplasmic tail 2 (KTR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSFi8 (GITRL); CD80 (B7-1), CD28; nectin cell adhesion molecule 2 (NECTIN2, CD 112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDIO680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIT4-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi-and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcTR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TriKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO—C101, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):Dl 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CHO1-04 (all of which bind V1V2-glycan), 2G12 (which binds to outer domain glycan); b12, HJ16, CH103-106, VRCO1-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):Dl 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LNO1 (all of which bind the MPER of gp41).

Examples of additional antibodies include, but are not limited to, bavituximab, UB-421, BF520.1, BiIA-SG, CHO1, CH59, C2F5, C4E10, C2F5+C2G12+C4E10, CAP256V2LS, 3BNC117, 3BNC117-LS, 3BNC60, DH270.1, DH270.6, D1D2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDXO10 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDXO10 (ipilimumab), VRCO1, VRC-01-LS, A32, 7B2, 10E8, VRC-07-523, VRC₀₇₋₅₂3LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMABO60-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRCO1.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsvl vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NacGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag I DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-BO1, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of Formula I) may be combined with one, two, three, or four additional therapeutic agents in any dosage amount of the compound of Formula I (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, doravirine, raltegravir, and long acting dolutegravir.

### VII. Compound Preparation

Some embodiments of the present disclosure are directed to processes and intermediates useful for preparing the compounds provided herein or pharmaceutically acceptable salts thereof.

Compounds described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography.

During any of the processes for preparation of the compounds provided herein, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used. Isolation of final compounds can be performed by various methods known to those skilled in the art but is optimally reverse phase HPLC followed by lyophilization from various organic solvents. Repeated lyophilization can optionally be performed to reduce the amount of residual acidic modifiers resulting from the purification process. In some embodiments, the final compounds provided herein were isolated as mono- or bis-trifluoracetic acid salts.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or diastereomerically pure.

Representative syntheses of compounds of the present disclosure are described in the schemes below, and the particular examples that follow.

The compounds of the present disclosure may be prepared using the methods disclosed herein and routine modifications thereof, which will be apparent to a skilled artisan given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers. In general, compounds described herein are typically stable and isolatable at room temperature and pressure.

Typical embodiments of compounds disclosed herein may be synthesized using the general reaction schemes described below. It will be apparent to a skilled artisan given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments disclosed in the present disclosure, inspection of the structure of the compound to be synthesized will provide the identity of each substituent group. The identity of the final product will generally render apparent the identity of the necessary starting materials by a simple process of inspection, given the examples herein.

The terms “solvent”, “inert organic solvent”, or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane), diethyl ether, methanol, and the like). Unless specified to the contrary, the solvents used in the reactions of the present disclosure are inert organic solvents, and the reactions are carried out under an inert gas, preferably nitrogen or argon.

### List of Abbreviations and Acronyms

| Abbreviation/Acronym | Meaning |
|---|---|
| Ac | Acetate |
| ACN or MeCN | Acetonitrile |
| AcOH | Acetic acid |
| aq. | Aqueous |
| Ar | Argon |
| bu | butyl |
| C | Celsius |
| d | doublet |
| DCE | 1,2-dichloroethane |
| DCM | Dichloromethane |
| dd | doublet of doublets |
| DIPEA | N,N-diisopropylethylamine |
| DMAP | 4-dimethylaminopyridine |
| DME | 1,2-dimethoxyethane |
| DMF | N,N-Dimethylformamide |
| DMSO | Dimethylsulfoxide |
| dq | doublet of quartets |
| EDC | N-(3-Dimethylaminopropyl)-N′-ethylcarbodiimide |
| | hydrochloride |
| EDTA | ethylenediamine tetraacetic acid |
| equiv | equivalent |
| Et | Ethyl |
| Et₃N | Triethylamine |
| EtOAc | Ethyl acetate |
| g | gram |
| h or hr(s) | Hour(s) |
| H | hydrogen |
| HATU | 1-[Bis(dimethylamino)methylene]-1H-1,2,3- |
| | triazolo[4,5-b]pyridinium 3-oxide |
| | hexafluorophosphate |
| HPLC | High pressure liquid chromatography |
| Hz | hertz |
| i-Pr or ⁱPr or iPr | Isopropyl |
| LCMS | Liquid chromatography mass spectrometry |
| m | multiplet |
| M | molarity |
| Me | Methyl |
| MeOH | Methanol |
| MHz | megahertz |
| min or mins | minute |
| μg | microgram |
| μl or μL | microliter |
| μM | micromolar |
| ml or mL | milliliter |
| mmol | millimole |
| MS | Mass spectrometry |
| m/z | Mass to charge ratio |
| N | normal |
| NaI | sodium iodide |
| n-bu | normal butyl |
| nM | nanomolar |
| NMR | Nuclear magnetic resonance spectroscopy |
| NMI | 1-methylimidazole |
| NMM | N-methylmorpholine |
| PBS | phosphate buffered saline |
| Pd(dba)₂ | bis(dibenzylideneacetone)palladium(0) |
| Pd(dppf)Cl₂ | [1,1′-bis(diphenylphosphino)ferrocene] |
| | dichloropalladium(II) |
| pH | potential of hydrogen |
| ppm | parts per million |
| q | quartet |
| Qphos | pentaphenyl(di-tert- |
| | butylphosphino)ferrocene |
| RP | Reverse phase |
| rpm | rotations per minute |
| RT or rt | Room temperature |
| s | singlet |
| sat. or satd. or sat'd | Saturated |
| t | triplet |
| t-Bu or ^{t}Bu or tBu | Tert-butyl |
| TBSCl | Tert-butyldimethylsilyl chloride |
| TCFH | N′-tetramethylformamidinium |
| | hexafluorophosphate |
| TEMPO | 2,2,6,6-tetramethylpiperidine 1-oxyl |
| TFA | Trifluoroacetic acid |
| THF | Tetrahydrofuran |
| TLC | thin layer chromatography |
| TMSBr | bromotrimethylsilane |
| tt | triplet of triplets |
| v/v | volume per volume |
| wt | weight |
| w/w | weight by weight |

### General Synthetic Schemes

General Reaction Schemes 1-6 are provided as further embodiments of the present disclosure and illustrate general methods which were used to prepare certain compounds of the present disclosure and which can be used to prepare additional compounds of the present disclosure. Each of the variables (e.g. R¹, R², R³, R⁴) of the compounds disclosed in General Reaction Schemes 1-6 are as defined herein.

The compounds of the present disclosure may be prepared using the methods disclosed herein and routine modifications thereof, which will be apparent to a skilled artisan given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers. In general, compounds described herein are typically stable and isolatable at room temperature and pressure.

Typical embodiments of compounds disclosed herein may be synthesized using the general reaction schemes described below. It will be apparent to a skilled artisan given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments disclosed in the present disclosure, inspection of the structure of the compound to be synthesized will provide the identity of each substituent group. The identity of the final product will generally render apparent the identity of the necessary starting materials by a simple process of inspection, given the examples herein.

The terms “solvent”, “inert organic solvent”, or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane), diethyl ether, methanol, and the like). Unless specified to the contrary, the solvents used in the reactions of the present disclosure are inert organic solvents, and the reactions are carried out under an inert gas, preferably nitrogen or argon.

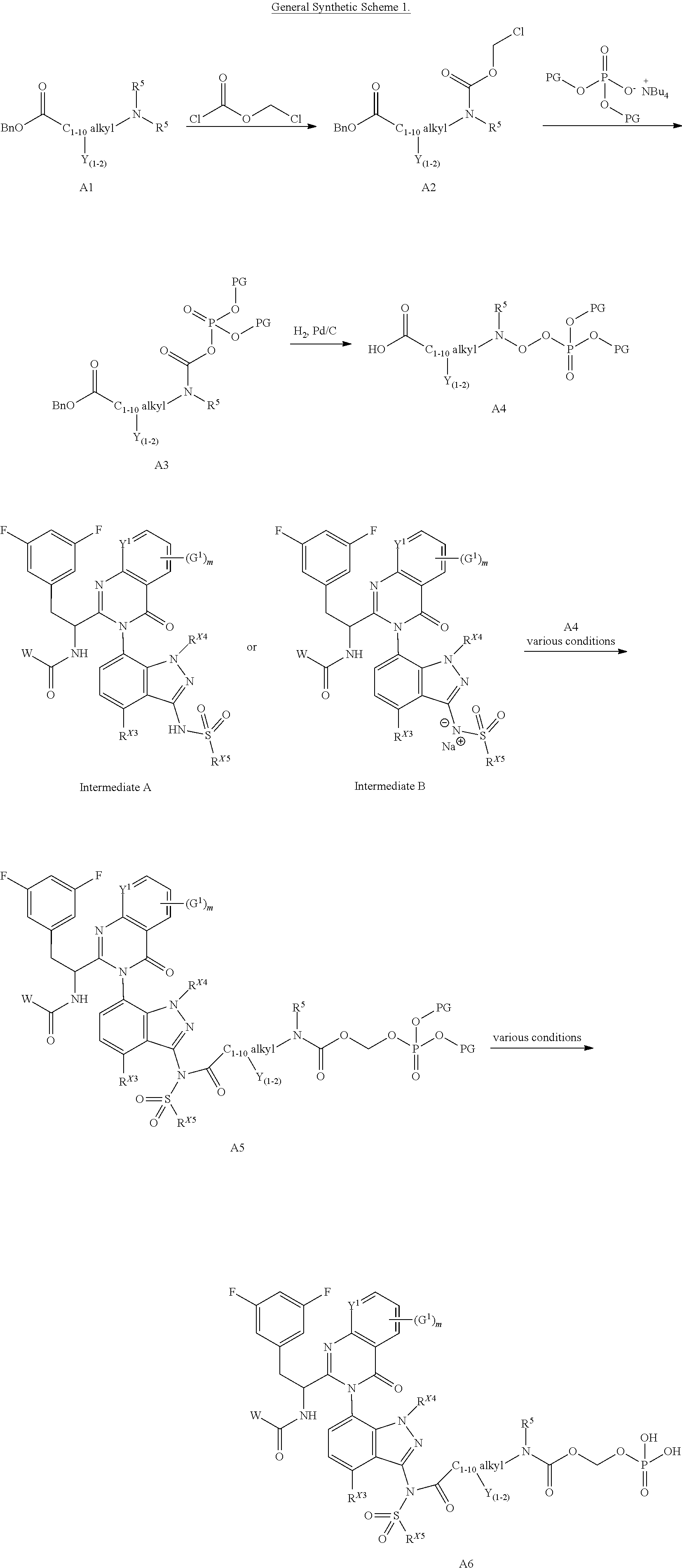

Compounds of formula A6 can be prepared according to General Synthetic Scheme 1, wherein R⁵, Y, Y¹, W, G¹, R^{X3}, R^{X4}, and R^{X5} are as defined herein and PG is a protecting group known in the art. In accordance with General Synthetic Scheme 1, a compound of formula A1 can be reacted with chloromethyl chloroformate in the presence of base in an appropriate solvent to generate a compound of Formula A2. A compound of formula A2 can be reacted with a di-tert-butyl phosphate equivalent, including but not limited to tetrabutylammonium di-tert-butyl phosphate or potassium di-tert-butyl phosphate, to generate a compound of formula A3. A compound of formula A3 can be converted to a compound of formula A4 under various reducing conditions such as using palladium on carbon under an atmosphere of hydrogen gas. A compound of formula A4 can be coupled through various conditions to Intermediate A or Intermediate B to generate a compound of formula A5. A skilled artisan will readily recognize others salts or protonation states of Intermediate A that can be used instead of Intermediate B. Non-limiting exemplary coupling conditions include coupling reagents such as HATU, COMU, TCFH, or EDC under appropriate solvent and temperature conditions in the presence of a base. A compound of formula A5 can be deprotected to produce a compound of Formula A6 under appropriate conditions, including but not limited to phosphoric acid, trifluoroacetic acid, hydrochloric acid, boron tribromide, or trimethylsilyl iodide in various solvents.

Some examples of Intermediate A, their methods of preparation, and their biological activities are disclosed and described in U.S. Ser. No. 10/954,252, U.S. Ser. No. 11/505,543, US2022089598, US2021323961, U.S. Ser. No. 11/541,055, US2021395262, US2021393633, US2021403465, US2021379071, US2021395248, US2022105096, US2022211704, US2021323967, US2022409619, US2022389007, US2023013823, US2022370451, US2023045509, US2023106880, and US2023149408, the contents of each of which are hereby incorporated by reference in their entireties. Additional examples of Intermediate A, their methods of preparation, and their biological activities are disclosed and described in WO2021/176366 and in Gillis, E. et al. J. Med. Chem. 2023, 66 (3), 1941-1954 (https://pubs.acs.org/doi/pdf/10.1021/acs.jmedchem.2c01732).

A compound of formula A1 can be obtained commercially or readily synthesized by those skilled in the art according to known methods. Methods for preparation include but are not limited to preparation of a benzyl ester under appropriate conditions, introduction of —NR⁵R⁵ using methods known in the art, including but not limited to alkylation or reductive amination. Where one of the R⁵ groups is —H, the amino group can be functionalized with additional R⁵ groups using methods known in the art, including but not limited to reaction with chloroformates, reductive amination with an aldehyde, or alkylation with an appropriate electrophile.

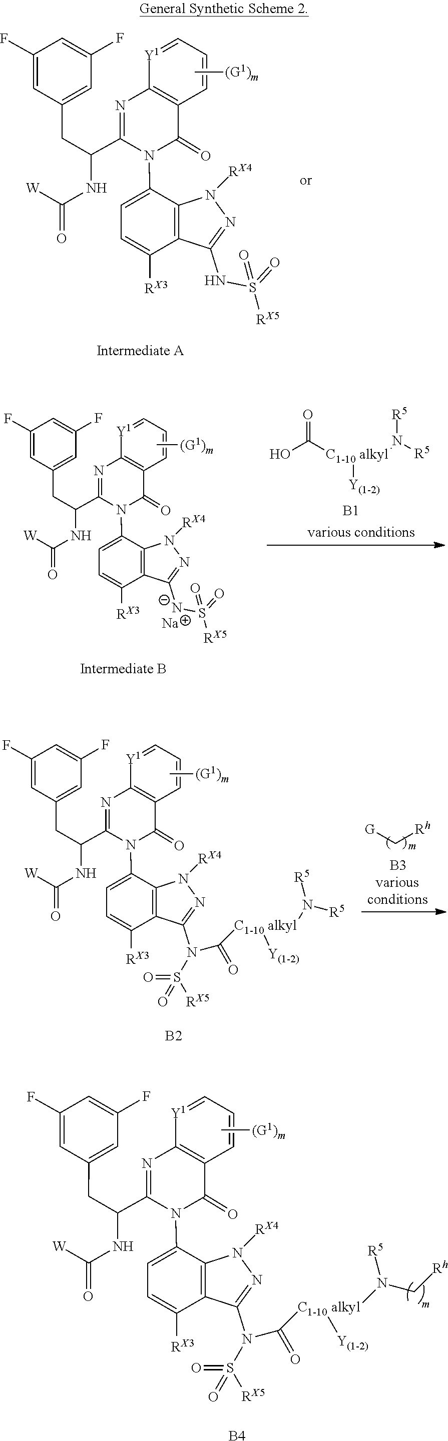

Compounds of formula B2 and B4 can be prepared according to General Synthetic Scheme 2, wherein R⁵, Y, Y¹, W, G¹, R^{X3}, R^{X4}, and R^{X5} are as defined herein; m is 0, 1, 2, 3, 4, 5, or 6; R^{h} is R^{a}, R^{b}, or R^{c} as defined herein; and G is a general leaving group including but not limited to —Cl, —Br, —I, —F, or —OTs. In accordance with General Synthetic Scheme 2, a compound of formula B1 can be reacted with Intermediate A or Intermediate B to generate compounds of formula B2. A compound of formula B2 can be converted to a compound of formula B4 by employing various conditions known in the art for alkylation or acylation using a compound of formula B3, with optional deprotection in cases where a protected functional group has been introduced. Compounds of formula B1 and B3 can be obtained commercially, or readily synthesized from known materials and reagents in one or more steps by those skilled in the art.

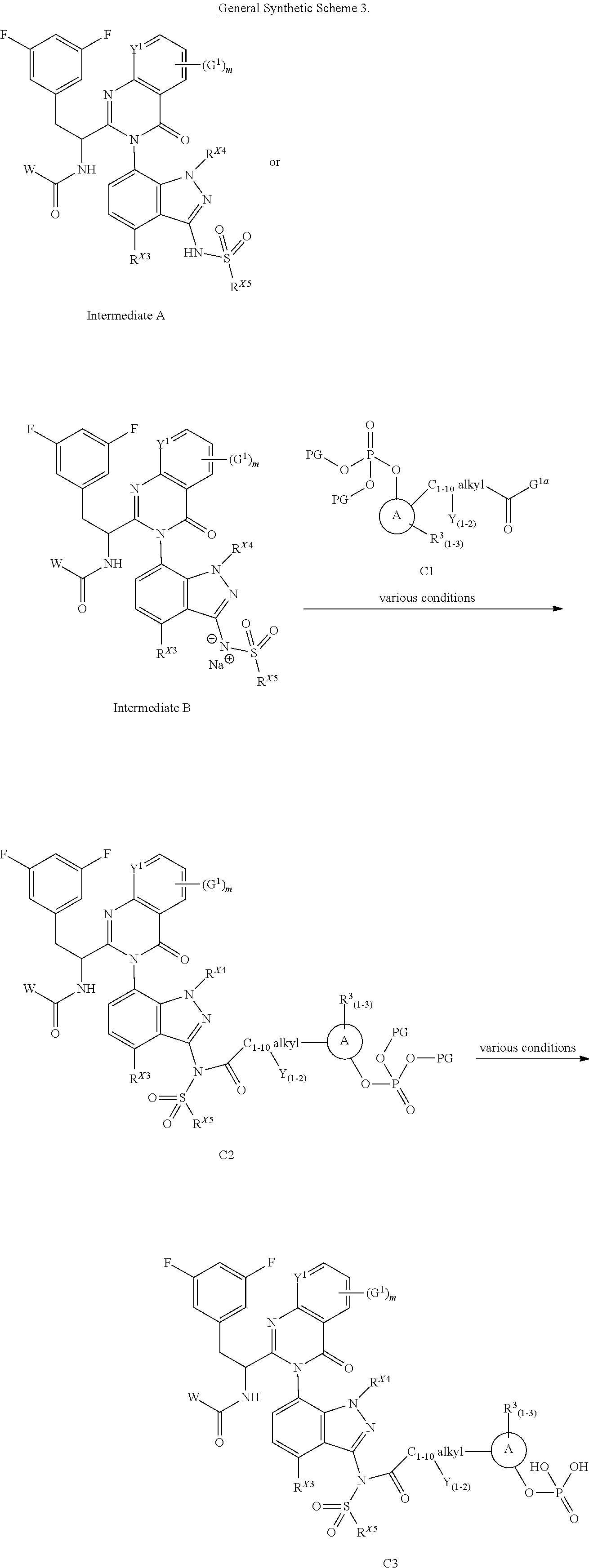

Compounds of formulas C2 and C3 can be prepared according to General Synthetic Scheme 3, wherein R¹, Y, Y¹, W, G¹, R^{X3}, R^{X4}, and R^{X5} are as defined herein; cyclic group A is phenyl, naphthalenyl, 5-6 membered monocyclic heteroaryl, or 8-10 membered fused bicyclic heteroaryl, each of which are optionally substituted with 1-3 R³ groups as defined herein; G^{1a} is a general leaving group that includes but is not limited to —Cl, —Br, —F, or —OH; and PG is a protecting group known in the art. In accordance with General Synthetic Scheme 3, a compound of formula C1 can be reacted with Intermediate A or Intermediate B to generate compounds of Formula C2. Non-limiting exemplary coupling conditions include the use of coupling reagents such as HATU, COMU, TCFH, or EDC under appropriate solvent and temperature conditions in the presence of a base. A compound of formula C2 can be converted to a compound of formula C3 by employing appropriate conditions for deprotection known to those of skill in the art. Compounds of formula C1 can be obtained commercially, or readily synthesized from known materials and reagents in one or more steps by those skilled in the art.

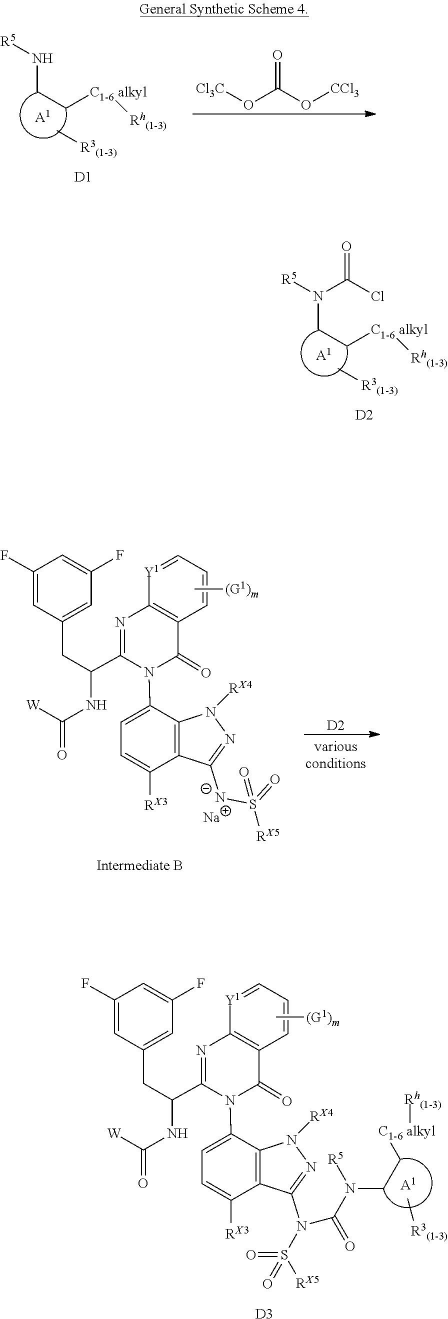

Compounds of formula D3 can be prepared according to General Synthetic Scheme 4, wherein R¹, R³, R⁵, Y¹, W, G¹, R^{X3}, R^{X4}, and R^{X5} are as defined herein; R^{h} is R^{a}, R^{b}, or R^{c} as defined herein; and cyclic group A¹ is phenyl, naphthalenyl, 5-6 membered monocyclic heteroaryl, or 8-10 membered fused bicyclic heteroaryl, each of which are optionally substituted with 1-3 R³ groups as defined herein. In accordance with General Synthetic Scheme 4, a compound of formula D1 can be reacted with a carbonyl group transfer reagent, including but not limited to triphosgene, to generate a compound of formula D2. A compound of formula D2 can be converted to a compound of Formula D3 by reacting under various conditions with Intermediate B, and following optional deprotection of any intermediate obtained. Non-limiting exemplary coupling conditions include incubation of a compound of formula D2 and Intermediate B under appropriate solvent and temperature conditions in the presence of a base. Compounds of formula D1 can be obtained commercially, or readily synthesized from known materials and reagents in one or more steps by those skilled in the art.

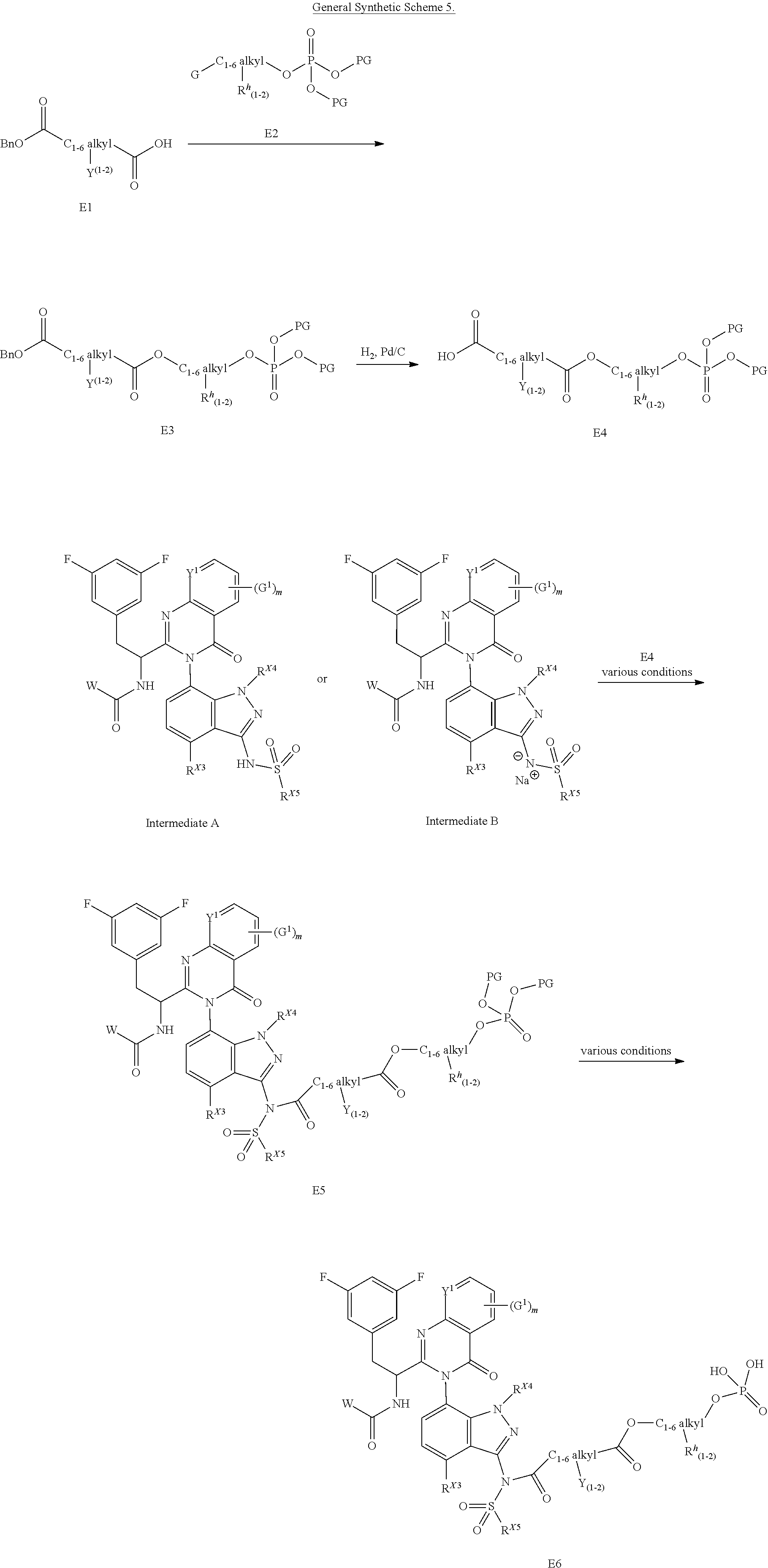

Compounds of formula E6 can be prepared according to General Synthetic Scheme 5, wherein Y, Y¹, W, G¹, R^{X3}, R^{X4}, and R^{X5} are as defined herein; R^{h} is R^{a}, R^{b}, or R^{c} as defined herein; and G is a general leaving group including but not limited to —Cl, —Br, —I, —F, or —OTs. In accordance with General Synthetic Scheme 5, a compound of formula E1 can be reacted with a compound of formula E2 in the presence of base to generate a compound of formula E3. A compound of Formula E4 can be prepared from hydrogenation of a compound of formula E3. A compound of formula E4 can be reacted with Intermediate A or Intermediate B, in the presence of base and an appropriate coupling reagent to generate a compound of formula E5, which can then be deprotected under acidic conditions to yield a compound of formula E6. Non-limiting exemplary coupling conditions include the use of coupling reagents such as HATU, COMU, TCFH, or EDC under appropriate solvent and temperature conditions in the presence of a base. Compounds of formula E1 and E2 can be obtained commercially, or readily synthesized from known materials and reagents in one or more steps by those skilled in the art.

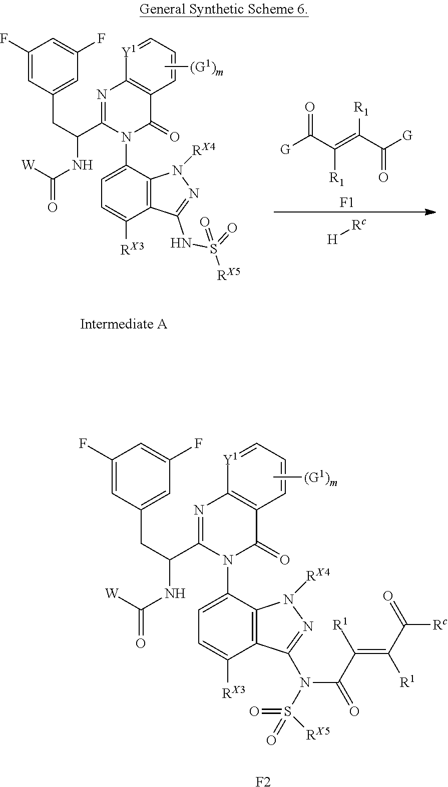

Compounds of formula F2 can be prepared according to General Synthetic Scheme 6, wherein R¹ R^{c}, Y¹, W, G¹, R^{X3}, R^{X4}, and R^{X5} are as defined herein. G denotes a general leaving group including but not limited to —Cl, —Br, —I, —F, or —OTs. In accordance with General Synthetic Scheme 5, Intermediate A can be reacted with a compound of formula F1 in the presence of base and nucleophilic R^{c} group to generate a compound of formula F2, which can optionally be deprotected under appropriate conditions in cases where R^{c} contains protected functionality. Compounds of formula F1 can be obtained commercially, or readily synthesized from known materials and reagents in one or more steps by those skilled in the art.

### VIII. Examples

Exemplary chemical entities of the present disclosure are provided in the specific examples that follow. Those skilled in the art will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

In some embodiments, the compounds of the Examples may be isolated as a mixture of rotational isomers. In some embodiments, the compounds of the Examples may be isolated as a mixture of atropisomers.

In the following description of the Examples, the specific embodiments described in present tense are prophetic.

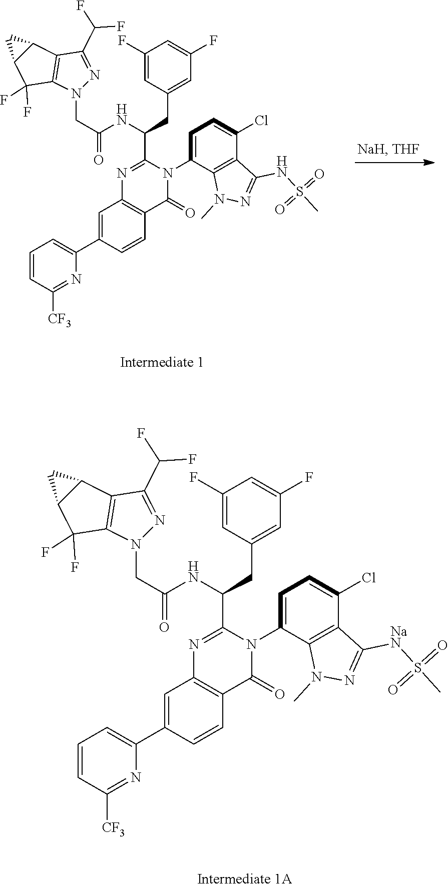

Intermediate 1A (prophetic synthesis): To a solution of Intermediate 1 (1.0 eq, can be prepared according to Example 59 in WO 2020/084492) in THF (0.1 M) is added sodium hydride (60% dispersion in mineral oil, 1.2 eq). The mixture is stirred for 10 min and concentrated to afford sodium (4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)(methylsulfonyl)amide which is used without further purification.

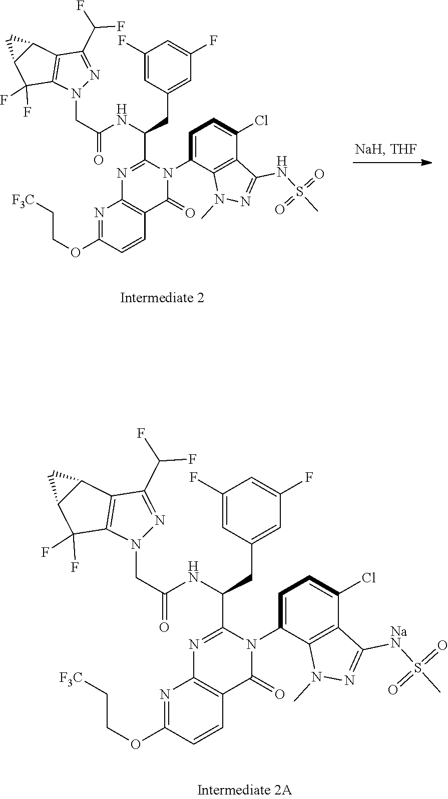

Intermediate 2A (prophetic synthesis): To a solution of Intermediate 2 (1.0 eq, can be prepared according to the procedure described in WO 2020/254985) in THE (0.1 M) is added sodium hydride (60% dispersion in mineral oil, 1.2 eq). The mixture is stirred for 10 min and concentrated to afford sodium (4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)(methylsulfonyl)amide which is used without further purification.

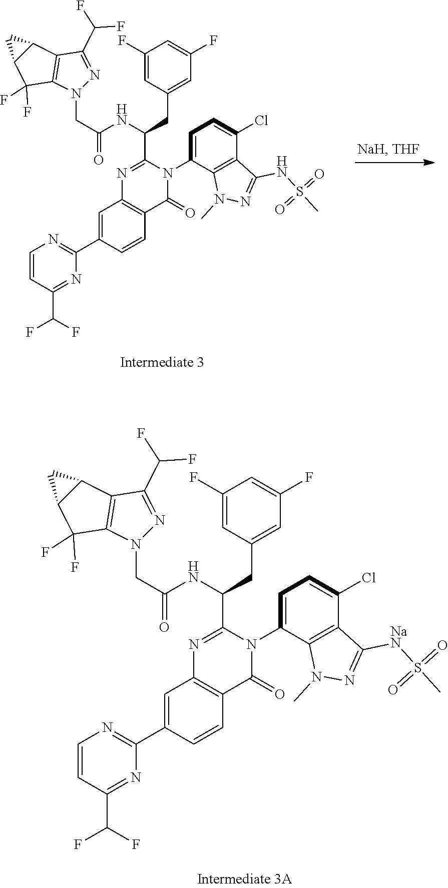

Intermediate 3A (prophetic synthesis): To a solution of Intermediate 3 (1.0 eq, can be prepared according to Example 56 in WO 2020/084492) in THF (0.1 M) is added sodium hydride (60% dispersion in mineral oil, 1.2 eq). The mixture is stirred for 10 min and concentrated to afford sodium (4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)(methylsulfonyl)amide which is used without further purification.

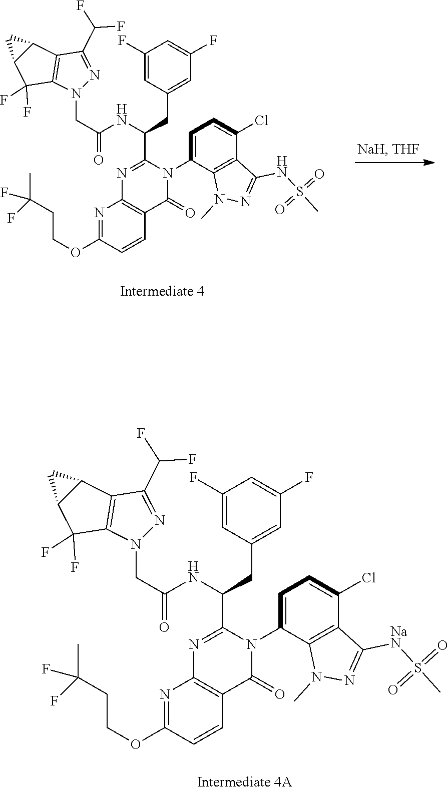

Intermediate 4A (prophetic synthesis): To a solution of Intermediate 4 (1.0 eq, can be prepared according to the procedure described in WO 2020/157692) in THE (0.1 M) is added sodium hydride (60% dispersion in mineral oil, 1.2 eq). The mixture is stirred for 10 min and concentrated to afford sodium (4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3, 5-difluorophenyl)ethyl)-4-oxopyrido[2,3-d]pyrimidin-3(4H)-yl)-1-methyl-1H-indazol-3-yl)(methylsulfonyl)amide which is used without further purification.

### Example 1. 4-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-methyl-5-(phosphonooxy)benzoic acid (Compound 1)

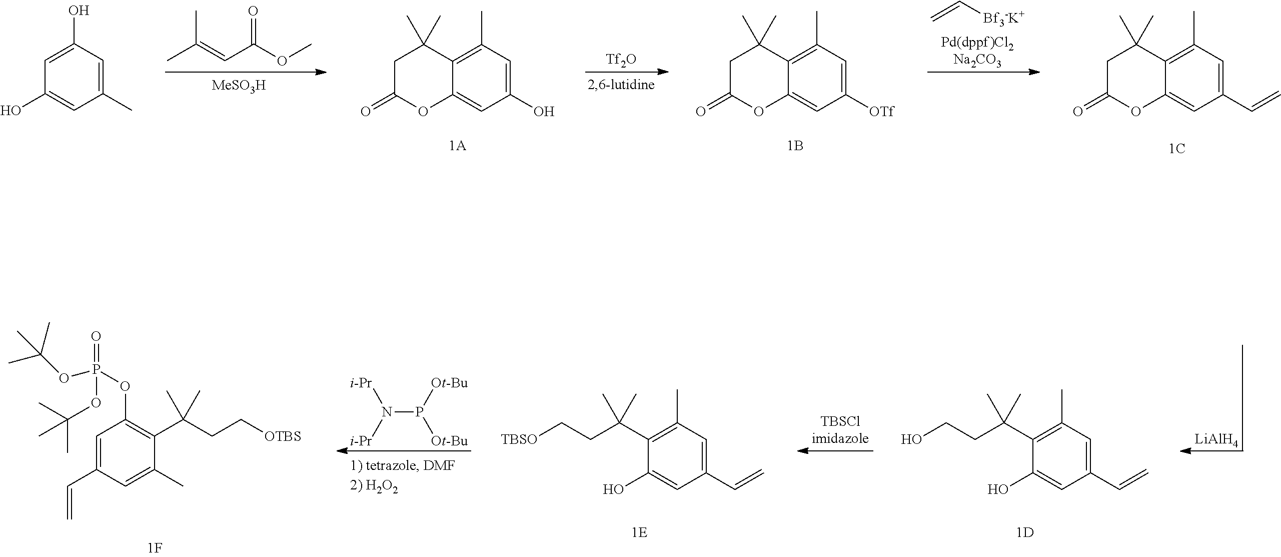

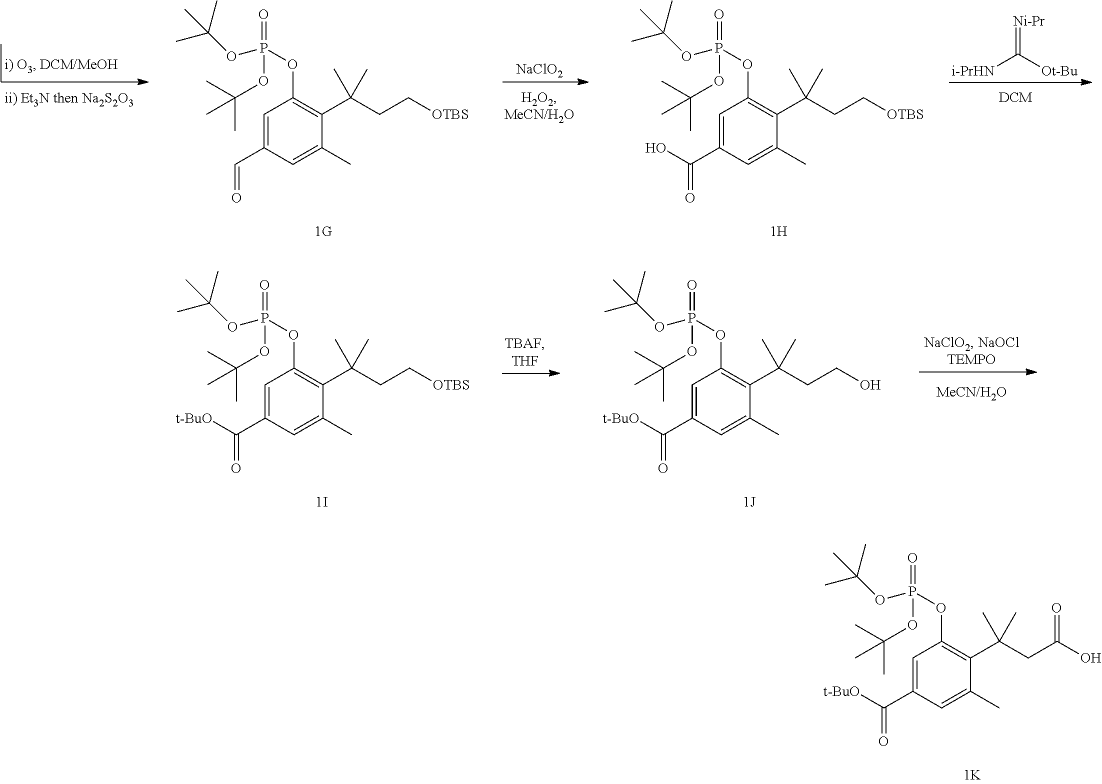

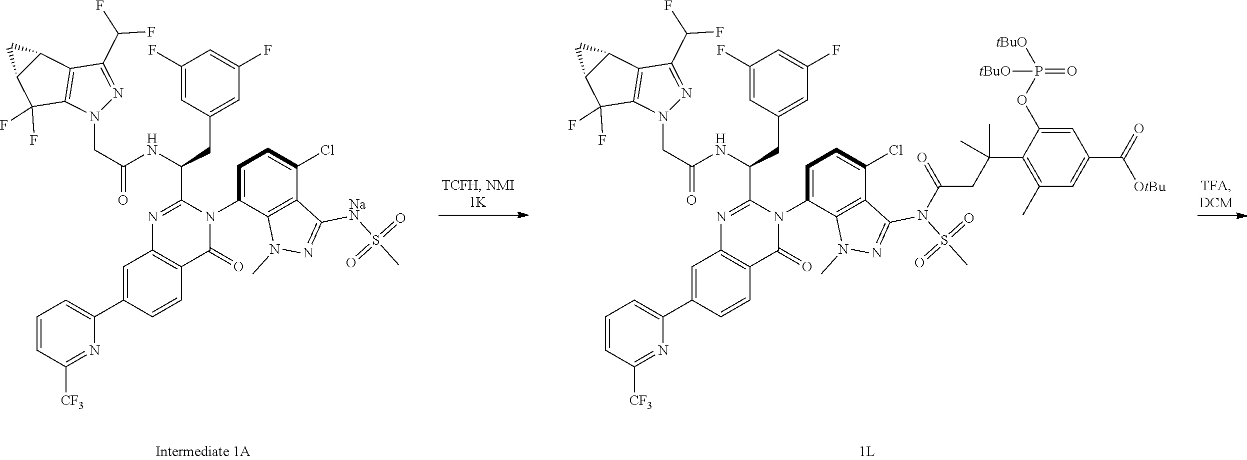

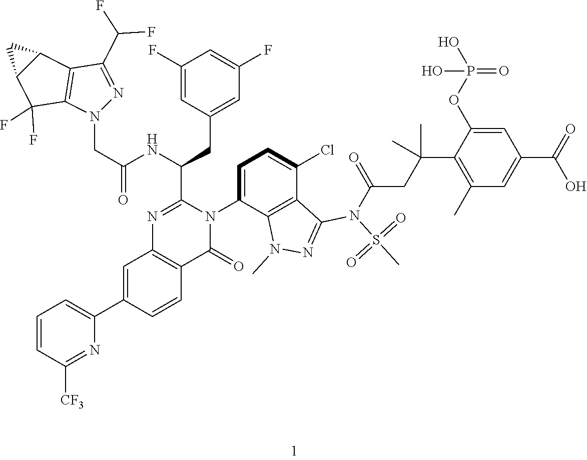

### Step 1. Synthesis of 7-hydroxy-4,4,5-trimethylchroman-2-one (1A)

To a stirred solution of 3,5-dihydroxytoluene (483 mmol) in methanesulfonic acid (62.8 mL) was added methyl 3-methylbut-2-enoate (532 mmol). The mixture was stirred at 80° C. for 3 h and monitored by LCMS. After completion, the reaction mixture was diluted with water and extracted with EtOAc. The combined organic layers were dried over anhydrous Na₂SO₄, filtered, and concentrated. The crude mixture was purified by column chromatography (0% to 30% EtOAc in hexanes) to afford title compound 1A. MS (m/z) 207.1 [M+H]⁺.

### Step 2. Synthesis of 4,4,5-trimethyl-2-oxochroman-7-145rifluoromethanesulfonateate (1B)

To a stirred solution 1A (249 mmol) in dichloromethane (20 mL) at 0° C. was added 2,6-lutidine (274 mmol) followed by triflic anhydride (262 mmol, 1.05 equiv) and the reaction mixture was stirred for 2 h and monitored by LCMS. After completion, the reaction mixture was diluted with dichloromethane, washed with 1M HCl and brine. The combined organic layers were dried over anhydrous Na₂SO₄, filtered, and concentrated. The crude mixture was purified by column chromatography (0% to 10% EtOAc in hexanes) to afford title compound 1B. MS (m/z) 339.1 [M+H]⁺.

### Step 3. Synthesis of 4,4,5-trimethyl-7-vinylchroman-2-one (1C)

To a stirred solution of 1B (161 mmol) in dioxane (130 mL) and water (15 mL) was added potassium vinyltrifluoroborate (193 mmol), sodium carbonate (483 mmol) and Pd(dppf)Cl₂ (16.1 mmol). The mixture was degassed and backfilled with argon (3×) and heated to 100° C. for 2 h and monitored by LCMS. After completion, the reaction mixture was diluted with EtOAc and washed with sat. aq. solutions of NH₄Cl and brine. The combined organic layers were dried over anhydrous Na₂SO₄, filtered, and concentrated. The crude mixture was purified by column chromatography (0% to 10% EtOAc in hexanes) to afford title compound 1C. MS (m/z) 217.2 [M+H]⁺.

### Step 4. Synthesis of 2-(4-hydroxy-2-methylbutan-2-yl)-3-methyl-5-vinylphenol (1D)

Compound 1C (83.2 mmol) was dissolved in THF (121 mL), cooled to 0° C. and treated with LiAlH₄ (53 mL, 2 M in THF). The reaction mixture was then gradually warmed to room temperature over 16 h. After completion, the reaction mixture was quenched with ice water, filtered through a pad of Celite®, and extracted with EtOAc. The combined organic layers were dried over anhydrous Na₂SO₄, filtered, and concentrated to yield title compound 1D which was used without purification.

### Step 5. Synthesis of 2-(4-((tert-butyldimethylsilyl)oxy)-2-methylbutan-2-yl)-3-methyl-5-vinylphenol (1E)

To a solution of 1D (20.1 mmol) in DMF (30 mL) was added imidazole (50.2 mmol) and TBSCl (26 mmol) sequentially. The reaction mixture was stirred for 2 h and monitored by LCMS. After completion, the reaction mixture was diluted with diethyl ether and washed with water. The combined organic layers were dried over anhydrous Na₂SO₄, filtered, and concentrated. The crude mixture was purified by column chromatography (0% to 10% EtOAc in hexanes) to afford title compound 1E. MS (m/z) 358.1 [M+Na]⁺.

### Step 6. Synthesis of di-tert-butyl (2-(4-((tert-butyldimethylsilyl)oxy)-2-methylbutan-2-yl)-3-methyl-5-vinylphenyl) phosphate (1F)

To a solution of 1E (18.7 mmol) in DMF (200 mL) was added di-t-butyl-N,N-diisopropylphosphoramidite (56.0 mmol) and 1-H-tetrazole (7.1 mmol) sequentially. The reaction mixture was stirred at room temperature while monitored by TLC and LCMS. After 6 h, the reaction mixture was cooled to 0° C. and treated with aq. H₂O₂ solution (24 mL, 50 wt. %). The reaction was gradually warmed to room temperature and stirred. After completion, the reaction mixture was diluted with EtOAc and washed with sat. aq. sodium thiosulfate solution. The combined organic layers were dried over anhydrous Na₂SO₄, filtered, and concentrated. The crude mixture was purified by column chromatography to afford title compound 1F. MS (m/z) 549.2 [M+Na]⁺.

### Step 7. Synthesis of di-tert-butyl (2-(4-((tert-butyldimethylsilyl)oxy)-2-methylbutan-2-yl)-5-formyl-3-methylphenyl) phosphate (1G)

To a solution of di-tert-butyl (2-(4-((tert-butyldimethylsilyl)oxy)-2-methylbutan-2-yl)-3-methyl-5-vinylphenyl) phosphate 1F (26.8 mmol) in 120 mL of a 1:1 mixture of DCM/MeOH was carefully bubbled O₃ gas at −78° C. When full conversion was observed, the reaction was sparged with Ar gas for 5 mins, after which Et₃N (10 mL) was carefully added. To the resulting mixture was then added 200 mL of saturated Na₂S₂O₃ and the reaction was stirred a further 1h. Upon completion of this time, the contents were transferred to a separatory funnel using DCM (200 mL) and H₂O (200 mL). The aqueous layer was extracted with DCM (2×100 mL) and the organic fractions were collected, dried over Na₂SO₄, concentrated under reduced pressure to yield title compound 1G, which was used without further purification. MS (m/z) 551.30 [M+Na]⁺.

### Step 8. Synthesis of 4-(4-((tert-butyldimethylsilyl)oxy)-2-methylbutan-2-yl)-3-((di-tert-butoxyphosphoryl)oxy)-5-methylbenzoic acid (1H)

To a solution of 1G (17.9 mmol), KH₂PO₄ (7.15 mmol, 0.4 equiv), hydrogen peroxide (30% aqueous solution, 26.8 mmol, 1.5 equiv) in 75 mL of 1:1 MeCN/H₂O was added a solution of sodium chlorite (35.7 mmol, 2 equiv) at 0° C. and the resulting mixture stirred for 30 mins at this temperature before slowly warming to room temperature. Upon full conversion, the reaction was quenched with saturated sodium sulfite solution and the contents were transferred to a separatory funnel using DCM (200 mL) and H₂O (200 mL) where the pH was carefully adjusted to 2 with 1M HCl solution. The aqueous layer was extracted with DCM (2×100 mL) and the organic fractions were collected, dried over Na₂SO₄, concentrated under reduced pressure to yield title compound 1H which was used without further purification. MS (m/z) 567.3 [M+Na]⁺.

### Step 9. Synthesis of tert-butyl 4-(4-((tert-butyldimethylsilyl)oxy)-2-methylbutan-2-yl)-3-((di-tert-(butoxyphosphoryl)oxy)-5-methylbenzoate (1I)

To a solution of 1H (23.3 mmol) in 50 mL of DCM was added 2-tert-butyl-1,3-diisopropylurea (117 mmol, 5 equiv) at rt and the resulting solution left to stir overnight. Upon completion, the reaction was diluted with DCM (100 mL), filtered, and the filter cake washed with further DCM (3×50 mL). The mother liquor was then concentrated under reduced pressure, and the residue purified by silica gel chromatography. Fractions containing the product were pooled and lyophilized to give title compound 1I. ¹H NMR (400 MHz, CDCl₃) δ 7.96-7.90 (m, 1H), 7.50 (d, J=1.9 Hz, 1H), 3.48 (t, J=7.2 Hz, 2H), 3.38 (dq, J=18.7, 6.8 Hz, 1H), 2.59 (s, 3H), 2.14 (t, J=7.1 Hz, 2H), 1.57 (s, 16H), 1.51 (s, 18H), 1.46 (s, 8H), 1.21 (d, J=6.7 Hz, 5H), 0.83 (s, 10H), −0.05 (s, 7H) ppm. MS (m/z) 601.4 [M+H]⁺.

### Step 10. Synthesis tert-butyl 3-((di-tert-butoxyphosphoryl)oxy)-4-(4-hydroxy-2-methylbutan-2-yl)-5-methylbenzoate (1J)

To a solution of 1I (14.7 mmol) in 50 mL of THF was added TBAF solution (1.OM in THF, 20 mmol, 1.5 equiv). When full conversion was observed, the reaction was concentrated under reduced pressure and transferred to a separatory funnel using DCM (200 mL) and water (200 mL). The organic layer was washed with brine (4×200 mL), dried over Na₂SO₄, concentrated under reduced pressure and the residue purified by silica gel chromatography. Fractions containing the product were pooled and concentrated under reduced pressure to give title compound 1J. MS (m/z) 486.21 [M+H]⁺.

### Step 11. Synthesis of 3-(4-(tert-butoxycarbonyl)-2-((di-tert-butoxyphosphoryl)oxy)-6-methylphenyl)-3-methylbutanoic acid (1K)

To a solution of 1J (9.68 mmol), TEMPO (0.484 mmol, 0.05 equiv), KHPO₄ (4.84 mmol, 0.5 equiv), and K₂HIPO₄ (4.84 mmol, 0.5 equiv) in 100 mL of H₂O/MeCN (1:1) at 0° C. were added sequentially sodium hypochlorite (8.25% aqueous solution, 11.8 mmol, 1.22 equiv) and sodium chlorite (14.5 mmol, 1.5 equiv). The solution was stirred for 1h at 0° C. and then warmed to rt. When full conversion was observed, the reaction was quenched with saturated sodium sulfite solution and the contents were transferred to a separatory funnel using DCM (150 mL) and H₂O (150 mL) where the pH was carefully adjusted to 2 with 1M HCl solution. The aqueous layer was extracted with DCM (2×100 mL) and the organic fractions were collected, dried over Na₂SO₄, concentrated under reduced pressure. The title compound was obtained by recrystallization from EtOAc/Hexanes to yield title compound 1K. MS (m/z) 523.29 [M+Na]⁺.

### Step 12. Prophetic Synthesis of tert-butyl 4-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((di-tert-butoxyphosphoryl)oxy)-5-methylbenzoate (Compound 1L)

To a suspension of Intermediate 1A (1.3 eq), 1-methylimidazole (3.5 eq), and 1K (1.0 eq) in MeCN (0.2 M) is added TCFH (1.2 eq) and the resulting mixture is stirred at rt. Upon completion of the reaction the solvent is removed under reduced pressure and the resulting residue is purified by silica gel chromatography. Fractions containing the product are pooled and concentrated under reduced pressure to give title compound 1L.

### Step 13. Prophetic Synthesis of 4-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-methyl-5-(phosphonooxy)benzoic acid (Compound 1)

A solution of 1L (1.0 eq) in TFA/DCM (1:4, 0.09 M) is stirred at room temperature. Upon reaction completion, the solvent is removed under reduced pressure and the residue is purified by reverse phase HPLC. Product-containing fractions are pooled and lyophilized to yield title compound 1.

### Example 2. 4-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-methyl-5-(phosphonooxy)benzoic acid (Compound 2)

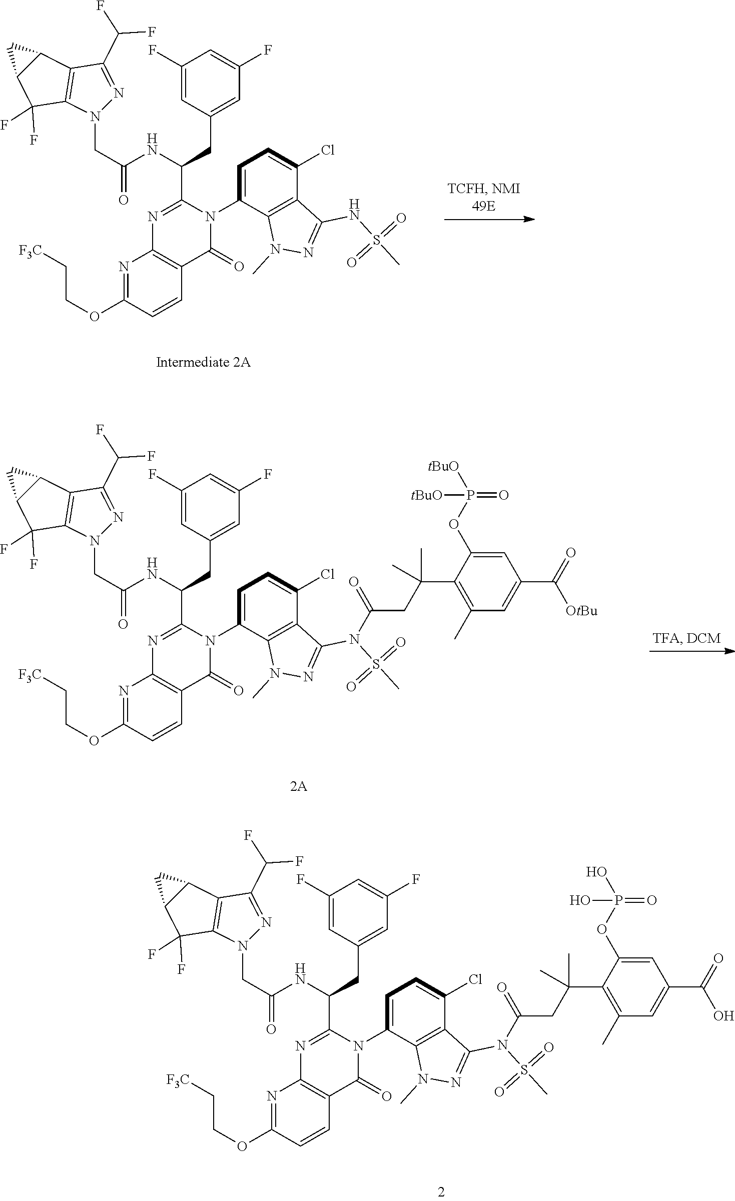

### Step 1. Prophetic Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (Compound 2A)

To a solution of crude 1K (1.0 eq) in ACN (0.1 M) is added N-methylimidazole (3.5 eq), Intermediate 2A (1.0 eq) and TCFH (1.0 eq). The reaction is stirred until completion. The reaction is quenched with sat. aqueous NH₄Cl and extracted with EtOAc (3×). The combined organic layers are washed with brine, dried over MgSO₄, filtered and concentrated under reduced pressure. The crude material is purified by reverse phase HPLC. Fractions containing title compound are pooled and concentrated to afford title compound 2A.

### Step 2. Prophetic Synthesis of 4-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-methyl-5-(phosphonooxy)benzoic acid (Compound 2)

Compound 2A (1.0 eq) is dissolved in DCM/TFA (3:1, 0.03 M). The reaction is stirred at RT until reaction completion. The reaction is concentrated and the crude product is purified by RP chromatography. Fractions containing the product are pooled and lyophilized to afford title compound 2.

### Example 3. 2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4)-yl)-1-methyl-1H-indazol-3-yl)methylsulfonamido)-2-methyl-4-oxobutan-2-yl)-5-methyl-3-(phosphonooxy)phenyl)acetic acid (Compound 3)

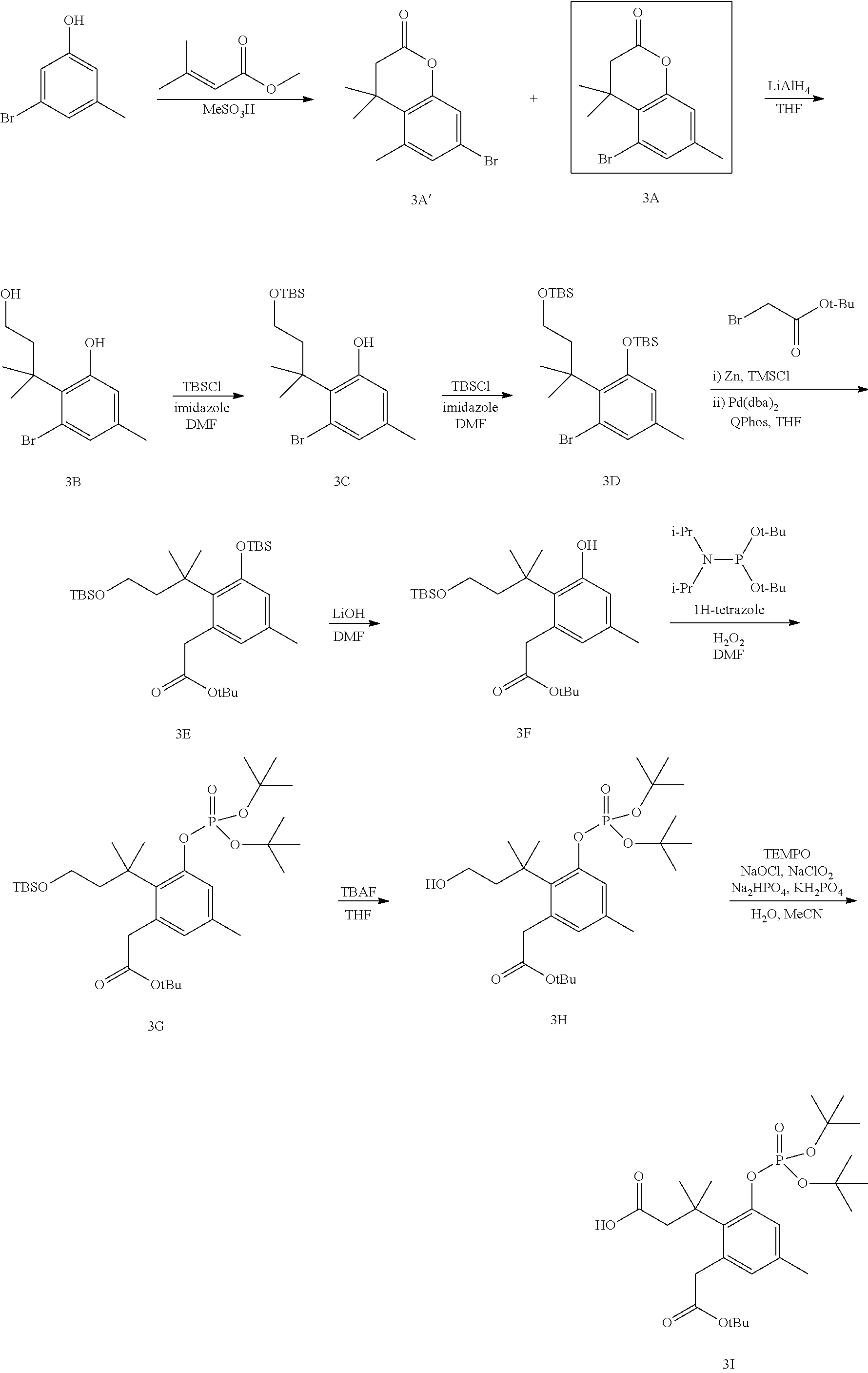

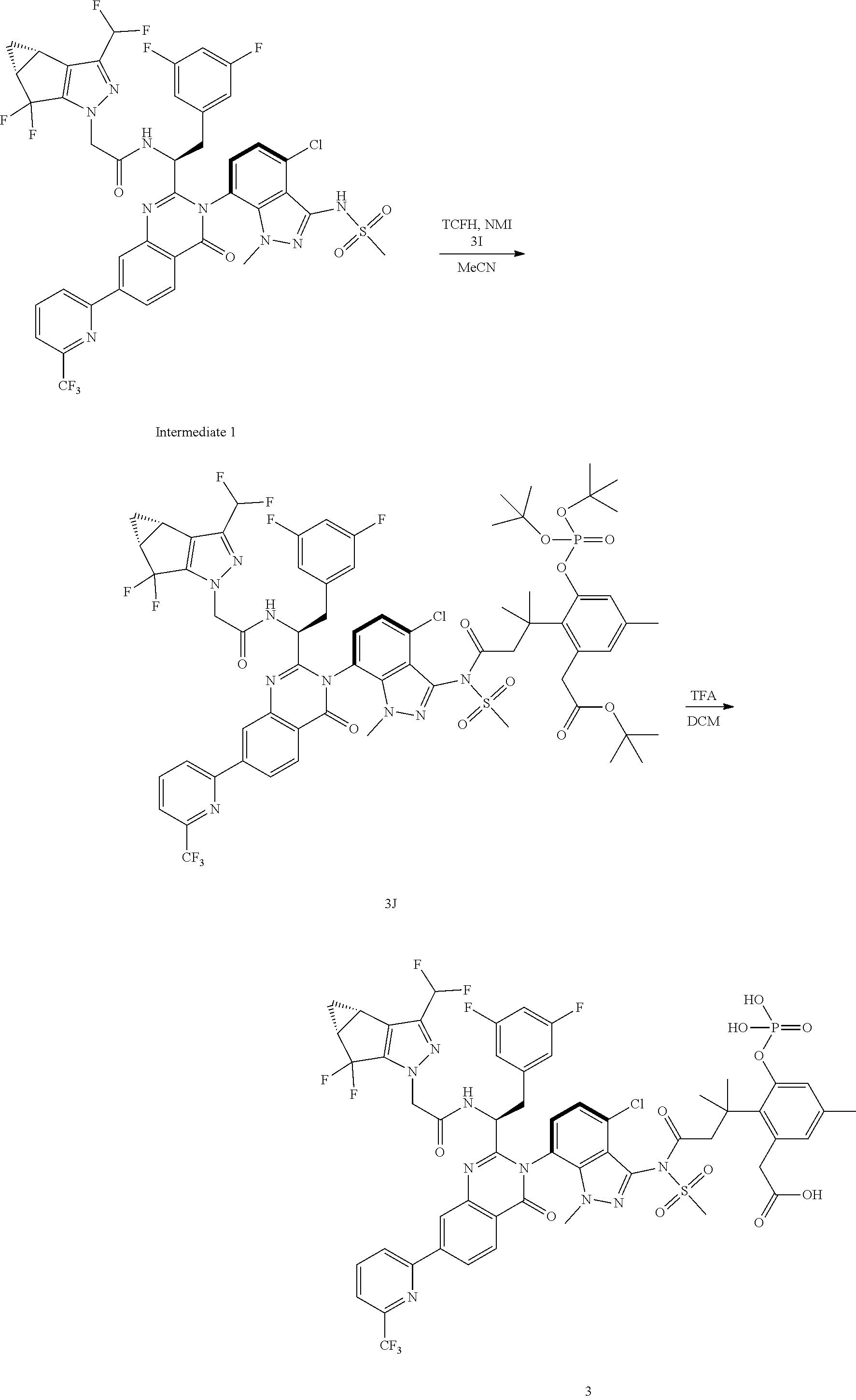

### Step 1. Synthesis of 5-Bromo-4,4, 7-trimethylchroman-2-one (3A)

A flask was charged with 3-bromo-5-methylphenol (26.7 mmol) and methanesulfonic acid (53.5 mmol) under nitrogen. Methyl 3-methylbut-2-enoate (29.4 mmol) was added, the flask was fitted with a reflux condenser and the reaction was heated at 90° C. for 30 minutes, 120° C. for 30 minutes and 150° C. for 3 hours. The reaction was cooled to RT, placed into an ice-water bath and diluted with water (150 mL). The aqueous layer was extracted with EtOAc (2×200 mL), washed with sat. NaHCO₃ (2×100 mL), washed with brine, dried over MgSO₄, filtered and concentrated under reduced pressure. The residue was purified by silica gel chromatography to afford title compound 3A. MS (m/z) 269.02/270.95 [M+H]⁺.

### Step 2. Synthesis of 3-Bromo-2-(4-hydroxy-2-methylbutan-2-yl)-5-methylphenol (3B)

A flask was charged with a solution of LAH (2.0M in THF, 8.17 mmol) and THE (10 mL). The solution was cooled to 0° C. A solution of 3A (7.43 mmol) in THE (20 mL) was added dropwise. The ice bath was removed and the reaction was stirred at RT for 2.5 hours. The reaction was cooled to 5° C. Water (310 μL) was added slowly, followed by 15% NaOH_{(aq)} (310 μL), then water again (930 μL). The mixture was warmed to RT and stirred overnight. The mixture was diluted with EtOAc, MgSO₄ was added and the mixture was filtered. The filter cake was rinsed with EtOAc. The crude product was purified by silica gel chromatography to afford title compound 3B. MS (m/z) 271.15/273.03 [M−H]⁻.

### Step 3. Synthesis of 3-Bromo-2-(4-((tert-butyldimethylsilyl)oxy)-2-methylbutan-2-yl)-5-methylphenol (3C)

A flask was charged with 3B (6.55 mmol), DMF (15.0 mL) and imidazole (16.4 mmol) under nitrogen. The solution was cooled to 0° C., TBSCl (8.51 mmol) was added in one portion, the ice bath was removed and the reaction was stirred at RT for 2 hours and then cooled to 0° C. The reaction was diluted with water (75 mL), extracted with EtOAc (3×), washed with 5% LiCl_{(aq)}, dried over MgSO₄, filtered and concentrated. The crude product was purified by silica gel chromatography to afford title compound 3C. MS (m/z) 385.31/386.9 [M−H]⁻.

### Step 4. Synthesis of (3-Bromo-2-(4-((tert-butyldimethylsilyl)oxy)-2-methylbutan-2-yl)-5-methylphenoxy)(tert-butyl)dimethylsilane (3D)

A flask was charged with 3C (2.55 mmol), DMF (12.0 mL) and imidazole (6.37 mmol) under nitrogen. TBSCl (3.31 mmol) was added in one portion and the flask was fitted with a reflux condenser. The reaction was heated at 65° C. for 8 hours. The reaction was concentrated, diluted with water, extracted with EtOAc (3×), washed with 5% LiCl_{(aq)}, dried over MgSO₄, filtered and concentrated. The mixture was purified by silica gel chromatography to afford title compound 3D. ¹H NMR (400 MHz, Chloroform-d) δ 7.04 (d, J=1.6 Hz, 1H), 6.53 (d, J=1.8 Hz, 1H), 3.52-3.43 (m, 2H), 2.29-2.20 (m, 2H), 2.17 (s, 3H), 1.60 (s, 6H), 1.00 (s, 9H), 0.83 (s, 9H), 0.29 (s, 6H), 0.03 (s, 6H) ppm.

### Step 5. Synthesis of tert-butyl 2-(3-((tert-butyldimethylsilyl)oxy)-2-(4-((tert-butyldimethylsilyl)oxy)-2-methylbutan-2-yl)-5-methylphenyl)acetate (3E)

A flask was charged with zinc dust (<10 μm, 9.00 mmol) and THE (3.6 mL) under nitrogen. The flask was fitted with an internal temperature probe. Chlorotrimethylsilane (0.60 mmol) was added and the mixture was stirred for 15 minutes (a 1-2° C. exotherm was recorded). A solution of tert-butyl 2-bromoacetate (6.00 mmol) in THE (2.66 mL) was added cautiously. The mixture was stirred until it cooled back to RT and then stirring was stopped. The supernatant was titrated using I₂ in a 0.5M solution of LiCl in THF. The concentration of the 2-tert-butoxy-2-oxoethylzinc bromide organozinc reagent was determined to be 0.63M. A separate flask was charged with 3D (1.35 mmol), Pd(dba)₂ (0.068 mmol) and QPhos (0.068 mmol). The flask was purged with nitrogen. THE (4.7 mL) was added, followed by 2-tert-butoxy-2-oxoethylzinc bromide (1.62 mmol). The flask was fitted with a reflux condenser and the reaction was heated at 55° C. for 15-30 minutes. The reaction was cooled to RT, quenched with sat. NH₄Cl_{(aq)}, extracted with EtOAc (3×), washed with brine, dried over MgSO₄, filtered and concentrated. The crude product was purified by silica gel chromatography to afford title compound 3E. MS (m/z) 559.13 [M+Na]⁺.

### Step 6. Synthesis of tert-butyl 2-(2-(4-((tert-butyldimethylsilyl)oxy)-2-methylbutan-2-yl)-3-hydroxy-5-methylphenyl)acetate (3F)

To a solution of 3E (5.14 mmol) in DMF (4.0 mL) was added LiOH (5.14 mmol) under nitrogen. The mixture was stirred at RT for 18 hours. The reaction was diluted with water, extracted with EtOAc (3×), the combined organic layers were sequentially washed with 5% aqueous LiCl and brine, then dried over MgSO₄, filtered and concentrated under reduced pressure. The residue was purified by silica gel chromatography to afford title compound 3F. MS (m/z) 421.16 [M−H]⁻.

### Step 7. Synthesis of tert-butyl 2-(2-(4-((tert-butyldimethylsilyl)oxy)-2-methylbutan-2-yl)-3-((di-tert-butoxyphosphoryl)oxy)-5-methylphenyl)acetate (3G)

To a solution of 3F (0.821 mmol) in DMF (3.0 mL) was added di-tert-butyl N,N-diisopropylphosphoramidite (2.46 mmol) followed by 1H-tetrazole (2.87 mmol). The solution was heated at 50° C. for 5 hours. The reaction was cooled to 0° C., 30% H₂O_{2(aq)}(3.28 mmol) was added, and the reaction was stirred for 1 hour then allowed to warm to RT. The reaction was diluted with water and extracted with 25% EtOAc/hexanes (3×). The combined organic layers were washed with 5% LiCl_{(aq)}, then sat. aqueous Na₂S₂O₃, dried over MgSO₄, filtered and concentrated. The residue was purified by silica gel chromatography to yield title compound 3G. MS (m/z) 637.05 [M+Na]⁺.

### Step 8. Synthesis of tert-butyl 2-(3-((di-tert-butoxyphosphoryl)oxy)-2-(4-hydroxy-2-methylbutan-2-yl)-5-methylphenyl)acetate (3H)

A solution of 3G (0.569 mmol) in THE (5.0 mL) was cooled to 0° C. TBAF (1.0 M in THF, 1.14 mmol) was added and the reaction was stirred for 18 hours, gradually warming up to RT. The reaction was concentrated and diluted with EtOAc. The organic layer was washed with water (2×) and brine, dried over MgSO₄, then filtered and concentrated under reduced pressure. The residue was purified by silica gel chromatography to yield title compound 3H. MS (m/z) 522.95 [M+Na]⁺.

### Step 9. Synthesis of 3-(2-(2-(Tert-butoxy)-2-oxoethyl)-6-((di-tert-butoxyphosphoryl)oxy)-4-methylphenyl)-3-methylbutanoic acid (3I)

To a solution of 3H (0.356 mmol) in ACN/water (1:1, 3.0 mL) was added TEMPO (0.018 mmol), potassium dihydrogen phosphate (0.178 mmol) and disodium hydrogen phosphate (0.178 mmol). The solution was cooled to 0° C. Sodium chlorite (0.533 mmol) was added, followed by sodium hypochlorite (8.25% NaOCl, 266 μL). The ice bath was removed and the reaction was stirred for 3.5 hours. The reaction was diluted with water, extracted with EtOAc (3×), the combined organic layers were washed with brine, dried over MgSO₄, filtered and concentrated under reduced pressure to yield title compound 31 which was used without further purification. MS (m/z) 536.85 [M+Na]⁺.

### Step 10. Prophetic Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (3J)

To a solution of crude 31 (1.0 eq) in ACN (0.1 M) is added N-methylimidazole (3.5 eq), Intermediate 1A (1.0 eq) and TCFH (1.0 eq). The reaction is stirred until completion. The reaction is quenched with sat. aqueous NH₄Cl and extracted with EtOAc (3×). The combined organic layers are washed with brine, dried over MgSO₄, filtered and concentrated under reduced pressure. The crude material is purified by reverse phase HPLC. Fractions containing title compound are pooled and concentrated to afford title compound 3J.

### Step 10. Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (3J)

To a solution of 3I (126 μmol) in ACN (0.5 mL) was added NMI (221 μmol), TCFH (63.1 μmol) and Intermediate 1 (63.1 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 3J. MS (m/z): 1278 [(M−3tBu)+H]⁺.

### Step 11. Prophetic Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 3)

Compound 3J (1.0 eq) is dissolved in DCM/TFA (3:1, 0.03 M). The reaction is stirred at RT until reaction completion. The reaction is concentrated and the crude product is purified by RP chromatography. Fractions containing the product are pooled and lyophilized to afford title compound 3.

### Step 11. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 3)

To a solution of 3J (27.7 μmol) in DCM (1.5 mL) was added TFA (0.5 mL). The mixture was stirred at 25° C. for 0.5 hour. The reaction mixture was concentrated and purified by reverse phase HPLC (ACN/water with 0.1% TFA). Fractions containing the product were pooled and concentrated to afford an atropisomeric mixture of title compound 3. ¹H NMR (400 MHz, DMSO-d₆) δ 8.89 (t, J=6.7 Hz), 8.57 (dd, J=21.6, 8.7 Hz), 8.44-8.27 (m), 8.02 (d, J=7.8 Hz), 7.80 (d, J=7.9 Hz), 7.62 (d, J=7.9 Hz), 7.52 (t, J=8.7 Hz), 7.27-7.08 (m), 7.08-6.95 (m), 6.89 (d, J=11.3 Hz), 6.79-6.53 (m), 4.78-3.90 (m), 3.85-3.38 (m), 3.28-3.01 (m), 2.74-2.57 (m), 2.48-2.37 (m), 2.17 (d, J=8.6 Hz), 1.57-1.10 (m), 0.97-0.82 (m) ppm. ³¹P NMR (162 MHz, DMSO-d₆) δ=−7.12, −7.62 ppm. MS (m/z): 1278.2 [M+H]⁺.

### Example 4. 2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 4)

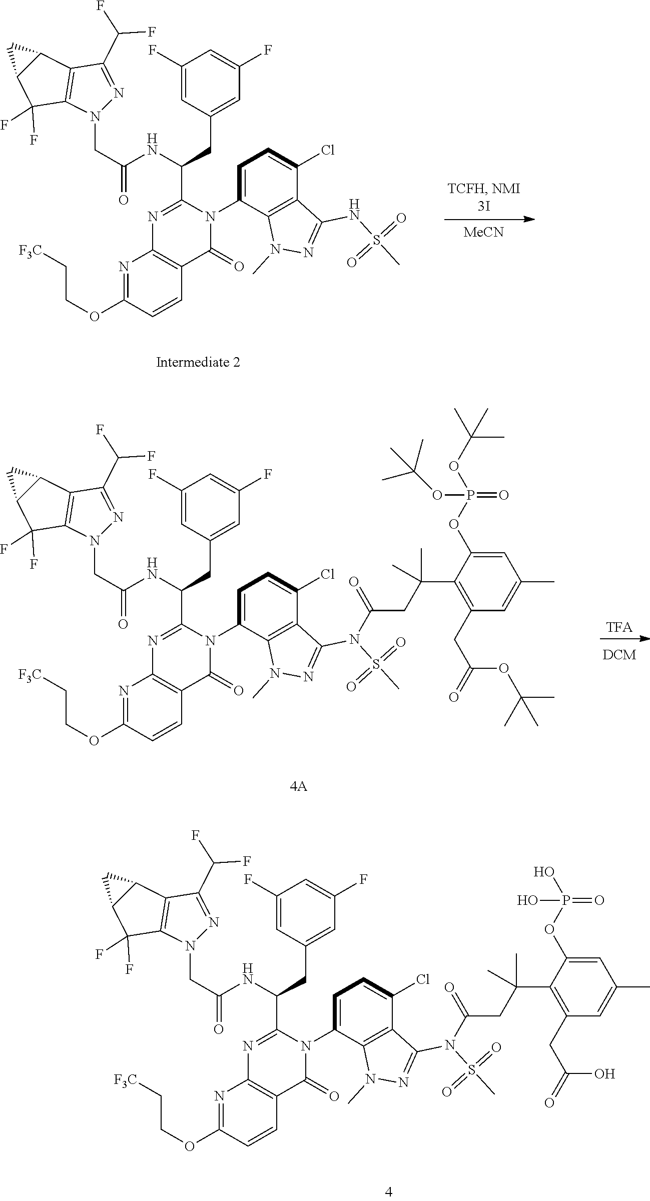

### Step 1. Prophetic Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cycopropa[3,4]cycopenta[, 2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (Compound 4A)

To a solution of crude 31 (1.0 eq) in ACN (0.1 M) is added N-methylimidazole (3.5 eq), Intermediate 2A (1.0 eq) and TCFH (1.0 eq). The reaction is stirred until completion. The reaction is quenched with sat. aqueous NH₄Cl and extracted with EtOAc (3×). The combined organic layers are washed with brine, dried over MgSO₄, filtered and concentrated under reduced pressure. The crude material is purified by reverse phase HPLC. Fractions containing title compound are pooled and concentrated to afford title compound 4A.

### Step 1. Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (Compound 4A)

To a solution of 31 (153 μmol) in ACN (1.0 mL) was added NMI (267 μmol), TCFH (76.2 μmol) and Intermediate 2 (76.2 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 4A. MS (m/z): 1246.3 [M−3[tBu]+H]⁺.

### Step 2. Prophetic Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 4)

Compound 4A (1.0 eq) is dissolved in DCM/TFA (3:1, 0.03 M). The reaction is stirred at RT until reaction completion. The reaction is concentrated and the crude product is purified by RP. Fractions containing the product are pooled and lyophilized to afford title compound 4.

### Step 2. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 4)

To a solution of 4A (28.3 μmol) in DCM (1.5 mL) was added TFA (0.5 mL). The mixture was stirred at 25° C. for 1 hour. The reaction mixture was concentrated and purified by reverse phase HPLC (ACN/water with 0.1% TFA). Fractions containing the product were pooled and concentrated to afford an atropisomeric mixture of title compound 4. ¹H NMR (400 MHz, DMSO-d₆) δ ppm 9.25-9.15 (m), 8.46 (dd, J=8.6, 1.7 Hz), 7.82 (d, J=7.9 Hz), 7.66 (d, J=7.9 Hz), 7.54 (t, J=8.5 Hz), 7.16 (bs), 7.14-7.07 (m), 7.07-6.96 (m), 6.89 (d, J=17.1 Hz), 6.79-6.62 (m), 6.58 (bs), 4.76-4.32 (m), 3.83-3.34 (m), 3.28-2.84 (m), 2.48-2.39 (m), 2.17 (d, J=7.3 Hz), 1.52-1.10 (m), 0.94-0.81 (m) ppm. ³¹P NMR (162 MHz, DMSO-d₆) δ=−7.12, −7.46 ppm. MS (m/z): 1246.3 [M+H]⁺.

### Example 5. (2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(411)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 5)

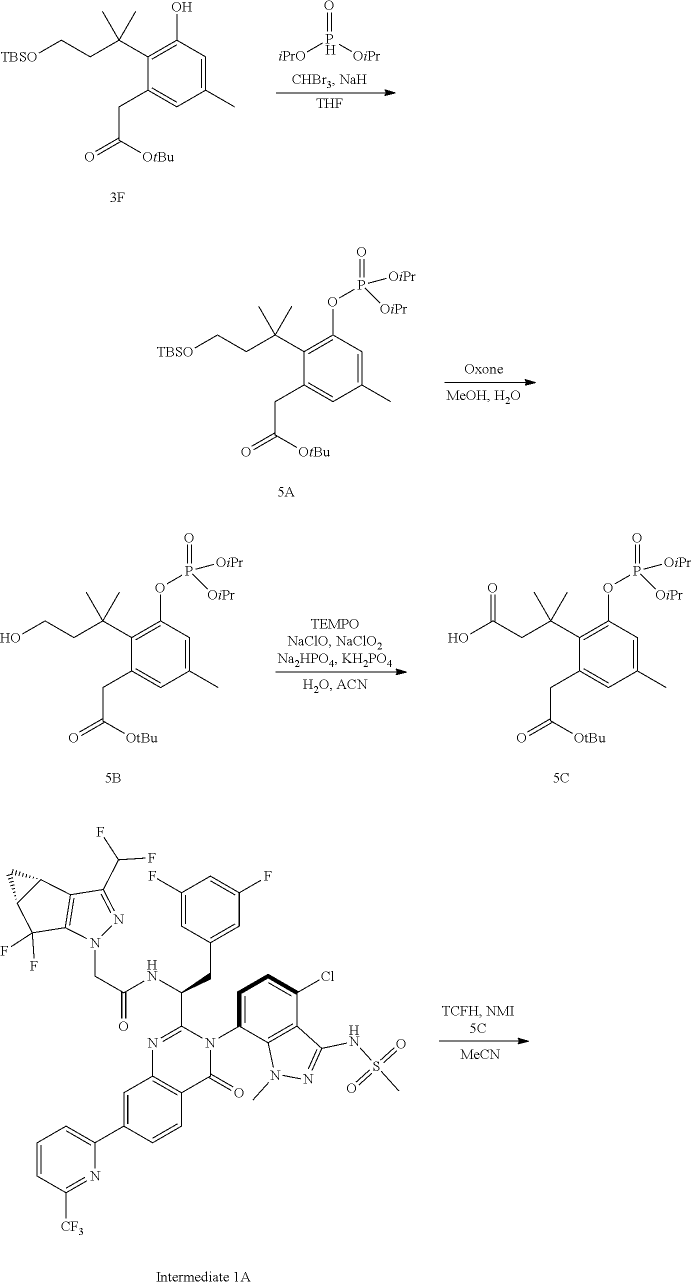

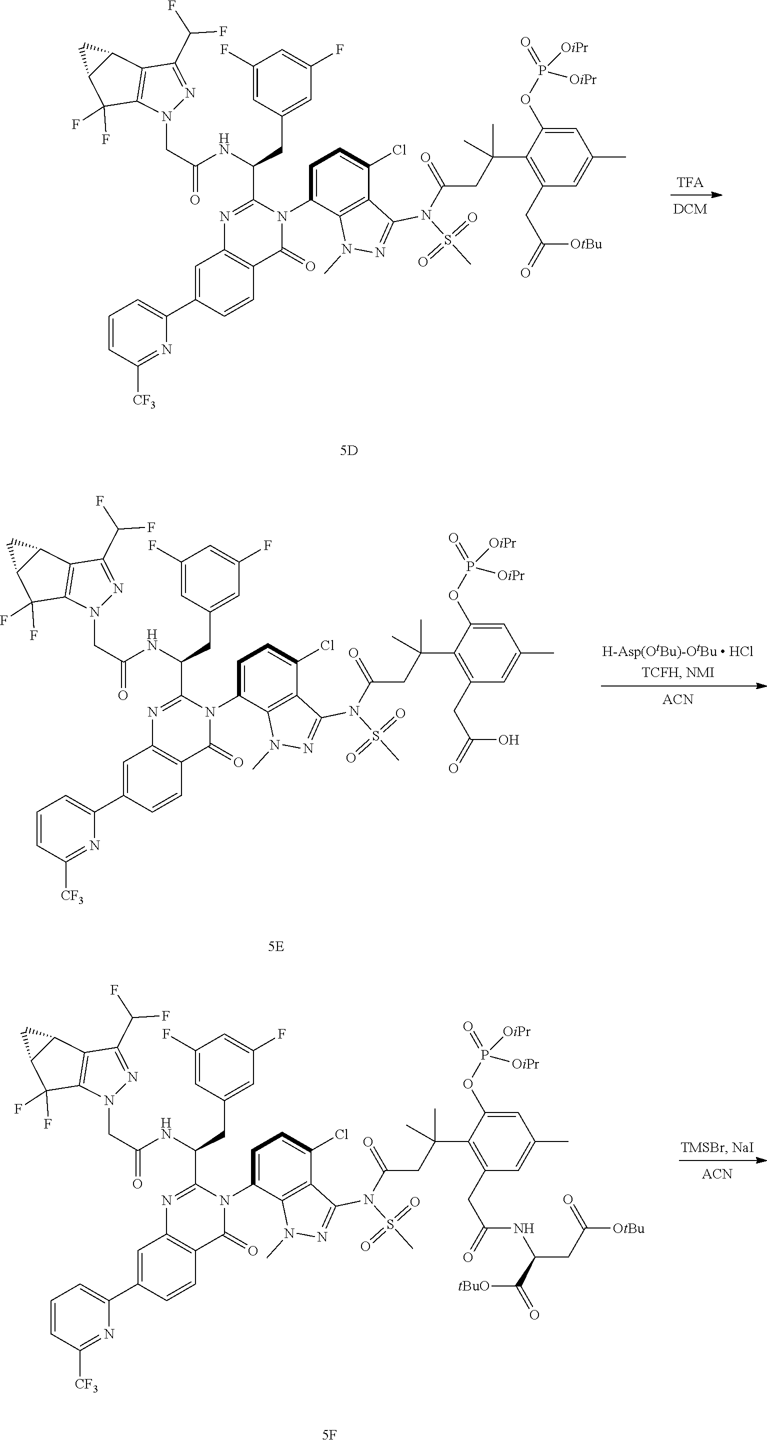

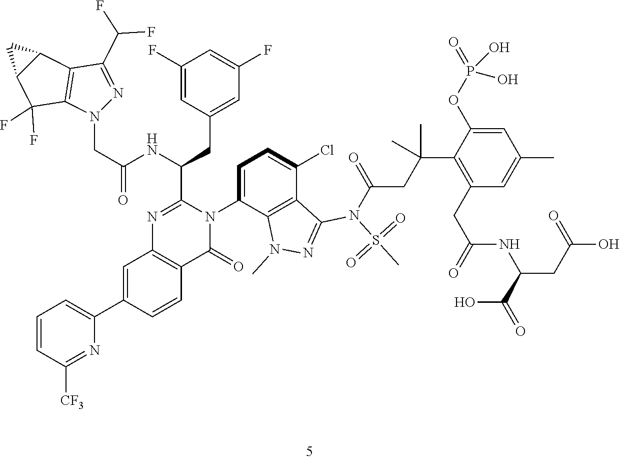

### Step 1. Synthesis of tert-butyl 2-(2-(4-((tert-butyldimethylsilyl)oxy)-2-methylbutan-2-yl)-3-((diisopropoxyphosphoryl)oxy)-5-methylphenyl)acetate (5A)

To a two-neck flask under argon and chilled on an ice bath was added 3F (1.01 mmol), THE (50.0 mL), diisopropyl phosphite (1.36 mmol) and bromoform (1.36 mmol). Sodium hydride (60% dispersion in mineral oil, 1.31 mmol) was added and the reaction was stirred at room temperature. Upon completion, the reaction mixture was diluted with DCM (150 mL), washed with water (1×50 mL), brine, dried over Na₂SO₄, concentrated and purified by silica gel chromatography. Fractions containing the product were pooled and concentrated to yield title compound 5A. MS (m/z) 587.40 [M+H]⁺.

### Step 2. Synthesis of tert-butyl 2-(3-((diisopropoxyphosphoryl)oxy)-2-(4-hydroxy-2-methylbutan-2-yl)-5-methylphenyl)acetate (5B)

To an ice-cold solution of 5A (0.256 mmol) in MeOH/H₂O (1:1, 1.3 mL) was added Oxone® (0.511 mmol). The reaction was stirred at room temperature. Upon completion, the reaction mixture was quenched with sat. Na₂SO₃ (aq) (1 mL) and stirred for 15 minutes. The reaction was filtered and the filtrate was concentrated. The remaining aqueous solution was extracted with EtOAc (5×10 mL). The organic fraction was collected, washed with brine, dried over Na₂SO₄, concentrated, and purified by silica gel chromatography. Fractions containing the product were pooled and concentrated to yield title compound 5B. MS (m/z) 473.30 [M+H]⁺.

### Step 3. Synthesis of 3-(2-(2-(tert-butoxy)-2-oxoethyl)-6-((diisopropoxyphosphoryl)oxy)-4-methylphenyl)-3-methylbutanoic acid (5C)

To an ice-cold solution of 5B (0.635 mmol), dipotassium hydrogen phosphate (0.317 mmol), potassium dihydrogen phosphate (0.317 mmol) and TEMPO (0.032 mmol) in MeCN/H₂O (1:1, 5.0 mL) was added sodium hypochlorite (8.25% NaOCl_{(aq)}, 0.775 mmol) and sodium chlorite (0.952 mmol). The reaction was stirred at room temperature. Upon completion, the reaction was diluted with water (10 mL), quenched with sat. Na₂SO₃ (aq) (1 mL), acidified to pH 2 with 1N HCl and extracted with EtOAc (3×20 mL). The organic fraction was collected, washed with brine, dried over Na₂SO₄, concentrated and purified by silica gel chromatography. Fractions containing the product were pooled and concentrated to yield title compound 5C which was used without further purification. MS (m/z) 509.20 [M+Na]⁺.

### Step 4. Prophetic Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetate (5D)

To a flask is added 5C (1.0 eq), Intermediate 1A (0.95 eq), MeCN (0.1 M), 1-methyl-1H-imidazole (3.5 eq) and TCFH (1.0 eq). The reaction is stirred at room temperature until completion. The reaction is diluted with sat. NH₄Cl (aq) and the aqueous layer is extracted with EtOAc. The organic fraction is dried over Na₂SO₄, concentrated and purified by silica gel chromatography. Fractions containing the product are pooled and concentrated to yield title compound 5D which may be used without further purification.

### Step 4. Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methy-4-oxobutan-2-yl)-3-((diisopropoxyphosphoryl)oxy)-5-methylphenyl)acetate (5D)

To a solution of 5C (211 μmol) in ACN (1.0 mL) was added NMI (368 μmol), TCFH (105 μmol) and Intermediate 1 (105 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 5D. MS (m/z): 1418.4 [M+H]⁺.

### Step 5. Prophetic Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (5E)

Compound 5D (1.0 eq) is dissolved in DCM/TFA (3:1, 0.03 M). The reaction is stirred at RT until reaction completion. The reaction is concentrated and the crude product is purified by RP chromatography. Fractions containing the product are pooled and lyophilized to afford title compound 5E.

### Step 5. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (5E)

To a solution of 5D (77.5 μmol) in DCM (1.5 mL) was added TFA (0.5 mL). The mixture was stirred at 25° C. for 20 mins. The reaction mixture was concentrated and purified by reverse phase HPLC (ACN/water with 0.1% TFA). Fractions containing the product were pooled and concentrated to afford an atropisomeric mixture of title compound 5E. MS (m/z): 1362.4 [M+H]⁺.

### Step 6. Prophetic Synthesis of di-tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (5F)

To a flask is added 5E (1.0 eq), di-tert-butyl L-aspartate hydrochloride (1.5 eq), MeCN (0.2 M), NMI (5.0 eq), and TCFH (1.2 eq). The reaction is stirred until completion. The reaction is diluted with EtOAc and washed with 0.1 N HCl. The organic fraction is washed with brine, dried over Na₂SO₄, concentrated and purified by silica gel chromatography. Fractions containing the product are pooled and concentrated to yield intermediate 5F.

### Step 6. Synthesis of di-tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (5F)

To a solution of 5E (40.6 μmol) in ACN (1.0 mL) was added di-tert-butyl L-aspartate hydrochloride (61.0 μmol), NMM (203 μmol) and TCFH (48.8 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 5F. MS (m/z): 1590.6 [M+H]⁺.

### Step 7. Prophetic Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 5)

A flask is charged with 5F (1.0 eq), sodium iodide (2.0 eq), MeCN (0.1 M), and bromotrimethylsilane (10 eq) and is stirred at 40° C. until completion. The reaction is brought to room temperature and quenched with a few drops of water. The solution is directly purified by reverse phase HPLC. Fractions containing the product are pooled and lyophilized to yield title compound 5.

### Step 7. Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 5)

To a solution of 5F (29.3 μmol) in ACN (0.5 mL) was added NaI (58.7 μmol) and TMSBr (235 μmol). The mixture was stirred at 45° C. for 1 hour under N₂. The reaction mixture was concentrated and purified by reverse phase HPLC (ACN/water with 0.1% TFA). Fractions containing the product were pooled and concentrated to afford an atropisomeric mixture of title compound 5. ¹H NMR (400 MHz, DMSO-d₆) δ 12.42 (bs), 8.88 (d, J=7.5 Hz), 8.57 (dd, J=16.7, 6.2 Hz), 8.46-8.27 (m), 8.15 (t, J=8.0 Hz), 8.02 (d, J=7.8 Hz), 7.79 (d, J=7.8 Hz), 7.66 (d, J=7.9 Hz), 7.56-7.46 (m), 7.26-6.94 (m), 6.90 (d, J=3.6 Hz), 6.80-6.55 (m), 4.74-4.23 (m), 3.84-3.33 (m), 3.32-2.91 (m), 2.81-2.38 (m), 2.16 (d, J=9.3 Hz), 1.57-1.11 (m), 0.96-0.81 (m) ppm. ³¹P NMR (162 MHz, DMSO-d₆) δ=−7.13, −7.80 ppm. MS (m/z): 1393.2 [M+H]⁺.

### Example 6. (2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 6)

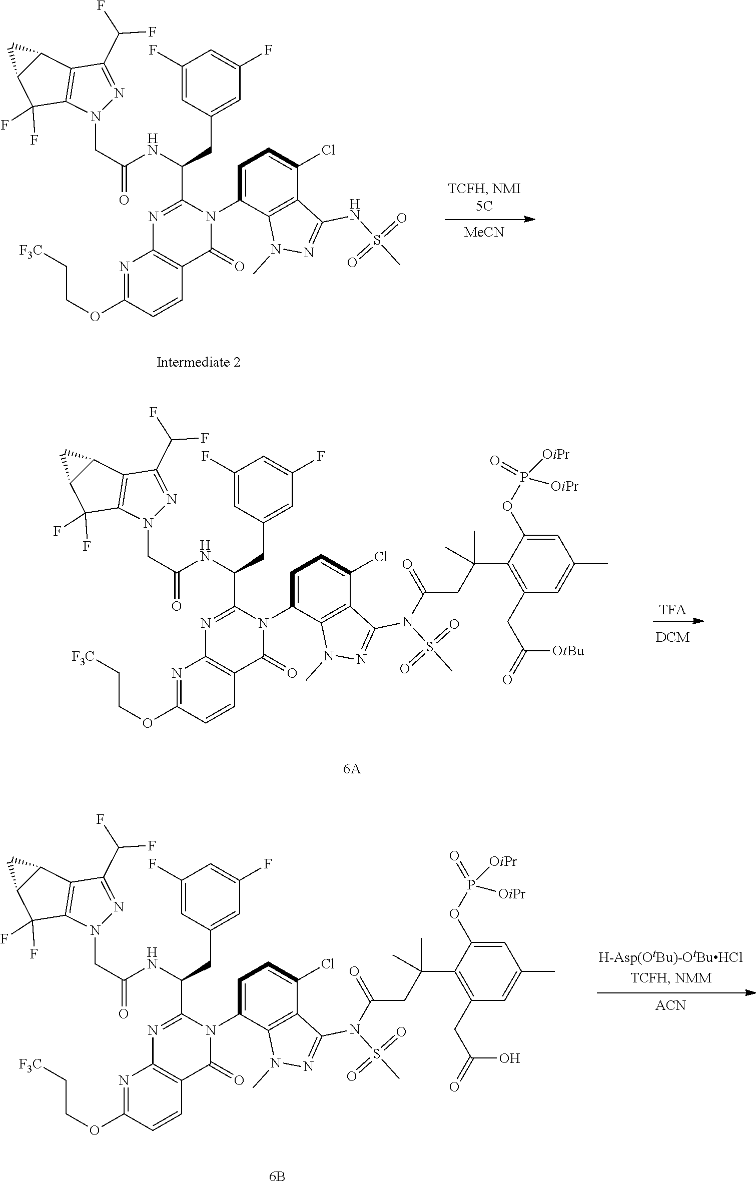

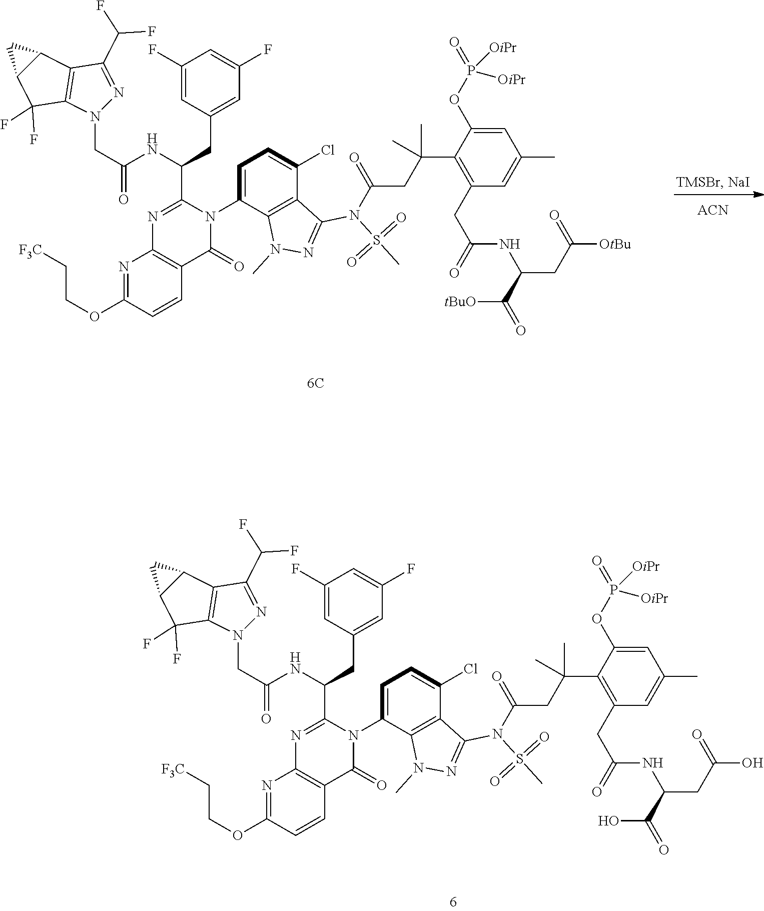

### Step 1. Prophetic Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetate (6A)

To a flask is added 5C (1.0 eq), Intermediate 2A (0.95 eq), MeCN (0.1 M), 1-methyl-1H-imidazole (3.5 eq) and TCFH (1.0 eq). The reaction is stirred at room temperature until completion. The reaction is diluted with sat. NH₄Cl (aq) and the aqueous layer is extracted with EtOAc. The organic fraction is dried over Na₂SO₄, concentrated and purified by silica gel chromatography. Fractions containing the product are pooled and concentrated to yield title compound 6A which may be used without further purification.

### Step 1. Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetate (6A)

To a solution of 5C (218 μmol) in ACN (1.0 mL) was added NMI (381 μmol), TCFH (109 μmol) and Intermediate 2 (109 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 6A. MS (m/z): 1386.5 [M+H]⁺.

### Step 2. Prophetic Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (6B)

Compound 6A (1.0 eq) is dissolved in DCM/TFA (3:1, 0.03 M). The reaction is stirred at RT until reaction completion. The reaction is concentrated and is purified by RP chromatography. Fractions containing the product are pooled and lyophilized to afford title compound 6B.

### Step 2. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (6B)

To a solution of 6A (79.3 μmol) in DCM (1.5 mL) was added TFA (0.5 mL). The mixture was stirred at 25° C. for 20 mins. The reaction mixture was concentrated and purified by reverse phase HPLC (ACN/water with 0.1% TFA). Fractions containing the product were pooled and concentrated to afford an atropisomeric mixture of title compound 6B. MS (m/z): 1330.4 [M+H]⁺.

### Step 3. Prophetic Synthesis of di-tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (6C)

To a flask is added Compound 6B (1.0 eq), di-tert-butyl L-aspartate hydrochloride (1.5 eq), MeCN (0.2 M), NMI (5.0 eq), and TCFH (1.2 eq). The reaction is stirred until completion. The reaction is diluted with EtOAc and washed with 0.1 N HCl. The organic fraction is washed with brine, dried over Na₂SO₄, concentrated and purified by silica gel chromatography. Fractions containing the product are pooled and concentrated to yield intermediate 6C.

### Step 3. Synthesis of di-tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (6C)

To a solution of 6B (41.5 μmol) in ACN (1.0 mL) was added di-tert-butyl L-aspartate hydrochloride (62.3 μmol), NMM (208 μmol) and TCFH (49.8 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 6C. MS (m/z): 1558.4 [M+H]⁺.

### Step 4. Prophetic Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 6)

A flask is charged with Compound 6C (1.0 eq), sodium iodide (2.0 eq), MeCN (0.1 M), and bromotrimethylsilane (10 eq) and is stirred at 40° C. until completion. The reaction is brought to room temperature and quenched with a few drops of water. The solution is directly purified by reverse phase HPLC. Fractions containing the product are pooled and lyophilized to yield title compound 6.

### Step 4. Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 6)

To a solution of 6C (38.3 μmol) in ACN (1.0 mL) was added NaI (76.6 μmol) and TMSBr (306 μmol). The mixture was stirred at 45° C. for 1 hour under N₂. The reaction mixture was concentrated and purified by reverse phase HPLC (ACN/water with 0.1% TFA). Fractions containing the product were pooled and concentrated to afford an atropisomeric mixture of title compound 6. ¹H NMR (400 MHz, DMSO-d₆) δ 9.21 (d, J=8.0 Hz), 8.45 (dd, J=8.6, 3.3 Hz), 8.18-8.03 (m), 7.82 (d, J=7.9 Hz), 7.69 (d, J=8.0 Hz), 7.61-7.43 (m), 7.30-6.93 (m), 6.90 (d, J=6.5 Hz), 6.81-6.53 (m), 4.82-4.26 (m), 3.96-3.34 (m), 3.31-2.83 (m), 2.79-2.37 (m), 2.15 (d, J=8.5 Hz), 1.59-1.08 (m), 0.95-0.81 (m) ppm. ³¹P NMR (162 MHz, DMSO-d₆) δ=−7.05, −7.62 ppm. MS (m/z): 1361.1 [M+H]⁺.

### Example 7. (2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 7)

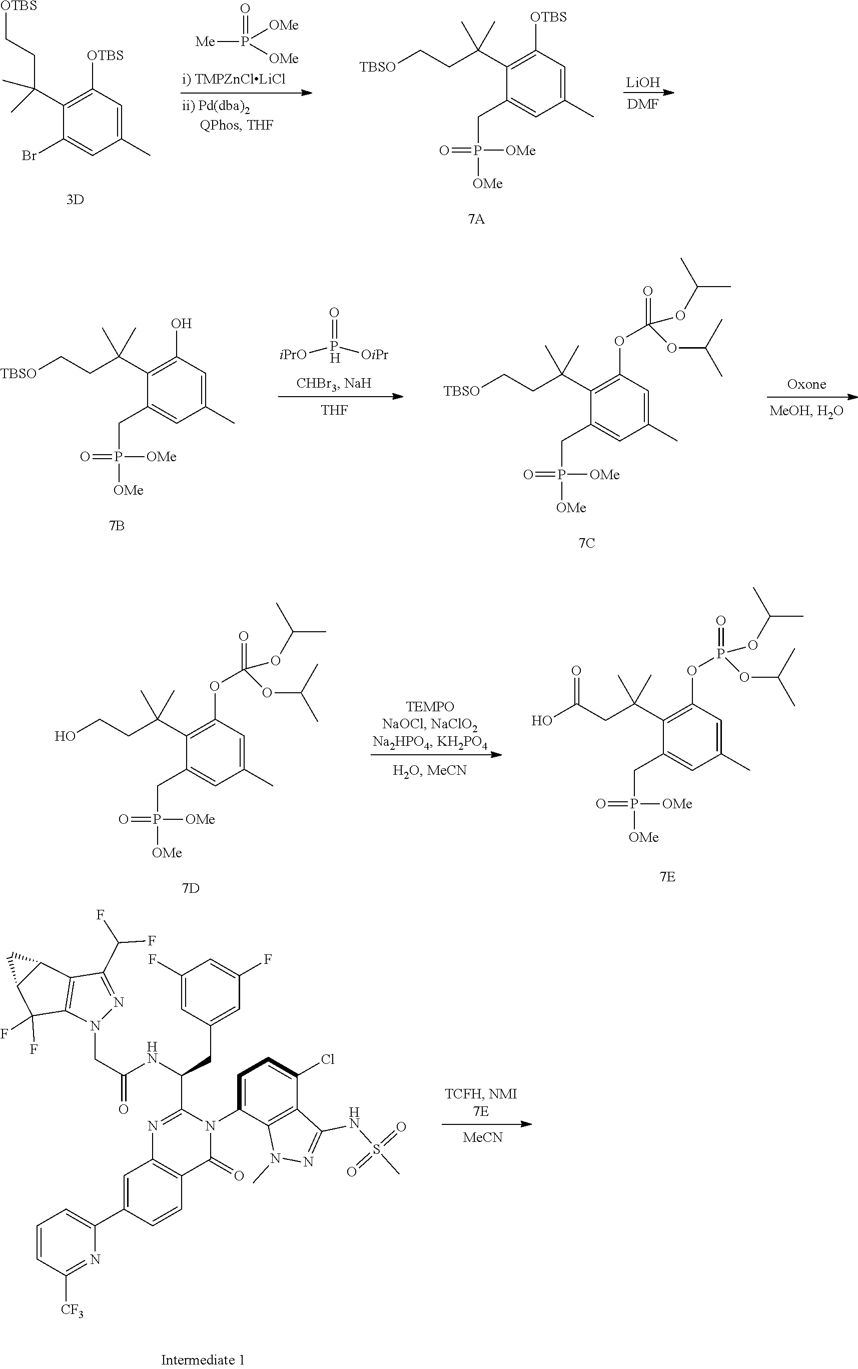

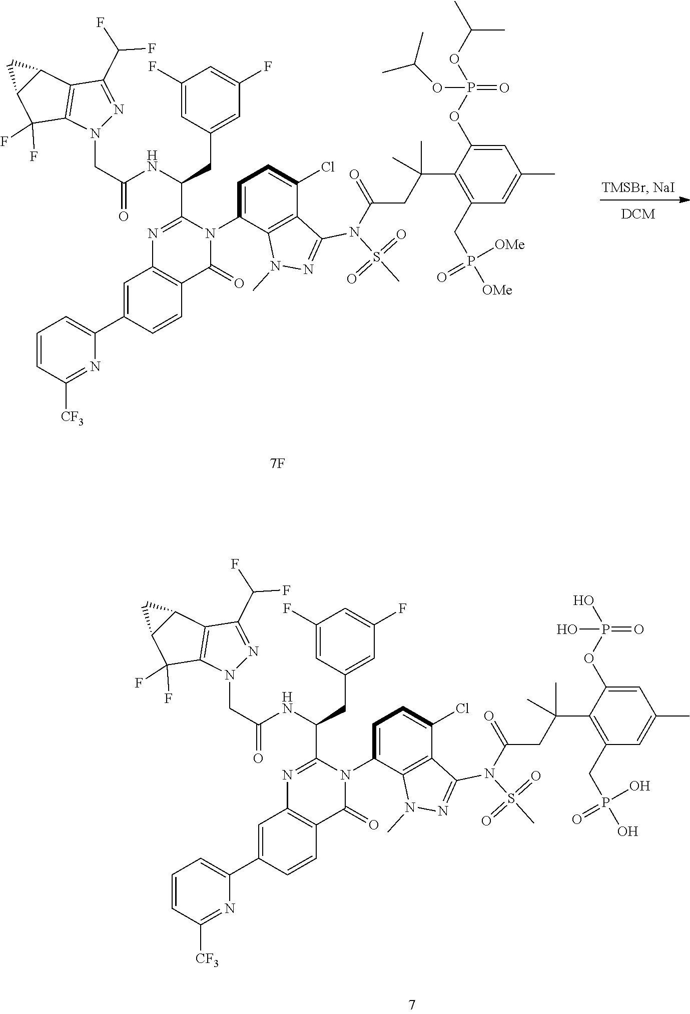

### Step 1. Synthesis of Dimethyl (3-((tert-butyldimethylsilyl)oxy)-2-(4-((tert-butyldimethylsilyl)oxy)-2-methylbutan-2-yl)-5-methylbenzyl)phosphonate (7A)

A flask was charged with dimethyl methylphosphonate (4.78 mmol) and THE (4.8 mL) under nitrogen. TMPZnCl·LiCl (5.98 mmol, 1.OM in THF) was added and the reaction was monitored by ³¹P NMR. Upon completion, 1.42 mL (0.598 mmol) of the resulting organozinc reagent in solution was added under nitrogen to a flask containing 3D (0.498 mmol), Pd(dba)₂ (0.05 mmol), QPhos (0.05 mmol) and THE (2.5 mL). The reaction was heated at 50° C. for 3 hours, quenched with sat. NH₄Cl (aq) and extracted with EtOAc (3×). The combined organic layers were washed with brine, dried over MgSO₄, filtered and concentrated under reduced pressure. The crude material was purified by silica gel chromatography to afford title compound 7A. MS (m/z) 544.93 [M+H]⁺.

### Step 2. Synthesis of Dimethyl (2-(4-((tert-butyldimethylsilyl)oxy)-2-methylbutan-2-yl)-3-hydroxy-5-methylbenzyl)phosphonate (7B)

A flask was charged with 7A (0.158 mmol), anhydrous LiOH (0.474 mmol) and DMF (0.8 mL). The mixture was heated at 60° C. for 3 hours. The reaction was diluted with water and sat. NH₄Cl (aq), then extracted with EtOAc (3×). The combined organic layers were washed with 5% LiCl_{(aq)} (2×), dried over MgSO₄, filtered and concentrated. The crude material was purified by silica gel chromatography to afford title compound 7B. MS (m/z) 430.99 [M+H]⁺

### Step 3. Synthesis of 2-(4-((tert-butyldimethylsilyl)oxy)-2-methylbutan-2-yl)-3-((dimethoxyphosphoryl)methyl)-5-methylphenyl diisopropyl phosphate (7C)

A flask charged with 7B (1.31 mmol) and THE (6.0 mL) under nitrogen was cooled to 0° C. Diisopropyl phosphite (1.52 mmol) and bromoform (1.52 mmol) were added. The solution was stirred for 5 minutes, sodium hydride (60% dispersion in mineral oil, 1.52 mmol) was added in one portion and the mixture was gradually warmed to RT. Upon completion, the reaction was quenched with water and extracted with EtOAc (3×). The combined organic layers were washed with brine, dried over MgSO₄, filtered and concentrated. The crude material was purified by silica gel chromatography to afford title compound 7C. MS (m/z) 594.77 [M+H]⁺.

### Step 4. Synthesis of 3-((Dimethoxyphosphoryl)methyl)-2-(4-hydroxy-2-methylbutan-2-yl)-5-methylphenyl diisopropyl phosphate (7D)

A flask charged with 7C (0.193 mmol), MeOH (1.0 mL) and water (1.0 mL) was cooled to 0° C. Oxone® (0.387 mmol) was added, the ice bath was removed and the reaction was stirred at RT for 1 hour. The reaction was quenched with sat. sodium sulfite (aq), concentrated, diluted with water and extracted with EtOAc (3×). The combined organic layers were washed with brine, dried over MgSO₄, filtered and concentrated. The crude material was purified by silica gel chromatography to afford title compound 7D. MS (m/z) 480.93 [M+H]⁺.

### Step 5. Synthesis of 3-(2-((Diisopropoxyphosphoryl)oxy)-6-((dimethoxyphosphoryl)methyl)-4-methylphenyl)-3-methylbutanoic acid (7E)

To a solution of 7D (2.84 mmol) in ACN/water (1:1, 30.0 mL) was added TEMPO (0.142 mmol), potassium dihydrogen phosphate (1.42 mmol) and disodium hydrogen phosphate (1.42 mmol). The solution was cooled to 0° C. Sodium chlorite (4.26 mmol) was added, followed by sodium hypochlorite (8.25% NaOCl (aq), 3.47 mmol). The ice bath was removed and the reaction was stirred for 2 hours. The reaction was diluted with water, quenched with sat. sodium sulfite_{(aq)} and acidified with 1N HCl (aq) to pH 2. The aqueous layer was extracted with EtOAc (3×). The combined organic layers were washed with brine, dried over MgSO₄, filtered and concentrated under reduced pressure to yield title compound 7E which was used without further purification. MS (m/z) 493.39 [M−H]⁻.

### Step 6. Prophetic Synthesis of 2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (7F)

To a solution of crude 7E (1.0 eq) in ACN (0.14 M) is added N-methylimidazole (3.5 eq), Intermediate 1A (1.0 eq) and TCFH (1.0 eq). The reaction is stirred until completion. The reaction is quenched with sat. NH₄Cl (aq) and extracted with EtOAc (3×). The combined organic layers are washed with brine, dried over MgSO₄, filtered and concentrated under reduced pressure. The crude material is purified by reverse phase HPLC. Fractions containing title compound are pooled and concentrated to afford title compound 7F.

### Step 6. Synthesis of 2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (7F)

To a solution of 7E (126 μmol) in ACN (0.5 mL) was added NMI (221 μmol), TCFH (63.1) and Intermediate 1 (63.1 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 7F. MS (m/z): 714.2 [M+2H]²⁺.

### Step 7. Prophetic Synthesis of (2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 7)

A flask charged with 7F (1.0 eq), sodium iodide (2.0 eq) and bromotrimethylsilane (8.0 eq) in DCM (0.16 M) is heated under nitrogen at 45° C. until completion. The reaction is diluted with water, extracted with EtOAc (3×) and the combined organic layers are concentrated. The crude product is purified by RP chromatography. Fractions containing the product are pooled and lyophilized to afford title compound 7.

### Step 7. Synthesis of (2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 7)

To a solution of 7F (29.9 μmol) in DCM (1.0 mL) was added TMSBr (299 mol). The mixture was stirred at 45° C. for 1 hour under N₂. The reaction mixture was concentrated and purified by reverse phase HPLC (ACN/water with 0.1% TFA). Fractions containing the product were pooled and concentrated to afford an atropisomeric mixture of title compound 7. ¹H NMR (400 MHz, DMSO-d₆) δ 8.87 (d, J=7.7 Hz), 8.62-8.51 (m), 8.44-8.27 (m), 8.02 (d, J=7.8 Hz), 7.79 (d, J=7.9 Hz), 7.60 (d, J=7.9 Hz), 7.50 (dd, J=19.2, 7.9 Hz), 7.15-6.96 (m), 6.91 (d, J=9.1 Hz), 6.82-6.64 (m), 4.74-4.18 (m), 3.69-3.43 (m), 3.42-2.99 (m), 2.52-2.38 (m), 2.16 (d, J=9.5 Hz), 1.66-1.42 (m), 1.39-1.24 (m), 0.94-0.85 (m) ppm. ³¹P NMR (162 MHz, DMSO-d₆) δ=21.63, 21.56, −7.15, −7.84 ppm. MS (m/z): 1314.1 [M+H]⁺.

### Example 8. (2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 8)

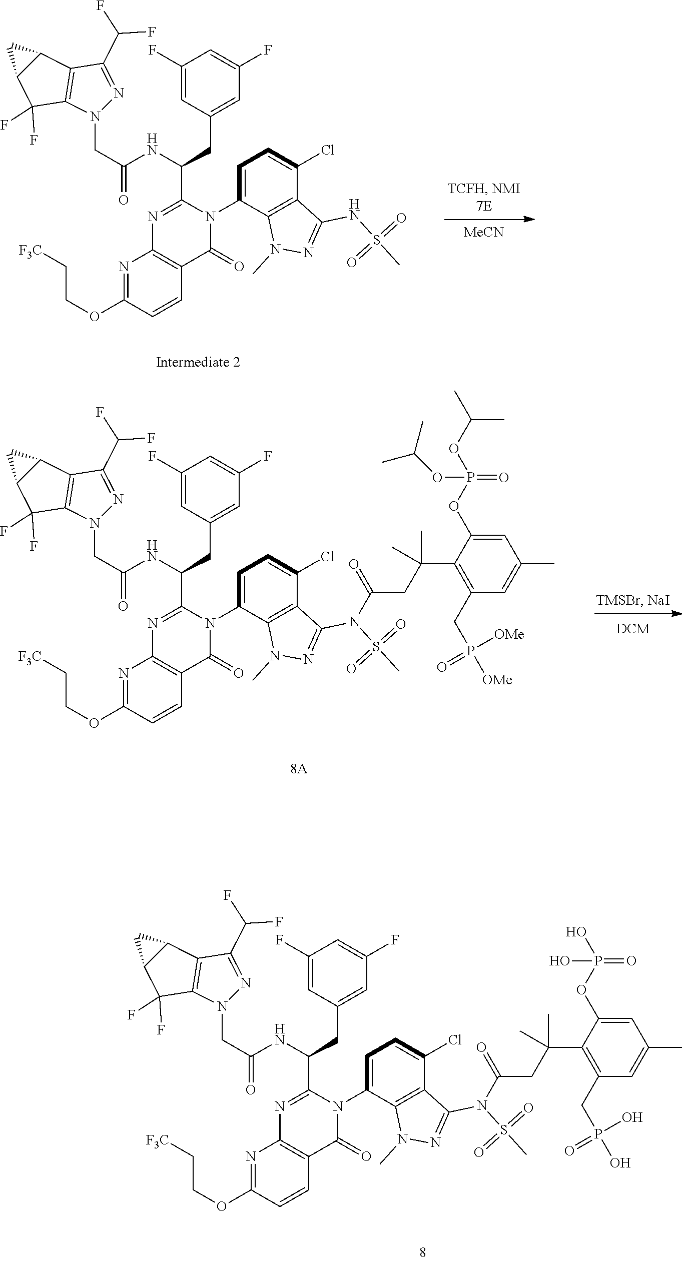

### Step 1. Prophetic Synthesis of 2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (8A)

To a solution of crude 7E (1.0 eq) in ACN (0.14 M) is added N-methylimidazole (3.5 eq), Intermediate 2A (1.0 eq) and TCFH (1.0 eq). The reaction is stirred until completion. The reaction is quenched with sat. NH₄Cl (aq) and extracted with EtOAc (3×). The combined organic layers are washed with brine, dried over MgSO₄, filtered and concentrated under reduced pressure. The crude material is purified by reverse phase HPLC. Fractions containing title compound are pooled and concentrated to afford title compound 8A.

### Step 1. Synthesis of 2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (8A)

To a solution of 7E (131 μmol) in ACN (1.0 mL) was added NMI (229 μmol), TCFH (65.4 μmol) and Intermediate 2 (65.4 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 8A. MS (m/z): 1394.4 [M+H]⁺.

### Step 2. Prophetic Synthesis of (2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 8)

A flask charged with 8A (1.0 eq), sodium iodide (2.0 eq) and bromotrimethylsilane (8.0 eq) in DCM (0.16 M) is heated under nitrogen at 45° C. until completion. The reaction is diluted with water, extracted with EtOAc (3×) and the combined organic layers are concentrated. The crude product is purified by RP chromatography. Fractions containing the product are pooled and lyophilized to afford title compound 8.

### Step 2. Synthesis of (2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 8)

To a solution of 8A (40.2 μmol) in DCM (1.0 mL) was added TMSBr (402 mol). The mixture was stirred at 45° C. for 2 hours under N₂. The reaction mixture was concentrated and purified by reverse phase HPLC (ACN/water with 0.1% TFA). Fractions containing the product were pooled and concentrated to afford an atropisomeric mixture of title compound 8. ¹H NMR (400 MHz, DMSO-d₆) δ 9.25-9.14 (m), 8.45 (dd, J=8.6, 2.7 Hz), 7.82 (d, J=7.9 Hz), 7.64 (d, J=8.0 Hz), 7.59-7.46 (m), 7.15-6.98 (m), 6.92 (d, J=6.2 Hz), 6.80-6.64 (m), 4.76-4.33 (m), 3.67-3.19 (m), 3.17-3.01 (m), 3.00-2.84 (m), 2.48-2.39 (m), 2.15 (d, J=8.3 Hz), 1.64-1.41 (m), 1.40-1.24 (m), 0.94-0.81 (m) ppm. ³¹P NMR (162 MHz, DMSO-d₆) δ=21.65, 21.56, −7.16, −7.64 ppm. MS (m/z): 1282.1 [M+H]⁺.

### Example 9. (2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)glycine (Compound 9)

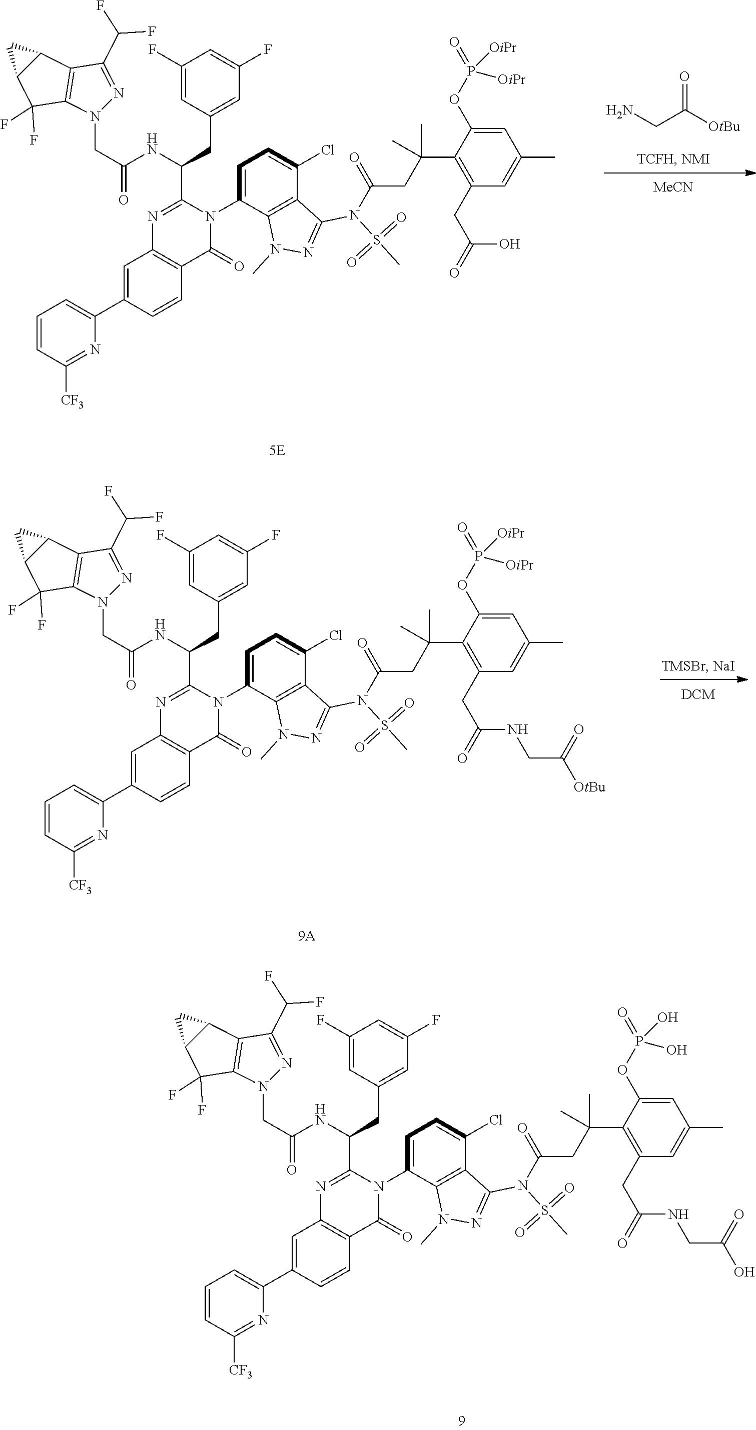

### Step 1. Prophetic Synthesis of tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)glycinate (9A)

To a vial is added 5E (1.0 eq), tert-butyl glycinate (2.0 eq), MeCN (0.1 M), NMI (5.0 eq) and TCFH (1.2 eq). The reaction is stirred at room temperature. Upon completion, the reaction is concentrated and is purified by silica gel chromatography. Fractions containing the product are pooled and concentrated to yield title compound 9A.

### Step 2. Prophetic Synthesis of (2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)glycine (Compound 9)

To a vial is added 9A (1.0 eq), sodium iodide (1.0 eq), MeCN (0.1 M) and bromotrimethylsilane (10 eq). The reaction is stirred at 40° C. Upon completion, the reaction is brought to room temperature and quenched with a few drops of water. The solution is directly purified by reverse phase HPLC. Fractions containing the product are pooled and lyophilized to yield title Compound 9.

### Example 10. (2-(2-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)glycine (Compound 10)

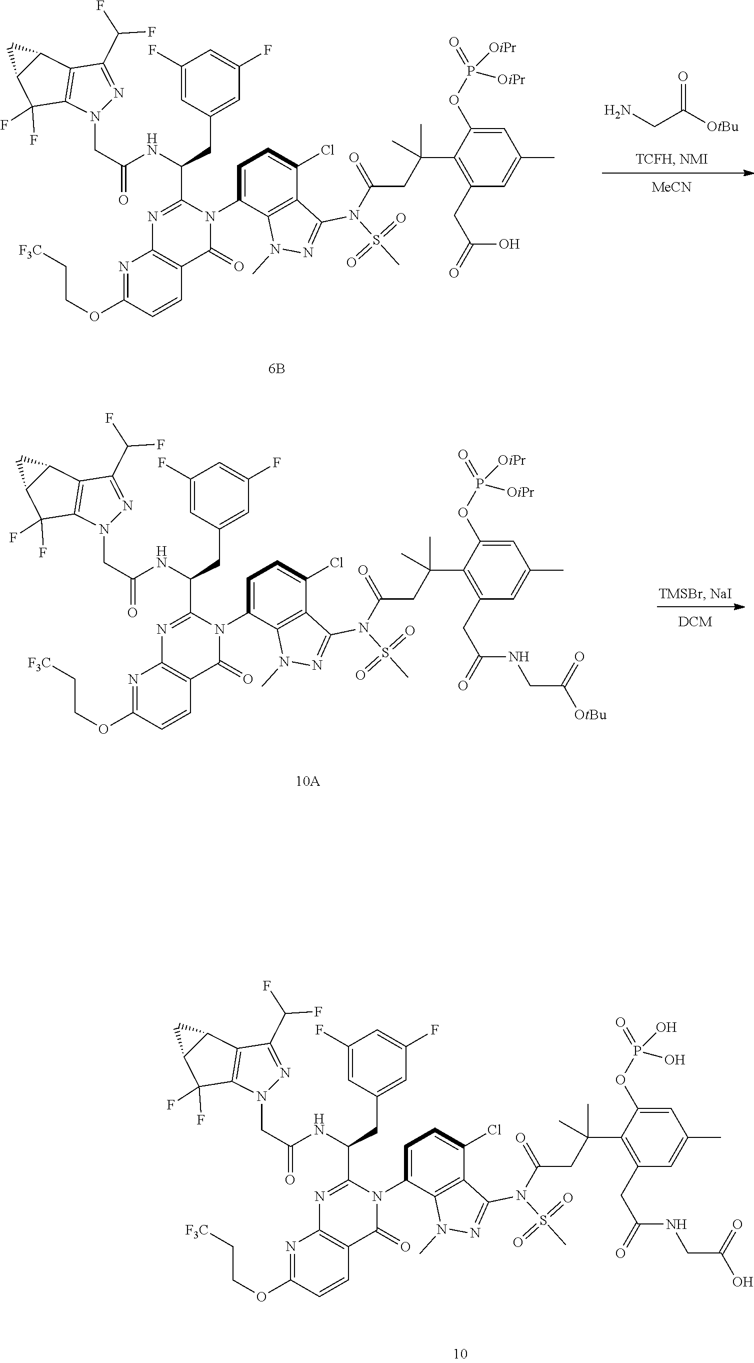

### Step 1. Prophetic Synthesis of tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)glycinate (10A)

To a vial is added 6B (1.0 eq), tert-butyl glycinate (2.0 eq), MeCN (0.1 M), NMI (5.0 eq) and TCFH (1.2 eq). The reaction is stirred at room temperature. Upon completion, the reaction is concentrated and is purified by silica gel chromatography. Fractions containing the product are pooled and concentrated to yield title compound 10A.

### Step 2. Prophetic Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)glycine (10)

To a vial is added 10A (1.0 eq), sodium iodide (1.0 eq), MeCN (0.1 M) and bromotrimethylsilane (10 eq). The reaction is stirred at 40° C. Upon completion, the reaction is brought to room temperature and quenched with a few drops of water. The solution is directly purified by reverse phase HPLC. Fractions containing the product are pooled and lyophilized to yield title compound 10.

### Example 11. N-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-4-oxobutyl)-N-(((phosphonooxy)methoxy)carbonyl)glycine (Compound 11)

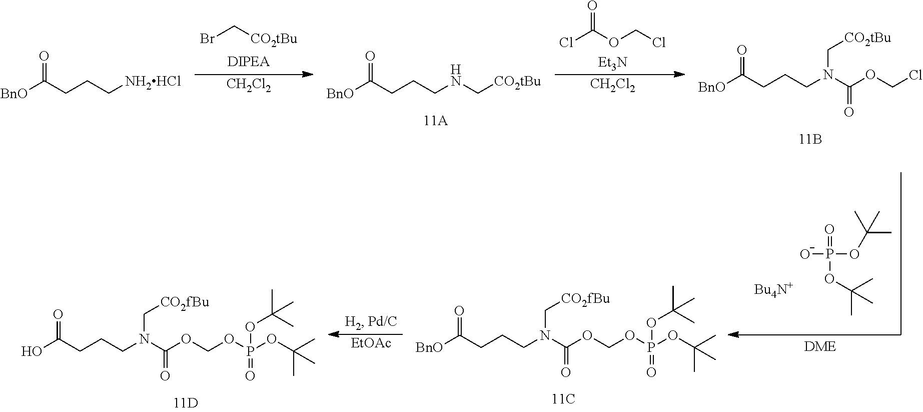

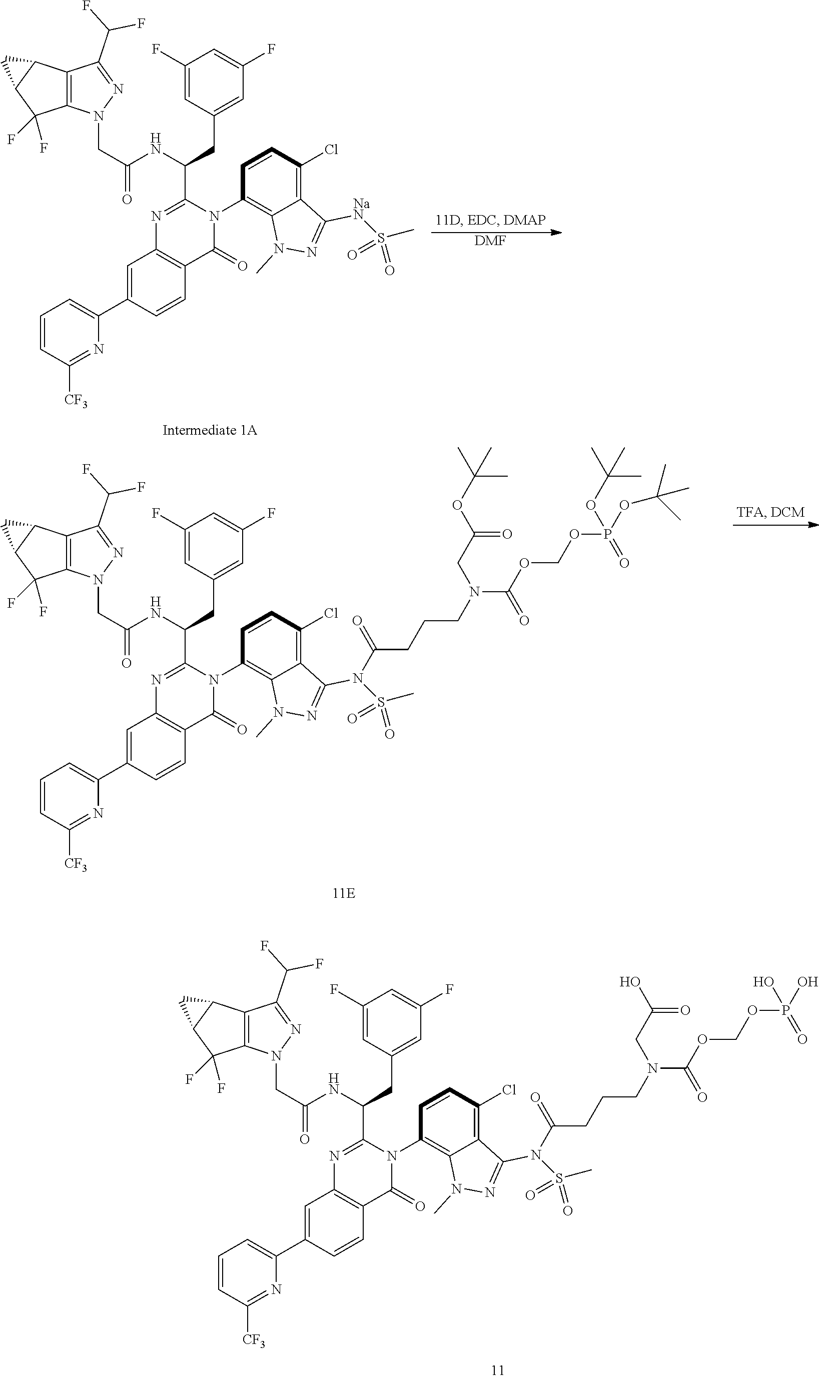

### Step 1. Synthesis of benzyl 4-((2-(tert-butoxy)-2-oxoethyl)amino)butanoate (HA)

To a stirred suspension of benzyl 4-aminobutanoate hydrochloride (113.2 mmol) in CH₂Cl₂ (560 mL) was added DIPEA (249 mmol), then the reaction mixture was cooled to 0° C. t-Butyl bromoacetate (56.6 mmol) in CH₂Cl₂ (25 mL) was added dropwise via syringe pump (1 mL/min). The resulting reaction mixture was stirred at 0° C. for 1 h then warmed to room temp. After stirring for an additional 1.5 h, the mixture was washed with 0.5 M aq. HCl solution (˜300 mL), water (˜200 mL), and brine. The combined organic layers were dried over anhydrous Na₂SO₄, filtered, and concentrated. The crude mixture was purified by column chromatography (0% to 10% MeOH in CH₂Cl₂) to afford title compound 11A. ¹H NMR (400 MHz, Chloroform-d) δ 9.45 (s, 1H), 7.40-7.29 (m, 5H), 5.13 (s, 2H), 3.77 (t, J=5.4 Hz, 2H), 3.28-3.19 (m, 2H), 2.61 (t, J=6.6 Hz, 2H), 2.24 (p, J=6.9 Hz, 2H), 1.50 (s, 9H) ppm. MS (m/z) 308.4 [M+H]⁺.

### Step 2. Synthesis of benzyl 4-((2-(tert-butoxy)-2-oxoethyl)((chloromethoxy)carbonyl)amino)butanoate (JIB)

To a stirred solution of 11A (23.4 mmol) in CH₂Cl₂ (234 mL) at 0° C. was added triethylamine (59 mmol). Chloromethyl chloroformate (35 mmol) was then added and the reaction mixture was stirred at 0° C. and monitored by TLC and LCMS. After completion, the reaction mixture was washed with sat. aq. solutions of NH₄Cl and brine. The combined organic layers were dried over anhydrous Na₂SO₄, filtered, and concentrated to afford title compound 11B, which was used without purification. MS (m/z) 401.5 [M+H]⁺.

### Step 3. Synthesis of benzyl 4-((2-(tert-butoxy)-2-oxoethyl)((((di-tert-butoxyphosphoryl)oxy)methoxy)carbonyl)amino)butanoate (11C)

Compound 11B (21 mmol) was dissolved in dimethoxyethane (87 mL) then di-t-butyl phosphate tetrabutylammonium salt (35 mmol) was added and the mixture was heated to 80° C. for 1 h. The mixture was cooled to room temperature and concentrated. The crude material was dissolved in EtOAc and washed with water (3×), brine, dried over Na₂SO₄, filtered, and concentrated. The crude mixture was purified by column chromatography (30% to 100% EtOAc in hexanes) to afford title compound 11C. ¹H NMR (400 MHz, Chloroform-d) δ 7.40-7.28 (m, 5H), 5.59 (t, J=12.5 Hz, 2H), 5.11 (s, 2H), 3.88 (d, J=2.4 Hz, 2H), 3.41-3.35 (m, 2H), 2.41 (q, J=7.6 Hz, 2H), 1.89 (m, 2H), 1.46 (d, J=10.0 Hz, 27H). MS (m/z) 574.6 [M+H]⁺.

### Step 4. Synthesis of 4-((2-(t-butoxy)-2-oxoethyl)((((di-t-butoxyphosphoryl)oxy)methoxy) carbonyl)amino)butanoic acid (11D)

Compound 11C (22 mmol) was dissolved in EtOAc then palladium on carbon (4.3 mmol) was added. After stirring for 1 h under hydrogen gas (1 atm), product formation was observed. The crude product was isolated after filtration over a pad of Celite®. The product was purified by column chromatography (0% to 10% MeOH in CH₂Cl₂) to afford title compound 11D. MS (m/z) 484.8 [M+H]⁺.

### Step 5. Prophetic Synthesis of tert-butyl N-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-4-oxobutyl)-N-((((di-tert-butoxyphosphoryl)oxy)methoxy)carbonyl)glycinate (11E)

To a solution of Intermediate 1A (1.0 eq), 11D (3.0 eq), and DMAP (2.0 eq) in DMF (0.1 M) is added EDC (3.0 eq). Upon reaction completion, the mixture is partitioned between EtOAc and water. The organic fraction is washed with 1N HCl and brine, then collected, dried over Na₂SO₄ and concentrated to afford an atropisomeric mixture of title compound 11E which may be used without purification.

### Step 6. Prophetic Synthesis of N-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-4-oxobutyl)-N-(((phosphonooxy)methoxy)carbonyl)glycine (Compound 11)

To a solution of 11E (1.0 eq) in DCM (0.1 M) is added trifluoroacetic acid (31 eq) with stirring. Upon reaction completion, the reaction is concentrated under reduced pressure and purified by reverse phase HPLC. Fractions containing the product are pooled and lyophilized to give title compound 11.

### Example 12. N-(4-(N-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-4-oxobutyl)-N-(((phosphonooxy)methoxy)carbonyl)glycine (Compound 12)

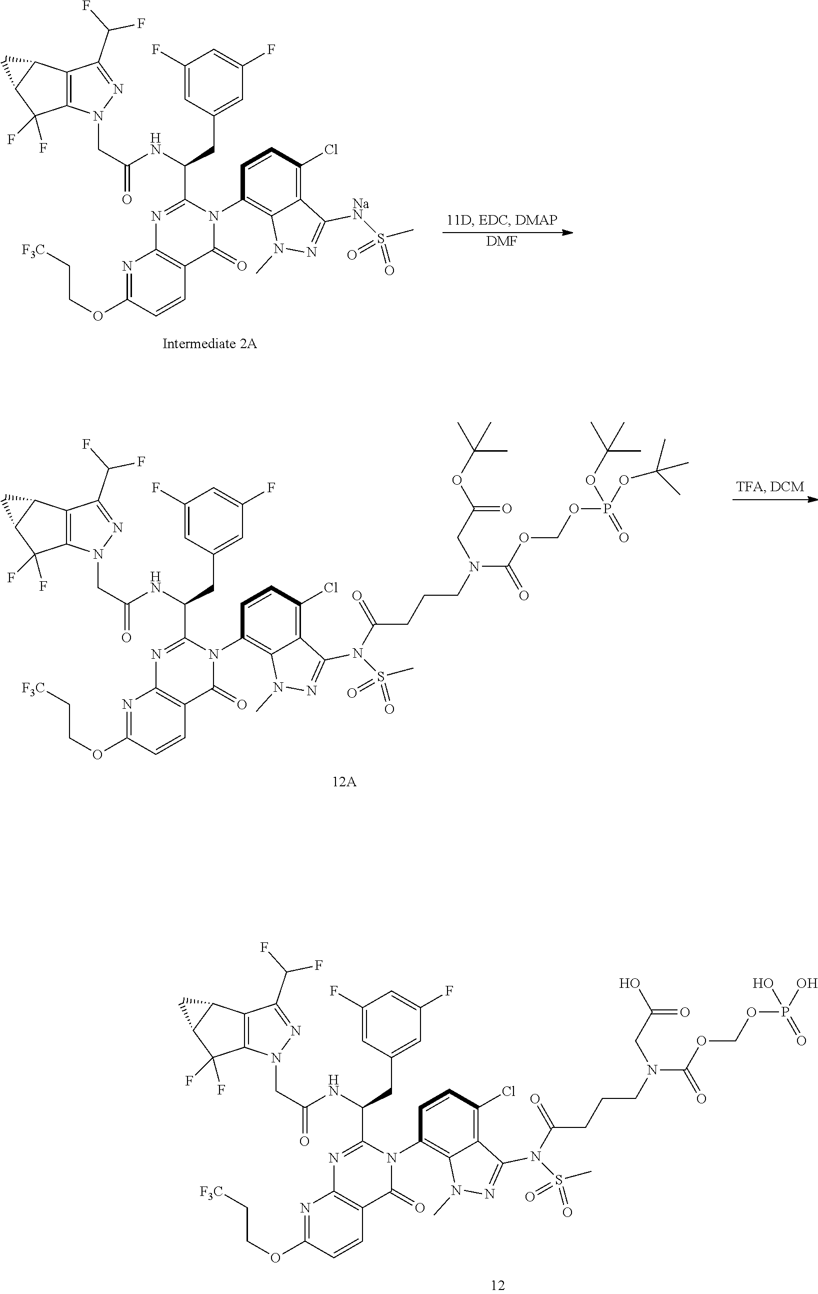

### Step 1. Prophetic Synthesis of tert-buyl N-(4-(N-(4-chloro-7-(2-((S)-1-(2-((3bS,4aR)-3-(difluoromethyl)-5,5-difluoro-3b,4,4a,5-tetrahydro-1H-cycopropa[3,4]cycopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-4-oxobutyl)-N-((((di-tert-butoxyphosphoryl)oxy)methoxy)carbonyl)glycinate (12A)

To a solution of Intermediate 2A (1.0 eq), 11D (3.0 eq), and DMAP (2.0 eq) in DMF (0.1 M) is added EDC (3.0 eq). Upon reaction completion, the mixture is partitioned between EtOAc and water. The organic fraction is washed with 1N HCl and brine, then collected, dried over Na₂SO₄ and concentrated to afford title compound 12A which may be used without purification.

### Step 2. Prophetic Synthesis of N-(4-(N-(4-chloro-7-(2-((S)-1-(2-((3bS,4aR)-3-(difluoromethyl)-5, 5-difluoro-3b, 4,4a, 5-tetrahydro-1H-cyclopropa[3, 4]cyclopenta[1, 2-c]pyrazol-yl)acetamido)-2-(3, 5-difluorophenyl)ethyl)-4-oxo-7-(3,3,3-trifluoropropoxy)pyrido[2, 3-d]pyrimidin-3(4H)-yl)-1-methyl-1H-indazol-3-yl)methylsulfonamido)-4-oxobutyl)-N-(((phosphonooxy)methoxy)carbonyl)glycine (Compound 12)

To a solution of 12A (1.0 eq) in DCM (0.1 M) is added trifluoroacetic acid (31 eq) with stirring. Upon reaction completion, the reaction is concentrated under reduced pressure and purified by reverse phase HPLC. Fractions containing the product are pooled and lyophilized to give title compound 12.

### Example 13. (Phosphonooxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2,2-dimethyl-4-oxobutyl)carbamate (Compound 13)

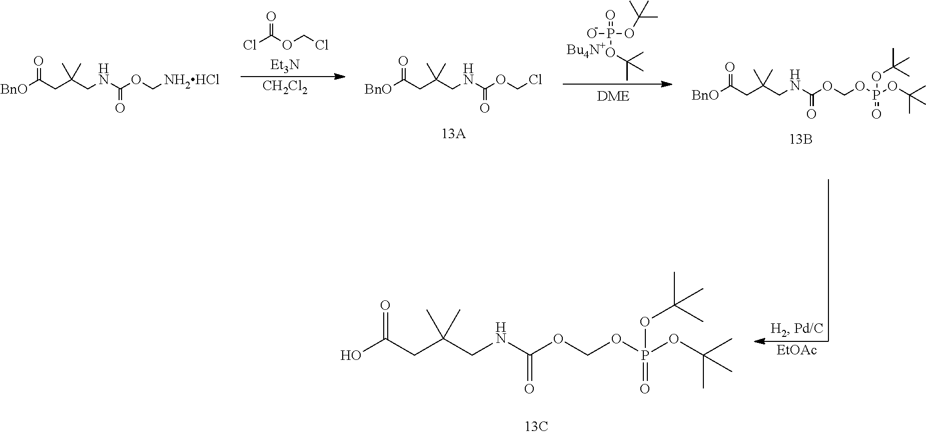

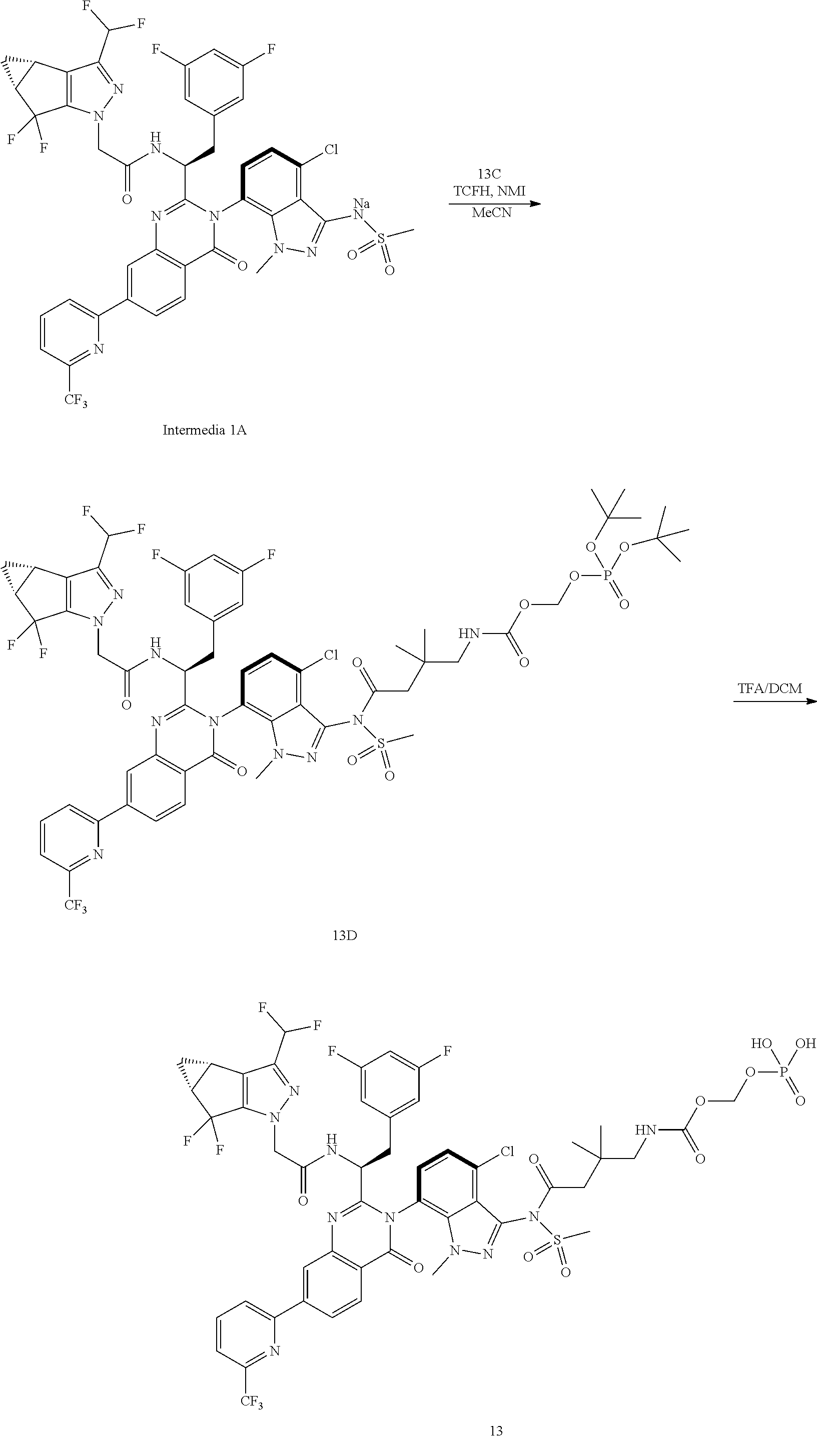

### Step 1. Synthesis of benzyl 4-(((chloromethoxy)carbonyl)amino)-3,3-dimethylbutanoate (13A)

To a stirred solution of benzyl 4-amino-3,3-dimethyl-butanoate hydrochloride (7.4 mmol) in CH₂Cl₂ (74 mL) at 0° C. was added triethylamine (24.0 mmol). Chloromethyl chloroformate (11 mmol) was then added and the reaction mixture was stirred at 0° C. and monitored by TLC and LCMS. After completion, the reaction mixture was washed with sat. aq. solutions of NH₄Cl and brine. The combined organic layers were dried over anhydrous Na₂SO₄, filtered, concentrated under reduced pressure, and purified by silica gel chromatography (eluting 0-25% EtOAc in hexanes). Fractions containing the product were pooled and concentrated under reduced pressure to give title compound 13A. MS (m/z) 336.2 [M+Na]⁺.

### Step 2. Synthesis of benzyl 4-(((((di-tert-butoxyphosphoryl)oxy)methoxy)carbonyl)amino)-3,3-dimethylbutanoate (13B)

The title compound was prepared according to the method presented for the synthesis of compound 11C of Example 11 utilizing 13A in the place of 11B. MS (m/z) 510.3 [M+Na]⁺.

### Step 3. Synthesis of 4-(((((di-t-butoxyphosphoryl)oxy)methoxy)carbonyl)amino)-3,3-dimethylbutanoic acid (13C)

The title compound was prepared according to the method presented for the synthesis of compound 11D of Example 30 utilizing 13B in the place of 11C. MS (m/z) 420.2 [M+Na]⁺.

### Step 4. Prophetic Synthesis of ((di-tert-butoxyphosphoryl)oxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2,2-dimethyl-4-oxobutyl)carbamate (13D)

To a suspension of Intermediate 1A (1.3 eq), 1-methylimidazole (3.5 eq), and 13C (1.0 eq) in MeCN (0.2 M) is added TCFH (1.20 eq) and the resulting mixture is stirred at rt. Upon completion of the reaction the solvent is removed under reduced pressure and the resulting residue is purified by silica gel chromatography. Fractions containing the product are pooled and concentrated under reduced pressure to give title compound 13D.

### Step 5. Prophetic Synthesis of (phosphonooxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)methylsulfonamido)-2,2-dimethyl-4-oxobutyl)carbamate (13)

A solution of 13D (1.0 eq) in TFA/DCM (1:4, 0.09 M) is stirred at room temperature. Upon reaction completion, the solvent is removed under reduced pressure and the residue is purified by reverse phase HPLC. Product-containing fractions are pooled and lyophilized to yield title compound 13.

### Example 14. (Phosphonooxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2,2-dimethyl-4-oxobutyl)carbamate (Compound 14)

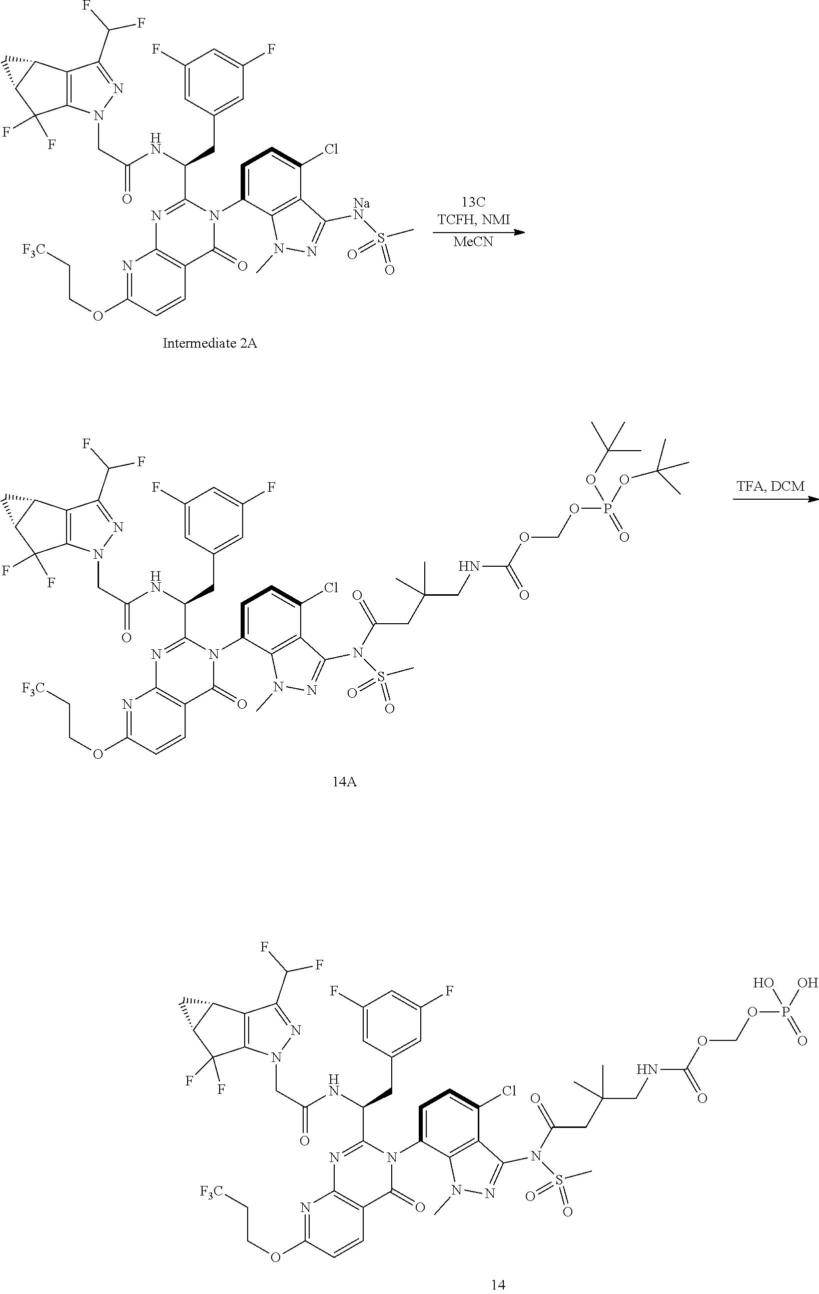

### Step 1. Prophetic Synthesis of ((di-tert-butoxyphosphoryl)oxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2,2-dimethyl-4-oxobutyl)carbamate (14A)

To a suspension of Intermediate 2A (1.3 eq), 1-methylimidazole (3.5 eq), and 13C (1.0 eq) in MeCN (0.2 M) is added TCFH (1.20 eq) and the resulting mixture is stirred at rt. Upon completion of the reaction, the solvent is removed under reduced pressure and the resulting residue is purified by silica gel chromatography. Fractions containing the product are pooled and concentrated under reduced pressure to give title compound 14A.

### Step 2. Prophetic Synthesis of (phosphonooxy)methyl (4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)methylsulfonamido)-2,2-dimethyl-4-oxobutyl)carbamate (Compound 14)

A solution of 14A (1.0 eq) in TFA/DCM (1:4, 0.09 M) is stirred at room temperature. Upon reaction completion, the solvent is removed under reduced pressure and the residue is purified by reverse phase HPLC. Product-containing fractions are pooled and lyophilized to yield title compound 14.

### Example 15. (2-(3-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl dihydrogen phosphate (Compound 15)

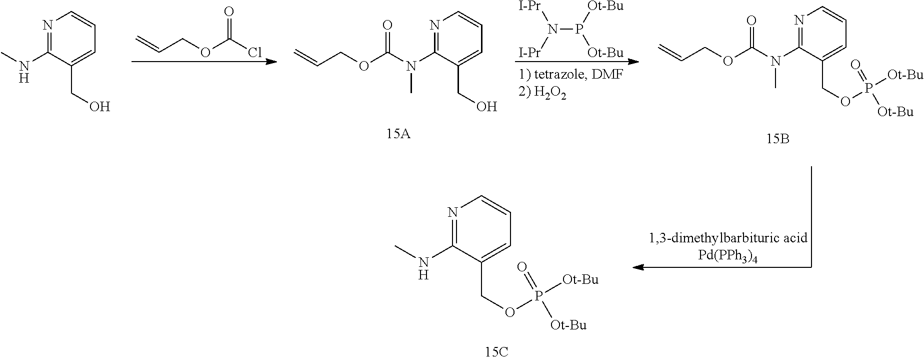

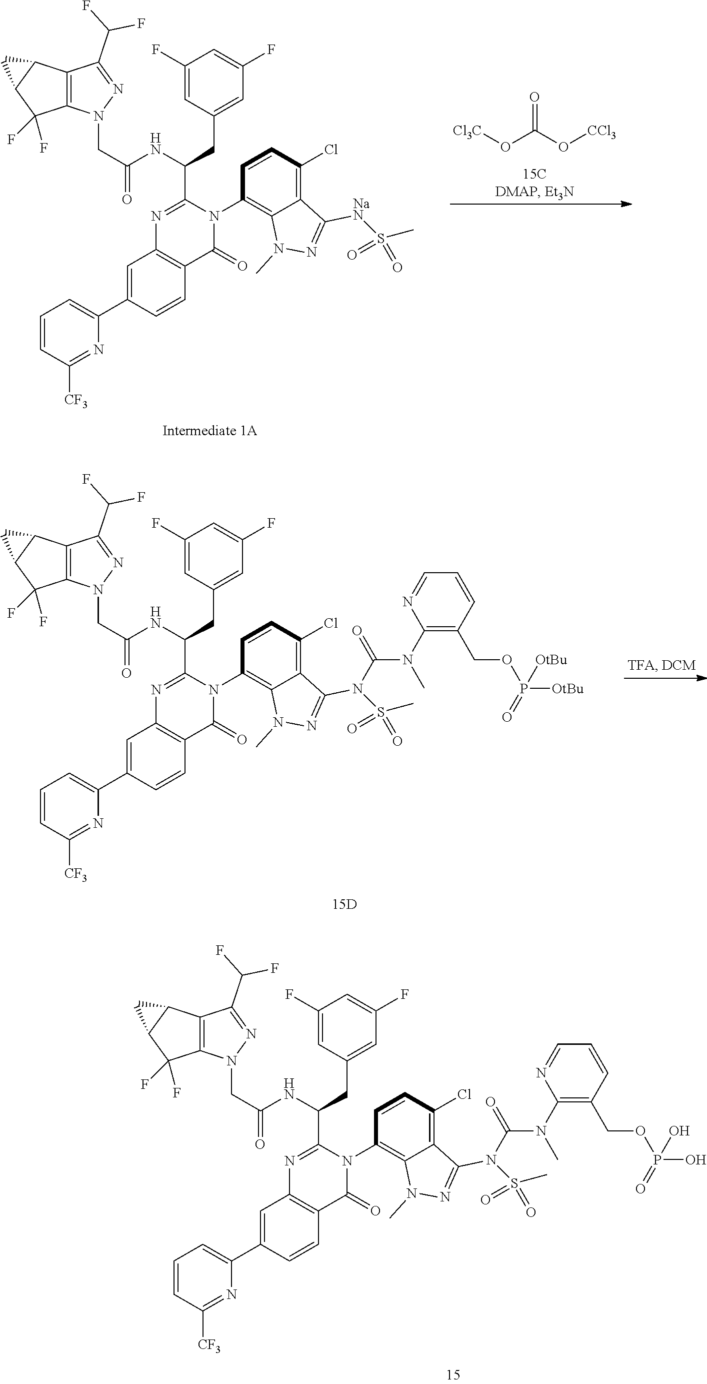

### Step 1. Synthesis of allyl (3-(hydroxymethyl)pyridin-2-yl)(methyl)carbamate (15A)

To a stirred suspension of [2-(methylamino)-3-pyridyl]methanol (151 mmol) in EtOAc (100 mL) and a sat. aq. solution of NaHCO₃ (100 mL) was added allyl chloroformate (181 mmol), then the reaction mixture was stirred at room temperature for 16 h. After completion, the reaction mixture was diluted with EtOAc and washed with water. The combined organic layers were dried over anhydrous Na₂SO₄, filtered, and concentrated. The crude mixture was purified by column chromatography (0% to 100% EtOAc in hexanes) to afford title compound 15A. MS (m/z): 223.1 [M+H]⁺.

### Step 2. Synthesis of allyl (3-(((di-tert-butoxyphosphoryl)oxy)methyl)pyridin-2-yl)(methyl)carbamate (15B)

To a solution of 15A (13.0 mmol) in DMF (100 mL) was added di-t-butyl-N,N-diisopropylphosphoramidite (39.0 mmol) and 1-H-tetrazole (52.0 mmol) sequentially. The reaction mixture was stirred at room temperature while monitored by TLC and LCMS. After 4 h, the reaction mixture was cooled to 0° C. and treated with aq. H₂O₂ solution (4.8 mL, 50 wt %). The reaction was gradually warmed to room temperature and stirred. After completion, the reaction mixture was diluted with ethyl ether and washed with sat. aq. sodium thiosulfate solution. The combined organic layers were dried over anhydrous Na₂SO₄, filtered, and concentrated. The crude mixture was purified by column chromatography (0% to 100% EtOAc in hexanes) to afford the title compound 15B. MS (m/z): 437.2 [M+Na]⁺.

### Step 3. Synthesis of di-tert-butyl ((2-(methylamino)pyridin-3-yl)methyl) phosphate (15C)

To a solution of 15B (6.5 mmol) in a mixture of ethyl acetate and dichloromethane (38 mL, 1:1) was added 1,3-dimethylbarbituric acid (8.4 mmol) followed by tetrakis(triphenylphosphine)palladium(0) (0.32 mmol). The resulting reaction mixture was stirred at room temperature while monitored by LCMS. Upon completion, the reaction mixture was diluted with EtOAc and washed with water. The combined organic layers were dried over anhydrous Na₂SO₄, filtered, and concentrated. The crude mixture was purified by column chromatography (0% to 5% MeOH in CH₂Cl₂) to afford title compound 15C. MS (m/z): 331.2 [M+H]⁺.

### Step 4. Prophetic Synthesis of di-tert-butyl ((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl) phosphate (15D)

To an ice-cold solution of 15C (1.0 eq) and triphosgene (0.33 eq) in DCE (0.5 M) is added triethylamine (2.2 eq). The reaction is sealed, brought to room temperature, and stirred for 30 minutes. To the flask is added sequentially Intermediate 1A (1.5 eq) and 4-dimethylaminopyridine (0.25 eq). The reaction is sealed and heated at 60° C. Upon completion, the reaction is brought to room temperature and diluted with DCM. The reaction is transferred to a separatory funnel and washed 1× with saturated aqueous NH₄Cl. The organic fraction is collected, dried over Na₂SO₄, concentrated, and is purified by silica gel chromatography. Fractions containing the product are pooled and concentrated to yield title compound 15D.

### Step 5. Prophetic Synthesis of (2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl dihydrogen phosphate (Compound 15)

To a flask is added 15D (1.0 eq) and TFA/DCM (4:6, 0.1 M). The reaction is sealed and stirred at room temperature. Upon completion, the reaction mixture is concentrated, diluted in DMF, filtered, and purified by reverse phase HPLC. Fractions containing the product are pooled and lyophilized to yield title compound 15.

### Example 16. (2-(3-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl dihydrogen phosphate (Compound 16)

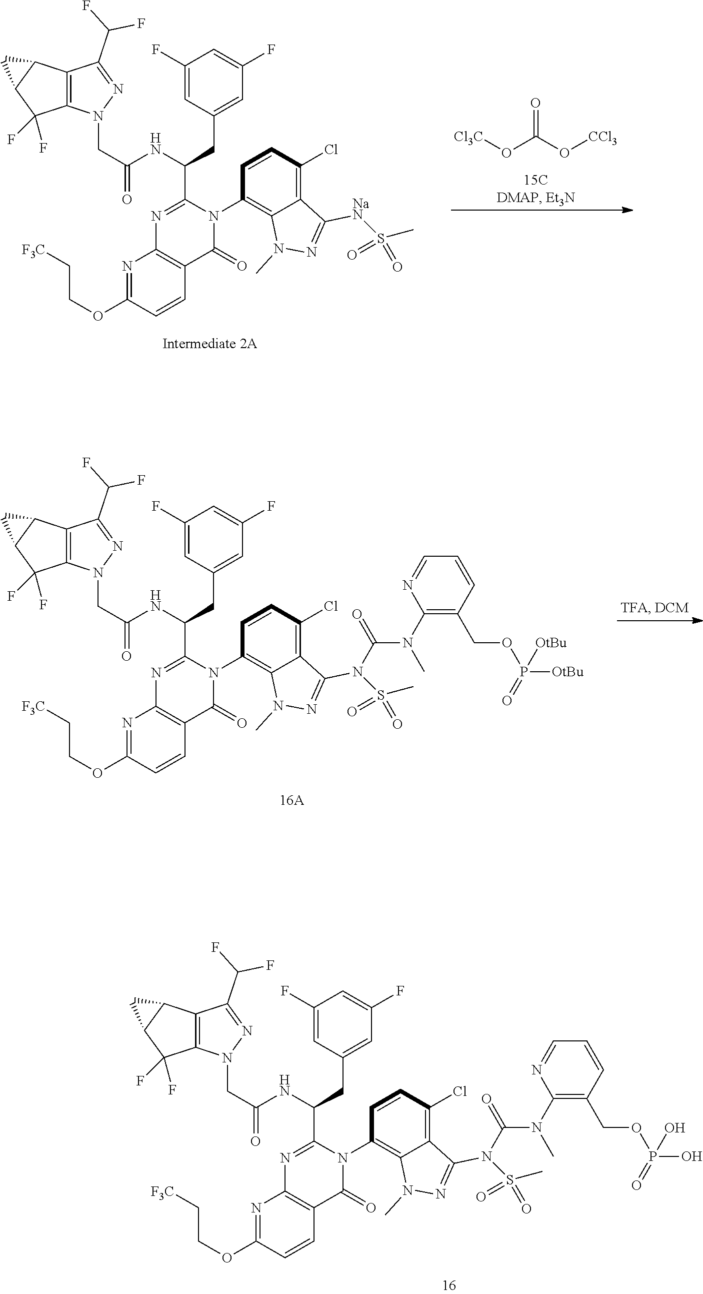

### Step 1. Prophetic Synthesis of di-tert-butyl ((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl) phosphate (16A)

To an ice-cold solution of 15C (1.0 eq) and triphosgene (0.33 eq) in DCE (0.5 M) is added triethylamine (2.2 eq). The reaction is sealed, brought to room temperature, and stirred for 30 minutes. To the flask is added sequentially Intermediate 2A (1.5 eq) and 4-dimethylaminopyridine (0.25 eq). The reaction is sealed and heated at 60° C. Upon completion, the reaction is brought to room temperature and diluted with DCM. The reaction is transferred to a separatory funnel and washed 1× with saturated aqueous NH₄Cl. The organic fraction is collected, dried over Na₂SO₄, concentrated, and is purified by silica gel chromatography. Fractions containing the product are pooled and concentrated to yield title compound 16A.

### Step 2. Prophetic Synthesis of (2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl dihydrogen phosphate (16)

To a flask is added 16A (1.0 eq) and TFA/DCM (4:6, 0.1 M). The reaction is sealed and stirred at room temperature. Upon completion, the reaction mixture is concentrated, diluted in DMF, filtered, and purified by reverse phase HPLC. Fractions containing the product are pooled and lyophilized to yield title compound 16.

### Example 17. N-((2-(3-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl)-N-(((phosphonooxy)methoxy)carbonyl)glycine (Compound 17)

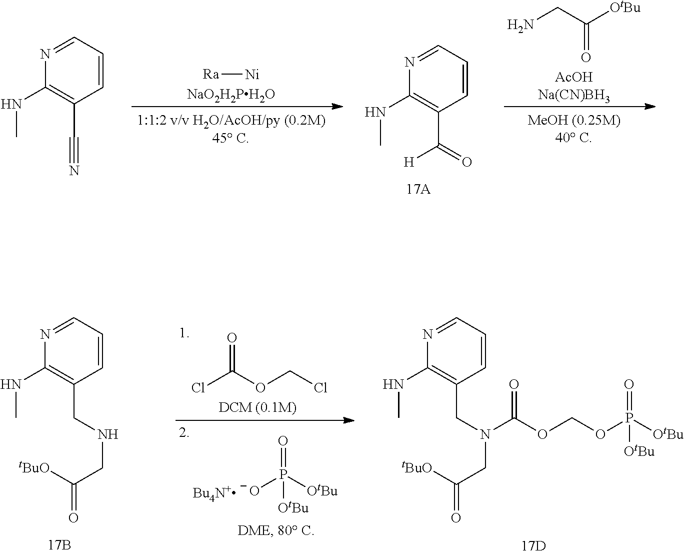

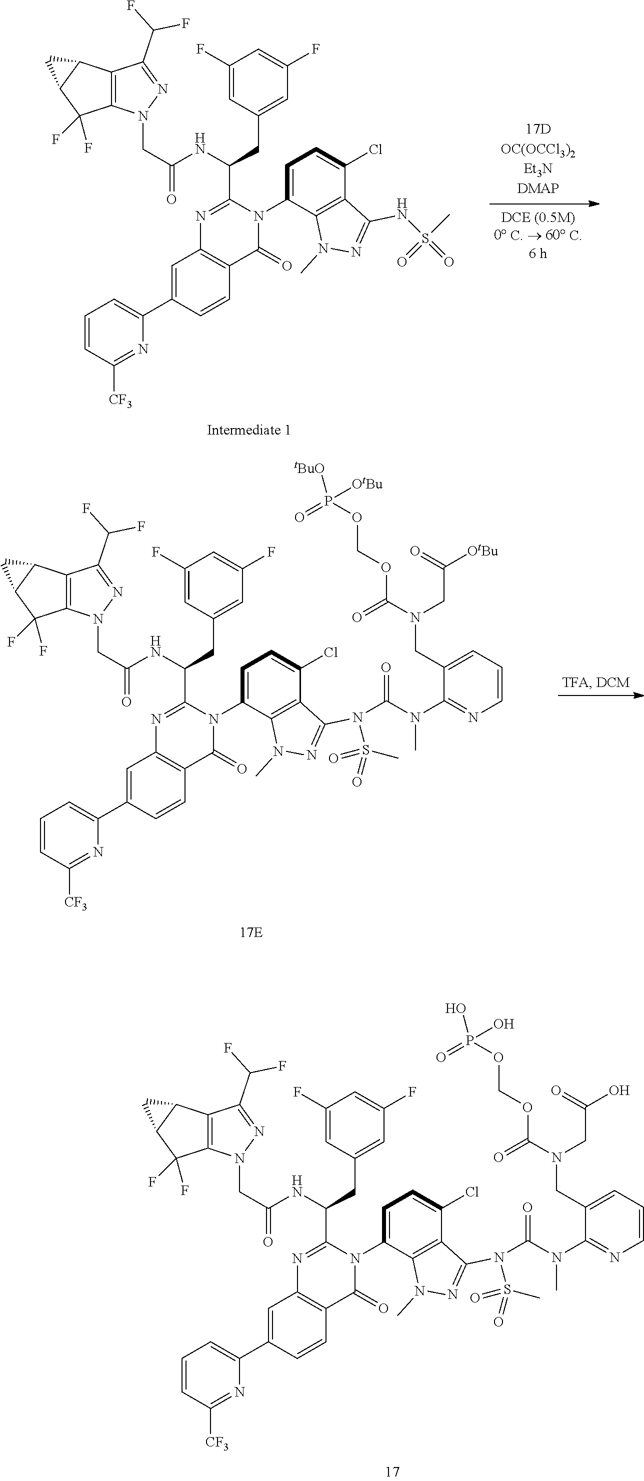

### Step 11. Synthesis of 2-(methylamino)pyridine-3-carbaldehyde (17A)

To a solution of sodium hypophosphite monohydrate (360 mmol) and Raney nickel (180 mmol) in 1:1:2 v/v H₂O/AcOH/pyridine (450 mL) was added 2-(methylamino)pyridine-3-carbonitrile (180 mmol). The reaction was then sealed, heated to 35° C., and stirred for 2 hours. Upon completion, the reaction was cooled to room temperature, diluted sequentially with water (110 mL), NaCl (80 g), and Celite (11 g) and filtered. The cake was washed with water (225 mL) and EtOAc (225 mL). The filtrate was then transferred to a separatory funnel and the aqueous layer was extracted 2× with EtOAc (225 mL). The organic fraction was collected, dried over Na₂SO₄, concentrated, and purified by silica chromatography. Fractions containing the product were pooled and concentrated under reduced pressure to yield title compound 17A. ¹H NMR (400 MHz, CDCl₃) δ 9.80 (s, 1H), 8.35 (dd, J=4.9, 1.9 Hz, 2H), 7.74 (dd, J=7.5, 1.9 Hz, 1H), 6.64 (dd, J=7.5, 4.9 Hz, 1H), 3.11 (d, J=4.9 Hz, 3H) ppm.

### Step 2. Synthesis of tert-butyl 2-[[2-(methylamino)-3-pyridyl]methylamino]acetate (17B)

To a flask was added sequentially 17A (64 mmol), tert-butyl 2-aminoacetate (192 mmol), acetic acid (192 mmol), and MeOH (256 mL). The reaction was then sealed, heated to 40° C., and stirred for 30 minutes. The reaction was then cooled to 0° C. and sodium cyanoborohydride (134 mmol) was added to the reaction in one portion. The reaction was then sealed, brought to room temperature, and stirred for 90 minutes. Upon completion, the reaction mixture was concentrated and dissolved in sat. NaHCO₃ (250 mL). The reaction was transferred to a separatory funnel and extracted 3x with DCM (125 mL). The organic fraction was collected, dried over Na₂SO₄, concentrated, and purified by silica chromatography. Fractions containing the product were pooled and concentrated to yield title compound 17B. ¹H NMR (400 MHz, CDCl₃) δ 8.08 (dd, J=5.1, 1.8 Hz, 1H), 7.16 (dd, J=7.0, 1.8 Hz, 1H), 6.53 (s, 1H), 6.45 (dd, J=7.1, 5.1 Hz, 1H), 3.68 (s, 2H), 3.24 (s, 2H), 3.00 (s, 3H), 1.83 (s, 1H), 1.47 (s, 9H). MS (m/z): 252.20 [M+H]⁺.

### Step 3. Synthesis of tert-butyl N-((chloromethoxy)carbonyl)-N-((2-(methylamino)pyridin-3-yl)methyl)glycinate (17C)

To an ice-cold solution of 17B (63 mmol) in DCM (630 mL) was added chloromethyl chloroformate (63 mmol). The reaction was sealed, cooled on ice, and stirred for 15 minutes. Upon completion, the reaction mixture was transferred to a separatory funnel and washed with sat. NaHCO₃ (315 mL). The organic fraction was collected, dried over Na₂SO₄, and concentrated to yield title compound 17C, which was used without further purification.

### Step 4. Synthesis of tert-butyl 2-[ditert-butoxyphosphoryloxymethoxycarbonyl-[[2-(methylamino)-3-pyridyl]methyl]amino]acetate (17D)

To 17C (63 mmol) was added tetrabutylammonium di-tert-butyl phosphate (95 mmol) and DME (300 mL). The reaction was then sealed, heated to 80° C., and stirred for 1 hour. Upon completion, the reaction was concentrated and dissolved in 4:1 EtOAc/Hexanes (300 mL). The solution was transferred to a separatory funnel and washed sequentially 2× with brine (150 mL), 1× with water (150 mL), and 1× with brine (150 mL). The organic fraction was collected, dried over Na₂SO₄, and concentrated to yield title compound 17D. ¹H NMR (400 MHz, CDCl3) δ 8.13 (d, 1H), 7.16 (d, 1H), 6.47 (t, J=6.3 Hz, 1H), 5.64 (d, 2H), 4.45 (s, 2H), 3.78 (s, 2H), 3.00 (s, 3H), 1.47 (s, 18H), 1.39 (s, 9H). MS (m/z): 518.30 [M+H]⁺.

### Step 5. Prophetic Synthesis of tert-butyl N-((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl)-N-((((di-tert-butoxyphosphoryl)oxy)methoxy)carbonyl)glycinate (17E)

To an ice-cold solution of 17D (1.0 eq) and triphosgene (0.33 eq) in DCE (0.5 M) is added triethylamine (2.2 eq). The reaction is sealed, brought to room temperature, and stirred for 30 minutes. To the flask is added sequentially Intermediate 1 (1.5 eq) and 4-dimethylaminopyridine (0.25 eq). The reaction is sealed and heated at 60° C. Upon completion, the reaction is brought to room temperature and diluted with DCM. The reaction is transferred to a separatory funnel and washed 1× with saturated aqueous NH₄Cl. The organic fraction is collected, dried over Na₂SO₄, concentrated, and is purified by silica gel chromatography. Fractions containing the product are pooled and concentrated to yield title compound 17E.

### Step 6. Prophetic Synthesis of N-((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(6-(trifluoromethyl)pyridin-2-yl)quinazolin-3(4H)-yl)-1-methyl-1H-indazol-3-yl)-1-methyl-3-(methylsulfonyl)ureido)pyridin-3-yl)methyl)-N-(((phosphonooxy)methoxy)carbonyl)glycine (Compound 17)

To a flask is added 17E (1.0 eq) and TFA/DCM (4:6, 0.1 M). The reaction is sealed and stirred at room temperature. Upon completion, the reaction mixture is concentrated, diluted in DMF, filtered, and purified by reverse phase HPLC. Fractions containing the product are pooled and lyophilized to yield title compound 17.

### Example 18. N-((2-(3-(4-Chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl)-N-(((phosphonooxy)methoxy)carbonyl)glycine (Compound 18)

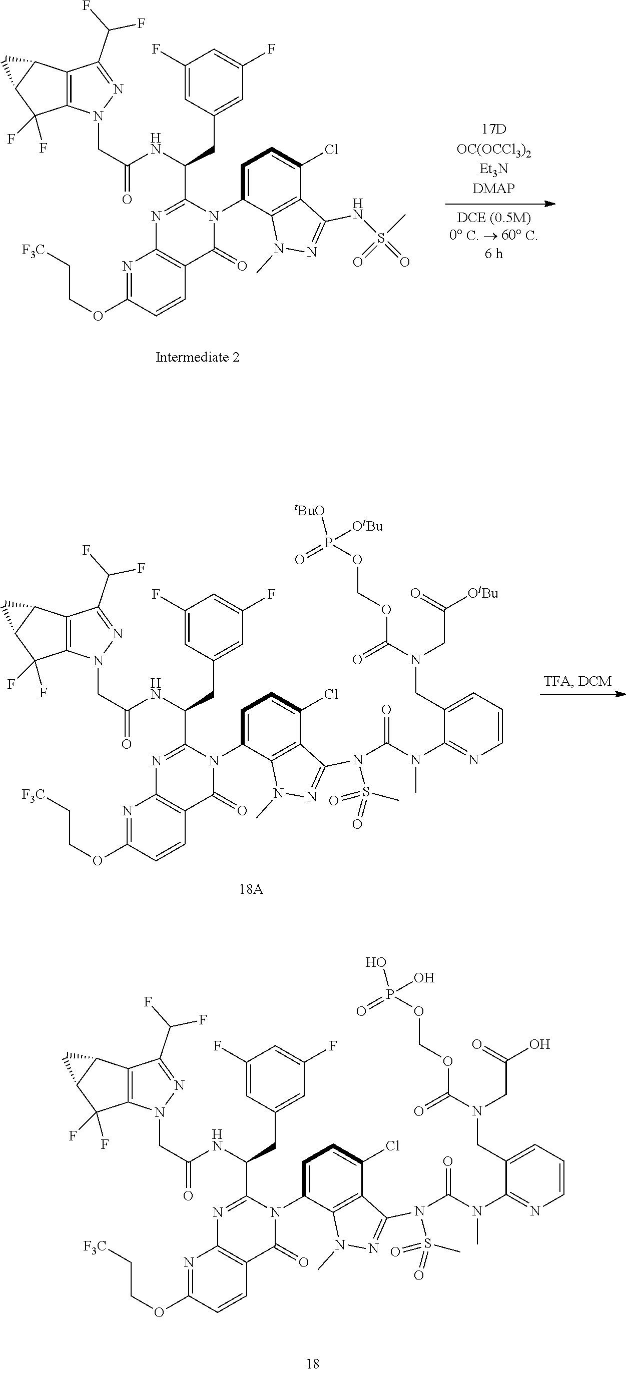

### Step 1. Prophetic Synthesis of tert-butyl N-((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl)-N-((((di-tert-butoxyphosphoryl)oxy)methoxy)carbonyl)glycinate (18A)

To an ice-cold solution of 17D (1.0 eq) and triphosgene (0.33 eq) in DCE (0.5 M) is added triethylamine (2.2 eq). The reaction is sealed, brought to room temperature, and stirred for 30 minutes. To the flask is added sequentially Intermediate 2 (1.5 eq) and 4-dimethylaminopyridine (0.25 eq). The reaction is sealed and heated at 60° C. Upon completion, the reaction is brought to room temperature and diluted with DCM. The reaction is transferred to a separatory funnel and washed 1× with saturated aqueous NH₄Cl. The organic fraction is collected, dried over Na₂SO₄, concentrated, and is purified by silica gel chromatography. Fractions containing the product are pooled and concentrated to yield title compound 18A.

### Step 2. Prophetic Synthesis of N-((2-(3-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-7-(3,3,3-trifluoropropoxy)pyrido[2,3-d]pyrimidin-3(4H)-yl)-1-methyl-1H-indazol-3-yl)-1-methyl-3-(methylsulfonyl)ureido)pyridin-3-yl)methyl)-N-(((phosphonooxy)methoxy)carbonyl)glycine (Compound 18)

To a flask is added 18A (1.0 eq) and TFA/DCM (4:6, 0.1 M). The reaction is sealed and stirred at room temperature. Upon completion, the reaction mixture is concentrated, diluted in DMF, filtered, and purified by reverse phase HPLC. Fractions containing the product are pooled and lyophilized to yield title compound 18.

### Example 19. 2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 19)

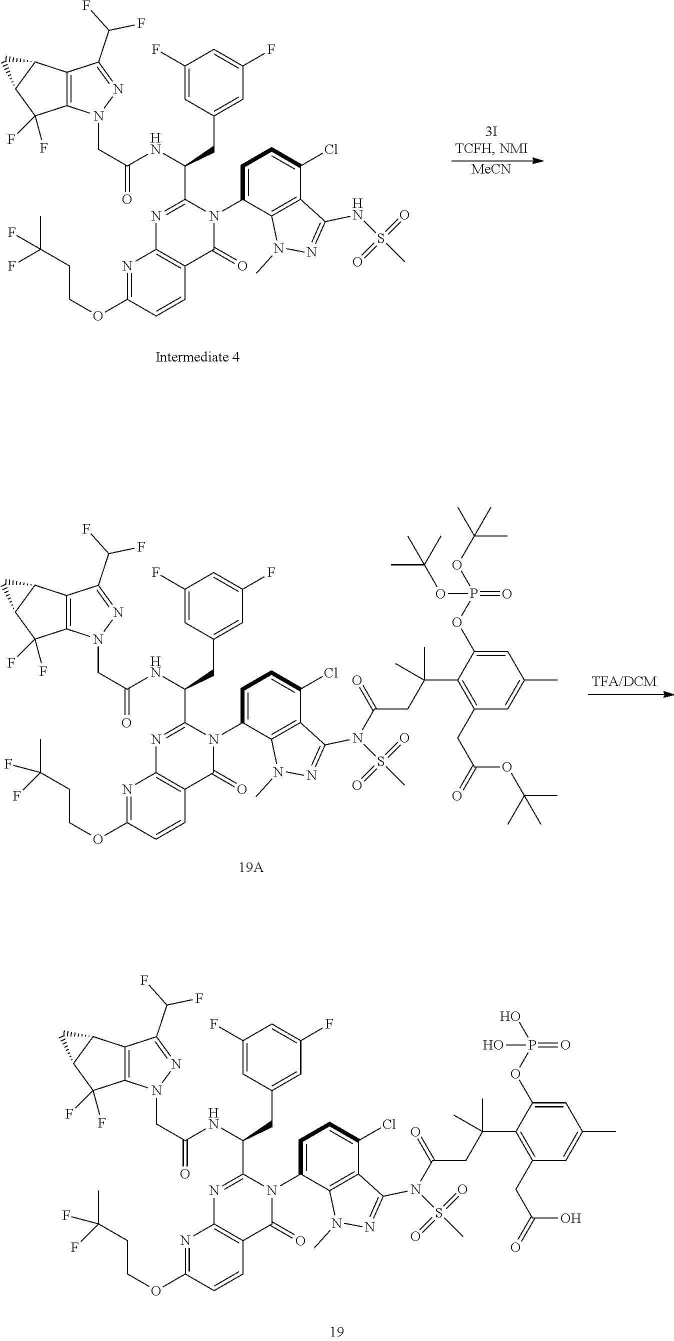

### Step 1. Synthesis of tert-butyl 2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (19A)

To a solution of 31 (85.5 μmol) in ACN (1.0 mL) was added NMI (150 μmol), TCFH (42.7 μmol) and Intermediate 4 (42.7 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 19A. MS (m/z): 1242.3 [M-3[tBu]+H]⁺.

### Step 2. Synthesis of 2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 19)

To a solution of 19A (19.7 μmol) in DCM (1.5 mL) was added TFA (0.5 mL). The mixture was stirred at 25° C. for 10 mins. The reaction mixture was concentrated and purified by reverse phase HPLC (ACN/water with 0.1% TFA). Fractions containing the product were pooled and concentrated to afford an atropisomeric mixture of title compound 19. ¹H NMR (400 MHz, DMSO-d₆) δ 9.20 (br d, J=7.5 Hz), 8.44 (dd, J=2.2, 8.6 Hz), 7.81 (d, J=7.9 Hz), 7.66 (d, J=7.9 Hz), 7.54 (t, J=8.2 Hz) 7.27-6.51 (m), 4.73-4.32 (m), 3.85-3.32 (m), 3.27-2.91 (m), 2.75-2.36 (m), 2.17 (d, J=7.2 Hz), 1.73 (t, J=19.2 Hz), 1.54-1.10 (m), 0.95-0.81 (m) ppm. ³¹P NMR (162 MHz, DMSO-d₆) δ=−7.11, −7.45 ppm. MS (m/z): 1242.1 [M+H]⁺.

### Example 20. (2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 20)

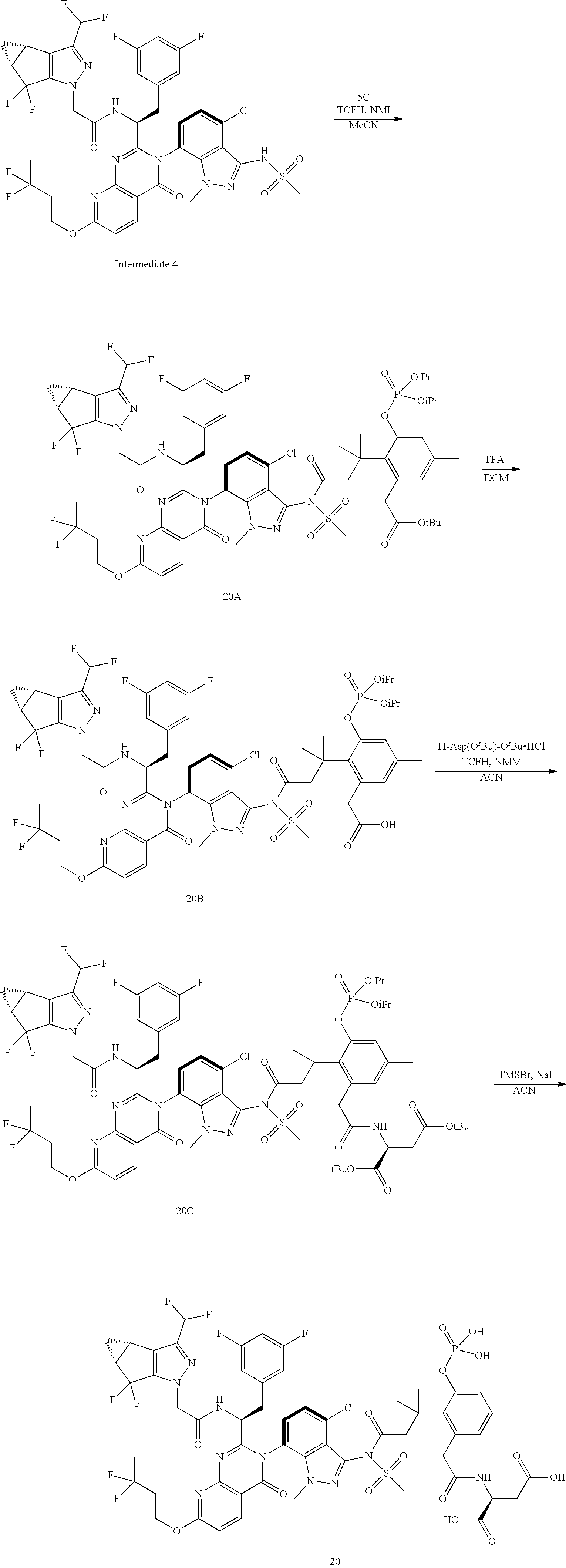

### Step 1. Synthesis of tert-butyl 2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetate (20A)

To a solution of 5C (165 μmol) was added NMI (383 μmol), Intermediate 4 (109 μmol) and TCFH (109 μmol). The mixture was stirred at 25° C. for 2 hours. The reaction mixture was concentrated and purified by reverse phase HPLC (ACN/water with 0.1% TFA). Fractions containing the product were pooled and concentrated to afford an atropisomeric mixture of title compound 20A. MS (m/z): 1382.5 [M+H]⁺.

### Step 2. Synthesis of 2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (20B)

To a solution of 20A (66.8 μmol) in DCM (1.5 mL) was added TFA (0.5 mL). The mixture was stirred at 25° C. for 1 hour. The reaction mixture was concentrated and purified by reverse phase HPLC (ACN/water with 0.1% TFA). Fractions containing the product were pooled and concentrated to afford an atropisomeric mixture of title compound 20B. MS (m/z): 1326.4 [M+H]⁺.

### Step 3. Synthesis of di-tert-butyl (2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (20C)

To a solution of 20B (69.4 μmol) in ACN (1.0 mL) was added di-tert-butyl L-aspartate hydrochloride (104 μmol), NMM (347 μmol) and TCFH (83.3 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 20C. MS (m/z): 1553.5 [M+H]⁺.

### Step 4. Synthesis of (2-(2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (20)

To a solution of 20C (30.0 μmol) in ACN (0.5 mL) was added NaI (60.0 μmol) and TMSBr (240 μmol). The mixture was stirred at 45° C. for 2 hours under N₂. The reaction mixture was concentrated and purified by reverse phase HPLC (ACN/water with 0.1% TFA). Fractions containing the product were pooled and concentrated to afford an atropisomeric mixture of title compound 20. ¹H NMR (400 MHz, DMSO-d₆) δ 9.39-9.06 (m), 8.52-8.35 (m), 8.09 (d, J=7.6 Hz), 7.81 (d, J=7.9 Hz), 7.68 (d, J=7.8 Hz), 7.52 (dd, J=19.2, 7.9 Hz), 7.40-6.84 (m), 6.84-6.48 (m), 4.79-4.38 (m), 4.37-2.86 (m), 2.82-2.35 (m), 2.15 (d, J=8.7 Hz), 1.73 (t, J=19.2 Hz), 1.54-1.08 (m), 0.95-0.73 (m) ppm. ³¹P NMR (162 MHz, DMSO-d₆) δ=−6.64, −7.86 ppm. MS (m/z): 1357.2 [M+H]⁺.

### Example 21. (2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 21)

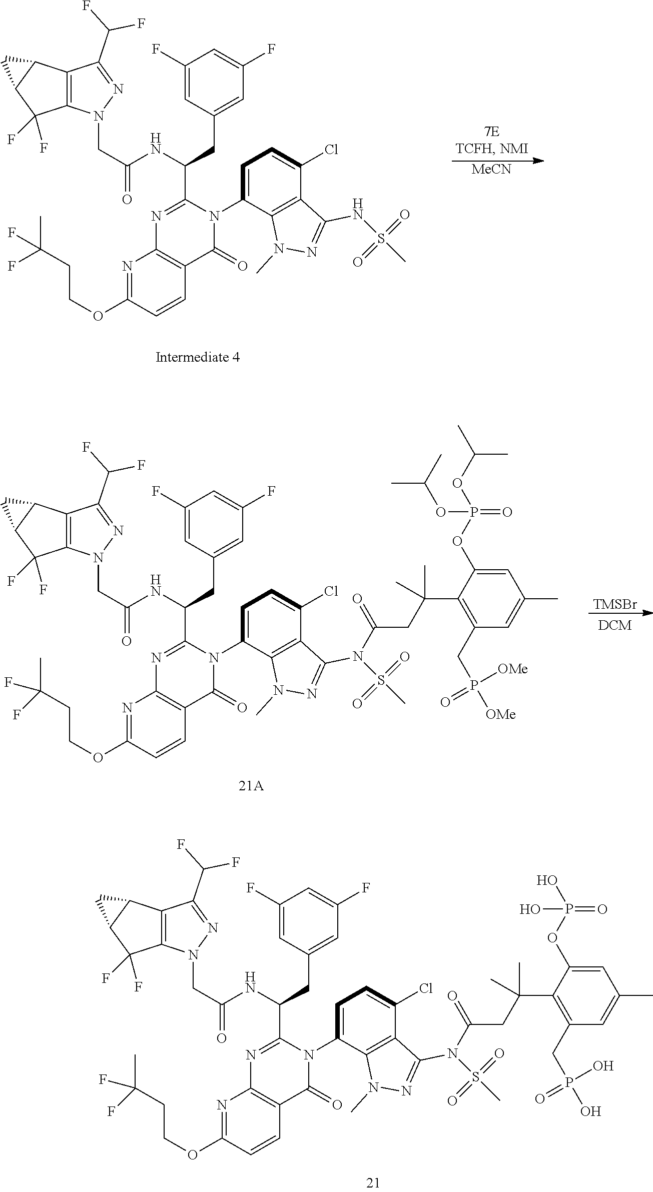

### Step 1. Synthesis of 2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (21A)

To a solution of 7E (219 μmol) in ACN (1.0 mL) was added NMI (383 μmol), TCFH (109 μmol) and Intermediate 4 (109 μmol). The mixture was stirred at 50° C. for 1 hour. The reaction mixture was concentrated and purified by reverse phase HPLC (ACN/water with 0.1% TFA). Fractions containing the product were pooled and concentrated to afford an atropisomeric mixture of title compound 21A. MS (m/z): 1390.5 [M+H]⁺.

### Step 2. Synthesis of (2-(4-(N-(4-chloro-7-(7-(3,3-difluorobutoxy)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxopyrido[2,3-d]pyrimidin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (21)

To a solution of 21A (50.3 μmol) in DCM (1.0 mL) was added TMSBr (503 mol). The mixture was stirred at 45° C. for 3 hours under N₂. The reaction mixture was concentrated and purified by reverse phase HPLC (ACN/water with 0.1% TFA). Fractions containing the product were pooled and concentrated to afford an atropisomeric mixture of title compound 21. ¹H NMR (400 MHz, DMSO-d₆) δ 9.31-9.08 (m), 8.49-8.41 (m), 7.82 (d, J=7.9 Hz), 7.69-7.43 (m), 7.18-6.85 (m), 6.84-6.60 (m), 4.85-4.22 (m), 3.70-2.99 (m), 2.62-2.37 (m), 2.16 (d, J=8.1 Hz), 1.74 (t, J=19.2 Hz), 1.66-1.43 (m), 1.42-1.20 (m), 0.95-0.83 (m) ppm. ³¹P NMR (162 MHz, DMSO-d₆) δ=21.62 (d), −7.15, −7.63 ppm. MS (m/z): 639.8 [M+2H]²⁺.

### Example 22. 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)phenyl)acetic acid (Compound 22)

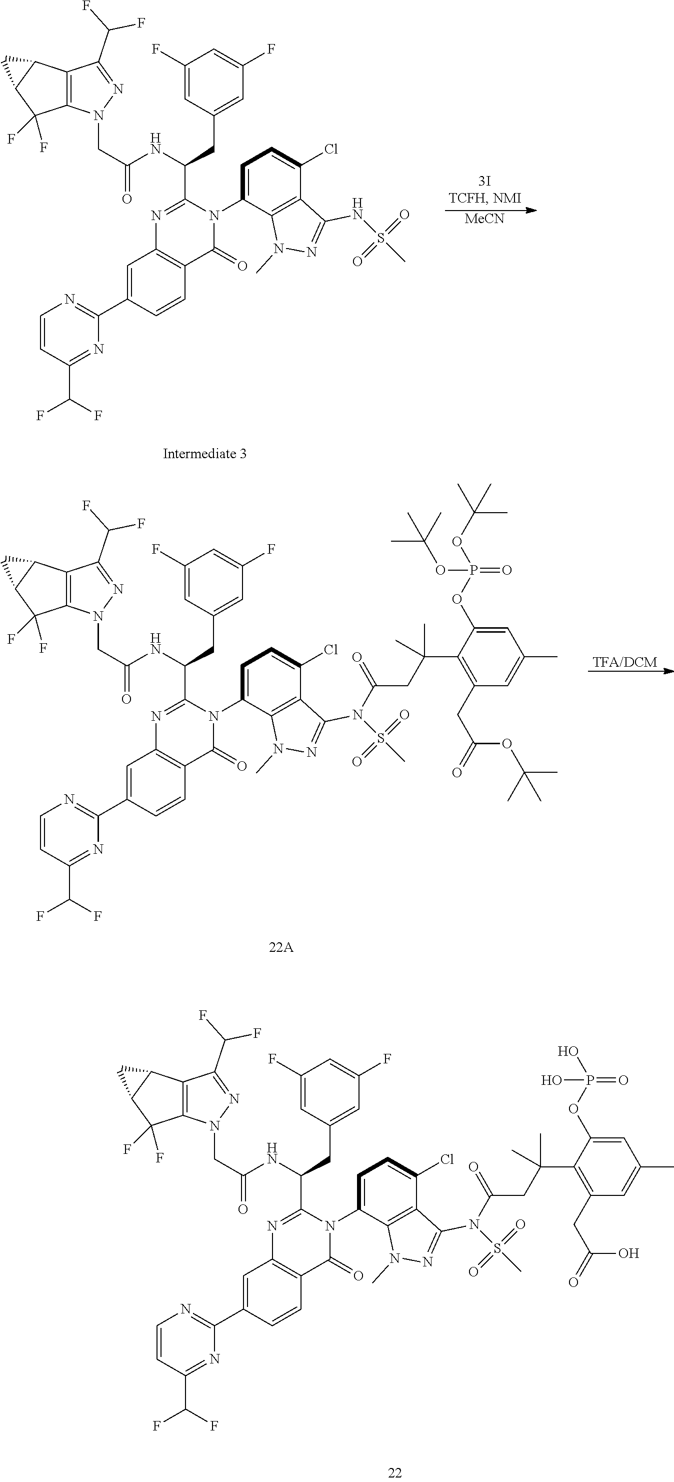

### Step 11. Synthesis of tert-butyl 2-(2-(4-(N-(4-chloro-7-(2-((S)-1-(2-((3bS,4aR)-3-(dfluoromethyl)-5, 5-difluoro-3b, 4,4a, 5-tetrahydro-1H-cyclopropa[3, 4]cyclopenta[1,2-c]pyrazol-1-yl)acetamido)-2-(3, 5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-3-((di-tert-butoxyphosphoryl)oxy)-5-methylphenyl)acetate (22A)

To a solution of 31 (129 μmol) in ACN (0.5 mL) was added NMI (225 μmol), TCFH (64.3 μmol, 1 eq) and Intermediate 3 (664 μmol). The mixture was stirred at 50° C. for 2 hours. The reaction mixture was concentrated and purified by reverse phase TIPLC (ACN/water with 0.1% TFA). Fractions containing the product were pooled and concentrated to afford an atropisomeric mixture of title compound 22A. MS (m/z): 1451.4 [M+Na]⁺.

### Step 2. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)phenyl)acetic acid (22)

To a solution of 22A (35.0 μmol) in DCM (1.5 mL) was added TFA (0.5 mL). The mixture was stirred at 25° C. for 0.5 hour. The reaction mixture was concentrated and purified by reverse phase HPLC (ACN/water with 0.1% TFA). Fractions containing the product were pooled and concentrated to afford an atropisomeric mixture of title compound 22. ¹H NMR (400 MHz, DMSO-d₆) δ 9.29 (d, J=5.0 Hz), 8.95-8.81 (m), 8.63 (ddd, J=8.4, 3.7, 1.4 Hz), 8.41 (d, J=9.4 Hz), 7.88 (d, J=5.1 Hz), 7.81 (d, J=7.9 Hz), 7.63 (d, J=7.9 Hz), 7.52 (t, J=8.4 Hz), 7.30 (s), 7.17 (s), 7.11 (s), 7.09-6.93 (m), 6.90 (d, J=13.9 Hz), 6.76 (d, J=13.9 Hz), 6.73-6.64 (m), 6.59 (bs), 4.72-4.62 (m), 4.62-4.51 (m), 4.49 (s), 4.37 (dd, J=16.5, 6.7 Hz), 3.86-3.38 (m), 3.33-2.90 (m), 2.75-2.58 (m), 2.51-2.36 (m), 2.17 (d, J=8.3 Hz), 1.46 (d, J=18.4 Hz), 1.38-1.08 (m), 0.96-0.82 (m) ppm. ³¹P NMR (162 MHz, DMSO-d₆) δ=−7.11, −7.58 ppm. MS (m/z): 1260.8 [M+H]⁺.

### Example 23. (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (Compound 23)

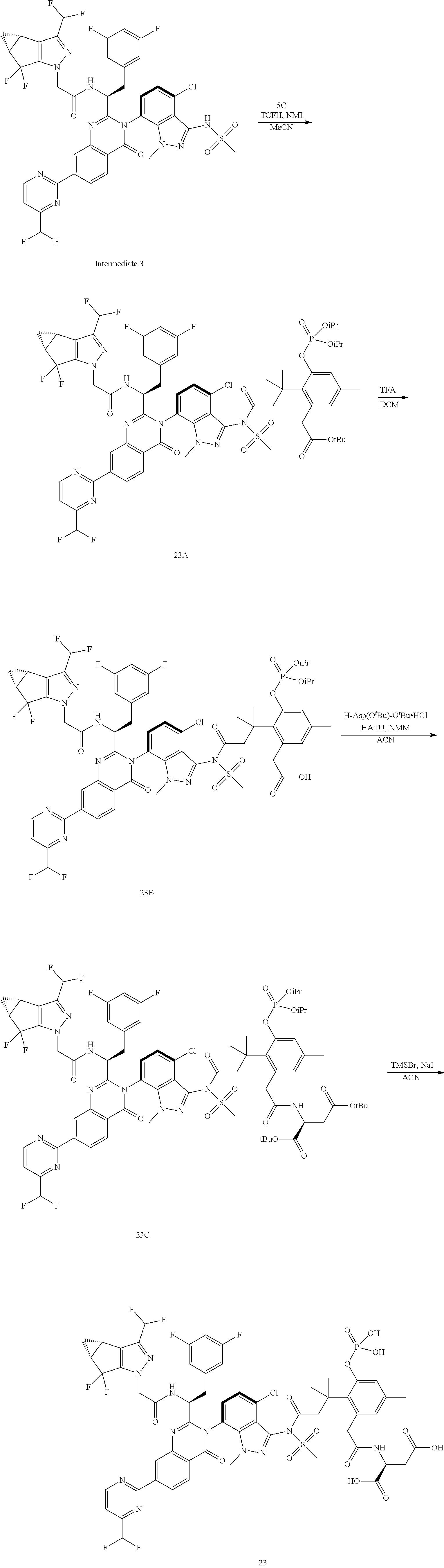

### Step 1. Synthesis of tert-butyl 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetate (23A)

To a solution of 5C (214 μmol) in ACN (1.0 mL) was added NMI (375 μmol), TCFH (107 μmol) and Intermediate 3 (107 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 23A. MS (m/z): 1423.4 [M+Na]⁺.

### Step 2. Synthesis of 2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetic acid (23B)

To a solution of 23A (79.2 μmol) in DCM (1.5 mL) was added TFA (0.5 mL). The mixture was stirred at 25° C. for 30 minutes. The reaction mixture was concentrated and purified by reverse phase HPLC (ACN/water with 0.1% TFA). Fractions containing the product were pooled and concentrated to afford an atropisomeric mixture of title compound 23B. MS (m/z): 1345.4 [M+H]⁺.

### Step 3. Synthesis of di-tert-butyl (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-3-((diisopropoxyphosphoryl)oxy)-5-methylphenyl)acetyl)-L-aspartate (23C)

To a solution of 23B (68.5 μmol) in ACN (1.0 mL) was added di-tert-butyl L-aspartate hydrochloride (103 μmol), NMM (343 μmol) and HATU (103 μmol). The mixture was stirred at 25° C. for 1 hour. The reaction mixture was concentrated and purified by reverse phase HPLC (ACN/water with 0.1% TFA). Fractions containing the product were pooled and concentrated to afford an atropisomeric mixture of title compound 23C. MS (m/z): 1572.5 [M+H]⁺.

### Step 4. Synthesis of (2-(2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)phenyl)acetyl)-L-aspartic acid (23)

To a solution of 23C (29.6 μmol) in ACN (1.0 mL) was added NaI (59.3 μmol) and TMSBr (237 μmol). The mixture was stirred at 45° C. for 1 hour under N₂. The reaction mixture was concentrated and purified by reverse phase HPLC (ACN/water with 0.1% TFA). Fractions containing the product were pooled and concentrated to afford an atropisomeric mixture of title compound 23. ¹H NMR (400 MHz, DMSO-d₆) δ 12.24 (bs), 9.29 (d, J=5.0 Hz), 8.92-8.83 (m), 8.62 (dd, J=8.3, 1.6 Hz), 8.40 (dd, J=8.3, 5.3 Hz), 8.14-8.00 (m), 7.88 (d, J=4.9 Hz), 7.80 (d, J=7.9 Hz), 7.67 (d, J=7.9 Hz), 7.49 (dd, J=20.7, 7.9 Hz), 7.33-6.87 (m), 6.82-6.49 (m), 4.76-4.64 (m), 4.63-4.33 (m), 4.31-4.16 (m), 3.86-3.40 (m), 3.34-2.96 (m), 2.75-2.36 (m), 2.15 (d, J=10.5 Hz), 1.56-1.10 (m), 0.94-0.81 (m). ³¹P NMR (162 MHz, DMSO-d₆) δ=−6.92, −7.99. MS (m/z): 1376.2 [M+H]⁺.

### Example 24. (2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (Compound 24)

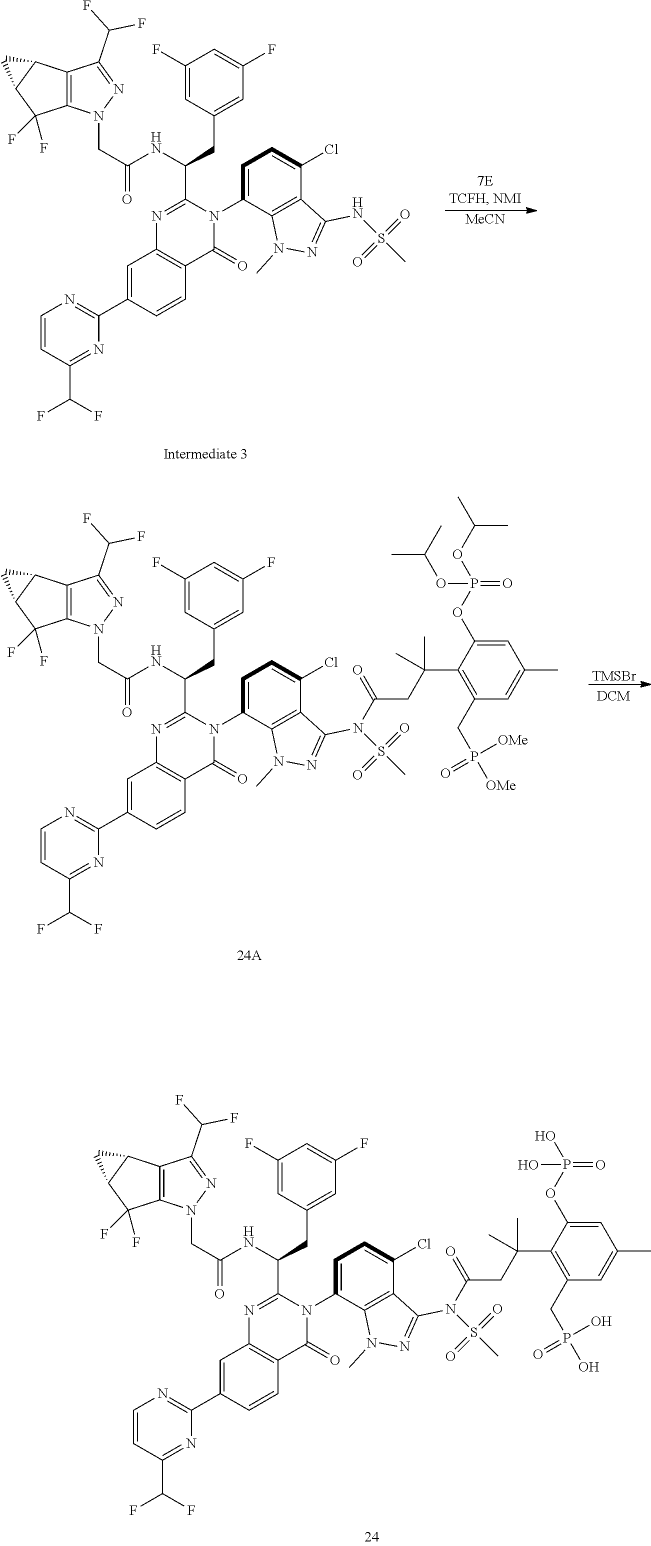

### Step 1. Synthesis of 2-(4-(N-(4-chloro-7-(2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-3-((dimethoxyphosphoryl)methyl)-5-methylphenyl diisopropyl phosphate (24A)

To a solution of 7E (129 μmol) in ACN (0.5 mL) was added NMI (225 μmol), TCFH (64.3 μmol) and Intermediate 3 (64.3 μmol). The mixture was stirred at 50° C. for 2 hours. The reaction mixture was concentrated and purified by reverse phase HPLC (ACN/water with 0.1% TFA). Fractions containing the product were pooled and concentrated to afford an atropisomeric mixture of title compound 24A. MS (m/z): 1431.4 [M+Na]⁺.

### Step 2. Synthesis of (2-(4-(N-(4-chloro-7-(2-((S)-1-(2-((3bS,4aR)-3-(difluoromethyl)-5, 5-difluoro-3b,4,4a,5-tetrahydro-1H-cyclopropa[3,4]cyclopenta[1,2-c]pyrazol-1-yl)acetamido)-2-(3,5-difluorophenyl)ethyl)-7-(4-(difluoromethyl)pyrimidin-2-yl)-4-oxoquinazolin-3(4H)-yl)-1-methyl-1H-indazol-3-yl)methylsulfonamido)-2-methyl-4-oxobutan-2-yl)-5-methyl-3-(phosphonooxy)benzyl)phosphonic acid (24)

To a solution of 24A (56.8 μmol) in DCM (1.0 mL) was added TMSBr (568 mol). The mixture was stirred at 45° C. for 2 hours under N₂. The reaction mixture was concentrated and purified by reverse phase HPLC (ACN/water with 0.1% TFA). Fractions containing the product were pooled and concentrated to afford an atropisomeric mixture of title compound 24. ¹H NMR (400 MHz, DMSO-d₆) δ 9.29 (d, J=5.1 Hz), 8.92-8.83 (m), 8.63 (d, J=8.3 Hz), 8.40 (dd, J=8.4, 3.2 Hz), 7.88 (d, J=5.1 Hz), 7.80 (d, J=7.9 Hz), 7.62 (d, J=7.9 Hz), 7.50 (dd, J=20.3, 7.9 Hz), 7.30 (s), 7.19-7.11 (m), 7.10-6.95 (m), 6.92 (d, J=8.1 Hz), 6.82-6.65 (m), 4.73-4.66 (m), 4.63-4.46 (m), 4.42-4.28 (m), 3.65 (d, J=16.5 Hz), 3.54-3.22 (m), 3.21-3.01 (m), 2.87-2.69 (m), 2.50-2.38 (m), 2.16 (d, J=9.2 Hz), 1.69-1.43 (m), 1.40-1.22 (m), 0.93-0.84 (m). ³¹P NMR (162 MHz, DMSO-d₆) δ 21.66, 21.59, −7.12, −7.82. MS (m/z): 1297.2 [M+H]⁺.

### Biological Assays

### 1. Solubility in Simulated Intestinal Fluids

For the solubility assay, approximately 1 to 10 mg of the compound being tested is added to 1.7 mL polypropylene centrifuge tubes. A sufficient volume of fasted state simulated intestinal fluid (FaSSIF) or fed state simulated intestinal fluid (FeSSIF) is then added to each tube to achieve a final concentration of approximately 1 to 20 mg/mL. FaSSIF and FeSSIF are prepared according to manufacturer instruction (catalog #FFF02, Biorelevant, London, UK). Samples are first vortexed for approximately 10 seconds to suspend solids in solution and immediately placed in a bench top vial mixer set to 25° C. and 1400 rpm. After predetermined incubation times, samples are removed from the vial mixer and centrifuged at 15,000 g. A sample of the supernatant is then diluted in a UPLC vial and stored at −20° C. until analysis. After sampling, tubes are vortexed for approximately 10 seconds to re-suspend any solids and returned to the vial mixer at 25° C. and 1400 rpm until the next predetermined timepoint. At completion of the study, samples are removed from the freezer, equilibrated to ambient temperature, and analyzed by reversed-phase UPLC to determine the concentration of compound in the supernatant at each timepoint.

### 2. Kinetic Solubility Analysis (CLND: Total Chemiluminescent Nitrogen Determination)

Kinetic Solubility from DMSO Stocks of each compound being tested: 100-fold dilutions of a 10 μM DMSO stock solution of each compound being tested are prepared in singleton by combining 3 μL of DMSO stock with 297 μL of the appropriate media (0.1N HCL (Alfa Aesar part number 35644-K2) and 1×PBS buffer (pH 7.4)) in a Millipore solubility filter plate with 0.45 μpolycarbonate filter membrane using Hamilton Starlet liquid handling. The final DMSO Concentration is 1.0% and maximum theoretical compound concentration is 100 M (assuming stock concentration of 10 mM). The filter plate is sealed. Following 24-hour incubation at ambient temperature (21.7-23.8° C.), the samples are vacuum filtered, and the filtrates are collected in a 96 well polypropylene plate for analysis. The collection plate is sealed for analysis.

Filtrates are injected into the nitrogen detector for quantification on Analiza's Automated Discovery Workstation. The equimolar nitrogen response of the detector is calibrated using standards which span the dynamic range of the instrument from 0.08 to 4500 μg/ml nitrogen. The filtrates are quantified with respect to this calibration curve. The calculated solubility values are corrected for background nitrogen present in the DMSO, and the media used to prepare the samples. The 1×PBS buffer (pH 7.4) is prepared by adding 50 mL of phosphate buffered saline solution 10×, PBS (Fisher Bioreagent part number BP399-500) to approximately 450 mL HPLC grade H₂O. The volume of the solution is then adjusted to 500 mL for a total dilution factor of 1:10 and a final PBS concentration of 1X. The pH of the final solution is measured and found to be 7.4.

The results are reported in μM in Table 1.

TABLE 1

| | pH 2 / pH 7 |
|---|---|
| Compound | Solubility (μM) |
| 1 | — |
| 2 | — |
| 3 | 1.4 / 95 |
| 4 | <1.0 / 81 |
| 5 | 1.1 / 76 |
| 6 | 3.9 / 77 |
| 7 | 1.3 / >100 |
| 8 | 2.7 / 72 |
| 9 | — |
| 10 | — |
| 11 | — |
| 12 | — |
| 13 | — |
| 14 | — |
| 15 | — |
| 16 | — |
| 17 | — |
| 18 | — |
| 19 | 2.1 / 65 |
| 20 | 7.5 / 98 |
| 21 | 2.7 / 69 |
| 22 | 2.0 / >100 |
| 23 | 5.8 / 58 |
| 24 | <1.0 / >100 |

### 3. Oral Bioavailability PK Samples

Oral dose (suspension and solution vehicle) of the compound being tested is administered via gavage in rat (Sprague Dawley) and dog (Beagle). Serial blood samples are collected via jugular vein into pre-chilled K₂EDTA with 2 mM dichlorvos (final concentration) for up to 168h. Whole blood is processed into plasma by centrifuge (3000 rpm for 10 minutes at 5C) within 30 minutes of collection.

Plasma samples are analyzed by direct protein precipitation with acetonitrile and further dilution with water before injecting onto Sciex API 5500 LC/MS/MS system for analysis.

AUC is calculated as Area under the plasma concentration vs. time curve from 0 h to infinity.

Bioavailability (% F) is calculated by comparing plasma concentration via oral dose vs. plasma concentration via IV dose (intravenous). % F=[(PO AUCinf·IV Dose)/(IV AUCinf·PO Dose)]·100.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter, and is not intended to limit the appended claims to the specific embodiments illustrated.

1. A compound of Formula I: 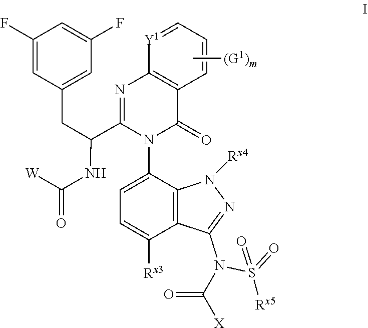
or a pharmaceutically acceptable salt thereof, wherein:
Y¹ is CH or N;
G¹ is C₁₋₆ alkyl, C₁₋₁₀ alkoxy, —O(phenyl substituted with 1-5 halogens), —N(R^{1a})₂, —SO₂R^{2a}, C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl,
wherein the C₁₋₆ alkyl and C₁₋₁₀ alkoxy are each optionally substituted with 1-10 R^{3a} groups;
wherein the C₃₋₇ monocyclic cycloalkyl, cyclopentenyl, cyclohexenyl, phenyl, naphthalenyl, 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl are each optionally substituted with 1-6 groups independently selected from —OH, —CN, halogen, —N(R^{1a})₂, —SO₂R^{2a}, R^{4a}, C₁₋₄ alkyl, C₁₋₄ alkoxy, and C₃₋₆ monocyclic cycloalkyl,
wherein the C₁₋₄ alkyl, C₁₋₄ alkoxy, and C₃₋₆ monocyclic cycloalkyl are each optionally substituted with 1-6 halogens;
each R^{1a} independently is H or C₁₋₆ alkyl optionally substituted with 1-6 groups independently selected from —OH, —CN, halogen, —SO₂(C₁₋₆ alkyl), and C₁₋₆ alkoxy;
each R^{2a} independently is C₁₋₆ alkyl optionally substituted with 1-6 halogens;
each R^{3a} independently is —OH, —CN, halogen, —N(R^{1a})₂, —SO₂R^{2a}, C₁₋₅ alkoxy, C₃₋₆ monocyclic cycloalkyl, phenyl, 5-6 membered monocyclic heteroaryl, or —O(C₃₋₆ monocyclic cycloalkyl substituted with 1-5 halogens),
wherein the C₁₋₅ alkoxy, C₃₋₆ monocyclic cycloalkyl, phenyl, and 5-6 membered monocyclic heteroaryl are each optionally substituted with 1-6 groups independently selected from halogen, C₁₋₃ alkyl, and C₁₋₃ alkoxy,
wherein the C₁₋₃ alkyl and C₁₋₃ alkoxy are each optionally substituted with 1-4 halogens,
each R^{4a} independently is C₁₋₆ alkyl optionally substituted with 1-6 groups independently selected from —OH, —CN, halogen, —SO₂(C₁₋₆ alkyl), and C₁₋₆ alkoxy;
m is 1, 2, 3, or 4;
R^{X3} is H, F, Cl, —CH₃ or —OCH₃;
R^{X4} is H or C₁₋₃ alkyl, wherein the C₁₋₃ alkyl is optionally substituted with 1 to 3 fluorines;
R^{X5} is C₁₋₆ alkyl or C₃₋₆ cycloalkyl;
W is selected from: 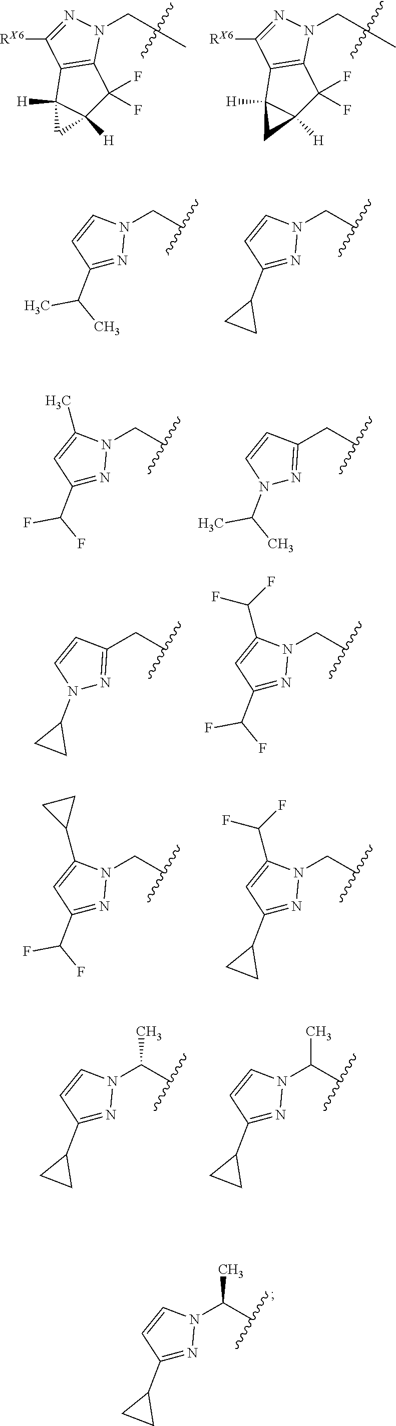
R^{X6} is methyl or C₃₋₅ monocyclic cycloalkyl, each of which is optionally substituted with 1 to 3 halogens;
X is —NR¹R², C₁₋₁₀ alkyl, or C₂₋₆ alkenyl,
wherein the C₁₋₁₀ alkyl and C₂₋₆ alkenyl are each independently substituted with 1-3 Y groups;
each Y independently is —B(OH)₂, —CN, halogen, R^{a}, R^{b}, R^{c}, phenyl, naphthalenyl, 5-6 membered monocyclic heteroaryl, or 8-10 membered fused bicyclic heteroaryl,
wherein the phenyl, naphthalenyl, 5-6 membered monocyclic heteroaryl, and 8-10 membered fused bicyclic heteroaryl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —CN, halogen, R^{a}, R^{b}, and R^{c};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
wherein the C₁₋₆ alkyl is optionally substituted with 1-3 groups independently selected from —CN, halogen, R^{a}, R^{b}, and R^{c};
each R³ independently is R^{a}, R^{b}, R^{c}, C₁₋₆ alkyl, or 5-6 membered monocyclic heteroaryl,
wherein the C₁₋₆ alkyl and 5-6 membered monocyclic heteroaryl are each independently optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, C₁₋₆ alkyl, or 5-6 membered monocyclic heteroaryl,
wherein the C₁₋₆ alkyl is optionally substituted with 1-3 groups independently selected from —CN, halogen, =NR^{5a}, R^{a}, R^{d}, R^{e}, phenyl, naphthalenyl, and 8-10 membered fused bicyclic heteroaryl,
wherein the 5-6 membered monocyclic heteroaryl is optionally substituted with 1-3 groups independently selected from —CN, halogen, R^{a}, R^{d}, and R^{e};
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{e} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from CN, halogen, R^{a}, R^{f}, and R^{g};
each R⁷ independently is H, R^{f}, or C₁₋₆ alkyl, wherein the C₁₋₆ alkyl is optionally substituted with 1-3 groups independently selected from —CN, halogen, R^{a}, R^{f}, and R^{g};
each R^{f} independently is —C(O)R⁸, —C(O)OR⁸, —C(O)NR⁸R⁸, —C(O)C(O)OR⁸, —S(O)₂R⁸, —S(O)₂NR⁸R⁸, or —S(O)₂OR⁸;
each R^{g} independently is —OR⁸, —OC(O)R⁸, —OC(O)C(O)OR⁸, —NR⁸R⁸, —NR⁸C(O)R⁸, —NR⁸C(O)NR⁸R⁸, —NR⁸C(O)OR⁸, —NR⁸C(O)C(O)OR⁸, or —NR'S(O)₂R⁸;
each R⁸ independently is H or C₁₋₆ alkyl, wherein the C₁₋₆ alkyl is optionally substituted with 1-3 groups independently selected from —OH, CN, halogen, —C(O)OH, and R^{a};
n is 1, 2, 3, 4, or 5; and
wherein each 5-8 membered monocyclic heterocyclyl, 5-6 membered monocyclic heteroaryl, 8-10 membered fused bicyclic heteroaryl, 8-10 membered fused bicyclic heterocyclyl, 8-10 membered bridged bicyclic heterocyclyl, and 7-10 membered spirocyclic heterocyclyl independently have 1-4 ring heteroatoms independently selected from N, O, and S.

2-3. (canceled)

4. The compound of claim 1, or a pharmaceutically acceptable salt thereof, wherein
G¹ is

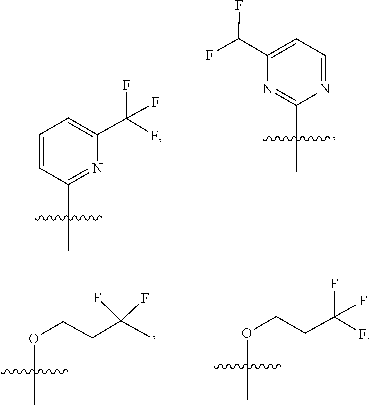

5. The compound of claim 1, or a pharmaceutically acceptable salt thereof, wherein
X is —NR¹R², C₁₋₁₀ alkyl, or C₂₋₆ alkenyl,
wherein the C₁₋₁₀ alkyl and C₂₋₆ alkenyl are each independently substituted with 1-3 Y groups;
each Y independently is —CN, halogen, R^{a}, R^{b}, R^{c}, phenyl, or naphthalenyl,
wherein the phenyl and naphthalenyl are each independently substituted with 1-5 R³ groups, or
two Y groups on the same carbon, together with the carbon to which they are attached, form a C₃₋₅ monocyclic cycloalkyl;
R¹ is H or C₁₋₆ alkyl, wherein the C₁₋₆ alkyl is optionally substituted with 1-3 groups independently selected from —OH, —CN, halogen, —C(O)OH, and R^{a};
R² is phenyl or 5-6 membered monocyclic heteroaryl, wherein the phenyl and 5-6 membered monocyclic heteroaryl are each independently optionally substituted with 1-3 groups independently selected from —CN, halogen, R^{a}, R^{b}, R^{c}, and C₁₋₆ alkyl,
wherein the C₁₋₆ alkyl is optionally substituted with 1-3 groups independently selected from —CN, halogen, R^{a}, R^{b}, and R^{c};
each R³ independently is R^{a}, R^{b}, R^{c}, or C₁₋₆ alkyl, wherein the C₁₋₆ alkyl is optionally substituted with 1-3 groups independently selected from —CN, halogen, R^{a}, R^{b}, and R^{c};
each R^{a} independently is —P(O)(OH)₂ or —OP(O)(OH)₂;
each R^{b} independently is —C(O)R⁴, —C(O)OR⁴, —C(O)NR⁵R⁵, —C(O)C(O)OR⁴, —S(O)₂R⁴, —S(O)₂NR⁵R⁵, or —S(O)₂OR⁴;
each R^{c} independently is —OR⁴, —OC(O)R⁴, —OC(O)C(O)OR⁴, —(O(C₁₋₄ alkyl))ₙOR⁴, —NR⁵R⁵, —N⁺R⁵R⁵R^{5a}, —NR⁵C(O)R⁴, —NR⁵C(O)NR⁵R⁵, —NR⁵C(O)OR⁴, —NR⁵C(O)C(O)OR⁴, or —NR⁵S(O)₂R⁴;
each R⁴ independently is H or C₁₋₆ alkyl, wherein the C₁₋₆ alkyl is optionally substituted with 1-3 groups independently selected from —CN, halogen, R^{a}, R^{d}, and R^{e};
each R⁵ independently is H, R^{d}, or C₁₋₆ alkyl, wherein the C₁₋₆ alkyl is optionally substituted with 1-3 groups independently selected from —CN, halogen, =NR^{5a}, R^{a}, R^{d}, R^{e}, phenyl, and naphthalenyl;
each R^{5a} independently is H or C₁₋₃ alkyl;
each R^{d} independently is —C(O)R⁶, —C(O)OR⁶, —C(O)NR⁷R⁷, —C(O)C(O)OR⁶, —S(O)₂R⁶, —S(O)₂NR⁷R⁷, or —S(O)₂OR⁶;
each R^{c} independently is —OR⁶, —OC(O)R⁶, —OC(O)C(O)OR⁶, —NR⁷R⁷, —NR⁷C(O)R⁷, —NR⁷C(O)NR⁷R⁷, —NR⁷C(O)OR⁶, —NR⁷C(O)C(O)OR⁶, or —NR⁷S(O)₂R⁶;
each R⁶ independently is H or C₁₋₆ alkyl, wherein the C₁₋₆ alkyl is optionally substituted with 1-3 groups independently selected from —OH, CN, halogen, —C(O)OH, and R^{a};
each R⁷ independently is H or C₁₋₆ alkyl, wherein the C₁₋₆ alkyl is optionally substituted with 1-3 groups independently selected from —OH, —CN, halogen, —C(O)OH, and R^{a};
n is 1, 2, 3, 4, or 5; and
wherein each 5-6 membered monocyclic heteroaryl and 8-10 membered fused bicyclic heteroaryl independently have 1-4 ring heteroatoms independently selected from N, O, and S.

6-17. (canceled)

18. The compound of claim 1, or a pharmaceutically acceptable salt thereof, wherein X is C₁₋₁₀ alkyl, wherein the C₁₋₁₀ alkyl is substituted with 1-3 Y groups.

19-20. (canceled)

21. The compound of claim 1, or a pharmaceutically acceptable salt thereof, wherein X substituted with Y is —CH₂Y, —CH₂CH₂Y, —CH₂CH₂CH₂Y, —CH₂CH₂CH₂CH₂Y,

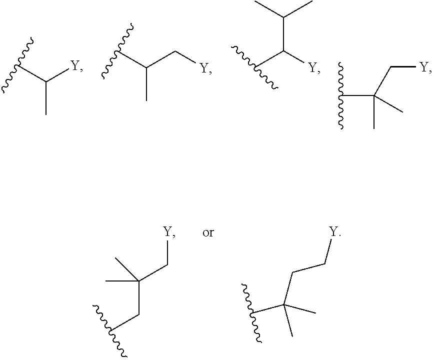

22-24. (canceled)

25. The compound of claim 1, or a pharmaceutically acceptable salt thereof, wherein X is substituted with three Y groups, wherein two of the three Y groups are on the same carbon and wherein the two Y groups on the same carbon, together with the carbon to which they are attached, form a cyclopropyl.

26-27. (canceled)

28. The compound of claim 1, or a pharmaceutically acceptable salt thereof, wherein each Y independently is —B(OH)₂, —C(O)OR⁴, —C(O)NR⁵R⁵, —OC(O)R⁴, —(O(C₁₋₄ alkyl))ₙOR⁴, —NR⁵R⁵, —N⁺R⁵R⁵R^{5a}, —S(O)₂R⁴, —S(O)₂NR⁵R⁵, —S(O)₂OR⁴, —NR⁵C(O)R⁴, —NR⁵C(O)NR⁵R⁵, —NR⁵S(O)₂R⁴, R^{a}, 5-6 membered monocyclic heteroaryl, or 8-10 membered fused bicyclic heteroaryl,
wherein the 5-6 membered monocyclic heteroaryl and 8-10 membered fused bicyclic heteroaryl are each independently substituted with 1-3 groups independently selected from —OH, —CN, halogen, —C(O)OR⁴, —C(O)NR⁵R⁵, and R^{a}.

29-30. (canceled)

31. The compound of claim 1, or a pharmaceutically acceptable salt thereof, wherein one Y is phenyl, wherein the phenyl is substituted with 1-5 R³ groups.

32-33. (canceled)

34. The compound of claim 1, or a pharmaceutically acceptable salt thereof, wherein each R³ independently is —C(O)OR⁴, —C(O)NR⁵R⁵, —S(O)₂R⁴, —S(O)₂NR⁵R⁵, —S(O)₂OR⁴, —NR⁵C(O)R⁴, —NR⁵C(O)NR⁵R⁵, —NR⁵S(O)₂R⁴, R^{a}, or C₁₋₆ alkyl,
wherein the C₁₋₆ alkyl is optionally substituted with 1-3 groups independently selected from —OH, —CN, halogen, —C(O)OR⁴, —C(O)NR⁵R⁵, and R^{a}.

35-42. (canceled)

43. The compound of claim 1, or a pharmaceutically acceptable salt thereof, wherein each R⁴ independently is H or C₁₋₆ alkyl, wherein the C₁₋₆ alkyl is optionally substituted with 1-2 groups independently selected from —C(O)OH, —NR⁷R⁷, and R^{a}.

44. (canceled)

45. The compound of claim 1, or a pharmaceutically acceptable salt thereof, wherein each R⁵ independently is H, —C(O)OR⁶, —C(O)C(O)OR⁶, or C₁₋₄ alkyl,
wherein the C₁₋₄ alkyl is optionally substituted with 1-2 groups independently selected from —C(O)OH, —C(O)NH₂, NR^{5a}, —NR⁷R⁷, R^{a}, and phenyl.

46-51. (canceled)

52. The compound of claim 1, or a pharmaceutically acceptable salt thereof, wherein each R⁶ independently is H or C₁₋₃ alkyl, wherein the C₁₋₃ alkyl is optionally substituted with 1-2 R^{a} groups.

53-54. (canceled)

55. The compound of claim 1, or a pharmaceutically acceptable salt thereof, wherein Y¹ is N.

56. The compound of claim 1, or a pharmaceutically acceptable salt thereof, wherein G¹ is

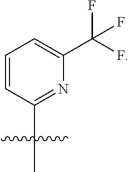 [SMILES: *c1cccc(C(F)(F)F)n1]

57. The compound of claim 1, or a pharmaceutically acceptable salt thereof, wherein G¹ is

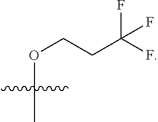 [SMILES: *OCCC(F)(F)F]

58. The compound of claim 1, or a pharmaceutically acceptable salt thereof, wherein G¹ is

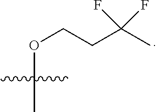 [SMILES: *OCCC(C)(F)F]

59. The compound of claim 1, or a pharmaceutically acceptable salt thereof, wherein G¹ is

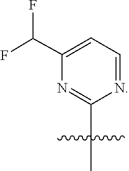 [SMILES: *c1nccc(C(F)F)n1]

60. (canceled)

61. The compound of claim 1, wherein the compound of Formula I is a compound of Formula IVa: 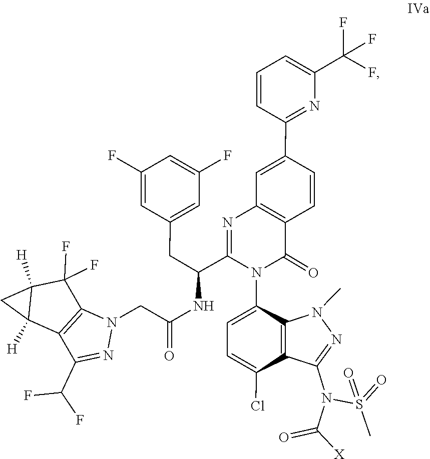
or a pharmaceutically acceptable salt thereof.

62. The compound of claim 1, wherein the compound of Formula I is a compound of Formula Va: 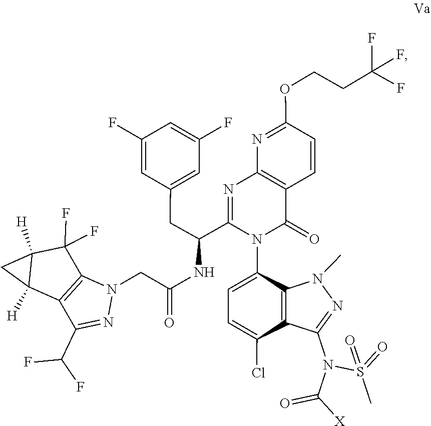
or a pharmaceutically acceptable salt thereof.

63-64. (canceled)

65. A compound selected from the group consisting of: 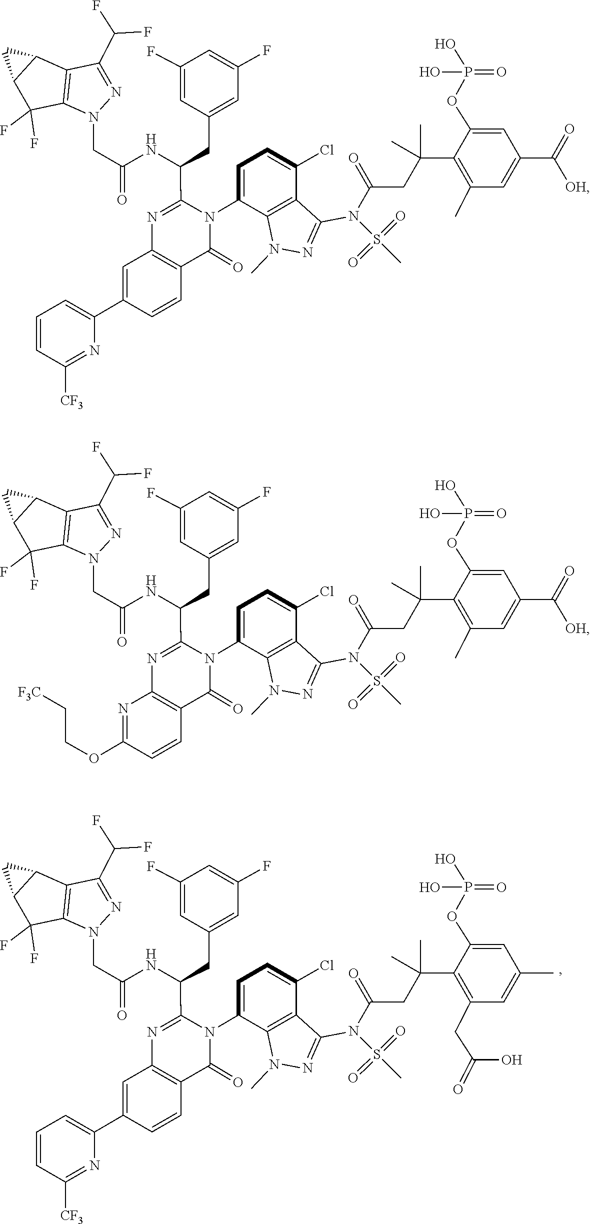 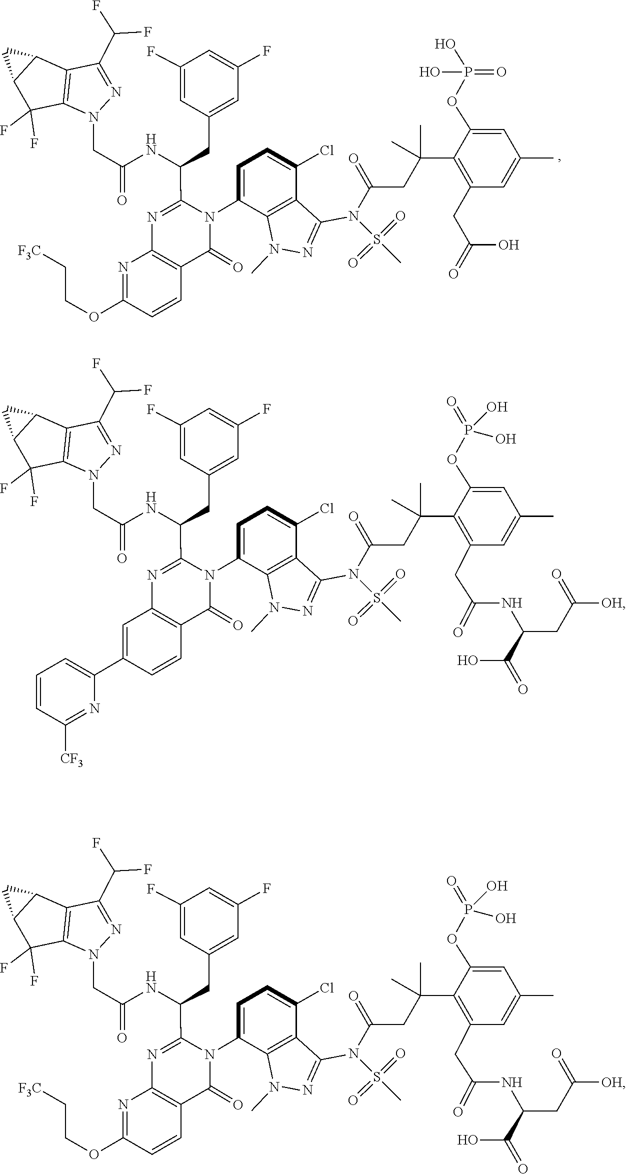 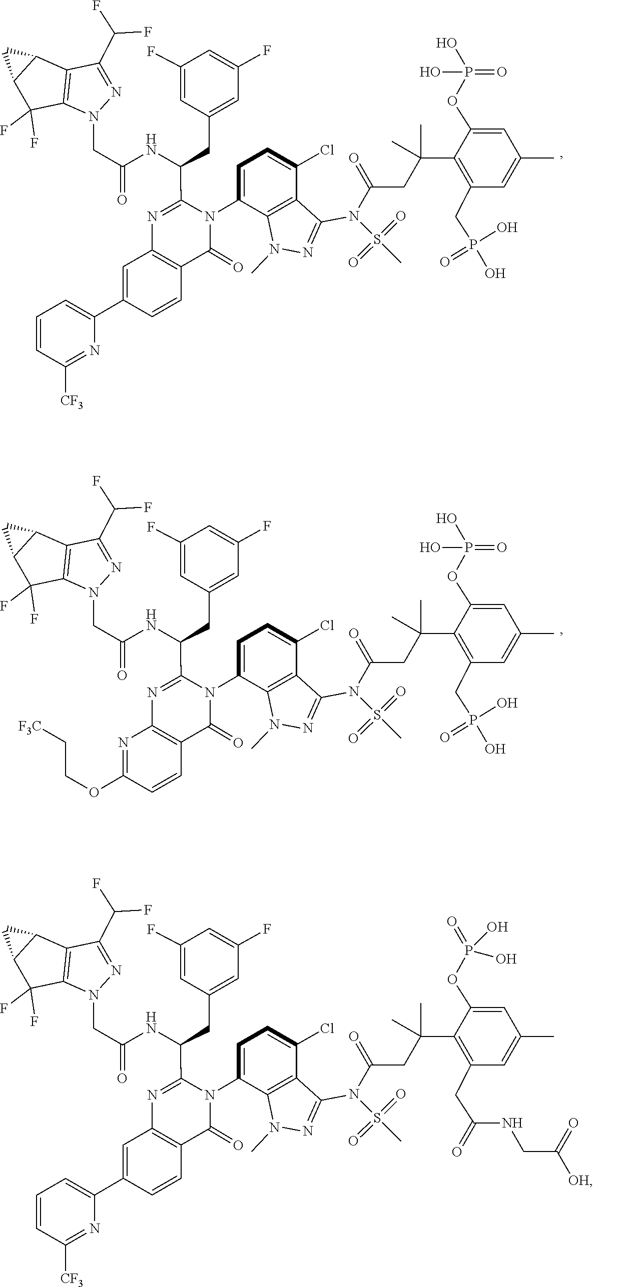 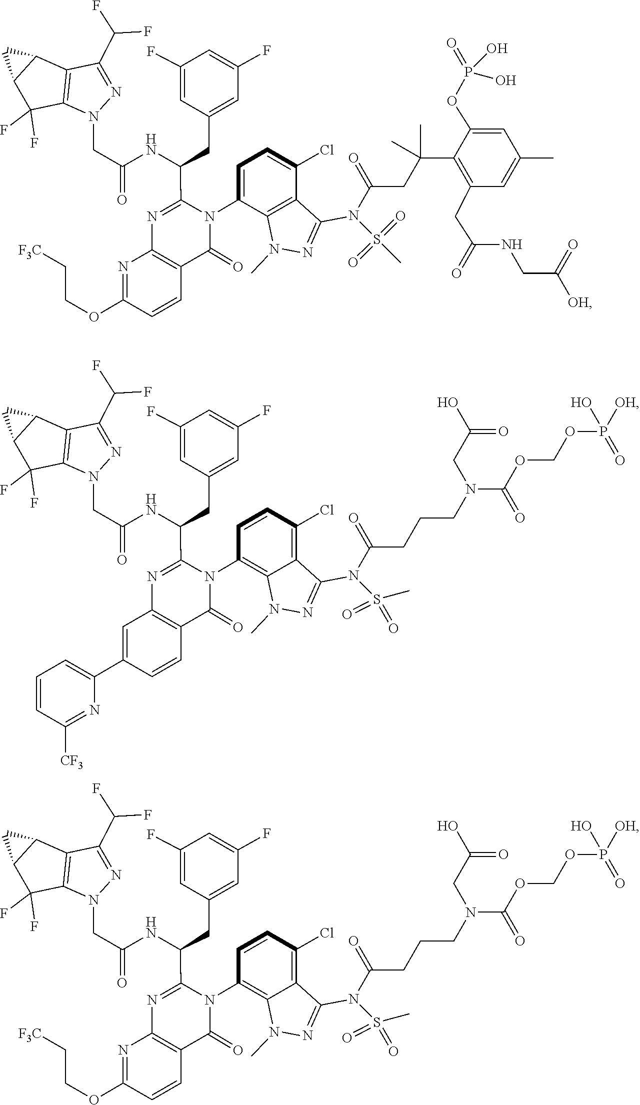 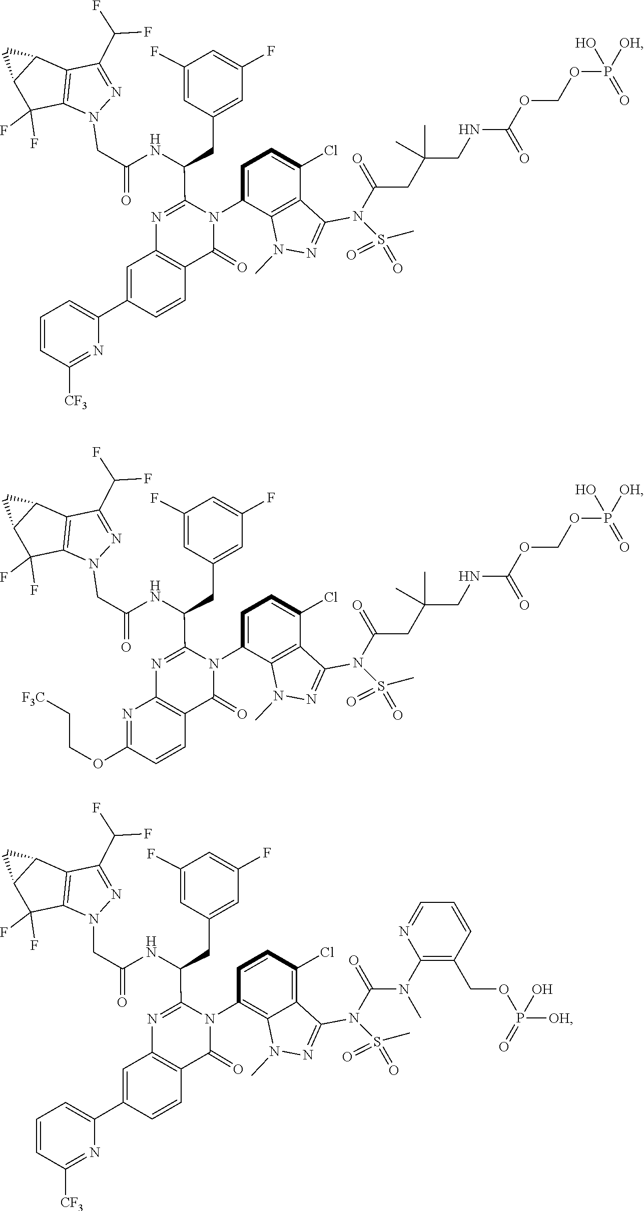 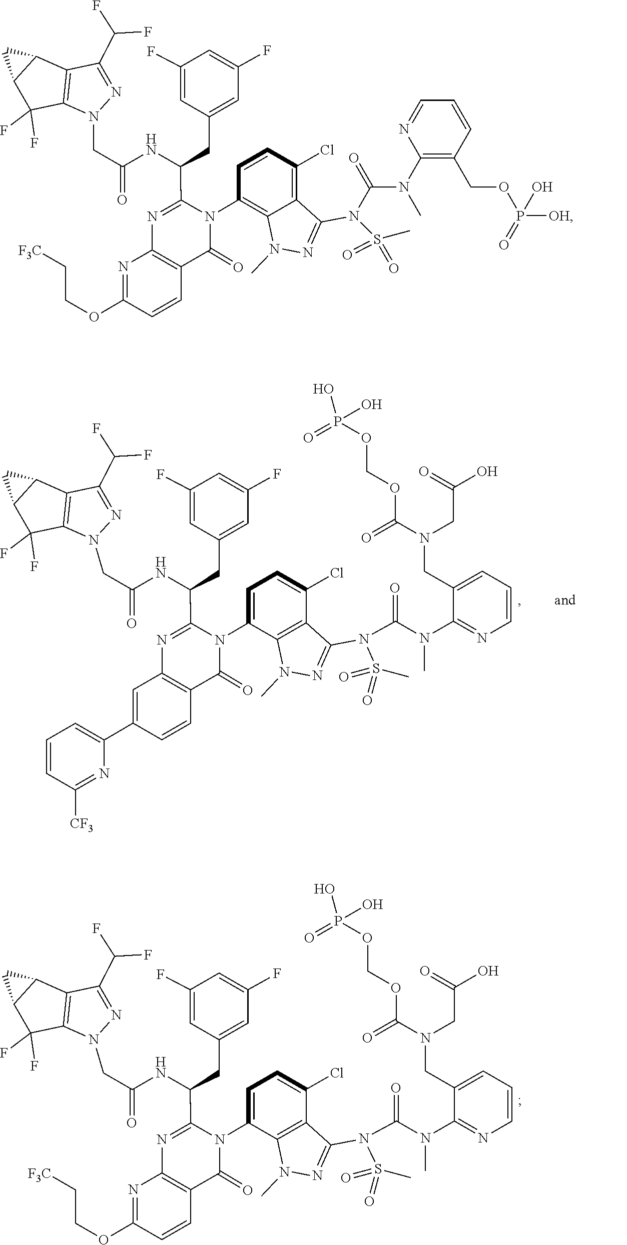
or a pharmaceutically acceptable salt thereof.

66. The compound of claim 1, which is selected from the group consisting of: 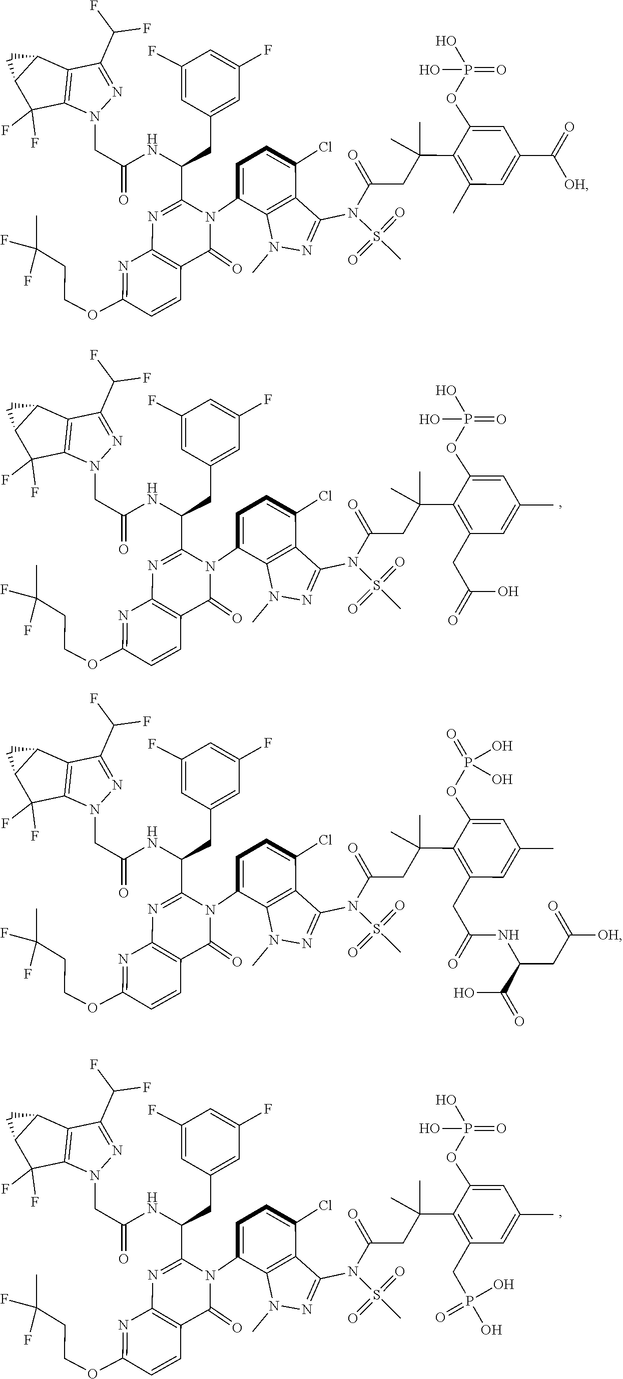 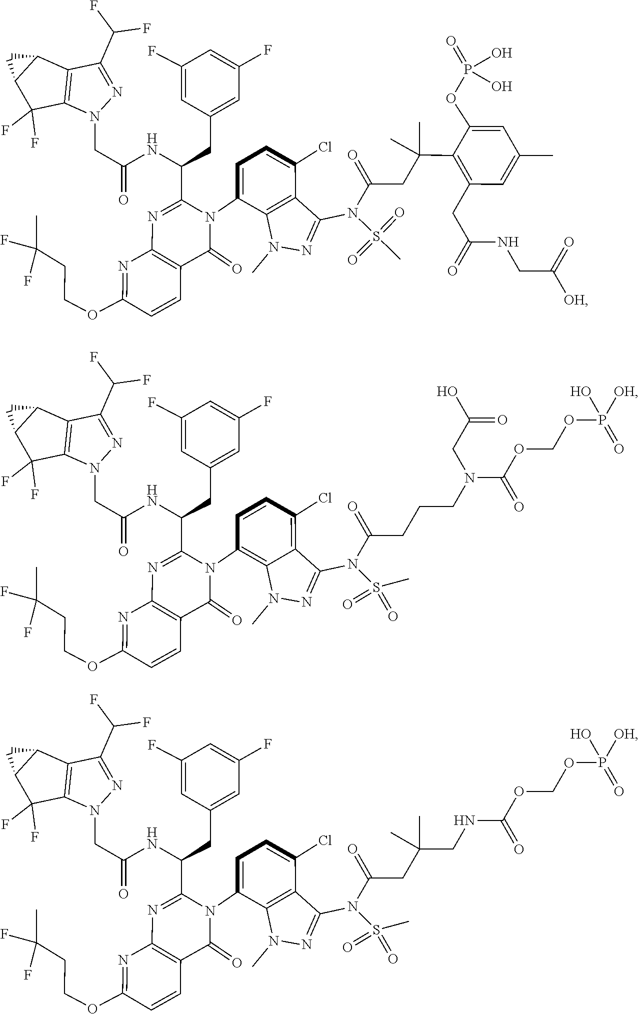 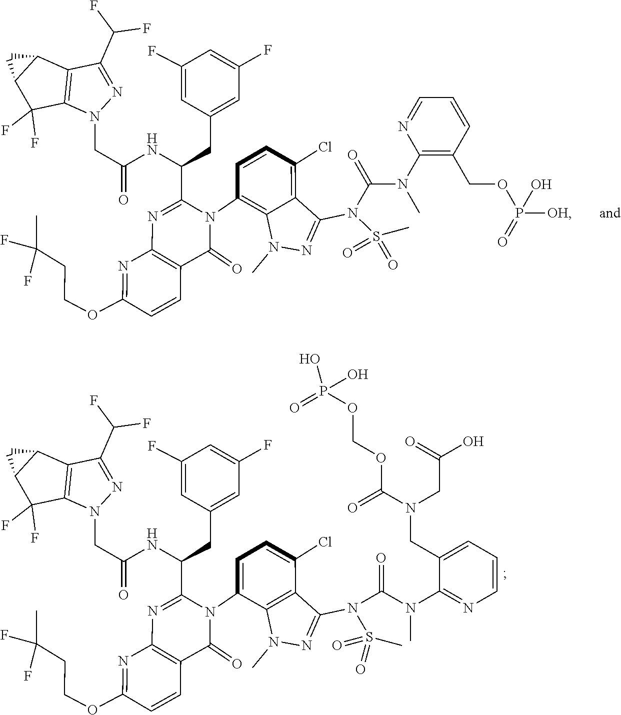
or a pharmaceutically acceptable salt thereof.

67. The compound of claim 1, which is selected from the group consisting of: 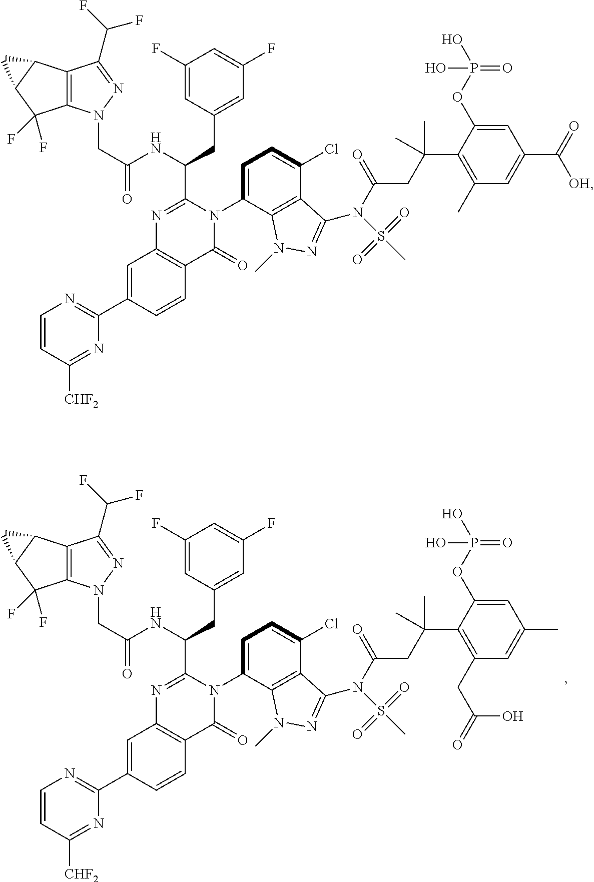 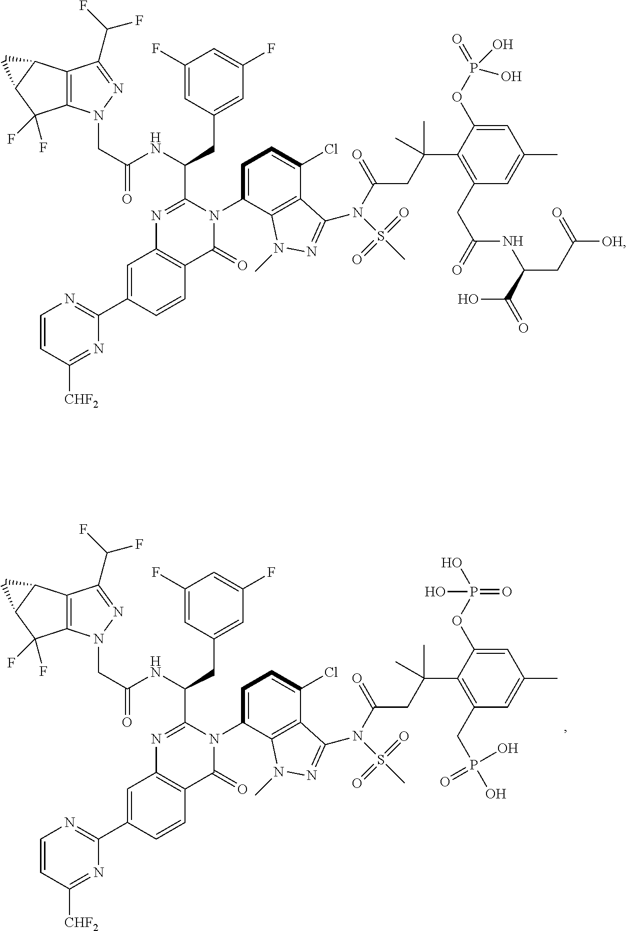 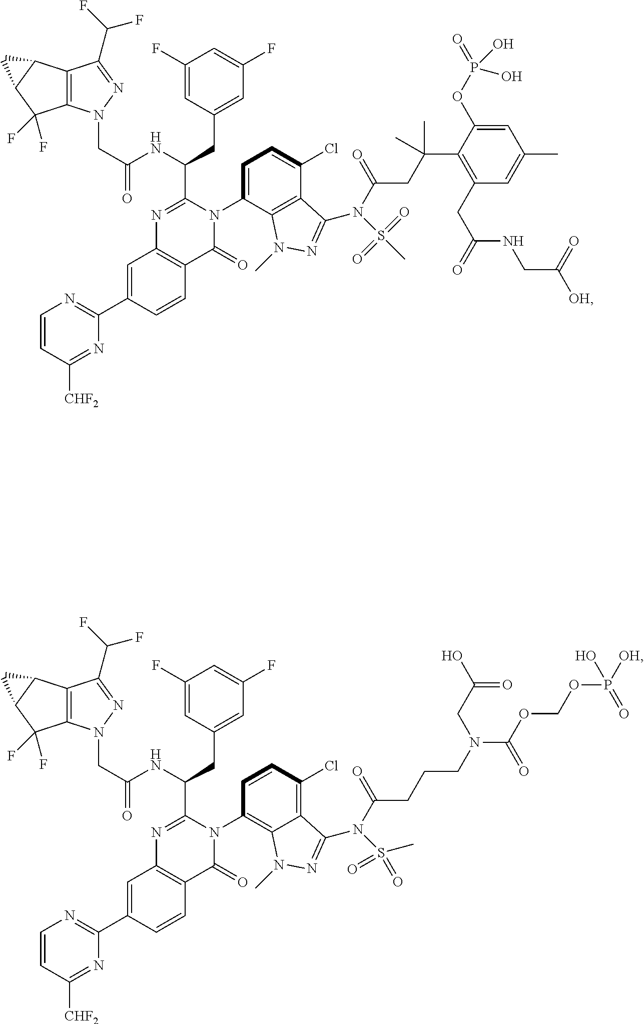 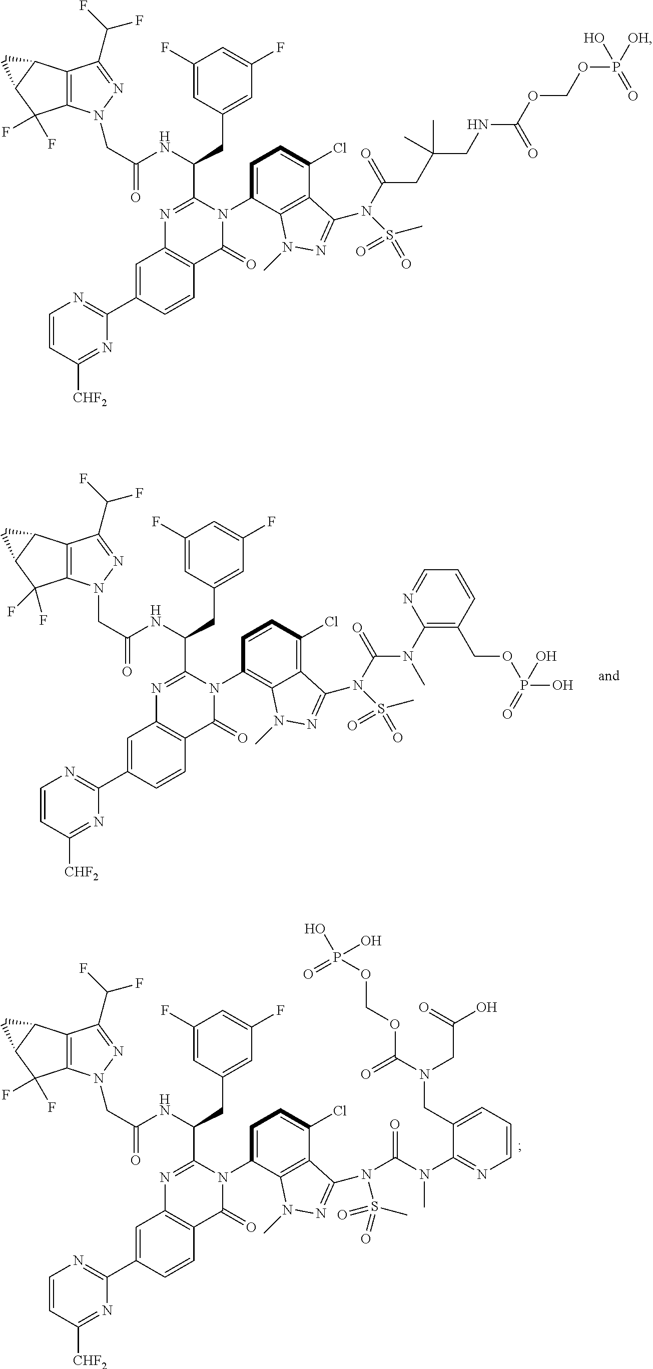
or a pharmaceutically acceptable salt thereof.

68. A pharmaceutical composition comprising a therapeutically effective amount of a compound of claim 1, or a pharmaceutically acceptable salt thereof, and a pharmaceutically acceptable excipient.

69. The pharmaceutical composition of claim 68, further comprising one, two, three, or four additional therapeutic agents.

70-72. (canceled)

73. A method of treating or preventing a human immunodeficiency virus (HIV) infection in a patient in need thereof comprising administering to the patient a therapeutically effective amount of the compound of claim 1, or a pharmaceutically acceptable salt thereof.

74. A method of treating a human immunodeficiency virus (HIV) infection in a heavily treatment-experienced patient, the method comprising administering to the patient a therapeutically effective amount of the compound of claim 1, or a pharmaceutically acceptable salt thereof.

75. The method of claim 73, wherein the method further comprises administering a therapeutically effective amount of one, two, three, or four additional therapeutic agents, or a pharmaceutically acceptable salt thereof.

76-87. (canceled)
